SEQUENCE LISTING (212 sequences)

SEQ ID NO: 1 - protein, 143 aa, Artificial Sequence
  Description of Artificial Sequence Synthetic polypeptide
HHHHHHNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 2 - protein, 121 aa, Artificial Sequence
  Description of Artificial Sequence Synthetic polypeptide
EVQLLESGGGLVQPGGSLRLSCAASGFTFSIYAMTWVRQAPGKGLEWVSIISGSGDHTFYADSVKGRFTISRDNSRNTLYLQMDSLRAEDTAVYYCAKEGKWVQLSHFANWGQGTLVTVSS

SEQ ID NO: 3 - protein, 108 aa, Artificial Sequence
  Description of Artificial Sequence Synthetic polypeptide
SYVLTQPPSVSVAPGQTARITCGGNNIGSKSVHWYQQKAGQAPVLVVYDDSDRPSGIPERFSGSNSGNTATLTISRVEAGDEADYYCQVWDGRSDHVVFGGGTKLTVL

SEQ ID NO: 4 - protein, 10 aa, Artificial Sequence
  Description of Artificial Sequence Synthetic peptide
GFTFSIYAMT

SEQ ID NO: 5 - protein, 10 aa, Artificial Sequence
  Description of Artificial Sequence Synthetic peptide
IISGSGDHTF

SEQ ID NO: 6 - protein, 12 aa, Artificial Sequence
  Description of Artificial Sequence Synthetic peptide
EGKWVQLSHFAN

SEQ ID NO: 7 - protein, 11 aa, Artificial Sequence
  Description of Artificial Sequence Synthetic peptide
GGNNIGSKSVH

SEQ ID NO: 8 - protein, 7 aa, Artificial Sequence
  Description of Artificial Sequence Synthetic peptide
DDSDRPS

SEQ ID NO: 9 - protein, 11 aa, Artificial Sequence
  Description of Artificial Sequence Synthetic peptide
QVWDGRSDHVV

SEQ ID NO: 10 - protein, 5 aa, Artificial Sequence
  Description of Artificial Sequence Synthetic peptide
IYAMT

SEQ ID NO: 11 - protein, 17 aa, Artificial Sequence
  Description of Artificial Sequence Synthetic peptide
IISGSGDHTFYADSVKG

SEQ ID NO: 12 - protein, 12 aa, Artificial Sequence
  Description of Artificial Sequence Synthetic peptide
EGKWVQLSHFAN

SEQ ID NO: 13 - protein, 11 aa, Artificial Sequence
  Description of Artificial Sequence Synthetic peptide
GGNNIGSKSVH

SEQ ID NO: 14 - protein, 7 aa, Artificial Sequence
  Description of Artificial Sequence Synthetic peptide
DDSDRPS

SEQ ID NO: 15 - protein, 11 aa, Artificial Sequence
  Description of Artificial Sequence Synthetic peptide
QVWDGRSDHVV

SEQ ID NO: 16 - protein, 7 aa, Artificial Sequence
  Description of Artificial Sequence Synthetic peptide
GFTFSIY

SEQ ID NO: 17 - protein, 6 aa, Artificial Sequence
  Description of Artificial Sequence Synthetic peptide
SGSGDH

SEQ ID NO: 18 - protein, 11 aa, Artificial Sequence
  Description of Artificial Sequence Synthetic peptide
EGKWVQLSHFA

SEQ ID NO: 19 - protein, 7 aa, Artificial Sequence
  Description of Artificial Sequence Synthetic peptide
NNIGSKS

SEQ ID NO: 20 - protein, 3 aa, Artificial Sequence
  Description of Artificial Sequence Synthetic peptide
DDS

SEQ ID NO: 21 - protein, 8 aa, Artificial Sequence
  Description of Artificial Sequence Synthetic peptide
WDGRSDHV

SEQ ID NO: 22 - protein, 6 aa, Artificial Sequence
  Description of Artificial Sequence Synthetic peptide
SIYAMT

SEQ ID NO: 23 - protein, 13 aa, Artificial Sequence
  Description of Artificial Sequence Synthetic peptide
WVSIISGSGDHTF

SEQ ID NO: 24 - protein, 13 aa, Artificial Sequence
  Description of Artificial Sequence Synthetic peptide
AKEGKWVQLSHFA

SEQ ID NO: 25 - protein, 7 aa, Artificial Sequence
  Description of Artificial Sequence Synthetic peptide
SKSVHWY

SEQ ID NO: 26 - protein, 10 aa, Artificial Sequence
  Description of Artificial Sequence Synthetic peptide
LVVYDDSDRP

SEQ ID NO: 27 - protein, 10 aa, Artificial Sequence
  Description of Artificial Sequence Synthetic peptide
QVWDGRSDHV

SEQ ID NO: 28 - protein, 8 aa, Artificial Sequence
  Description of Artificial Sequence Synthetic peptide
GFTFSIYA

SEQ ID NO: 29 - protein, 8 aa, Artificial Sequence
  Description of Artificial Sequence Synthetic peptide
ISGSGDHT

SEQ ID NO: 30 - protein, 14 aa, Artificial Sequence
  Description of Artificial Sequence Synthetic peptide
AKEGKWVQLSHFAN

SEQ ID NO: 31 - protein, 6 aa, Artificial Sequence
  Description of Artificial Sequence Synthetic peptide
NIGSKS

SEQ ID NO: 32 - protein, 3 aa, Artificial Sequence
  Description of Artificial Sequence Synthetic peptide
DDS

SEQ ID NO: 33 - protein, 11 aa, Artificial Sequence
  Description of Artificial Sequence Synthetic peptide
QVWDGRSDHVV

SEQ ID NO: 34 - protein, 123 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSYTRYSPSFQGQVTISADKSISTAYLQWSSLKASDTAMYYCARGGVSASGNYRALDYWGQGTLVTVSS

SEQ ID NO: 35 - protein, 113 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIK

SEQ ID NO: 36 - protein, 10 aa, Artificial Sequence
  Description of Artificial Sequence Synthetic peptide
GYSFTSYWIG

SEQ ID NO: 37 - protein, 10 aa, Artificial Sequence
  Description of Artificial Sequence Synthetic peptide
IIYPGDSYTR

SEQ ID NO: 38 - protein, 14 aa, Artificial Sequence
  Description of Artificial Sequence Synthetic peptide
GGVSASGNYRALDY

SEQ ID NO: 39 - protein, 17 aa, Artificial Sequence
  Description of Artificial Sequence Synthetic peptide
KSSQSVLYSSNNKNYLA

SEQ ID NO: 40 - protein, 7 aa, Artificial Sequence
  Description of Artificial Sequence Synthetic peptide
WASTRES

SEQ ID NO: 41 - protein, 9 aa, Artificial Sequence
  Description of Artificial Sequence Synthetic peptide
QQYYSTPLT

SEQ ID NO: 42 - protein, 5 aa, Artificial Sequence
  Description of Artificial Sequence Synthetic peptide
SYWIG

SEQ ID NO: 43 - protein, 17 aa, Artificial Sequence
  Description of Artificial Sequence Synthetic peptide
IIYPGDSYTRYSPSFQG

SEQ ID NO: 44 - protein, 14 aa, Artificial Sequence
  Description of Artificial Sequence Synthetic peptide
GGVSASGNYRALDY

SEQ ID NO: 45 - protein, 17 aa, Artificial Sequence
  Description of Artificial Sequence Synthetic peptide
KSSQSVLYSSNNKNYLA

SEQ ID NO: 46 - protein, 7 aa, Artificial Sequence
  Description of Artificial Sequence Synthetic peptide
WASTRES

SEQ ID NO: 47 - protein, 9 aa, Artificial Sequence
  Description of Artificial Sequence Synthetic peptide
QQYYSTPLT

SEQ ID NO: 48 - protein, 7 aa, Artificial Sequence
  Description of Artificial Sequence Synthetic peptide
GYSFTSY

SEQ ID NO: 49 - protein, 6 aa, Artificial Sequence
  Description of Artificial Sequence Synthetic peptide
YPGDSY

SEQ ID NO: 50 - protein, 13 aa, Artificial Sequence
  Description of Artificial Sequence Synthetic peptide
GGVSASGNYRALD

SEQ ID NO: 51 - protein, 13 aa, Artificial Sequence
  Description of Artificial Sequence Synthetic peptide
SQSVLYSSNNKNY

SEQ ID NO: 52 - protein, 3 aa, Artificial Sequence
  Description of Artificial Sequence Synthetic peptide
WAS

SEQ ID NO: 53 - protein, 6 aa, Artificial Sequence
  Description of Artificial Sequence Synthetic peptide
YYSTPL

SEQ ID NO: 54 - protein, 6 aa, Artificial Sequence
  Description of Artificial Sequence Synthetic peptide
TSYWIG

SEQ ID NO: 55 - protein, 13 aa, Artificial Sequence
  Description of Artificial Sequence Synthetic peptide
WMGIIYPGDSYTR

SEQ ID NO: 56 - protein, 15 aa, Artificial Sequence
  Description of Artificial Sequence Synthetic peptide
ARGGVSASGNYRALD

SEQ ID NO: 57 - protein, 13 aa, Artificial Sequence
  Description of Artificial Sequence Synthetic peptide
LYSSNNKNYLAWY

SEQ ID NO: 58 - protein, 10 aa, Artificial Sequence
  Description of Artificial Sequence Synthetic peptide
LLIYWASTRE

SEQ ID NO: 59 - protein, 8 aa, Artificial Sequence
  Description of Artificial Sequence Synthetic peptide
QQYYSTPL

SEQ ID NO: 60 - protein, 8 aa, Artificial Sequence
  Description of Artificial Sequence Synthetic peptide
GYSFTSYW

SEQ ID NO: 61 - protein, 8 aa, Artificial Sequence
  Description of Artificial Sequence Synthetic peptide
IYPGDSYT

SEQ ID NO: 62 - protein, 16 aa, Artificial Sequence
  Description of Artificial Sequence Synthetic peptide
ARGGVSASGNYRALDY

SEQ ID NO: 63 - protein, 12 aa, Artificial Sequence
  Description of Artificial Sequence Synthetic peptide
QSVLYSSNNKNY

SEQ ID NO: 64 - protein, 3 aa, Artificial Sequence
  Description of Artificial Sequence Synthetic peptide
WAS

SEQ ID NO: 65 - protein, 9 aa, Artificial Sequence
  Description of Artificial Sequence Synthetic peptide
QQYYSTPLT

SEQ ID NO: 66 - protein, 467 aa, Artificial Sequence
  Description of Artificial Sequence Synthetic polypeptide
QQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPDTWGTYLLTTETGLQKDHALWDHTAQNIEGRMDPSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAEWESNGQPENNYKTTPPVLDSDGSFFLYSKLTVDKSRWQQGNVFSCSVMHEALHNHYTQKLSLSPGK

SEQ ID NO: 67 - protein, 121 aa, Artificial Sequence
  Description of Artificial Sequence Synthetic polypeptide
QLQLQESGPGPVRPSETLSLTCTVSGDSIRSSSYYWGWIRQPPGKGLDWIGSIYYTGSTYYNPSLMSRVTISVDTSKNQFSLKLSSVTAADTAVYYCASPGYSSGWSIDYWGQGTLVTVSS

SEQ ID NO: 68 - protein, 108 aa, Artificial Sequence
  Description of Artificial Sequence Synthetic polypeptide
DIQLTQSPSFLSASVGDRVTITCRASQGISSYLAWYQQKPGKAPKLLIYVASTLQSGVPSRFSGSGSGTEFTLTISSLQPEDFATYYCQQLNSYPRMTFGGGTKVEIK

SEQ ID NO: 69 - protein, 12 aa, Artificial Sequence
  Description of Artificial Sequence Synthetic peptide
GDSIRSSSYYWG

SEQ ID NO: 70 - protein, 9 aa, Artificial Sequence
  Description of Artificial Sequence Synthetic peptide
SIYYTGSTY

SEQ ID NO: 71 - protein, 11 aa, Artificial Sequence
  Description of Artificial Sequence Synthetic peptide
PGYSSGWSIDY

SEQ ID NO: 72 - protein, 11 aa, Artificial Sequence
  Description of Artificial Sequence Synthetic peptide
RASQGISSYLA

SEQ ID NO: 73 - protein, 7 aa, Artificial Sequence
  Description of Artificial Sequence Synthetic peptide
VASTLQS

SEQ ID NO: 74 - protein, 10 aa, Artificial Sequence
  Description of Artificial Sequence Synthetic peptide
QQLNSYPRMT

SEQ ID NO: 75 - protein, 7 aa, Artificial Sequence
  Description of Artificial Sequence Synthetic peptide
SSSYYWG

SEQ ID NO: 76 - protein, 16 aa, Artificial Sequence
  Description of Artificial Sequence Synthetic peptide
SIYYTGSTYYNPSLMS

SEQ ID NO: 77 - protein, 11 aa, Artificial Sequence
  Description of Artificial Sequence Synthetic peptide
PGYSSGWSIDY

SEQ ID NO: 78 - protein, 11 aa, Artificial Sequence
  Description of Artificial Sequence Synthetic peptide
RASQGISSYLA

SEQ ID NO: 79 - protein, 7 aa, Artificial Sequence
  Description of Artificial Sequence Synthetic peptide
VASTLQS

SEQ ID NO: 80 - protein, 10 aa, Artificial Sequence
  Description of Artificial Sequence Synthetic peptide
QQLNSYPRMT

SEQ ID NO: 81 - protein, 9 aa, Artificial Sequence
  Description of Artificial Sequence Synthetic peptide
GDSIRSSSY

SEQ ID NO: 82 - protein, 5 aa, Artificial Sequence
  Description of Artificial Sequence Synthetic peptide
YYTGS

SEQ ID NO: 83 - protein, 10 aa, Artificial Sequence
  Description of Artificial Sequence Synthetic peptide
PGYSSGWSID

SEQ ID NO: 84 - protein, 7 aa, Artificial Sequence
  Description of Artificial Sequence Synthetic peptide
SQGISSY

SEQ ID NO: 85 - protein, 3 aa, Artificial Sequence
  Description of Artificial Sequence Synthetic peptide
VAS

SEQ ID NO: 86 - protein, 7 aa, Artificial Sequence
  Description of Artificial Sequence Synthetic peptide
LNSYPRM

SEQ ID NO: 87 - protein, 8 aa, Artificial Sequence
  Description of Artificial Sequence Synthetic peptide
RSSSYYWG

SEQ ID NO: 88 - protein, 12 aa, Artificial Sequence
  Description of Artificial Sequence Synthetic peptide
WIGSIYYTGSTY

SEQ ID NO: 89 - protein, 12 aa, Artificial Sequence
  Description of Artificial Sequence Synthetic peptide
ASPGYSSGWSID

SEQ ID NO: 90 - protein, 7 aa, Artificial Sequence
  Description of Artificial Sequence Synthetic peptide
SSYLAWY

SEQ ID NO: 91 - protein, 10 aa, Artificial Sequence
  Description of Artificial Sequence Synthetic peptide
LLIYVASTLQ

SEQ ID NO: 92 - protein, 9 aa, Artificial Sequence
  Description of Artificial Sequence Synthetic peptide
QQLNSYPRM

SEQ ID NO: 93 - protein, 10 aa, Artificial Sequence
  Description of Artificial Sequence Synthetic peptide
GDSIRSSSYY

SEQ ID NO: 94 - protein, 7 aa, Artificial Sequence
  Description of Artificial Sequence Synthetic peptide
IYYTGST

SEQ ID NO: 95 - protein, 13 aa, Artificial Sequence
  Description of Artificial Sequence Synthetic peptide
ASPGYSSGWSIDY

SEQ ID NO: 96 - protein, 6 aa, Artificial Sequence
  Description of Artificial Sequence Synthetic peptide
QGISSY

SEQ ID NO: 97 - protein, 3 aa, Artificial Sequence
  Description of Artificial Sequence Synthetic peptide
VAS

SEQ ID NO: 98 - protein, 10 aa, Artificial Sequence
  Description of Artificial Sequence Synthetic peptide
QQLNSYPRMT

SEQ ID NO: 99 - protein, 123 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 100 - protein, 109 aa, Artificial Sequence
  Description of Artificial Sequence Synthetic polypeptide
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIK

SEQ ID NO: 101 - protein, 10 aa, Artificial Sequence
  Description of Artificial Sequence Synthetic peptide
GFTFSSYAMS

SEQ ID NO: 102 - protein, 10 aa, Artificial Sequence
  Description of Artificial Sequence Synthetic peptide
AISGSGGSTY

SEQ ID NO: 103 - protein, 14 aa, Artificial Sequence
  Description of Artificial Sequence Synthetic peptide
DEGYSSGHYYGMDV

SEQ ID NO: 104 - protein, 12 aa, Artificial Sequence
  Description of Artificial Sequence Synthetic peptide
RASQSISSSFLT

SEQ ID NO: 105 - protein, 7 aa, Artificial Sequence
  Description of Artificial Sequence Synthetic peptide
GASSRAT

SEQ ID NO: 106 - protein, 10 aa, Artificial Sequence
  Description of Artificial Sequence Synthetic peptide
QHYGSSPMYT

SEQ ID NO: 107 - protein, 5 aa, Artificial Sequence
  Description of Artificial Sequence Synthetic peptide
SYAMS

SEQ ID NO: 108 - protein, 17 aa, Artificial Sequence
  Description of Artificial Sequence Synthetic peptide
AISGSGGSTYYADSVKG

SEQ ID NO: 109 - protein, 14 aa, Artificial Sequence
  Description of Artificial Sequence Synthetic peptide
DEGYSSGHYYGMDV

SEQ ID NO: 110 - protein, 12 aa, Artificial Sequence
  Description of Artificial Sequence Synthetic peptide
RASQSISSSFLT

SEQ ID NO: 111 - protein, 7 aa, Artificial Sequence
  Description of Artificial Sequence Synthetic peptide
GASSRAT

SEQ ID NO: 112 - protein, 10 aa, Artificial Sequence
  Description of Artificial Sequence Synthetic peptide
QHYGSSPMYT

SEQ ID NO: 113 - protein, 7 aa, Artificial Sequence
  Description of Artificial Sequence Synthetic peptide
GFTFSSY

SEQ ID NO: 114 - protein, 6 aa, Artificial Sequence
  Description of Artificial Sequence Synthetic peptide
SGSGGS

SEQ ID NO: 115 - protein, 13 aa, Artificial Sequence
  Description of Artificial Sequence Synthetic peptide
DEGYSSGHYYGMD

SEQ ID NO: 116 - protein, 8 aa, Artificial Sequence
  Description of Artificial Sequence Synthetic peptide
SQSISSSF

SEQ ID NO: 117 - protein, 3 aa, Artificial Sequence
  Description of Artificial Sequence Synthetic peptide
GAS

SEQ ID NO: 118 - protein, 7 aa, Artificial Sequence
  Description of Artificial Sequence Synthetic peptide
YGSSPMY

SEQ ID NO: 119 - protein, 6 aa, Artificial Sequence
  Description of Artificial Sequence Synthetic peptide
SSYAMS

SEQ ID NO: 120 - protein, 13 aa, Artificial Sequence
  Description of Artificial Sequence Synthetic peptide
WVSAISGSGGSTY

SEQ ID NO: 121 - protein, 15 aa, Artificial Sequence
  Description of Artificial Sequence Synthetic peptide
AKDEGYSSGHYYGMD

SEQ ID NO: 122 - protein, 8 aa, Artificial Sequence
  Description of Artificial Sequence Synthetic peptide
SSSFLTWY

SEQ ID NO: 123 - protein, 10 aa, Artificial Sequence
  Description of Artificial Sequence Synthetic peptide
LLIYGASSRA

SEQ ID NO: 124 - protein, 9 aa, Artificial Sequence
  Description of Artificial Sequence Synthetic peptide
QHYGSSPMY

SEQ ID NO: 125 - protein, 8 aa, Artificial Sequence
  Description of Artificial Sequence Synthetic peptide
GFTFSSYA

SEQ ID NO: 126 - protein, 8 aa, Artificial Sequence
  Description of Artificial Sequence Synthetic peptide
ISGSGGST

SEQ ID NO: 127 - protein, 16 aa, Artificial Sequence
  Description of Artificial Sequence Synthetic peptide
AKDEGYSSGHYYGMDV

SEQ ID NO: 128 - protein, 7 aa, Artificial Sequence
  Description of Artificial Sequence Synthetic peptide
QSISSSF

SEQ ID NO: 129 - protein, 3 aa, Artificial Sequence
  Description of Artificial Sequence Synthetic peptide
GAS

SEQ ID NO: 130 - protein, 10 aa, Artificial Sequence
  Description of Artificial Sequence Synthetic peptide
QHYGSSPMYT

SEQ ID NO: 131 - protein, 119 aa, Artificial Sequence
  Description of Artificial Sequence Synthetic polypeptide
QVTLKESGPVLVKPTETLTLTCTVSGFSLTNIRMSVSWIRQPPGKALEWLAHIFSNDEKSYSTSLKSRLTISRDTSKSQVVLTLTNVDPVDTATYYCARMRLPYGMDVWGQGTTVTVSS

SEQ ID NO: 132 - protein, 112 aa, Artificial Sequence
  Description of Artificial Sequence Synthetic polypeptide
DIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIK

SEQ ID NO: 133 - protein, 12 aa, Artificial Sequence
  Description of Artificial Sequence Synthetic peptide
GFSLTNIRMSVS

SEQ ID NO: 134 - protein, 9 aa, Artificial Sequence
  Description of Artificial Sequence Synthetic peptide
HIFSNDEKS

SEQ ID NO: 135 - protein, 9 aa, Artificial Sequence
  Description of Artificial Sequence Synthetic peptide
MRLPYGMDV

SEQ ID NO: 136 - protein, 16 aa, Artificial Sequence
  Description of Artificial Sequence Synthetic peptide
RSSQSLVHSDGNTYLS

SEQ ID NO: 137 - protein, 7 aa, Artificial Sequence
  Description of Artificial Sequence Synthetic peptide
KISNRFF

SEQ ID NO: 138 - protein, 9 aa, Artificial Sequence
  Description of Artificial Sequence Synthetic peptide
MQATQFPHT

SEQ ID NO: 139 - protein, 7 aa, Artificial Sequence
  Description of Artificial Sequence Synthetic peptide
NIRMSVS

SEQ ID NO: 140 - protein, 16 aa, Artificial Sequence
  Description of Artificial Sequence Synthetic peptide
HIFSNDEKSYSTSLKS

SEQ ID NO: 141 - protein, 9 aa, Artificial Sequence
  Description of Artificial Sequence Synthetic peptide
MRLPYGMDV

SEQ ID NO: 142 - protein, 16 aa, Artificial Sequence
  Description of Artificial Sequence Synthetic peptide
RSSQSLVHSDGNTYLS

SEQ ID NO: 143 - protein, 7 aa, Artificial Sequence
  Description of Artificial Sequence Synthetic peptide
KISNRFF

SEQ ID NO: 144 - protein, 9 aa, Artificial Sequence
  Description of Artificial Sequence Synthetic peptide
MQATQFPHT

SEQ ID NO: 145 - protein, 9 aa, Artificial Sequence
  Description of Artificial Sequence Synthetic peptide
GFSLTNIRM

SEQ ID NO: 146 - protein, 5 aa, Artificial Sequence
  Description of Artificial Sequence Synthetic peptide
FSNDE

SEQ ID NO: 147 - protein, 8 aa, Artificial Sequence
  Description of Artificial Sequence Synthetic peptide
MRLPYGMD

SEQ ID NO: 148 - protein, 12 aa, Artificial Sequence
  Description of Artificial Sequence Synthetic peptide
SQSLVHSDGNTY

SEQ ID NO: 149 - protein, 3 aa, Artificial Sequence
  Description of Artificial Sequence Synthetic peptide
KIS

SEQ ID NO: 150 - protein, 6 aa, Artificial Sequence
  Description of Artificial Sequence Synthetic peptide
ATQFPH

SEQ ID NO: 151 - protein, 8 aa, Artificial Sequence
  Description of Artificial Sequence Synthetic peptide
TNIRMSVS

SEQ ID NO: 152 - protein, 12 aa, Artificial Sequence
  Description of Artificial Sequence Synthetic peptide
WLAHIFSNDEKS

SEQ ID NO: 153 - protein, 10 aa, Artificial Sequence
  Description of Artificial Sequence Synthetic peptide
ARMRLPYGMD

SEQ ID NO: 154 - protein, 12 aa, Artificial Sequence
  Description of Artificial Sequence Synthetic peptide
VHSDGNTYLSWL

SEQ ID NO: 155 - protein, 10 aa, Artificial Sequence
  Description of Artificial Sequence Synthetic peptide
LLIYKISNRF

SEQ ID NO: 156 - protein, 8 aa, Artificial Sequence
  Description of Artificial Sequence Synthetic peptide
MQATQFPH

SEQ ID NO: 157 - protein, 10 aa, Artificial Sequence
  Description of Artificial Sequence Synthetic peptide
GFSLTNIRMS

SEQ ID NO: 158 - protein, 7 aa, Artificial Sequence
  Description of Artificial Sequence Synthetic peptide
IFSNDEK

SEQ ID NO: 159 - protein, 11 aa, Artificial Sequence
  Description of Artificial Sequence Synthetic peptide
ARMRLPYGMDV

SEQ ID NO: 160 - protein, 11 aa, Artificial Sequence
  Description of Artificial Sequence Synthetic peptide
QSLVHSDGNTY

SEQ ID NO: 161 - protein, 3 aa, Artificial Sequence
  Description of Artificial Sequence Synthetic peptide
KIS

SEQ ID NO: 162 - protein, 9 aa, Artificial Sequence
  Description of Artificial Sequence Synthetic peptide
MQATQFPHT

SEQ ID NO: 163 - protein, 215 aa, Artificial Sequence
  Description of Artificial Sequence Synthetic polypeptide
DIQLTQSPSFLSASVGDRVTITCRASQGISSYLAWYQQKPGKAPKLLIYVASTLQSGVPSRFSGSGSGTEFTLTISSLQPEDFATYYCQQLNSYPRMTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 451 aa, Artificial Sequence
  Description of Artificial Sequence Synthetic polypeptide
QLQLQESGPGPVRPSETLSLTCTVSGDSIRSSSYYWGWIRQPPGKGLDWIGSIYYTGSTYYNPSLMSRVTISVDTSKNQFSLKLSSVTAADTAVYYCASPGYSSGWSIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 165 - protein, 484 aa, Artificial Sequence
  Description of Artificial Sequence Synthetic polypeptide
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 166 - protein, 722 aa, Artificial Sequence
  Description of Artificial Sequence Synthetic polypeptide
QLQLQESGPGPVRPSETLSLTCTVSGDSIRSSSYYWGWIRQPPGKGLDWIGSIYYTGSTYYNPSLMSRVTISVDTSKNQFSLKLSSVTAADTAVYYCASPGYSSGWSIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSEGKSSGSGSESKSTGGSEIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 167 - protein, 721 aa, Artificial Sequence
  Description of Artificial Sequence Synthetic polypeptide
QLQLQESGPGPVRPSETLSLTCTVSGDSIRSSSYYWGWIRQPPGKGLDWIGSIYYTGSTYYNPSLMSRVTISVDTSKNQFSLKLSSVTAADTAVYYCASPGYSSGWSIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSEGKSSGSGSESKSTGGSDIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSTSLKSRLTISRDTSKSQVVLTLTNVDPVDTATYYCARMRLPYGMDVWGQGTTVTVSS

SEQ ID NO: 168 - protein, 451 aa, Artificial Sequence
  Description of Artificial Sequence Synthetic polypeptide
EVQLLESGGGLVQPGGSLRLSCAASGFTFSIYAMTWVRQAPGKGLEWVSIISGSGDHTFYADSVKGRFTISRDNSRNTLYLQMDSLRAEDTAVYYCAKEGKWVQLSHFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 169 - protein, 214 aa, Artificial Sequence
  Description of Artificial Sequence Synthetic polypeptide
SYVLTQPPSVSVAPGQTARITCGGNNIGSKSVHWYQQKAGQAPVLVVYDDSDRPSGIPERFSGSNSGNTATLTISRVEAGDEADYYCQVWDGRSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 170 - protein, 220 aa, Artificial Sequence
  Description of Artificial Sequence Synthetic polypeptide
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 724 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSYTRYSPSFQGQVTISADKSISTAYLQWSSLKASDTAMYYCARGGVSASGNYRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSEGKSSGSGSESKSTGGSEIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 172 - protein, 722 aa, Artificial Sequence
  Description of Artificial Sequence Synthetic polypeptide
EVQLLESGGGLVQPGGSLRLSCAASGFTFSIYAMTWVRQAPGKGLEWVSIISGSGDHTFYADSVKGRFTISRDNSRNTLYLQMDSLRAEDTAVYYCAKEGKWVQLSHFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSEGKSSGSGSESKSTGGSEIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSS

SEQ ID NO: 173 - protein, 453 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSYTRYSPSFQGQVTISADKSISTAYLQWSSLKASDTAMYYCARGGVSASGNYRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 174 - protein, 483 aa, Artificial Sequence
  Description of Artificial Sequence Synthetic polypeptide
DIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSTSLKSRLTISRDTSKSQVVLTLTNVDPVDTATYYCARMRLPYGMDVWGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 175 - protein, 723 aa, Artificial Sequence
  Description of Artificial Sequence Synthetic polypeptide
EVQLVQSGAEVKKPGESLKISCKGSGYSFTSYWIGWVRQMPGKGLEWMGIIYPGDSYTRYSPSFQGQVTISADKSISTAYLQWSSLKASDTAMYYCARGGVSASGNYRALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSEGKSSGSGSESKSTGGSDIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSTSLKSRLTISRDTSKSQVVLTLTNVDPVDTATYYCARMRLPYGMDVWGQGTTVTVSS

SEQ ID NO: 176 - protein, 721 aa, Artificial Sequence
  Description of Artificial Sequence Synthetic polypeptide
EVQLLESGGGLVQPGGSLRLSCAASGFTFSIYAMTWVRQAPGKGLEWVSIISGSGDHTFYADSVKGRFTISRDNSRNTLYLQMDSLRAEDTAVYYCAKEGKWVQLSHFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSEGKSSGSGSESKSTGGSDIVMTQTPLSSPVTLGQPASISCRSSQSLVHSDGNTYLSWLQQRPGQPPRLLIYKISNRFFGVPDRFSGSGAGTDFTLKISRVEAEDVGVYYCMQATQFPHTFGQGTKLEIKGGSEGKSSGSGSESKSTGGSQVTLKESGPVLVKPTETLTLTCTVSGFSLTNIRMSVSWIRQPPGKALEWLAHIFSNDEKSYSTSLKSRLTISRDTSKSQVVLTLTNVDPVDTATYYCARMRLPYGMDVWGQGTTVTVSS

SEQ ID NO: 177 - protein, 451 aa, Artificial Sequence
  Description of Artificial Sequence Synthetic polypeptide
QLQLQESGPGPVRPSETLSLTCTVSGDSIRSSSYYWGWIRQPPGKGLDWIGSIYYTGSTYYNPSLMSRVTISVDTSKNQFSLKLSSVTAADTAVYYCASPGYSSGWSIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 178 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 179 - protein, 107 aa, Artificial Sequence [sequence omitted]

SEQ ID NO: 180 - protein, 484 aa, Artificial Sequence
  Description of Artificial Sequence Synthetic polypeptide
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 181 - protein, 20 aa, Artificial Sequence
  Description of Artificial Sequence Synthetic peptide
GGSEGKSSGSGSESKSTGGS

SEQ ID NO: 182 - protein, 232 aa, Artificial Sequence
  Description of Artificial Sequence Synthetic polypeptide
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 183 - DNA, 1353 bp, Artificial Sequence
  Description of Artificial Sequence Synthetic polynucleotide
cagctccagttgcaggaatcaggtcccggtcccgtccgcccctctgagaccctttctttgacctgcacagtatccggcgactctatccgcagctcatcatactactggggttggattcggcagcctccaggaaaaggtctcgactggatcggatctatttactatactggctctacctactacaatcctagcttgatgagcagggttaccatttctgtcgacacctcaaaaaatcagttcagtcttaaactgtccagcgtcactgctgcagacactgcagtgtattattgtgcctcacccggatattcatccgggtggagcattgattattggggccaaggtacactcgtaacagtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 184 - DNA, 363 bp, Artificial Sequence
  Description of Artificial Sequence Synthetic polynucleotide
cagctccagttgcaggaatcaggtcccggtcccgtccgcccctctgagaccctttctttgacctgcacagtatccggcgactctatccgcagctcatcatactactggggttggattcggcagcctccaggaaaaggtctcgactggatcggatctatttactatactggctctacctactacaatcctagcttgatgagcagggttaccatttctgtcgacacctcaaaaaatcagttcagtcttaaactgtccagcgtcactgctgcagacactgcagtgtattattgtgcctcacccggatattcatccgggtggagcattgattattggggccaaggtacactcgtaacagtctcttca

SEQ ID NO: 185 - DNA, 990 bp, Artificial Sequence
  Description of Artificial Sequence Synthetic polynucleotide
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccggttcacgcagaagagcctctccctgtctccgggtaaa

SEQ ID NO: 186 - DNA, 645 bp, Artificial Sequence
  Description of Artificial Sequence Synthetic polynucleotide
gacatccagctgacccagagccccagctttctgagcgccagcgtgggcgacagggtgaccatcacctgcagggccagccagggcatcagcagctacctggcctggtaccagcagaagcccggcaaagcccccaagctgctgatctacgtggccagcacactgcagagcggcgtgcctagcagatttagcggcagcggcagcggcacagagttcaccctgaccatctccagcctgcagcccgaggacttcgccacctactactgccagcagctgaacagctaccccaggatgaccttcggcggcggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 187 - DNA, 324 bp, Artificial Sequence
  Description of Artificial Sequence Synthetic polynucleotide
gacatccagctgacccagagccccagctttctgagcgccagcgtgggcgacagggtgaccatcacctgcagggccagccagggcatcagcagctacctggcctggtaccagcagaagcccggcaaagcccccaagctgctgatctacgtggccagcacactgcagagcggcgtgcctagcagatttagcggcagcggcagcggcacagagttcaccctgaccatctccagcctgcagcccgaggacttcgccacctactactgccagcagctgaacagctaccccaggatgaccttcggcggcggcaccaaggtggagatcaag

SEQ ID NO: 188 - DNA, 321 bp, Artificial Sequence
  Description of Artificial Sequence Synthetic polynucleotide
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 189 - DNA, 1452 bp, Artificial Sequence
  Description of Artificial Sequence Synthetic polynucleotide
gagatcgtgctgactcagagccctggcactctgagcctgagccctggcgagagggccactctgagctgtagggccagccagagcatcagcagcagcttcctgacttggtaccagcagaagcctggccaggcccctaggctgctgatctacggcgccagcagcagggccactggcatccctgataggttcagcggcggcggcagcggcactgatttcactctgactatcagcaggctggagcctgaggatttcgccgtgtactactgtcagcactacggcagcagccctatgtacactttcggccagggcactaagctggagatcaagggcggctccgagggcaagagcagcggcagcggcagcgagagcaagagcaccggcggcagcgaagtgcagctgctggagtcagggggcggtctggtacaaccgggcggcagcctgaggctgagctgtgccgccagcggcttcactttcagcagctacgccatgagctgggtgaggcaggcccctggcaagggcctggagtgggtgagcgccatcagcggcagcggcggcagcacttactacgccgatagcgtgaagggcaggttcactatcagcagggataatagcaagaatactctgtacctgcagatgaatagcctgagggccgaggatactgccgtgtactactgtgccaaggatgagggctacagcagcggccactactacggcatggatgtgtggggccagggcactactgtgactgtgagcagcgagcccaaatctagcgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 190 - DNA, 369 bp, Artificial Sequence
  Description of Artificial Sequence Synthetic polynucleotide
gaagtgcagctgctggagtcagggggcggtctggtacaaccgggcggcagcctgaggctgagctgtgccgccagcggcttcactttcagcagctacgccatgagctgggtgaggcaggcccctggcaagggcctggagtgggtgagcgccatcagcggcagcggcggcagcacttactacgccgatagcgtgaagggcaggttcactatcagcagggataatagcaagaatactctgtacctgcagatgaatagcctgagggccgaggatactgccgtgtactactgtgccaaggatgagggctacagcagcggccactactacggcatggatgtgtggggccagggcactactgtgactgtgagcagc

SEQ ID NO: 191 - DNA, 60 bp, Artificial Sequence
  Description of Artificial Sequence Synthetic nucleotide
ggcggctccgagggcaagagcagcggcagcggcagcgagagcaagagcaccggcggcagc

SEQ ID NO: 192 - DNA, 327 bp, Artificial Sequence
  Description of Artificial Sequence Synthetic polynucleotide
gagatcgtgctgactcagagccctggcactctgagcctgagccctggcgagagggccactctgagctgtagggccagccagagcatcagcagcagcttcctgacttggtaccagcagaagcctggccaggcccctaggctgctgatctacggcgccagcagcagggccactggcatccctgataggttcagcggcggcggcagcggcactgatttcactctgactatcagcaggctggagcctgaggatttcgccgtgtactactgtcagcactacggcagcagccctatgtacactttcggccagggcactaagctggagatcaag

SEQ ID NO: 193 - DNA, 696 bp, Artificial Sequence
  Description of Artificial Sequence Synthetic polynucleotide
gagcccaaatctagcgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 194 - protein, 451 aa, Artificial Sequence
  Description of Artificial Sequence Synthetic polypeptide
QLQLQESGPGPVRPSETLSLTCTVSGDSIRSSSYYWGWIRQPPGKGLDWIGSIYYTGSTYYNPSLMSRVTISVDTSKNQFSLKLSSVTAADTAVYYCASPGYSSGWSIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 195 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 196 - protein, 484 aa, Artificial Sequence
  Description of Artificial Sequence Synthetic polypeptide
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 197 - protein, 232 aa, Artificial Sequence
  Description of Artificial Sequence Synthetic polypeptide
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 198 - DNA, 1353 bp, Artificial Sequence
  Description of Artificial Sequence Synthetic polynucleotide
cagctccagttgcaggaatcaggtcccggtcccgtccgcccctctgagaccctttctttgacctgcacagtatccggcgactctatccgcagctcatcatactactggggttggattcggcagcctccaggaaaaggtctcgactggatcggatctatttactatactggctctacctactacaatcctagcttgatgagcagggttaccatttctgtcgacacctcaaaaaatcagttcagtcttaaactgtccagcgtcactgctgcagacactgcagtgtattattgtgcctcacccggatattcatccgggtggagcattgattattggggccaaggtacactcgtaacagtctcttcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 199 - DNA, 990 bp, Artificial Sequence
  Description of Artificial Sequence Synthetic polynucleotide
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 200 - DNA, 1452 bp, Artificial Sequence
  Description of Artificial Sequence Synthetic polynucleotide
gagatcgtgctgactcagagccctggcactctgagcctgagccctggcgagagggccactctgagctgtagggccagccagagcatcagcagcagcttcctgacttggtaccagcagaagcctggccaggcccctaggctgctgatctacggcgccagcagcagggccactggcatccctgataggttcagcggcggcggcagcggcactgatttcactctgactatcagcaggctggagcctgaggatttcgccgtgtactactgtcagcactacggcagcagccctatgtacactttcggccagggcactaagctggagatcaagggcggctccgagggcaagagcagcggcagcggcagcgagagcaagagcaccggcggcagcgaagtgcagctgctggagtcagggggcggtctggtacaaccgggcggcagcctgaggctgagctgtgccgccagcggcttcactttcagcagctacgccatgagctgggtgaggcaggcccctggcaagggcctggagtgggtgagcgccatcagcggcagcggcggcagcacttactacgccgatagcgtgaagggcaggttcactatcagcagggataatagcaagaatactctgtacctgcagatgaatagcctgagggccgaggatactgccgtgtactactgtgccaaggatgagggctacagcagcggccactactacggcatggatgtgtggggccagggcactactgtgactgtgagcagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 201 - DNA, 696 bp, Artificial Sequence
  Description of Artificial Sequence Synthetic polynucleotide
gagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagtctagatggcagcaggggaacgtcttctcatgctccgtgatgcatgaggctctgcacaaccactacacgcagaagagcctctccctgtctccgggtaaa

SEQ ID NO: 202 - protein, 227 aa, Artificial Sequence
  Description of Artificial Sequence Synthetic polypeptide
DKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYRQKSLSLSPGK

SEQ ID NO: 203 - protein, 453 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEGYSSGHYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFRQKSLSLSPGK

SEQ ID NO: 204 - protein, 330 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPEDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFRQKSLSLSPGK

SEQ ID NO: 205 - protein, 216 aa, Artificial Sequence
  Description of Artificial Sequence Synthetic polypeptide
EIVLTQSPGTLSLSPGERATLSCRASQSISSSFLTWYQQKPGQAPRLLIYGASSRATGIPDRFSGGGSGTDFTLTISRLEPEDFAVYYCQHYGSSPMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 107 aa, Artificial Sequence [sequence omitted]

SEQ ID NO: 207 - DNA, 681 bp, Artificial Sequence
  Description of Artificial Sequence Synthetic polynucleotide
gacaaaactcacacatgtccaccgtgcccagcacctgaactgctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactaccggcagaagagcctctccctgtctccgggtaaa

SEQ ID NO: 208 - DNA, 1359 bp, Artificial Sequence
  Description of Artificial Sequence Synthetic polynucleotide
gaagtgcagctgctggagtcagggggcggtctggtacaaccgggcggcagcctgaggctgagctgtgccgccagcggcttcactttcagcagctacgccatgagctgggtgaggcaggcccctggcaagggcctggagtgggtgagcgccatcagcggcagcggcggcagcacttactacgccgatagcgtgaagggcaggttcactatcagcagggataatagcaagaatactctgtacctgcagatgaatagcctgagggccgaggatactgccgtgtactactgtgccaaggatgagggctacagcagcggccactactacggcatggatgtgtggggccagggcactactgtgactgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagtctagatggcagcaggggaacgtcttctcatgctccgtgatgcatgaggctctgcacaaccggttccgccagaagagcctctccctgtctccgggtaaa

SEQ ID NO: 209 - DNA, 990 bp, Artificial Sequence
  Description of Artificial Sequence Synthetic polynucleotide
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgtccaccgtgcccagcacctgaactgctggggggaccgtcagtcttcctcttccccccaaaacccgaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagtctagatggcagcaggggaacgtcttctcatgctccgtgatgcatgaggctctgcacaaccggttccgccagaagagcctctccctgtctccgggtaaa

SEQ ID NO: 210 - DNA, 648 bp, Artificial Sequence
  Description of Artificial Sequence Synthetic polynucleotide
gagatagttcttacccaatctcctggaaccctcagtctcagtccaggtgagagagctactctctcatgtagagcaagccaatctattagttctagctttctcacctggtatcagcaaaagccaggacaagctcctcgtttgcttatttatggagcatcatctagggccactggaatacctgatcgtttcagtggtggcggttcagggaccgactttacactgactataagcagattggaaccagaagactttgctgtctactattgtcaacattacggatcctccccaatgtacacatttggtcaaggcacaaaattggagataaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

SEQ ID NO: 211 - DNA, 327 bp, Artificial Sequence
  Description of Artificial Sequence Synthetic polynucleotide
gagatagttcttacccaatctcctggaaccctcagtctcagtccaggtgagagagctactctctcatgtagagcaagccaatctattagttctagctttctcacctggtatcagcaaaagccaggacaagctcctcgtttgcttatttatggagcatcatctagggccactggaatacctgatcgtttcagtggtggcggttcagggaccgactttacactgactataagcagattggaaccagaagactttgctgtctactattgtcaacattacggatcctccccaatgtacacatttggtcaaggcacaaaattggagataaag

SEQ ID NO: 212 - DNA, 321 bp, Artificial Sequence
  Description of Artificial Sequence Synthetic polynucleotide
cggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgt

(19) United States
(12) Patent Application Publication
(10) Pub. No.: US 2024/0182582 A1
(43) Pub. Date: Jun. 6, 2024

(54) MATERIALS AND METHODS FOR IMMUNE EFFECTOR CELLS REDIRECTION

(71) Applicant: JANSSEN BIOTHECH, INC., Horsham, PA (US)

(72) Inventors: Xiefan Lin-Schmidt, Ambler, PA (US); Ian White, Spring House, PA (US); Adam Zwolak, Spring House, PA (US); Rajkumar Ganesan, Thousand Oaks, CA (US); Jason Ho, Spring House, NJ (US)

(21) Appl. No.: 18/284,586

(22) PCT Filed: Mar. 30, 2022
(86) PCT No.: PCT/US2022/022500

§ 371 Date: Sep. 28, 2023

Related U.S. Application Data
(60) Provisional application No. 63/168,605, filed on Mar. 31, 2021.
(60) Provisional application No. 63/168,611, filed on Mar. 31, 2021.
(60) Provisional application No. 63/168,618, filed on Mar. 31, 2021.
(60) Provisional application No. 63/168,621, filed on Mar. 31, 2021.
(60) Provisional application No. 63/168,628, filed on Mar. 31, 2021.

(51) Int. Cl. C07K 16/28

(52) U.S. Cl. CPC C07K 16/2851; C07K 16/2803; C07K 16/2866; C07K 16/2878; C07K 2317/31; C07K 2317/569

(57) ABSTRACT

A molecule comprising a first means for engaging or activating a Natural Killer (NK) cell, and a second means for binding a tumor cell, wherein the molecule is capable of inducing NK cell dependent cytotoxicity against the tumor cell.

### CROSS-REFERENCE TO RELATED APPLICATIONS

This application claims the benefit of U.S. Ser. No. 63/168,605, filed Mar. 31, 2021; U.S. Ser. No. 63/168,611, filed Mar. 31, 2021; U.S. Ser. No. 63/168,618, filed Mar. 31, 2021; U.S. Ser. No. 63/168,621, filed Mar. 31, 2021; U.S. Ser. No. 63/168,628, filed Mar. 31, 2021, each of which is herein incorporated by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file “14620-648-228_SEQ_LISTING” and a creation date of Mar. 24, 2022 and having a size of 181,332 bytes. The sequence listing submitted via EFS-Web is part of the specification and is herein incorporated by reference in its entirety.

### 1. FIELD

This present disclosure relates to, among other things, natural killer cell engagers including anti-NKG2d molecules, anti-NKp46 molecules, and multispecific molecules comprising same or fragments thereof, as well as nucleic acids and expression vectors encoding the molecules, recombinant cells containing the vectors, and compositions comprising the molecules. Methods of making, and methods of using the molecules to redirect immune effector cells against tumor cells, are also provided.

### 2. BACKGROUND

Tumor cells can be therapeutically targeted for destruction by antibodies. Therapeutic antibodies can engage immune effector cells to target tumor cells for destruction using a number of mechanisms. Effector cells can be redirected against tumor cells using bispecific antibodies (bsAbs) which bind tumor cells and effector cells bringing them into close proximity. Alternatively, monoclonal antibodies (mAbs) can engage tumor cells via their variable regions and recruit effector cells via interactions between the Fc region and Fc g receptors expressed primarily on a monocytes, macrophages, and NK cells.

### 3. SUMMARY

The inventors were the first to discover the multispecific antibodies described herein comprising a first binding domain that binds to a first antigen expressed on a Natural Killer (NK) cell, and a second binding domain that binds to a second antigen expressed on a tumor cell.

In one aspect, provided herein is a multispecific antibody comprising a first binding domain that binds to a first antigen expressed on a Natural Killer (NK) cell, and a second binding domain that binds to a second antigen.

In some embodiments of the multispecific antibody provided herein, the first antigen is an NK cell activating receptor.

In some embodiments of the multispecific antibody provided herein, the first antigen is an NKG2d.

In some embodiments of the multispecific antibody provided herein, the first binding domain comprises: a heavy chain variable region (VH) comprising (a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6; (b) a VH complementarity determining region (CDR) 1 having an amino acid sequence of SEQ ID NO:10, a VH CDR2 having an amino acid sequence of SEQ ID NO:11, and a VH CDR3 having an amino acid sequence of SEQ ID NO:12; (c) a VH complementarity determining region (CDR) 1 having an amino acid sequence of SEQ ID NO:16, a VH CDR2 having an amino acid sequence of SEQ ID NO:17, and a VH CDR3 having an amino acid sequence of SEQ ID NO:18; (d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or (e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30; and a light chain variable region (VL) comprising (a) a VL CDR1 having an amino acid sequence of SEQ ID NO:7, a VL CDR2 having an amino acid sequence of SEQ ID NO:8, and a VL CDR3 having an amino acid sequence of SEQ ID NO:9; (b) a VL CDR1 having an amino acid sequence of SEQ ID NO:13, a VL CDR2 having an amino acid sequence of SEQ ID NO:14, and a VL CDR3 having an amino acid sequence of SEQ ID NO:15; (c) a VL CDR1 having an amino acid sequence of SEQ ID NO:19, a VL CDR2 having an amino acid sequence of SEQ ID NO:20, and a VL CDR3 having an amino acid sequence of SEQ ID NO:21; (d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or (e) a VL CDR1 having an amino acid sequence of SEQ ID NO:31, a VL CDR2 having an amino acid sequence of SEQ ID NO:32, and a VL CDR3 having an amino acid sequence of SEQ ID NO:33.

In some embodiments of the multispecific antibody provided herein, the first binding domain comprises: a VH having an amino acid sequence of SEQ ID NO:2, and a VL having an amino acid sequence of SEQ ID NO:3.

In some embodiments of the multispecific antibody provided herein, the first binding domain comprises: (i) a heavy chain variable region (VH) comprising (a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38; (b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44; (c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50; (d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or (e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62; and (ii) a light chain variable region (VL) comprising (a) a VL CDR1 having an amino acid sequence of SEQ ID NO:39, a VL CDR2 having an amino acid sequence of SEQ ID NO:40, and a VL CDR3 having an amino acid sequence of SEQ ID NO:41; (b) a VL CDR1 having an amino acid sequence of SEQ ID NO:45, a VL CDR2 having an amino acid sequence of SEQ ID NO:46, and a VL CDR3 having an amino acid sequence of SEQ ID NO:47; (c) a VL CDR1 having an amino acid sequence of SEQ ID NO:51, a VL CDR2 having an amino acid sequence of SEQ ID NO:52, and a VL CDR3 having an amino acid sequence of SEQ ID NO:53; (d) a VL CDR1 having an amino acid sequence of SEQ ID NO:57, a VL CDR2 having an amino acid sequence of SEQ ID NO:58, and a VL CDR3 having an amino acid sequence of SEQ ID NO:59; or (e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.

In some embodiments of the multispecific antibody provided herein, the first binding domain comprises: a VH having an amino acid sequence of SEQ ID NO:34, and a VL having an amino acid sequence of SEQ ID NO:35.

In some embodiments of the multispecific antibody provided herein, the first antigen is an NKp46.

In some embodiments of the multispecific antibody provided herein, the first binding domain comprises: (i) a heavy chain variable region (VH) comprising (a) a VH complementarity determining region (CDR) 1 having an amino acid sequence of SEQ ID NO:69, a VH CDR2 having an amino acid sequence of SEQ ID NO:70, and a VH CDR3 having an amino acid sequence of SEQ ID NO:71; (b) a VH complementarity determining region (CDR) 1 having an amino acid sequence of SEQ ID NO:75, a VH CDR2 having an amino acid sequence of SEQ ID NO:76, and a VH CDR3 having an amino acid sequence of SEQ ID NO:77; (c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83; (d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or (e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95; and (ii) a light chain variable region (VL) comprising (a) a VL CDR1 having an amino acid sequence of SEQ ID NO:72, a VL CDR2 having an amino acid sequence of SEQ ID NO:73, and a VL CDR3 having an amino acid sequence of SEQ ID NO:74; (b) a VL CDR1 having an amino acid sequence of SEQ ID NO:78, a VL CDR2 having an amino acid sequence of SEQ ID NO:79, and a VL CDR3 having an amino acid sequence of SEQ ID NO:80; (c) a VL CDR1 having an amino acid sequence of SEQ ID NO:84, a VL CDR2 having an amino acid sequence of SEQ ID NO:85, and a VL CDR3 having an amino acid sequence of SEQ ID NO:86; (d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or (e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.

In some embodiments of the multispecific antibody provided herein, the first binding domain comprises: a VH having an amino acid sequence of SEQ ID NO:67, and a VL having an amino acid sequence of SEQ ID NO:68.

In some embodiments of the multispecific antibody provided herein, the second antigen is on a cell surface.

In some embodiments of the multispecific antibody provided herein, the second antigen is expressed on a tumor cell.

In some embodiments of the multispecific antibody provided herein, the second antigen is a tumor specific antigen (TSA) or a tumor associated antigen (TAA).

In some embodiments of the multispecific antibody provided herein, the second antigen is BCMA.

In some embodiments of the multispecific antibody provided herein, the second antigen is GPRC5d.

In some embodiments of the multispecific antibody provided herein, the first binding domain is humanized. In some embodiments of the multispecific antibody provided herein, the second binding domain is humanized. In some embodiments of the multispecific antibody provided herein, both the first binding domain and the second binding domain are humanized.

In some embodiments of the multispecific antibody provided herein, the multispecific antibody is an IgG antibody.

In some embodiments of the multispecific antibody provided herein, the IgG antibody is an IgG1, IgG2, IgG3, or IgG4 antibody.

In some embodiments of the multispecific antibody provided herein, the IgG antibody is an IgG1 antibody.

In some embodiments of the multispecific antibody provided herein, the IgG1 comprises silent mutations. In some embodiments of the multispecific antibody provided herein, the IgG1 comprises AAS mutations. In some embodiments, the multispecific antibody that comprises the AAS mutations can induce NK cell dependent cytotoxicity of the tumor cell.

In some embodiments of the multispecific antibody provided herein, the IgG1 comprises mutations for enhancement of an effector function of the antibody. In some embodiments of the multispecific antibody provided herein, the IgG1 comprises K248E/T437R mutations. In some embodiments, the multispecific antibody that comprises the K248E/T437R mutations is lack of anti-NK cell cytotoxicity.

In some embodiments of the multispecific antibody provided herein, the Fc region is afucosylated.

In some embodiments of the multispecific antibody provided herein, the multispecific antibody is a bispecific antibody.

In some embodiments of the multispecific antibody provided herein, the bispecific antibody is in a bipod-scaffold configuration.

In some embodiments of the multispecific antibody provided herein, the first binding domain is a Fab region, and the second binding domain is a scFv region.

In some embodiments of the multispecific antibody provided herein, the bispecific antibody is in a Morrison-scaffold configuration.

In some embodiments of the multispecific antibody provided herein, the first binding domain comprises two Fab regions and the second binding domain comprises two scFv regions.

In some embodiments of the multispecific antibody provided herein, the multispecific antibody is lack of anti-NK cell cytotoxicity.

In some embodiments of the multispecific antibody provided herein, the multispecific antibody induces NK cell dependent cytotoxicity of the tumor cell in vitro with an IC₅₀ of less than about 500 pM. In some embodiments, the multispecific antibody induces NK cell dependent cytotoxicity of the tumor cell in vitro with an IC₅₀ of less than about 300 pM. In some embodiments, the multispecific antibody induces NK cell dependent cytotoxicity of the tumor cell in vitro with an IC₅₀ of less than about 100 pM. In some embodiments, the multispecific antibody induces NK cell dependent cytotoxicity of the tumor cell in vitro with an IC₅₀ of less than about 50 pM. In some embodiments, the multispecific antibody induces NK cell dependent cytotoxicity of the tumor cell in vitro with an IC₅₀ of less than about 20 pM. In some embodiments, the multispecific antibody induces NK cell dependent cytotoxicity of the tumor cell in vitro with an IC₅₀ of less than about 15 pM. In some embodiments, the multispecific antibody induces NK cell dependent cytotoxicity of the tumor cell in vitro with an IC₅₀ of less than about 10 pM.

In some embodiments of the multispecific antibody provided herein, the IC₅₀ is assessed with a mixture of NK effector cells and target cells expressing the second antigen.

In some embodiments of the multispecific antibody provided herein, the effector cell to target cell ratio is about 0.01 to 1 to about 10 to 1. In some embodiments of the multispecific antibody provided herein, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In another aspect, provided is a nucleic acid encoding a multispecific antibody provided herein. Also provided is a vector comprising the nucleic acid encoding a multispecific antibody provided herein. Also provided is a host cell comprising a vector comprising a nucleic acid encoding a multispecific antibody provided herein. Also provided is a kit comprising a vector comprising a nucleic acid encoding a multispecific antibody provided herein, and packaging for the same.

In another aspect, provided herein is an antibody that binds NKG2d, comprising: (i) a heavy chain variable region (VH) comprising (a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6; (b) a VH complementarity determining region (CDR) 1 having an amino acid sequence of SEQ ID NO:10, a VH CDR2 having an amino acid sequence of SEQ ID NO:11, and a VH CDR3 having an amino acid sequence of SEQ ID NO:12; (c) a VH complementarity determining region (CDR) 1 having an amino acid sequence of SEQ ID NO:16, a VH CDR2 having an amino acid sequence of SEQ ID NO:17, and a VH CDR3 having an amino acid sequence of SEQ ID NO:18; (d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or (e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30; and (ii) a light chain variable region (VL) comprising (a) a VL CDR1 having an amino acid sequence of SEQ ID NO:7, a VL CDR2 having an amino acid sequence of SEQ ID NO:8, and a VL CDR3 having an amino acid sequence of SEQ ID NO:9; (b) a VL CDR1 having an amino acid sequence of SEQ ID NO:13, a VL CDR2 having an amino acid sequence of SEQ ID NO:14, and a VL CDR3 having an amino acid sequence of SEQ ID NO:15; (c) a VL CDR1 having an amino acid sequence of SEQ ID NO:19, a VL CDR2 having an amino acid sequence of SEQ ID NO:20, and a VL CDR3 having an amino acid sequence of SEQ ID NO:21; (d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or (e) a VL CDR1 having an amino acid sequence of SEQ ID NO:31, a VL CDR2 having an amino acid sequence of SEQ ID NO:32, and a VL CDR3 having an amino acid sequence of SEQ ID NO:33.

In some embodiments of the antibody that binds NKG2d provided herein, the first binding domain comprises a VH having an amino acid sequence of SEQ ID NO:2, and a VL having an amino acid sequence of SEQ ID NO:3.

In another aspect, provided herein is an antibody that binds NKG2d, comprising: (i) a heavy chain variable region (VH) comprising (a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38; (b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44; (c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50; (d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or (e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62; and (ii) a light chain variable region (VL) comprising (a) a VL CDR1 having an amino acid sequence of SEQ ID NO:39, a VL CDR2 having an amino acid sequence of SEQ ID NO:40, and a VL CDR3 having an amino acid sequence of SEQ ID NO:41; (b) a VL CDR1 having an amino acid sequence of SEQ ID NO:45, a VL CDR2 having an amino acid sequence of SEQ ID NO:46, and a VL CDR3 having an amino acid sequence of SEQ ID NO:47; (c) a VL CDR1 having an amino acid sequence of SEQ ID NO:51, a VL CDR2 having an amino acid sequence of SEQ ID NO:52, and a VL CDR3 having an amino acid sequence of SEQ ID NO:53; (d) a VL CDR1 having an amino acid sequence of SEQ ID NO:57, a VL CDR2 having an amino acid sequence of SEQ ID NO:58, and a VL CDR3 having an amino acid sequence of SEQ ID NO:59; or (e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.

In some embodiments of the antibody that binds NKG2d provided herein, the first binding domain comprises: a VH having an amino acid sequence of SEQ ID NO:34, and a VL having an amino acid sequence of SEQ ID NO:35.

In another aspect, provided herein is an antibody that binds NKp46, comprising: (i) a heavy chain variable region (VH) comprising (a) a VH complementarity determining region (CDR) 1 having an amino acid sequence of SEQ ID NO:69, a VH CDR2 having an amino acid sequence of SEQ ID NO:70, and a VH CDR3 having an amino acid sequence of SEQ ID NO:71; (b) a VH complementarity determining region (CDR) 1 having an amino acid sequence of SEQ ID NO:75, a VH CDR2 having an amino acid sequence of SEQ ID NO:76, and a VH CDR3 having an amino acid sequence of SEQ ID NO:77; (c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83; (d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or (e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95; and (ii) a light chain variable region (VL) comprising (a) a VL CDR1 having an amino acid sequence of SEQ ID NO:72, a VL CDR2 having an amino acid sequence of SEQ ID NO:73, and a VL CDR3 having an amino acid sequence of SEQ ID NO:74; (b) a VL CDR1 having an amino acid sequence of SEQ ID NO:78, a VL CDR2 having an amino acid sequence of SEQ ID NO:79, and a VL CDR3 having an amino acid sequence of SEQ ID NO:80; (c) a VL CDR1 having an amino acid sequence of SEQ ID NO:84, a VL CDR2 having an amino acid sequence of SEQ ID NO:85, and a VL CDR3 having an amino acid sequence of SEQ ID NO:86; (d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or (e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.

In some embodiments of the antibody that binds NKp46 provided herein, the first binding domain comprises: a VH having an amino acid sequence of SEQ ID NO:67, and a VL having an amino acid sequence of SEQ ID NO:68.

In another aspect, provided herein is a nucleic acid encoding an antibody provided herein. Also provided is a vector comprising the nucleic acid encoding an antibody provided herein. Also provided is a host cell comprising a vector comprising a nucleic acid encoding an antibody provided herein. Also provided is a kit comprising a vector comprising a nucleic acid encoding an antibody provided herein, and packaging for the same.

In yet another aspect, provided herein is a pharmaceutical composition comprising a multispecific antibody provided herein, and a pharmaceutically acceptable carrier, wherein the multispecific antibody comprises: a first binding domain that binds to a first antigen expressed on a Natural Killer (NK) cell, and a second binding domain that binds to a second antigen.

In some embodiments of the pharmaceutical composition provided herein, the first antigen is an NK cell activating receptor.

In some embodiments of the pharmaceutical composition provided herein, the first antigen is an NKG2d.

In some embodiments of the pharmaceutical composition provided herein, the first antigen is an NKp46.

In some embodiments of the pharmaceutical composition provided herein, the second antigen is on a cell surface.

In some embodiments of the pharmaceutical composition provided herein, the second antigen is expressed on a tumor cell.

In some embodiments of the pharmaceutical composition provided herein, the second antigen is a tumor specific antigen (TSA) or a tumor associated antigen (TAA).

In some embodiments of the pharmaceutical composition provided herein, the second antigen is BCMA.

In some embodiments of the pharmaceutical composition provided herein, the second antigen is GPRC5d.

In some embodiments of the pharmaceutical composition provided herein, the first binding domain is humanized. In some embodiments of the pharmaceutical composition provided herein, the second binding domain is humanized. In some embodiments of the pharmaceutical composition provided herein, both the first binding domain and the second binding domain are humanized.

In some embodiments of the pharmaceutical composition provided herein, the multispecific antibody is an IgG antibody.

In some embodiments of the pharmaceutical composition provided herein, the IgG antibody is an IgG1, IgG2, IgG3, or IgG4 antibody.

In some embodiments of the pharmaceutical composition provided herein, the IgG antibody is an IgG1 antibody.

In some embodiments of the pharmaceutical composition provided herein, the IgG1 comprises silent mutations. In some embodiments of the pharmaceutical composition provided herein, the IgG1 comprise AAS mutations.

In some embodiments of the pharmaceutical composition provided herein, the IgG1 comprises mutations for enhancement of an effector function of the antibody. In some embodiments of the pharmaceutical composition provided herein, the IgG1 comprises K248E/T437R mutations.

In some embodiments of the pharmaceutical composition provided herein, the Fc region is afucosylated.

In some embodiments of the pharmaceutical composition provided herein, the multispecific antibody is a bispecific antibody.

In some embodiments of the pharmaceutical composition provided herein, the bispecific antibody is in a bipod-scaffold configuration.

In some embodiments of the pharmaceutical composition provided herein, the first binding domain is a Fab region, and the second binding domain is a scFv region.

In some embodiments of the pharmaceutical composition provided herein, the bispecific antibody is in a Morrison-scaffold configuration.

In some embodiments of the pharmaceutical composition provided herein, the first binding domain comprises two Fab regions and the second binding domain comprises two scFv regions.

In some embodiments of the pharmaceutical composition provided herein, the multispecific antibody induces NK cell dependent cytotoxicity of the tumor cell in vitro with an IC₅₀ of less than about 500 pM. In some embodiments, the multispecific antibody induces NK cell dependent cytotoxicity of the tumor cell in vitro with an IC₅₀ of less than about 300 pM. In some embodiments, the multispecific antibody induces NK cell dependent cytotoxicity of the tumor cell in vitro with an IC₅₀ of less than about 100 pM. In some embodiments, the multispecific antibody induces NK cell dependent cytotoxicity of the tumor cell in vitro with an IC₅₀ of less than about 50 pM. In some embodiments, the multispecific antibody induces NK cell dependent cytotoxicity of the tumor cell in vitro with an IC₅₀ of less than about 20 pM. In some embodiments, the multispecific antibody induces NK cell dependent cytotoxicity of the tumor cell in vitro with an IC₅₀ of less than about 15 pM. In some embodiments, the multispecific antibody induces NK cell dependent cytotoxicity of the tumor cell in vitro with an IC₅₀ of less than about 10 pM.

In some embodiments of the pharmaceutical composition provided herein, the IC₅₀ is assessed with a mixture of NK effector cells and target cells expressing the second antigen.

In some embodiments of the pharmaceutical composition provided herein, the effector cell to target cell ratio is about 0.01 to 1 to about 10 to 1. In some embodiments of the pharmaceutical composition provided herein, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In yet another aspect, provided herein is a process for making a multispecific antibody comprising introducing into a host cell one or more nucleic acids encoding a first binding domain that binds to a first antigen expressed on an NK cell, and a second binding domain that binds to a second antigen.

In some embodiments of the process for making a multispecific antibody provided herein, the first antigen is an NK cell activating receptor.

In some embodiments of the process for making a multispecific antibody provided herein, wherein the multispecific antibody is a multispecific antibody provided herein.

In some embodiments of the process for making a multispecific antibody provided herein, the first antigen is NKp46. In some embodiments of the process for making a multispecific antibody provided herein, the first antigen is NKG2d.

In some embodiments of the process for making a multispecific antibody provided herein, the second antigen is on a cell surface.

In some embodiments of the process for making a multispecific antibody provided herein, the second antigen is expressed on a tumor cell.

In some embodiments of the process for making a multispecific antibody provided herein, the second antigen is a tumor specific antigen (TSA) or a tumor associated antigen (TAA).

In some embodiments of the process for making a multispecific antibody provided herein, the second antigen is BCMA.

In some embodiments of the process for making a multispecific antibody provided herein, the second antigen is GPRC5d.

In some embodiments of the process for making a multispecific antibody provided herein, the first binding domain is humanized. In some embodiments of the process for making a multispecific antibody provided herein, the second binding domain is humanized. In some embodiments of the process for making a multispecific antibody provided herein, both the first binding domain and the second binding domain are humanized.

In some embodiments of the process for making a multispecific antibody provided herein, the multispecific antibody is an IgG antibody.

In some embodiments of the process for making a multispecific antibody provided herein, the IgG antibody is an IgG1, IgG2, IgG3, or IgG4 antibody.

In some embodiments of the process for making a multispecific antibody provided herein, the IgG antibody is an IgG1 antibody.

In some embodiments of the process provided herein, the IgG1 comprises silent mutations. In some embodiments of the process provided herein, the IgG1 comprises AAS mutations.

In some embodiments of the process provided herein, the IgG1 comprises mutations for enhancement of an effector function of the antibody. In some embodiments of the process provided herein, the IgG1 comprises K248E/T437R mutations.

In some embodiments of the process provided herein, the Fc region is afucosylated.

In some embodiments of the process for making a multispecific antibody provided herein, the multispecific antibody is a bispecific antibody.

In some embodiments of the process for making a multispecific antibody provided herein, the bispecific antibody is in a bipod-scaffold configuration.

In some embodiments of the process for making a multispecific antibody provided herein, the first binding domain is a Fab region, and the second binding domain is a scFv region.

In some embodiments of the process for making a multispecific antibody provided herein, the bispecific antibody is in a Morrison-scaffold configuration.

In some embodiments of the process for making a multispecific antibody provided herein, the first binding domain comprises two Fab regions and the second binding domain comprises two scFv regions.

In some embodiments of the process for making a multispecific antibody provided herein, the multispecific antibody induces NK cell dependent cytotoxicity of the tumor cell in vitro with an IC₅₀ of less than about 500 pM. In some embodiments, the multispecific antibody induces NK cell dependent cytotoxicity of the tumor cell in vitro with an IC₅₀ of less than about 300 pM. In some embodiments, the multispecific antibody induces NK cell dependent cytotoxicity of the tumor cell in vitro with an IC₅₀ of less than about 100 pM. In some embodiments, the multispecific antibody induces NK cell dependent cytotoxicity of the tumor cell in vitro with an IC₅₀ of less than about 50 pM. In some embodiments, the multispecific antibody induces NK cell dependent cytotoxicity of the tumor cell in vitro with an IC₅₀ of less than about 20 pM. In some embodiments, the multispecific antibody induces NK cell dependent cytotoxicity of the tumor cell in vitro with an IC₅₀ of less than about 15 pM. In some embodiments, the multispecific antibody induces NK cell dependent cytotoxicity of the tumor cell in vitro with an IC₅₀ of less than about 10 pM.

In some embodiments of the process for making a multispecific antibody provided herein, the IC₅₀ is assessed with a mixture of NK effector cells and target cells expressing the second antigen.

In some embodiments of the process for making a multispecific antibody provided herein, the effector cell to target cell ratio is about 0.01 to 1 to about 10 to 1. In some embodiments of the process for making a multispecific antibody provided herein, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1.

In another aspect, provided herein is a method of directing an NK cell to a target cell, comprising contacting the NK cell with a multispecific antibody, thereby directing the NK cell to the target cell, wherein the multispecific antibody comprises a first binding domain that binds to a first antigen on an NK cell and a second binding domain that binds to a second antigen on a target cell.

In another aspect, provided herein is use of a multispecific antibody to direct an NK cell to a target cell, comprising contacting the NK cell with the multispecific antibody, thereby directing the NK cell to the target cell, wherein the multispecific antibody comprises a first binding domain that binds to a first antigen on an NK cell and a second binding domain that binds to a second antigen on a target cell.

In another aspect, provided herein is a method of activating an NK cell, comprising contacting the NK with a multispecific antibody, wherein the multispecific antibody comprises a first binding domain that binds to a first antigen on the NK cell and a second binding domain that binds to a second antigen on a target cell.

In another aspect, provided herein is use of a multispecific antibody to activate an NK cell, comprising contacting the NK with the multispecific antibody, wherein the multispecific antibody comprises a first binding domain that binds to a first antigen on the NK cell and a second binding domain that binds to a second antigen on a target cell.

In another aspect, provided herein is a method of inhibiting growth or proliferation of target cells expressing a second antigen on the cell surface, the method comprising contacting the target cells with a multispecific antibody, wherein the multispecific antibody comprises a first binding domain that binds to a first antigen on an NK cell and a second binding domain that binds to the second antigen.

In another aspect, provided herein is use of a multispecific antibody to inhibit growth or proliferation of target cells expressing a second antigen on the cell surface, the use of the multispecific antibody comprising contacting the target cells with the multispecific antibody, wherein the multispecific antibody comprises a first binding domain that binds to a first antigen on an NK cell and a second binding domain that binds to the second antigen.

In another aspect, provided herein is a method for eliminating target cells expressing a second antigen or treating a disease or disorder caused all or in part by target cells expressing the second antigen in a subject, comprising administering an effective amount of a multispecific antibody to the subject, wherein the multispecific antibody comprises a first binding domain that binds to a first antigen on an NK cell and a second binding domain that binds to the second antigen.

In another aspect, provided herein is use of a multispecific antibody for eliminating target cells expressing a second antigen or treating a disease or disorder caused all or in part by target cells expressing the second antigen in a subject, comprising administering an effective amount of the multispecific antibody to the subject, wherein the multispecific antibody comprises a first binding domain that binds to a first antigen on an NK cell and a second binding domain that binds to the second antigen.

In some embodiments, the subject is a subject in need thereof. In some embodiments, the subject is a human.

In some embodiments, the disease or disorder is cancer. In some embodiments, the cancer is a blood cancer. In some embodiments, the cancer is a solid tumor cancer.

In some embodiments of the method provided herein, the first antigen is an NK cell activating receptor.

In some embodiments of the method provided herein, the first antigen is NKG2d. In some embodiments of the method provided herein, the first antigen is NKp46. In some embodiments, the multispecific antibody is a multispecific antibody provided herein.

In some embodiments of the method provided herein, the second antigen is on a cell surface.

In some embodiments of the method provided herein, the second antigen is expressed on a tumor cell.

In some embodiments of the method provided herein, the second antigen is a tumor specific antigen (TSA) or a tumor associated antigen (TAA).

In some embodiments of the method provided herein, the second antigen is BCMA.

In some embodiments of the method provided herein, the second antigen is GPRC5d.

In some embodiments of the method provided herein, the first binding domain is humanized. In some embodiments of the method provided herein, the second binding domain is humanized. In some embodiments of the method provided herein, both the first binding domain and the second binding domain are humanized.

In some embodiments of the method provided herein, the multispecific antibody is an IgG antibody.

In some embodiments of the method provided herein, the IgG antibody is an IgG1, IgG2, IgG3, or IgG4 antibody.

In some embodiments of the method provided herein, the IgG antibody is an IgG1 antibody.

In some embodiments of the method provided herein, the IgG1 comprise silent mutations. In some embodiments of the method provided herein, the IgG1 comprise AAS mutations.

In some embodiments of the method provided herein, the IgG1 comprise mutations for enhancement of an effector function of the antibody. In some embodiments of the method provided herein, the IgG1 comprise K248E/T437R mutations.

In some embodiments of the method provided herein, the Fc region is afucosylated.

In some embodiments of the method provided herein, the multispecific antibody is a bispecific antibody.

In some embodiments of the method provided herein, the bispecific antibody is in a bipod-scaffold configuration.

In some embodiments of the method provided herein, the first binding domain is a Fab region, and the second binding domain is a scFv region.

In some embodiments of the method provided herein, the bispecific antibody is in a Morrison-scaffold configuration.

In some embodiments of the method provided herein, the first binding domain comprises two Fab regions and the second binding domain comprises two scFv regions.

In some embodiments of the method provided herein, the multispecific antibody induces NK cell dependent cytotoxicity of the tumor cell in vitro with an IC50 of less than about 500 pM. In some embodiments, the multispecific antibody induces NK cell dependent cytotoxicity of the tumor cell in vitro with an IC50 of less than about 300 pM. In some embodiments, the multispecific antibody induces NK cell dependent cytotoxicity of the tumor cell in vitro with an IC50 of less than about 100 pM. In some embodiments, the multispecific antibody induces NK cell dependent cytotoxicity of the tumor cell in vitro with an IC50 of less than about 50 pM. In some embodiments, the multispecific antibody induces NK cell dependent cytotoxicity of the tumor cell in vitro with an IC50 of less than about 20 pM. In some embodiments, the multispecific antibody induces NK cell dependent cytotoxicity of the tumor cell in vitro with an IC50 of less than about 15 pM. In some embodiments, the multispecific antibody induces NK cell dependent cytotoxicity of the tumor cell in vitro with an IC50 of less than about 10 pM.

In some embodiments of the method provided herein, the IC50 is assessed with a mixture of NK effector cells and target cells expressing the second antigen.

In some embodiments of the method provided herein, the effector cell to target cell ratio is about 0.01 to 1 to about 10 to 1. In some embodiments of the method provided herein, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 0.5 to 1. In some embodiments, the effector cell to target cell ratio is about 1:1.

In another aspect, provided herein is a molecule comprising a first means for engaging or activating an NK cell, and a second means for binding a tumor cell, wherein the molecule is capable of inducing NK cell dependent cytotoxicity against the tumor cell.

In some embodiments of the molecule provided herein, the first means comprises a first binding domain that binds to a first antigen expressed on the NK cell, and the second means comprises a second binding domain that binds to a second antigen expressed on the tumor cell.

In some embodiments of the molecule provided herein, the first antigen is an NK cell activating receptor.

In some embodiments of the molecule provided herein, the first antigen is NKG2d.

In some embodiments of the molecule provided herein, the first antigen is NKp46.

In some embodiments of the molecule provided herein, the second antigen is a tumor specific antigen (TSA) or a tumor associated antigen (TAA).

In some embodiments of the molecule provided herein, the second antigen is BCMA.

In some embodiments of the molecule provided herein, the second antigen is GPRC5d.

In another aspect, provided herein is a process for making a molecule that binds to more than one target molecule, comprising: a step for performing a function of obtaining a binding domain capable of binding to a first antigen on an NK cell; a step for performing a function of obtaining a binding domain capable of binding to a second antigen on a tumor cell; and a step for performing a function of providing an molecule capable of binding to the first antigen and the second antigen.

In another aspect, provided herein is a method of directing an NK cell to a target cell, comprising contacting the NK cell with a molecule provided herein.

In another aspect, provided herein is a method of activating an NK cell, comprising contacting the NK cell with a molecule provided herein

In another aspect, provided herein is a method of inhibiting growth or proliferation of target cells, the method comprising contacting the target cells with molecule provided herein.

In another aspect, provided herein is a method for eliminating target cells expressing a second antigen or treating a disease or disorder caused all or in part by target cells expressing the second antigen in a subject, comprising administering an effective amount of molecule provided herein.

### 4. BRIEF DESCRIPTION OF THE FIGURES

FIG. 1. illustrates results of Serum Titers for Omnirats immunized with NKGW1.

FIG. 2. illustrates bead based assay for NK cell agonism.

FIG. 3. illustrates results of Serum Titers for Omnirats immunized with NKp46.

FIG. 4. illustrates the configuration of the bsAbs.

FIGS. 5A-5D. illustrate results of analysis of the abilities of BsAbs to mediate NK cell-based cytotoxicity.

FIG. 6 illustrates the configuration of additional BCMA-binding monovalent mAb and bsAbs.

FIG. 7 illustrates the depotentiation of NK cells in ADCC by 72-hour pre-treatment with 1 ng/ml TGFb and functional rescue of ADCC by incorporation of the NKp46 binding arm in the effector molecule N46B10.AFU.

FIGS. 8A-8C illustrate the potentiation of NK cells in ADCC under hypoxic conditions by incorporation of the NKp46 binding arm in the effector molecule N46B10.AFU compared to BCMB1106.AFU which does not contain the NKp46 binding arm.

FIG. 9 illustrates the lack of anti-NK CDC activity by the BCMA x NKp46 bispecific molecules in the presence of human serum, in contrast to the anti-CD38 positive control mAb which shows titratable anti-NK CDC killing.

### 5. DETAILED DESCRIPTION

The present disclosure is based in part on the novel molecules that bind to an antigen on an NK cell and multispecific binding molecules comprising same or fragment thereof, and the advanced properties of these novel molecules, such as molecules comprising a first means capable of binding to a first antigen present on an NK cell; and a second means capable of binding to a second antigen, e.g., on a tumor cell.

### 5.1. Definitions

Techniques and procedures described or referenced herein include those that are generally well understood and/or commonly employed using conventional methodology by those skilled in the art, such as, for example, the widely utilized methodologies described in Molecular Cloning: A Laboratory Manual (Sambrook, et al., 3d ed. 2001); Current Protocols in Molecular Biology (Ausubel, et al. eds., 2003); Therapeutic Monoclonal Antibodies: From Bench to Clinic (An, ed. 2009); Monoclonal Antibodies: Methods and Protocols (Albitar, ed. 2010); and Antibody Engineering Vols 1 and 2 (Kontermann and Dübel, eds., 2d ed. 2010).

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multispecific antibodies (e.g., bispecific antibodies so long as they exhibit the desired biological activity), formed from at least two intact antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See. e.g., Antibody Engineering (Borrebaeck, ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies or their humanized variants, intrabodies, and anti-idiotypic (anti-Id) antibodies. The term “antibody” as used herein also comprises any binding molecule having a Fc region and a functional fragment (e.g., an antigen-binding fragment) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers, ed., 1995); Huston, et al., 1993, Cell Biophysics 22:189-224; Plückthun and Skerra, 1989, Meth. Enzymol. 178:497-515; and Day, Advanced Immunochemisry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen binding site as well as a constant domain (CL) and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the antibody described herein, the terms such as “bind to,” “that specifically bind to,” and analogous terms are also used interchangeably herein and refer to antibodies of antigen binding domains that specifically bind to an antigen, such as a polypeptide. An antibody or antigen binding domain that binds to or specifically binds to an antigen may be cross-reactive with related antigens. In certain embodiments, an antibody or antigen binding domain that binds to or specifically binds to an antigen does not cross-react with other antigens. An antibody or antigen binding domain that binds to or specifically binds to an antigen can be identified, for example, by immunoassays, Octet®, Biacore®, or other techniques known to those of skill in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul, ed., 2d ed. 1989) for a discussion regarding binding specificity. In certain embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. With regard to terms such as “specific binding,” “specifically binds to,” or “is specific for” means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. An antibody or antigen binding domain that binds to an antigen includes one that is capable of binding the antigen with sufficient affinity such that the antibody is useful, for example, as a diagnostic or therapeutic agent in targeting the antigen. In certain embodiments, an antibody or antigen binding domain that binds to an antigen has a dissociation constant (K_{D}) of less than or equal to 1000 nM, 800 nM, 500 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, an antibody or antigen binding domain binds to an epitope of an antigen that is conserved among the antigen from different species (e.g., between human and cynomolgus macaque species).

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen, et al., J. Mol Biol, 1999, 293:865-81). The K_{D} or K_{D} value may also be measured by using biolayer interferometry (BLI) or surface plasmon resonance (SPR) assays by Octet®, using, for example, an Octet® Red96 system, or by Biacore®, using, for example, a Biacore® 2000 or a Biacore® 3000. An “on-rate” or “rate of association” or “association rate” or “k” may also be determined with the same biolayer interferometry (BLI) or surface plasmon resonance (SPR) techniques described above using, for example, the Octet® Red96, the Biacore® 2000, or the Biacore® 3000 system.

In certain embodiments, the antibodies can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, so long as they exhibit the desired biological activity (see U.S. Pat. No. 4,816,567; and Morrison, et al., Proc. Natl. Acad. Sci. USA, 1984, 81:6851-55). Chimeric sequences may include humanized sequences.

In certain embodiments, the antibodies can comprise portions of “humanized” forms of nonhuman (e.g., murine) antibodies that are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones, et al., Nature, 1986, 321:522-25; Riechmann, et al., Nature, 1988, 332:323-29; Presta, Curr. Op. Struct. Biol., 1992, 2:593-96; Carter, et al., Proc. Natl. Acad. Sci. USA, 1992, 89:4285-89; U.S. Pat. Nos. 6,800,738; 6,719,971; 6,639,055; 6,407,213; and 6,054,297.

In certain embodiments, the antibodies can comprise portions of a “fully human antibody” or “human antibody,” wherein the terms are used interchangeably herein and refer to an antibody that comprises a human variable region and, for example, a human constant region. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat, et al. (see Kabat, et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol., 1991, 227:381; Marks, et al., 1991, J. Mol. Biol., 1991, 222:581) and yeast display libraries (Chao, et al., Nature Protocols, 2006, 1: 755-68). Also available for the preparation of human monoclonal antibodies are methods described in Cole, et al., Monoclonal Antibodies and Cancer Therapy 77 (1985); Boerner, et al., J. Immunol., 1991, 147(1):86-95; and van Dijk and van de Winkel, Curr. Opin. Pharmacol., 2001, 5: 368-74. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol., 1995, 6(5):561-66; Brüggemann and Taussing, Curr. Opin. Biotechnol., 1997, 8(4):455-58; and U.S. Pat. Nos. 6,075,181 and 6,150,584 regarding XENOMOUSE™ technology). See also, for example, Li, et al., Proc. Natl. Acad. Sci. USA, 2006, 103:3557-62, regarding human antibodies generated via a human B-cell hybridoma technology.

In certain embodiments, the antibodies can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D., et al., Nucl. Acids Res., 1992 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the antibodies can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson, et al., Nature, 1991, 352:624-28 and Marks, et al., J. Mol. Biol., 1991, 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for p and F isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites, et al. eds., 8th ed. 1994); and Immunobiology (Janeway, et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CH1 regions, and the variable region and the constant region of the light chain in a Fab region are VL and CL regions. The VH, CH1, VL, and CL in a Fab region can be arranged in various ways to confer an antigen binding capability according to the present disclosure. For example, VH and CH1 regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see. e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat, et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat, et al., supra). The “EU numbering system” or “EU index” is generally used when referring to a residue in an immunoglobulin heavy chain constant region (e.g., the EU index reported in Kabat, et al., supra). The “EU index as in Kabat” refers to the residue numbering of the human IgG1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see. e.g., Kabat, et al., supra). Chothia refers instead to the location of the structural loops (see, e.g., Chothia and Lesk, J. Mol. Biol., 1987, 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel, eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc, et al., Dev. Comp. Immunol., 2003, 27(1):55-77). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol., 2001, 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc, et al., supra). The residues from each of these hypervariable regions or CDRs are noted below.

| Loop | Kabat | AbM | Chothia | Contact | IMGT |
|---|---|---|---|---|---|
| CDR L1 | L24--L34 | L24--L34 | L24--L34 | L30--L36 | L27--L38 |
| CDR L2 | L50--L56 | L50--L56 | L50--L56 | L46--L55 | L56--L65 |
| CDR L3 | L89--L97 | L89--L97 | L89--L97 | L89--L96 | L105-L117 |
| CDR H1 | H31--H35B | H26--H35B | H26--H32 . . . 34 | H30--H35B | H27--H38 |
| | (Kabat | | | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H52--H56 | H47--H58 | H56--H65 |
| CDR H3 | H95--H102 | H95--H102 | H95--H102 | H93--H101 | H105-H117 |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the Kabat, Chothia, or Contact method. In other cases, the particular amino acid sequence of a CDR is given.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues. There are typically four FR regions in each of VH and VL regions. The FR regions in VH are VH FR1, VH FR2, VH FR3, and VH FR4 (or FR H1, FR H2, FR H3 and FR H4). The FR regions in VL are VL FR1, VL FR2, VL FR3 and VL FR4 (or FR L1, FR L2, FR L3 and FR L4).

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “variant” when used in relation to an antigen or an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

As used herein, an “epitope” is a term in the art and refers to a localized region of an antigen to which an antibody can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

The term “vector” refers to a substance that is used to carry or include a nucleic acid sequence, including for example, a nucleic acid sequence encoding an antibody as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide,” “nucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antibody of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on an NKG2d or an NKp46 antigen) and a second immunoglobulin variable domain sequence that has binding specificity for a second epitope (e.g., an epitope on a tumor-associated antigen (e.g., a BCMA or a GPRC5d antigen). In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on NKG2d and the second epitope is located on BCMA. In an embodiment, the first epitope is located on NKG2d and the second epitope is located on GPRC5d. In an embodiment, the first epitope is located on NKp46 and the second epitope is located on BCMA. In an embodiment, the first epitope is located on NKp46 and the second epitope is located on GPRC5d.

As used herein, the term “NKG2d” refers to NKG2-D type II integral membrane protein. NKG2d can also be referred to as the “NK cell receptor D”, “NKG2-D-activating NK receptor”, “CD314.” The term “NKG2d” includes any NKG2d variant, isoform, and species homolog, which is naturally expressed by cells (including NK cells) or can be expressed on cells transfected with genes or cDNA encoding those polypeptides, unless noted, in specific embodiments the “NKG2d” is a human NKG2d.

As used herein, the term “NKp46” refers to Natural killer cell p46-related protein. NKp46 can also be referred to as the “Natural cytotoxicity triggering receptor 1”, “NK-p46”, “CD335.” The term “NKp46” includes any NKp46 variant, isoform, and species homolog, which is naturally expressed by cells (including NK cells) or can be expressed on cells transfected with genes or cDNA encoding those polypeptides, unless noted, in specific embodiments the “NKp46” is a human NKp46.

As used herein, the term “BCMA” refers to B-cell maturation antigen, also referred to as TNFRSF17 or CD269, is a member of the tumor necrosis factor receptor (TNFR) superfamily. The term “BCMA” includes any BCMA variant, isoform, and species homolog, which is naturally expressed by cells (including cancer cells) or can be expressed on cells transfected with genes or cDNA encoding those polypeptides, unless noted, in specific embodiments the “BCMA” is a human BCMA.

As used herein, the term “GPRC5d” refers to G-protein coupled receptor family C group 5 member D. The term “GPRC5d” includes any GPRC5d variant, isoform, and species homolog, which is naturally expressed by cells (including cancer cells) or can be expressed on cells transfected with genes or cDNA encoding those polypeptides, unless noted, in specific embodiments the “GPRC5d” is a human GPRC5d.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete)), or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain an antibody, for example, in isolated or purified form, together with a suitable amount of excipients.

The term “effective amount” or “therapeutically effective amount” as used herein refers to the amount of an antibody or pharmaceutical composition provided herein which is sufficient to result in the desired outcome.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular, subcutaneous delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s).

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Binding Molecules

### 5.2.1. NK Cell Binding Molecules

In one aspect, provided herein is binding molecule, such as an antibody or fragment thereof, that binds to a cell surface antigen of NK cells. In some embodiments, the antigen is NKG2d. In some embodiments, the antigen is NKp46.

In one aspect, provided herein is an antibody that binds to NKG2d. In some embodiments, the antibody comprises a heavy chain variable region and a light chain variable region. In some embodiments, the NKG2d antibody is not a single domain antibody or nanobody. In some embodiments, the NKG2d antibody is a humanized antibody.

In some embodiments, provided herein are antibodies that specifically bind to NKG2d and can modulate NK cell activity. In some embodiments, the NKG2d antibody provided herein can modulate the engagement of an NK cell. In some embodiments, the NKG2d antibody provided herein can active an NK cell. In specific embodiments, the NK cells are human NK cells.

In certain embodiments, provided herein is an anti-NKG2d antibody comprising a VH region, VL region, VH CDR1, VH CDR2, VH CDR3, VL CDR1, VL CDR2, and/or VL CDR3 of any one of the antibodies described herein. In some embodiments, provided herein is an anti-NKG2d antibody comprising a VH region of any one of the antibodies described herein. In some embodiments, provided herein is an anti-NKG2d antibody comprising a VL region of any one of the antibodies described herein. In some embodiments, provided herein is an anti-NKG2d antibody comprising a VH region of any one of the antibodies described herein, and a VL region of any one of the antibodies described herein. In some embodiments, provided herein is an anti-NKG2d antibody comprising a VH CDR1, VH CDR2, and VH CDR3 of any one of the antibodies described herein. In some embodiments, provided herein is an anti-NKG2d antibody comprising a VL CDR1, VL CDR2, and VL CDR3 of any one of the antibodies described herein. In some embodiments, provided herein is an anti-NKG2d antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of NKG2d antibodies provided herein are provided in the Sequence Listing, as well as Tables 3, 4, 7 and 8.

In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In certain embodiments, provided is an anti-NKG2d antibody that is an intact antibody. In other embodiments, provided is an anti-NKG2d antibody is an antigen binding fragment of the anti-NKG2d antibody. In some embodiments, the antigen binding fragment of the anti-NKG2d antibody is a functional fragment. In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)₂. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In specific embodiments, the NKG2d antibody comprises a VH region and a VL region. In some embodiments, the NKG2d antibody is a single chain antibody. In some embodiments, the NKG2d antibody is a single domain antibody. In some embodiments, the antigen binding fragment is a camelized single domain antibody. In some embodiments, the NKG2d antibody is a nanobody. In certain embodiments, the NKG2d antibody is a VHH antibody. In certain embodiments, the NKG2d antibody is a llama antibody. In some embodiments, the NKG2d antibody is not a single chain antibody. In some embodiments, the NKG2d antibody is not a single domain antibody. In some embodiments, the NKG2d antibody is not a nanobody. In certain embodiments, the NKG2d antibody is not a VHH antibody. In certain embodiments, the NKG2d antibody is not a llama antibody. In some embodiments, the NKG2d antibody is a multispecific antibody. In other embodiments, the NKG2d is a bispecific antibody. In certain embodiments, the multispecific antibody comprises an antigen binding fragment of an NKG2d antibody provided herein. In other embodiments, the bispecific antibody comprises an antigen binding fragment of an NKG2d antibody provided herein. In some embodiments, the NKG2d antibody is an agonistic antibody. In certain embodiments, the NKG2d antibody activates NK cells. In some embodiments, the NKG2d antibody modulates the activity of NK cells. In some embodiments, the NKG2d antibody neither activates or inactivates the activity of NK cells. In specific embodiments, the NK cells are human NK cells.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In some embodiments, the antibody provided herein binds NKG2d. In some embodiments, the antibody that binds NKG2d comprises a VH comprising a VH CDR1, a VH CDR2, and a VH CDR3 having an amino acid sequence of a VH CDR1, a VH CDR2, and a VH CDR3, respectively, of SEQ ID NO:2. In some embodiments, the antibody that binds NKG2d comprises a VL comprising a VL CDR1, a VL CDR2, and a VL CDR3 having an amino acid sequence of a VL CDR1, a VL CDR2, and a VL CDR3, respectively, of SEQ ID NO:3. In some embodiments, the antibody that binds NKG2d comprises: (i) a VH comprising a VH CDR1, a VH CDR2, and a VH CDR3 having an amino acid sequence of a VH CDR1, a VH CDR2, and a VH CDR3, respectively, of SEQ ID NO:2; and (ii) a VL comprising a VL CDR1, a VL CDR2, and a VL CDR3 having an amino acid sequence of a VL CDR1, a VL CDR2, and a VL CDR3, respectively, of SEQ ID NO:3. In some embodiments, the CDR1, CDR2 or CDR3 are determined according to the Kabat numbering scheme, the IMGT numbering scheme, the AbM numbering scheme, the Chothia numbering scheme, the Contact numbering scheme, or a combination thereof. In some embodiments, the antibody that binds NKG2d comprises a VH having an amino acid sequence of SEQ ID NO:2. In some embodiments, the antibody that binds NKG2d comprises a VL having an amino acid sequence of SEQ ID NO:3. In some embodiments, the antibody that binds NKG2d comprises a VH having an amino acid sequence of SEQ ID NO:2, and a VL having an amino acid sequence of SEQ ID NO:3.

In some embodiments, the antibody that binds NKG2d comprises: (i) a VH comprising a VH CDR1, a VH CDR2, and a VH CDR3 having an amino acid sequence of SEQ ID NOs:4, 5, and 6, respectively, and (ii) a VL comprising a VL CDR1, VL CDR2, and VL CDR3 having an amino acid sequence of SEQ ID NOs:7, 8, and 9, respectively. In some embodiments, the antibody that binds NKG2d comprises: (i) a VH comprising a VH CDR1, a VH CDR2, and a VH CDR3 having an amino acid sequence of SEQ ID NOs:10, 11, and 12, respectively, and (ii) a VL comprising a VL CDR1, VL CDR2, and VL CDR3 having an amino acid sequence of SEQ ID NOs:13, 14, and 15, respectively. In some embodiments, the antibody that binds NKG2d comprises: (i) a VH comprising a VH CDR1, a VH CDR2, and a VH CDR3 having an amino acid sequence of SEQ ID NOs:16, 17, and 18, respectively, and (ii) a VL comprising a VL CDR1, VL CDR2, and VL CDR3 having an amino acid sequence of SEQ ID NOs:19, 20, and 21, respectively. In some embodiments, the antibody that binds NKG2d comprises: (i) a VH comprising a VH CDR1, a VH CDR2, and a VH CDR3 having an amino acid sequence of SEQ ID NOs:22, 23, and 24, respectively, and (ii) a VL comprising a VL CDR1, VL CDR2, and VL CDR3 having an amino acid sequence of SEQ ID NOs:25, 26, and 27, respectively. In some embodiments, the antibody that binds NKG2d comprises: (i) a VH comprising a VH CDR1, a VH CDR2, and a VH CDR3 having an amino acid sequence of SEQ ID NOs:28, 29, and 30, respectively, and (ii) a VL comprising a VL CDR1, VL CDR2, and VL CDR3 having an amino acid sequence of SEQ ID NOs:31, 32, and 33, respectively.

In some embodiments, the antibody further comprises one or more framework region(s) of SEQ ID NO:2 and/or SEQ ID NO:3. In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody that binds NKG2d comprises a VH comprising a VH CDR1, a VH CDR2, and a VH CDR3 having an amino acid sequence of a VH CDR1, a VH CDR2, and a VH CDR3, respectively, of SEQ ID NO:34. In some embodiments, the antibody that binds NKG2d comprises a VL comprising a VL CDR1, a VL CDR2, and a VL CDR3 having an amino acid sequence of a VL CDR1, a VL CDR2, and a VL CDR3, respectively, of SEQ ID NO:35. In some embodiments, the antibody that binds NKG2d comprises: (i) a VH comprising a VH CDR1, a VH CDR2, and a VH CDR3 having an amino acid sequence of a VH CDR1, a VH CDR2, and a VH CDR3, respectively, of SEQ ID NO:34; and (ii) a VL comprising a VL CDR1, a VL CDR2, and a VL CDR3 having an amino acid sequence of a VL CDR1, a VL CDR2, and a VL CDR3, respectively, of SEQ ID NO:35. In some embodiments, the CDR1, CDR2 or CDR3 are determined according to the Kabat numbering scheme, the IMGT numbering scheme, the AbM numbering scheme, the Chothia numbering scheme, the Contact numbering scheme, or a combination thereof. In some embodiments, the antibody that binds NKG2d comprises a VH having an amino acid sequence of SEQ ID NO:34. In some embodiments, the antibody that binds NKG2d comprises a VL having an amino acid sequence of SEQ ID NO:35. In some embodiments, the antibody that binds NKG2d comprises a VH having an amino acid sequence of SEQ ID NO:34, and a VL having an amino acid sequence of SEQ ID NO:35.

In some embodiments, the antibody that binds NKG2d comprises: (i) a VH comprising a VH CDR1, a VH CDR2, and a VH CDR3 having an amino acid sequence of SEQ ID NOs:36, 37, and 38, respectively, and (ii) a VL comprising a VL CDR1, VL CDR2, and VL CDR3 having an amino acid sequence of SEQ ID NOs:39, 40, and 41, respectively. In some embodiments, the antibody that binds NKG2d comprises: (i) a VH comprising a VH CDR1, a VH CDR2, and a VH CDR3 having an amino acid sequence of SEQ ID NOs:42, 43, and 44, respectively, and (ii) a VL comprising a VL CDR1, VL CDR2, and VL CDR3 having an amino acid sequence of SEQ ID NOs:45, 46, and 47, respectively. In some embodiments, the antibody that binds NKG2d comprises: (i) a VH comprising a VH CDR1, a VH CDR2, and a VH CDR3 having an amino acid sequence of SEQ ID NOs:48, 49, and 50, respectively, and (ii) a VL comprising a VL CDR1, VL CDR2, and VL CDR3 having an amino acid sequence of SEQ ID NOs:51, 52, and 53, respectively. In some embodiments, the antibody that binds NKG2d comprises: (i) a VH comprising a VH CDR1, a VH CDR2, and a VH CDR3 having an amino acid sequence of SEQ ID NOs:54, 55, and 56, respectively, and (ii) a VL comprising a VL CDR1, VL CDR2, and VL CDR3 having an amino acid sequence of SEQ ID NOs:57, 58, and 59, respectively. In some embodiments, the antibody that binds NKG2d comprises: (i) a VH comprising a VH CDR1, a VH CDR2, and a VH CDR3 having an amino acid sequence of SEQ ID NOs:60, 61, and 62, respectively, and (ii) a VL comprising a VL CDR1, VL CDR2, and VL CDR3 having an amino acid sequence of SEQ ID NOs:63, 64, and 65, respectively.

In some embodiments, the antibody further comprises one or more framework region(s) of SEQ ID NO:34 and/or SEQ ID NO:35. In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In certain embodiments, an antibody described herein or an antigen-binding fragment thereof comprises amino acid sequences with certain percent identity relative to any one of the above described antibodies.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, there is provided an anti-NKG2d antibody comprising a VH having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:2. In some embodiments, there is provided an anti-NKG2d antibody comprising a VL having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:3. In some embodiments, there is provided an anti-NKG2d antibody comprising a VH having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:2, and a VL having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:3.

In some embodiments, there is provided an anti-NKG2d antibody comprising a VH having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:34. In some embodiments, there is provided an anti-NKG2d antibody comprising a VL having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:35. In some embodiments, there is provided an anti-NKG2d antibody comprising a VH having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:34, and a VL having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:35.

In some embodiments, a VH or a VL sequence having at least about any one of 75%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the antibody comprising that sequence retains the ability to bind to NKG2d. In some embodiments, a total of 1 to 10 amino acids have been substituted, inserted and/or deleted. In some embodiments, substitutions, insertions, or deletions occur in regions outside the CDRs (i.e., in the FRs).

In another aspect, provided herein is an antibody that competes for binding to NKG2d with any of the NKG2d antibodies described herein. In another aspect, provided herein is an antibody that binds to the same epitope as any of the NKG2d antibodies described herein. In another aspect, provided is an NKG2d antibody that binds an epitope on NKG2d that overlaps with the epitope on NKG2d bound by an NKG2d antibody described herein.

In one aspect, provided is an antibody that competes for binding to NKG2d with an NKG2d reference antibody. In another aspect, provided is an NKG2d antibody that binds to the same NKG2d epitope as an NKG2d reference antibody. In another aspect, provided is an NKG2d antibody that binds an epitope on NKG2d that overlaps with the epitope on NKG2d bound by an NKG2d reference antibody.

In one embodiment, the NKG2d reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3. In one embodiment, the NKG2d reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:35.

In one aspect, provided herein is an antibody that binds to NKp46. In some embodiments, the antibody comprises a heavy chain variable region and a light chain variable region. In some embodiments, the NKp46 antibody is not a single domain antibody or nanobody. In some embodiments, the NKp46 antibody is a humanized antibody.

In some embodiments, provided herein are antibodies that specifically bind to NKp46 and can modulate NK cell activity. In some embodiments, the NKp46 antibody provided herein can modulate the engagement of an NK cell. In some embodiments, the NKp46 antibody provided herein can active an NK cell. In specific embodiments, the NK cells are human NK cells.

In certain embodiments, provided herein is an anti-NKp46 antibody comprising a VH region, VL region, VH CDR1, VH CDR2, VH CDR3, VL CDR1, VL CDR2, and/or VL CDR3 of any one of the antibodies described herein. In some embodiments, provided herein is an anti-NKp46 antibody comprising a VH region of any one of the antibodies described herein. In some embodiments, provided herein is an anti-NKp46 antibody comprising a VL region of any one of the antibodies described herein. In some embodiments, provided herein is an anti-NKp46 antibody comprising a VH region of any one of the antibodies described herein, and a VL region of any one of the antibodies described herein. In some embodiments, provided herein is an anti-NKp46 antibody comprising a VH CDR1, VH CDR2, and VH CDR3 of any one of the antibodies described herein. In some embodiments, provided herein is an anti-NKp46 antibody comprising a VL CDR1, VL CDR2, and VL CDR3 of any one of the antibodies described herein. In some embodiments, provided herein is an anti-NKp46 antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of NKp46 antibodies provided herein are provided in the Sequence Listing, as well as Tables 14-15.

In some embodiments, the antibody is a humanized antibody. In certain embodiments, the antibody is an IgG antibody. In other embodiments, the IgG antibody is an IgG1, IgG2, IgG3, or IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In certain embodiments, provided is an NKp46 antibody that is an intact antibody. In other embodiments, provided is an NKp46 antibody is an antigen binding fragment of the NKp46 antibody. In some embodiments, the antigen binding fragment of the NKp46 antibody is a functional fragment. In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)₂. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In specific embodiments, the NKp46 antibody comprises a VH region and a VL region. In some embodiments, the NKp46 antibody is a single chain antibody. In some embodiments, the NKp46 antibody is a single domain antibody. In some embodiments, the antigen binding fragment is a camelized single domain antibody. In some embodiments, the NKp46 antibody is a nanobody. In certain embodiments, the NKp46 antibody is a VHH antibody. In certain embodiments, the NKp46 antibody is a llama antibody. In some embodiments, the NKp46 antibody is not a single chain antibody. In some embodiments, the NKp46 antibody is not a single domain antibody. In some embodiments, the NKp46 antibody is not a nanobody. In certain embodiments, the NKp46 antibody is not a VHH antibody. In certain embodiments, the NKp46 antibody is not a llama antibody. In some embodiments, the NKp46 antibody is a multispecific antibody. In other embodiments, the NKp46 is a bispecific antibody. In certain embodiments, the multispecific antibody comprises an antigen binding fragment of an NKp46 antibody provided herein. In other embodiments, the bispecific antibody comprises an antigen binding fragment of an NKp46 antibody provided herein. In some embodiments, the NKp46 antibody is an agonistic antibody. In certain embodiments, the NKp46 antibody activates NK cells. In some embodiments, the NKp46 antibody modulates the activity of NK cells. In some embodiments, the NKp46 antibody neither activates or inactivates the activity of NK cells. In specific embodiments, the NK cells are human NK cells.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In some embodiments, the antibody provided herein binds NKp46. In some embodiments, the antibody that binds NKp46 comprises a VH comprising a VH CDR1, a VH CDR2, and a VH CDR3 having an amino acid sequence of a VH CDR1, a VH CDR2, and a VH CDR3, respectively, of SEQ ID NO:67. In some embodiments, the antibody that binds NKp46 comprises a VL comprising a VL CDR1, a VL CDR2, and a VL CDR3 having an amino acid sequence of a VL CDR1, a VL CDR2, and a VL CDR3, respectively, of SEQ ID NO:68. In some embodiments, the antibody that binds NKp46 comprises: (i) a VH comprising a VH CDR1, a VH CDR2, and a VH CDR3 having an amino acid sequence of a VH CDR1, a VH CDR2, and a VH CDR3, respectively, of SEQ ID NO:67; and (ii) a VL comprising a VL CDR1, a VL CDR2, and a VL CDR3 having an amino acid sequence of a VL CDR1, a VL CDR2, and a VL CDR3, respectively, of SEQ ID NO:68. In some embodiments, the CDR1, CDR2 or CDR3 are determined according to the Kabat numbering scheme, the IMGT numbering scheme, the AbM numbering scheme, the Chothia numbering scheme, the Contact numbering scheme, or a combination thereof. In some embodiments, the antibody that binds NKp46 comprises a VH having an amino acid sequence of SEQ ID NO:67. In some embodiments, the antibody that binds NKp46 comprises a VL having an amino acid sequence of SEQ ID NO:68. In some embodiments, the antibody that binds NKp46 comprises a VH having an amino acid sequence of SEQ ID NO:67, and a VL having an amino acid sequence of SEQ ID NO:68.

In some embodiments, the antibody that binds NKp46 comprises: (i) a VH comprising a VH CDR1, a VH CDR2, and a VH CDR3 having an amino acid sequence of SEQ ID NOs:69, 70, and 71, respectively, and (ii) a VL comprising a VL CDR1, VL CDR2, and VL CDR3 having an amino acid sequence of SEQ ID NOs:72, 73, and 74, respectively. In some embodiments, the antibody that binds NKp46 comprises: (i) a VH comprising a VH CDR1, a VH CDR2, and a VH CDR3 having an amino acid sequence of SEQ ID NOs:75, 76, and 77, respectively, and (ii) a VL comprising a VL CDR1, VL CDR2, and VL CDR3 having an amino acid sequence of SEQ ID NOs:78, 79, and 80, respectively. In some embodiments, the antibody that binds NKp46 comprises: (i) a VH comprising a VH CDR1, a VH CDR2, and a VH CDR3 having an amino acid sequence of SEQ ID NOs:81, 82, and 83, respectively, and (ii) a VL comprising a VL CDR1, VL CDR2, and VL CDR3 having an amino acid sequence of SEQ ID NOs:84, 85, and 86, respectively. In some embodiments, the antibody that binds NKp46 comprises: (i) a VH comprising a VH CDR1, a VH CDR2, and a VH CDR3 having an amino acid sequence of SEQ ID NOs:87, 88, and 89, respectively, and (ii) a VL comprising a VL CDR1, VL CDR2, and VL CDR3 having an amino acid sequence of SEQ ID NOs:90, 91, and 92, respectively. In some embodiments, the antibody that binds NKp46 comprises: (i) a VH comprising a VH CDR1, a VH CDR2, and a VH CDR3 having an amino acid sequence of SEQ ID NOs:93, 94, and 95, respectively, and (ii) a VL comprising a VL CDR1, VL CDR2, and VL CDR3 having an amino acid sequence of SEQ ID NOs:96, 97, and 98 respectively.

In some embodiments, the antibody further comprises one or more framework region(s) of SEQ ID NO:67 and/or SEQ ID NO:68. In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In certain embodiments, an antibody described herein or an antigen-binding fragment thereof comprises amino acid sequences with certain percent identity relative to any one of the above described antibodies. In some embodiments, there is provided an anti-NKp46 antibody comprising a VH having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:67. In some embodiments, there is provided an anti-NKp46 antibody comprising a VL having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:68. In some embodiments, there is provided an anti-NKp46 antibody comprising a VH having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:67, and a VL having at least about any one of 75%, 80%, 85%, 86%, 87%, 88%, 89%, 90%, 91%, 92%, 93%, 94%, 95%, 96%, 97%, 98%, 99%, or 100% sequence identity to an amino acid sequence of SEQ ID NO:68.

In some embodiments, a VH or a VL sequence having at least about any one of 75%, 80%, 85%, 86%, 87%, 88%, 89%, 90%, 91%, 92%, 93%, 94%, 95%, 96%, 97%, 98%, or 99% identity contains substitutions (e.g., conservative substitutions), insertions, or deletions relative to the reference sequence, but the antibody comprising that sequence retains the ability to bind to NKp46. In some embodiments, a total of 1 to 10 amino acids have been substituted, inserted and/or deleted. In some embodiments, substitutions, insertions, or deletions occur in regions outside the CDRs (i.e., in the FRs).

In another aspect, provided herein is an antibody that competes for binding to NKp46 with any of the NKp46 antibodies described herein. In another aspect, provided herein is an antibody that binds to the same epitope as any of the NKp46 antibodies described herein. In another aspect, provided is an NKp46 antibody that binds an epitope on NKp46 that overlaps with the epitope on NKp46 bound by an NKp46 antibody described herein.

In one aspect, provided is an antibody that competes for binding to NKp46 with an NKp46 reference antibody. In another aspect, provided is an NKp46 antibody that binds to the same NKp46 epitope as an NKp46 reference antibody. In another aspect, provided is an NKp46 antibody that binds an epitope on NKp46 that overlaps with the epitope on NKp46 bound by an NKp46 reference antibody.

In one embodiment, the NKp46 reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68.

The antibodies provided herein may be from any animal origin including birds and mammals (e.g., human, monkey, murine, donkey, sheep, rabbit, goat, guinea pig, camel, horse, or chicken). In certain embodiments, the antibodies provided herein are human or humanized monoclonal antibodies. As used herein, “human” antibodies include antibodies having the amino acid sequence of a human immunoglobulin and include antibodies isolated from human immunoglobulin libraries or from mice that express antibodies from human genes.

In certain embodiments, the antibodies are full mouse antibodies. In certain embodiments, the antibodies are mouse-human chimeric antibodies. In certain embodiments, the antibodies are humanized antibodies. In certain embodiments, the antibodies are fully human antibodies. In other embodiments, the antibodies provided herein are humanized antibodies (e.g., comprising human constant and framework regions). The antibodies provided herein may be bispecific, trispecific or of greater multispecificity.

In some embodiments, the antibody or antigen binding fragment provided herein binds NKG2d with a K_{D} of less than 1000 nM. In some embodiments, the antibody or antigen binding fragment provided herein binds NKG2d with a K_{D} of less than 100 nM. In some embodiments, the antibody or antigen binding fragment provided herein binds NKG2d with a K_{D} of less than 50 nM. In some embodiments, the antibody or antigen binding fragment provided herein binds NKG2d with a K_{D} of less than 40 nM. In some embodiments, the antibody or antigen binding fragment provided herein binds NKG2d with a K_{D} of less than 30 nM. In some embodiments, the antibody or antigen binding fragment provided herein binds NKG2d with a K_{D} of less than 20 nM. In some embodiments, the antibody or antigen binding fragment provided herein binds NKG2d with a K_{D} of less than 10 nM. In some embodiments, the antibody or antigen binding fragment provided herein binds NKG2d with a K_{D} of less than 9 nM. In some embodiments, the antibody or antigen binding fragment provided herein binds NKG2d with a K_{D} of less than 8 nM. In some embodiments, the antibody or antigen binding fragment provided herein binds NKG2d with a K_{D} of less than 7 nM. In some embodiments, the antibody or antigen binding fragment provided herein binds NKG2d with a K_{D} of less than 6 nM. In some embodiments, the antibody or antigen binding fragment provided herein binds NKG2d with a K_{D} of less than 5 nM. In some embodiments, the antibody or antigen binding fragment provided herein binds NKG2d with a K_{D} of less than 4 nM. In some embodiments, the antibody or antigen binding fragment provided herein binds NKG2d with a K_{D} of less than 3 nM. In some embodiments, the antibody or antigen binding fragment provided herein binds NKG2d with a K_{D} of less than 2 nM. In some embodiments, the antibody or antigen binding fragment provided herein binds NKG2d with a K_{D} of less than 1 nM. In some embodiments, the antibody or antigen binding fragment provided herein binds NKG2d with a K_{D} of less than 0.1 nM. In some embodiments, the antibody or antigen binding fragment provided herein binds NKG2d with a K_{D} of less than 0.01 nM. The K_{D} or K_{D} value may also be measured by any known methods in the art, for example, using biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system. In a specific embodiment, the K_{D} is determined by a Biacore® assay. In some embodiments, NKG2d is a human NKG2d. In some embodiments, NKG2d is a cynomolgus macaque NKG2d. In some embodiments, NKG2d is a rat NKG2d. In other embodiments, NKG2d is mouse NKG2d.

In other embodiments, the antibody or antigen binding fragment provided herein binds NKp46 with a K_{D} of less than 1000 nM. In some embodiments, the antibody or antigen binding fragment provided herein binds NKp46 with a K_{D} of less than 100 nM. In some embodiments, the antibody or antigen binding fragment provided herein binds NKp46 with a K_{D} of less than 50 nM. In some embodiments, the antibody or antigen binding fragment provided herein binds NKp46 with a K_{D} of less than 40 nM. In some embodiments, the antibody or antigen binding fragment provided herein binds NKp46 with a K_{D} of less than 30 nM. In some embodiments, the antibody or antigen binding fragment provided herein binds NKp46 with a K_{D} of less than 20 nM. In some embodiments, the antibody or antigen binding fragment provided herein binds NKp46 with a K_{D} of less than 10 nM. In some embodiments, the antibody or antigen binding fragment provided herein binds NKp46 with a K_{D} of less than 9 nM. In some embodiments, the antibody or antigen binding fragment provided herein binds NKp46 with a K_{D} of less than 8 nM. In some embodiments, the antibody or antigen binding fragment provided herein binds NKp46 with a K_{D} of less than 7 nM. In some embodiments, the antibody or antigen binding fragment provided herein binds NKp46 with a K_{D} of less than 6 nM. In some embodiments, the antibody or antigen binding fragment provided herein binds NKp46 with a K_{D} of less than 5 nM. In some embodiments, the antibody or antigen binding fragment provided herein binds NKp46 with a K_{D} of less than 4 nM. In some embodiments, the antibody or antigen binding fragment provided herein binds NKp46 with a K_{D} of less than 3 nM. In some embodiments, the antibody or antigen binding fragment provided herein binds NKp46 with a K_{D} of less than 2 nM. In some embodiments, the antibody or antigen binding fragment provided herein binds NKp46 with a K_{D} of less than 1 nM. In some embodiments, the antibody or antigen binding fragment provided herein binds NKp46 with a K_{D} of less than 0.1 nM. In some embodiments, the antibody or antigen binding fragment provided herein binds NKp46 with a K_{D} of less than 0.01 nM. The K_{D} or K_{D} value may also be measured by any known methods in the art, for example, using biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system. In a specific embodiment, the K_{D} is determined by a Biacore® assay. In some embodiments, NKp46 is a human NKp46. In some embodiments, NKp46 is a cynomolgus macaque NKp46. In some embodiments, NKp46 is a rat NKp46. In other embodiments, NKp46 is mouse NKp46.

In some embodiments, provided herein are antibodies that specifically bind to NKG2d and can modulate NK cell activity. In some embodiments, provided herein are antibodies that specifically bind to NKG2d and can modulate NKG2d-expressing immune effector cells activity.

In some embodiments, provided herein are antibodies that specifically bind to NKp46 and can modulate NK cell activity.

In some embodiments, provided herein are antibodies that specifically bind to NKp46 and can modulate NKp46-expressing immune effector cells activity. In some embodiments, the NKp46-expressing immune effector cells are T cells. In some embodiments, the T cells are gamma delta T cells. In some embodiments, the T cells are mucosal population of innate lymphoid cells.

In some embodiments, provided herein are antibodies that specifically bind to NKp46 and can modulate T cells activity. In some embodiments, the T cells are gamma delta T cells. In some embodiments, the T cells are a mucosal population of innate lymphoid cells.

In some embodiments, the antibodies described herein can activate an NK cell. In some embodiments, the antibodies described herein activate an NK cell activity by at least about 10%. In some embodiments, the antibodies described herein activate an NK cell activity by at least about 20%. In some embodiments, the antibodies described herein activate an NK cell activity by at least about 30%. In some embodiments, the antibodies described herein activate an NK cell activity by at least about 40%. In some embodiments, the antibodies described herein activate an NK cell activity by at least about 50%. In some embodiments, the antibodies described herein activate an NK cell activity by at least about 60%. In some embodiments, the antibodies described herein activate an NK cell activity by at least about 70%. In some embodiments, the antibodies described herein activate an NK cell activity by at least about 80%. In some embodiments, the antibodies described herein activate an NK cell activity by at least about 90%. In some embodiments, the antibodies described herein activate an NK cell activity by at least about 95%. In certain embodiments, the antibodies described herein activate an NK cell activity by at least about 15% to about 65%. In certain embodiments, the antibodies described herein activate an NK cell activity by at least about 20% to about 65%. In certain embodiments, the antibodies described herein activate an NK cell activity by at least about 30% to about 65%. In specific embodiments, the NK cells are human NK cells.

In some embodiments, the antibodies described herein can promote IFNg production by NK cells. In some embodiments, the antibodies described herein promote IFNg production by NK cells by at least 10%. In some embodiments, the antibodies described herein promote IFNg production by NK cells by at least 20%. In some embodiments, the antibodies described herein promote IFNg production by NK cells by at least 30%. In some embodiments, the antibodies described herein promote IFNg production by NK cells by at least 40%. In some embodiments, the antibodies described herein promote IFNg production by NK cells by at least 50%. In some embodiments, the antibodies described herein promote IFNg production by NK cells by at least 60%. In some embodiments, the antibodies described herein promote IFNg production by NK cells by at least 70%. In some embodiments, the antibodies described herein promote IFNg production by NK cells by at least 80%. In some embodiments, the antibodies described herein promote IFNg production by NK cells by at least 90%. In some embodiments, the antibodies described herein promote IFNg production by NK cells by at least 95%. In certain embodiments, the antibodies described herein promote IFNg production by NK cells by at least about 15% to about 65%. In certain embodiments, the antibodies described herein promote IFNg production by NK cells by at least about 20% to about 65%. In certain embodiments, the antibodies described herein promote IFNg production by NK cells by at least about 30% to about 65%. In specific embodiments, the NK cells are human NK cells.

### 5.2.2. Multispecific Molecules

The multispecific molecules provided herein comprise a binding domain capable of binding to an antigen present on an NK cell. In some embodiments, the antigen is NKG2d. In some embodiments, the antigen is NKp46. In some embodiments, the first binding domain is as described or derived from the antibodies described above.

In addition to the domain described above, the multispecific molecules provided herein comprises an additional domain capable of binding to a second antigen. In some embodiments, the second binding domain is capable of binding to an antigen expressed on a tumor cell. In some embodiments, the second binding domain is capable of binding to a tumor specific antigen (TSA) or a tumor associated antigen (TAA). In some embodiments, the second binding domain is capable of binding to BCMA. In some embodiments, the second binding domain is capable of binding to GPRC5d.

Tumor antigens are proteins that are produced by tumor cells that can elicit an immune response, particularly T-cell mediated immune responses. Exemplary tumor antigens include, but not limited to, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, insulin growth factor (IGF)-I, IGF-II, IGF-I receptor, and mesothelin.

In some embodiments, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA).

In some embodiments, the tumor antigen is a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development, when the immune system is immature, and unable to respond or they may be antigens that are normally present at extremely low levels on normal cells, but which are expressed at much higher levels on tumor cells.

Non-limiting examples of TSA or TAA antigens include: differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7.

In some embodiments, the multispecific molecule provided herein is a multispecific antibody. The antibodies provided herein include, but are not limited to, synthetic antibodies, monoclonal antibodies, recombinantly produced antibodies, human antibodies, humanized antibodies, chimeric antibodies, etc.

In particular, the antibodies provided herein include immunoglobulin molecules and immunologically active portions of immunoglobulin molecules, i.e., molecules that contain an antigen binding site that immunospecifically binds to an antigen. The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule. In some embodiments, the antibody is an IgG antibody. In some embodiments, the IgG antibody is an IgG1 antibody. In some embodiments, the IgG antibody is an IgG2, IgG3, or IgG4 antibody.

In some embodiments of the various multispecific molecules provided herein comprises a variant and/or derivative of antibodies include antibody fragments that retain the ability to specifically bind to an epitope. In other embodiments of the various multispecific molecules provided herein, the first binding domain and/or the second binding domain is a variant and/or derivative of antibodies include antibody fragments that retain the ability to specifically bind to an epitope. Exemplary fragments include Fab fragments (an antibody fragment that contains the antigen-binding domain and comprises a light chain and part of a heavy chain bridged by a disulfide bond); Fab′ (an antibody fragment containing a single anti-binding domain comprising an Fab and an additional portion of the heavy chain through the hinge region); F(ab′)₂ (two Fab′ molecules joined by interchain disulfide bonds in the hinge regions of the heavy chains; the Fab′ molecules may be directed toward the same or different epitopes); a bispecific Fab (a Fab molecule having two antigen binding domains, each of which may be directed to a different epitope); a single chain Fab chain comprising a variable region, also known as, a scFv (the variable, antigen-binding determinative region of a single light and heavy chain of an antibody linked together by a chain of 10-25 amino acids); a disulfide-linked Fv, or dsFv (the variable, antigen-binding determinative region of a single light and heavy chain of an antibody linked together by a disulfide bond); a camelized VH (the variable, antigen-binding determinative region of a single heavy chain of an antibody in which some amino acids at the VH interface are those found in the heavy chain of naturally occurring camel antibodies); a bispecific scFv (a scFv or a dsFv molecule having two antigen-binding domains, each of which may be directed to a different epitope); a diabody (a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (a trimerized scFv, formed in a manner similar to a diabody, but in which three antigen-binding domains are created in a single complex; the three antigen binding domains may be directed towards the same or different epitopes); and a tetrabody (a tetramerized scFv, formed in a manner similar to a diabody, but in which four antigen-binding domains are created in a single complex; the four antigen binding domains may be directed towards the same or different epitopes). Derivatives of antibodies also include one or more CDR sequences of an antibody combining site. The CDR sequences may be linked together on a scaffold when two or more CDR sequences are present. In certain embodiments, an antibody provided herein comprises a single-chain Fv (“scFv”). scFvs are antibody fragments comprising the VH and VL domains of an antibody, wherein these domains are present in a single polypeptide chain. Generally, the scFv polypeptide further comprises a polypeptide linker between the VH and VL domains which enables the scFv to form the desired structure for antigen binding. For a review of scFvs see Pluckthun in The Pharmacology of Monoclonal Antibodies, vol. 113, Rosenburg and Moore eds. Springer-Verlag, New York, pp. 269-315 (1994).

In specific embodiments, the antibody that binds to NKG2d comprises a VH region and a VL region. In some embodiments, the NKG2d antibody is a single chain antibody. In some embodiments, the NKG2d antibody is a single domain antibody. In some embodiments, the NKG2d antibody is a nanobody. In certain embodiments, the NKG2d antibody is a VHH antibody. In certain embodiments, the NKG2d antibody is a llama antibody. In some embodiments, the NKG2d antibody is not a single chain antibody. In some embodiments, the NKG2d antibody is not a single domain antibody. In some embodiments, the NKG2d antibody is not a nanobody. In certain embodiments, the NKG2d antibody is not a VHH antibody. In certain embodiments, the NKG2d antibody is not a llama antibody. In some embodiments, the NKG2d antibody is a multispecific antibody. In other embodiments, the NKG2d antibody is a bispecific antibody. In certain embodiments, the multispecific antibody comprises an antigen binding fragment of an NKG2d antibody provided herein. In other embodiments, the bispecific antibody comprises an antigen binding fragment of an NKG2d antibody provided herein.

In specific embodiments, provided herein is a multispecific antibody that binds NKG2d. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific NKG2d antibody comprises: (a) a first binding domain that binds NKG2d, and (b) a second binding domain that binds to a second target. In one embodiment, the multispecific NKG2d antibody comprises: (a) a first binding domain that binds NKG2d, and (b) a second binding domain that binds to a second target, and (c) a third binding domain that binds to a third target. In one embodiment, the multispecific NKG2d antibody comprises: (a) a first binding domain that binds NKG2d, and (b) a second binding domain that binds to a second target, (c) a third binding domain that binds to a third target, and (d) a fourth binding domain that binds to a fourth target.

In certain embodiments, provided herein is a bispecific antibody comprising: (a) a first binding domain that binds to NKG2d, and (b) a second binding domain that binds to a second target that is not NKG2d. In another aspect, provided herein is a bispecific antibody comprising: (a) a first binding domain that binds to NKG2d, and (b) a second binding domain that binds to a second target that binds to an antigen expressed on a tumor cell. In some embodiments, the second binding domain binds to BCMA. In some embodiments, the second binding domain binds to GPRC5d.

In certain embodiments, provided herein is an anti-NKG2d bispecific antibody comprising a binding domain that binds to NKG2d having a VH region, VL region, VH CDR1, VH CDR2, VH CDR3, VL CDR1, VL CDR2, and/or VL CDR3 of any one of the antibodies described herein. In some embodiments, provided herein is an anti-NKG2d bispecific antibody comprising a binding domain that binds to NKG2d having a VH region of any one of the antibodies described herein. In some embodiments, provided herein is an anti-NKG2d bispecific antibody comprising a binding domain that binds to NKG2d having a VL region of any one of the antibodies described herein. In some embodiments, provided herein is an anti-NKG2d bispecific antibody comprising a binding domain that binds to NKG2d having a VH region of any one of the antibodies described herein, and a VL region of any one of the antibodies described herein. In some embodiments, provided herein is an anti-NKG2d bispecific antibody comprising a binding domain that binds to NKG2d having a VH CDR1, VH CDR2, and VH CDR3 of any one of the antibodies described. In some embodiments, provided herein is an anti-NKG2d bispecific antibody comprising a binding domain that binds to NKG2d having a VL CDR1, VL CDR2, and VL CDR3 of any one of the antibodies described herein. In some embodiments, provided herein is an anti-NKG2d bispecific antibody comprising a binding domain that binds to NKG2d having a VH CDR1, VH CDR2, and VH CDR3 of any one of the antibodies described herein; and a VL CDR1, VL CDR2, and VL CDR3 of any one of the antibodies described herein.

In certain embodiments, the anti-NKG2d antibody is a bispecific antibody. In some embodiments, the anti-NKG2d bispecific antibody further comprises a second binding domain that binds to BCMA having a VH region, VL region, VH CDR1, VH CDR2, VH CDR3, VL CDR1, VL CDR2, and/or VL CDR3 of an anti-BCMA antibody provided herein. In some embodiments, the anti-NKG2d bispecific antibody further comprises a second binding domain that binds to BCMA having a VH region of an anti-BCMA antibody provided herein. In some embodiments, the anti-NKG2d bispecific antibody further comprises a second binding domain that binds to BCMA having a VL region of an anti-BCMA antibody provided herein. In some embodiments, the anti-NKG2d bispecific antibody further comprises a second binding domain that binds to BCMA having a VH region of an anti-BCMA antibody provided herein, and a VL region of an anti-BCMA antibody provided herein. In some embodiments, the anti-NKG2d bispecific antibody further comprises a second binding domain that binds to BCMA having a VH CDR1, VH CDR2, and VH CDR3 of an anti-BCMA antibody provided herein. In some embodiments, the anti-NKG2d bispecific antibody further comprises a second binding domain that binds to BCMA having a VL CDR1, VL CDR2, and VL CDR3 of an anti-BCMA antibody provided herein. In some embodiments, the anti-NKG2d bispecific antibody further comprises a second binding domain that binds to BCMA having a VH CDR1, VH CDR2, and VH CDR3 of an anti-BCMA antibody provided herein, and a VL CDR1, VL CDR2, and VL CDR3 of an anti-BCMA antibody provided herein.

In certain embodiments, the anti-NKG2d antibody is a bispecific antibody. In some embodiments, the anti-NKG2d bispecific antibody further comprises a second binding domain that binds to GPRC5d having a VH region, VL region, VH CDR1, VH CDR2, VH CDR3, VL CDR1, VL CDR2, and/or VL CDR3 of an anti-GPRC5d antibody provided herein. In some embodiments, the anti-NKG2d bispecific antibody further comprises a second binding domain that binds to GPRC5d having a VH region of an anti-GPRC5d antibody provided herein. In some embodiments, the anti-NKG2d bispecific antibody further comprises a second binding domain that binds to GPRC5d having a VL region of an anti-GPRC5d antibody provided herein. In some embodiments, the anti-NKG2d bispecific antibody further comprises a second binding domain that binds to GPRC5d having a VH region of an anti-GPRC5d antibody provided herein, and a VL region of an anti-GPRC5d antibody provided herein. In some embodiments, the anti-NKG2d bispecific antibody further comprises a second binding domain that binds to GPRC5d having a VH CDR1, VH CDR2, and VH CDR3 of an anti-GPRC5d antibody provided herein. In some embodiments, the anti-NKG2d bispecific antibody further comprises a second binding domain that binds to GPRC5d having a VL CDR1, VL CDR2, and VL CDR3 of an anti-GPRC5d antibody provided herein. In some embodiments, the anti-NKG2d bispecific antibody further comprises a second binding domain that binds to GPRC5d having a VH CDR1, VH CDR2, and VH CDR3 of an anti-GPRC5d antibody provided herein, and a VL CDR1, VL CDR2, and VL CDR3 of an anti-GPRC5d antibody provided herein.

In some embodiments, the first binding domain that binds NKG2d is as described or derived from the antibodies described above. In some specific embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:4, 5, and 6, respectively, and (ii) a VL comprising a VL CDR1, VL CDR2, and VL CDR3 having an amino acid sequence of SEQ ID NOs:7, 8, and 9, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:10, 11, and 12, respectively, and (ii) a VL comprising a VL CDR1, VL CDR2, and VL CDR3 having an amino acid sequence of SEQ ID NOs:13, 14, and 15, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:16, 17, and 18, respectively, and (ii) a VL comprising a VL CDR1, VL CDR2, and VL CDR3 having an amino acid sequence of SEQ ID NOs:19, 20, and 21, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:22, 23, and 24, respectively, and (ii) a VL comprising a VL CDR1, VL CDR2, and VL CDR3 having an amino acid sequence of SEQ ID NOs:25, 26, and 27, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:28, 29, and 30, respectively, and (ii) a VL comprising a VL CDR1, VL CDR2, and VL CDR3 having an amino acid sequence of SEQ ID NOs:31, 32, and 33, respectively. In some embodiments of the multispecific antibodies provided herein, the first binding domain that binds NKG2d comprises a VH comprising a VH CDR1, a VH CDR2, and a VH CDR3 having an amino acid sequence of a VH CDR1, a VH CDR2, and a VH CDR3, respectively, of SEQ ID NO:2. In some embodiments of the multispecific antibodies provided herein, the first binding domain that binds NKG2d comprises a VL comprising a VL CDR1, a VL CDR2, and a VL CDR3 having an amino acid sequence of a VL CDR1, a VL CDR2, and a VL CDR3, respectively, of SEQ ID NO:3. In some embodiments of the multispecific antibodies provided herein, the first binding domain that binds NKG2d comprises: (i) a VH comprising a VH CDR1, a VH CDR2, and a VH CDR3 having an amino acid sequence of a VH CDR1, a VH CDR2, and a VH CDR3, respectively, of SEQ ID NO:2; and (ii) a VL comprising a VL CDR1, a VL CDR2, and a VL CDR3 having an amino acid sequence of a VL CDR1, a VL CDR2, and a VL CDR3, respectively, of SEQ ID NO:3. In some embodiments of the multispecific antibodies provided herein, the first binding domain that binds NKG2d comprises a VH having an amino acid sequence of SEQ ID NO:2. In some embodiments, the first binding domain that binds NKG2d comprises a VL having an amino acid sequence of SEQ ID NO:3. In some embodiments of the multispecific antibodies provided herein, the first binding domain that binds NKG2d comprises a VH having an amino acid sequence of SEQ ID NO:2, and a VL having an amino acid sequence of SEQ ID NO:3. In some embodiments of the multispecific antibodies provided herein, the first binding domain that binds NKG2d comprises a VH comprising an amino acid sequence having at least 95% identity to the amino acid sequence of SEQ ID NO:2. In some embodiments of the multispecific antibodies provided herein, the first binding domain that binds NKG2d comprises a VL comprising an amino acid sequence having at least 95% identity to the amino acid sequence of SEQ ID NO:3. In some embodiments of the multispecific antibodies provided herein, the first binding domain that binds NKG2d comprises a VH comprising an amino acid sequence having at least 95% identity to the amino acid sequence of SEQ ID NO:2, and a VL comprising an amino acid sequence having at least 95% identity to the amino acid sequence of SEQ ID NO:3.

In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:36, 37, and 38, respectively, and (ii) a VL comprising a VL CDR1, VL CDR2, and VL CDR3 having an amino acid sequence of SEQ ID NOs:39, 40, and 41, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:42, 43, and 44, respectively, and (ii) a VL comprising a VL CDR1, VL CDR2, and VL CDR3 having an amino acid sequence of SEQ ID NOs:45, 46, and 47, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:48, 49, and 50, respectively, and (ii) a VL comprising a VL CDR1, VL CDR2, and VL CDR3 having an amino acid sequence of SEQ ID NOs:51, 52, and 53, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:54, 55, and 56, respectively, and (ii) a VL comprising a VL CDR1, VL CDR2, and VL CDR3 having an amino acid sequence of SEQ ID NOs:57, 58, and 59, respectively. In some embodiments of the multispecific antibodies provided herein, the first binding domain that binds NKG2d comprises: (i) a VH comprising a VH CDR1, a VH CDR2, and a VH CDR3 having an amino acid sequence of SEQ ID NOs:60, 61, and 62, respectively, and (ii) a VL comprising a VL CDR1, VL CDR2, and VL CDR3 having an amino acid sequence of SEQ ID NOs:63, 64, and 65, respectively. In some embodiments of the multispecific antibodies provided herein, the first binding domain that binds NKG2d comprises a VH comprising a VH CDR1, a VH CDR2, and a VH CDR3 having an amino acid sequence of a VH CDR1, a VH CDR2, and a VH CDR3, respectively, of SEQ ID NO:34. In some embodiments of the multispecific antibodies provided herein, the first binding domain that binds NKG2d comprises a VL comprising a VL CDR1, a VL CDR2, and a VL CDR3 having an amino acid sequence of a VL CDR1, a VL CDR2, and a VL CDR3, respectively, of SEQ ID NO:35. In some embodiments of the multispecific antibodies provided herein, the first binding domain that binds NKG2d comprises: (i) a VH comprising a VH CDR1, a VH CDR2, and a VH CDR3 having an amino acid sequence of a VH CDR1, a VH CDR2, and a VH CDR3, respectively, of SEQ ID NO:34; and (ii) a VL comprising a VL CDR1, a VL CDR2, and a VL CDR3 having an amino acid sequence of a VL CDR1, a VL CDR2, and a VL CDR3, respectively, of SEQ ID NO:35. In some embodiments of the multispecific antibodies provided herein, the first binding domain that binds NKG2d comprises a VH having an amino acid sequence of SEQ ID NO:34. In some embodiments of the multispecific antibodies provided herein, the first binding domain that binds NKG2d comprises a VL having an amino acid sequence of SEQ ID NO:35. In some embodiments of the multispecific antibodies provided herein, the first binding domain that binds NKG2d comprises a VH having an amino acid sequence of SEQ ID NO:34, and a VL having an amino acid sequence of SEQ ID NO:35. In some embodiments of the multispecific antibodies provided herein, the first binding domain that binds NKG2d comprises a VH comprising an amino acid sequence having at least 95% identity to the amino acid sequence of SEQ ID NO:34. In some embodiments of the multispecific antibodies provided herein, the first binding domain that binds NKG2d comprises a VL comprising an amino acid sequence having at least 95% identity to the amino acid sequence of SEQ ID NO:35. In some embodiments of the multispecific antibodies provided herein, the first binding domain that binds NKG2d comprises a VH comprising an amino acid sequence having at least 95% identity to the amino acid sequence of SEQ ID NO:34, and a VL comprising an amino acid sequence having at least 95% identity to the amino acid sequence of SEQ ID NO:35.

In some embodiments, the VH CDR1, VH CDR2, VH CDR3, VL CDR1, VL CDR2, and VL CDR3 amino acid sequences of the first binding domain that binds NKG2d are according to the Kabat numbering system. In some embodiments, the VH CDR1, VH CDR2, VH CDR3, VL CDR1, VL CDR2, and VL CDR3 amino acid sequences of the first binding domain that binds NKG2d are according to the Chothia numbering system. In some embodiments, the VH CDR1, VH CDR2, VH CDR3, VL CDR1, VL CDR2, and VL CDR3 amino acid sequences of the first binding domain that binds NKG2d are according to the AbM numbering system. In some embodiments, the VH CDR1, VH CDR2, VH CDR3, VL CDR1, VL CDR2, and VL CDR3 amino acid sequences of the first binding domain that binds NKG2d are according to the Contact numbering system. In some embodiments, the VH CDR1, VH CDR2, VH CDR3, VL CDR1, VL CDR2, and VL CDR3 amino acid sequences of the first binding domain that binds NKG2d are according to the IMGT numbering system.

In some embodiments of the multispecific NKG2d antibodies provided herein, the first binding domain binds an NKG2d antigen. In some embodiments, the first binding domain binds an NKG2d epitope. In some embodiments, the first binding domain specifically binds to NKG2d. In some embodiments, the VH CDR1, VH CDR2, VH CDR3, VL CDR1, VL CDR2 and VL CDR3 of the first binding domain form a binding site for an antigen of the NKG2d. In some embodiments, the VH CDR1, VH CDR2, VH CDR3, VL CDR1, VL CDR2 and VL CDR3 of the first binding domain form a binding site for an epitope of the NKG2d. In some embodiments, the NKG2d is present on the surface of an NK cell.

In another aspect, provided herein is a multispecific antibody that competes for binding to NKG2d with any of the NKG2d antibodies described herein. In another aspect, provided herein is a multispecific antibody that binds to the same epitope as any of the NKG2d antibodies described herein. In another aspect, provided is a multispecific NKG2d antibody that binds an epitope on NKG2d that overlaps with the epitope on NKG2d bound by an NKG2d antibody described herein.

In one aspect, provided is a multispecific antibody that competes for binding to NKG2d with an NKG2d reference antibody. In another aspect, provided is a multispecific NKG2d antibody that binds to the same NKG2d epitope as an NKG2d reference antibody. In another aspect, provided is a multispecific NKG2d antibody that binds an epitope on NKG2d that overlaps with the epitope on NKG2d bound by an NKG2d reference antibody.

In one embodiment, the NKG2d reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3. In one embodiment, the NKG2d reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:35.

In some embodiments of the multispecific NKG2d antibodies provided herein, the second target is not an NKG2d antigen. In some embodiments of the multispecific NKG2d antibodies provided herein, the third target is not an NKG2d antigen. In some embodiments of the multispecific NKG2d antibodies provided herein, the fourth target is not an NKG2d antigen. In some embodiments of the multispecific NKG2d antibodies provided herein, the second target is not an NKG2d antigen, and the third target is not an NKG2d antigen. In some embodiments of the multispecific NKG2d antibodies provided herein, the second target is not an NKG2d antigen, and the fourth target is not an NKG2d antigen. In some embodiments of the multispecific NKG2d antibodies provided herein, the third target is not an NKG2d antigen, and the fourth target is not an NKG2d antigen. In some embodiments of the multispecific NKG2d antibodies provided herein, the second target is not an NKG2d antigen, the third target is not an NKG2d antigen, and the fourth target is not an NKG2d antigen. In some embodiments of the multispecific NKG2d antibodies provided herein, the second target is not an NKG2d epitope. In some embodiments of the multispecific NKG2d antibodies provided herein, the third target is not an NKG2d epitope. In some embodiments of the multispecific NKG2d antibodies provided herein, the fourth target is not an NKG2d epitope. In some embodiments of the multispecific NKG2d antibodies provided herein, the second target is not an NKG2d epitope, and the third target is not an NKG2d epitope. In some embodiments of the multispecific NKG2d antibodies provided herein, the second target is not an NKG2d epitope, and the fourth target is not an NKG2d epitope. In some embodiments of the multispecific NKG2d antibodies provided herein, the third target is not an NKG2d epitope, and the fourth target is not an NKG2d epitope. In some embodiments of the multispecific NKG2d antibodies provided herein, the second target is not an NKG2d epitope, the third target is not an NKG2d epitope, and the fourth target is not an NKG2d epitope. In some embodiments of the multispecific NKG2d antibodies provided herein, the second target is BCMA. In some embodiments of the multispecific NKG2d antibodies provided herein, the second target is GPRC5d.

The binding of the multispecific antibody provided herein to NKG2d present on the surface of the NK cell, and the binding of the second target antigen present on the surface of the second target cell can, for example, result in the killing of the second target cell. In other embodiment, the binding of the multispecific antibody provided herein to NKG2d present on the surface of the NK cell, and the binding of a second target antigen can, for example, result in the activation of the NK cell. In some embodiments, provided herein is a bispecific antibody comprising: (a) a first binding domain that binds to NKG2d, and (b) a second binding domain that binds to a cancer antigen present on the surface of a cancer cell. In some embodiments, the antigen on the surface of the cancer cell is a tumor-specific antigen. In some embodiments, the antigen on the surface of the cancer cell is a tumor associated antigen. In some embodiments, the antigen on the surface of the cancer cell is a neoantigen. In certain embodiments, the first binding domain of the bispecific antibody specifically binds NKG2d. In some embodiments, the NKG2d is present on the surface of an NK cell. In some embodiments, the cancer cell is killed when the bispecific antibody binds to the NKG2d on the surface of the NK cell and the antigen on the surface of the cancer cell.

In another aspect, provided herein is a bispecific antibody comprising: (a) a first binding domain that binds to NKG2d, and (b) a second binding domain that binds to BCMA. In certain embodiments, the first binding domain of the bispecific antibody specifically binds NKG2d. In some embodiments, the NKG2d is present on the surface of an NK cell. In some embodiments, the BCMA is on the surface of a cell. In certain embodiments, the NKG2d is present on the surface of an NK cell, and the BCMA is on the surface of a cell. In some embodiments, the cell having the BCMA on the surface is killed when the bispecific antibody binds to the NKG2d on the surface of the NK cell and the BCMA on the surface of the cell. In some embodiments, the BCMA is on the surface of a cancer cell. In certain embodiments, the NKG2d is present on the surface of an NK cell, and the BCMA is on the surface of a cancer cell. In some embodiments, the cancer cell is killed when the bispecific antibody binds to the NKG2d on the surface of the NK cell and the BCMA on the surface of the cancer cell. Bispecific antibodies comprising any of the NKG2d antibodies provided herein as the first binding domain are contemplated, in certain embodiments. In addition, bispecific antibodies comprising any of the NKG2d antibodies provided herein as the first binding domain, and a second binding domain that binds to BCMA are also contemplated in certain embodiments.

In some embodiments, the multispecific antibodies provided herein is a bispecific antibody comprising: (a) a first binding domain that binds to NKG2d, and (b) a second binding domain that binds to BCMA, wherein the second binding domain that binds BCMA comprises: (i) a VH comprising a VH CDR1, a VH CDR2, and a VH CDR3 having an amino acid sequence of SEQ ID NOs:101, 102, and 103, respectively, and (ii) a VL comprising a VL CDR1, VL CDR2, and VL CDR3 having an amino acid sequence of SEQ ID NOs:104, 105, and 106, respectively. In some embodiments of the multispecific antibodies provided herein, the second binding domain that binds BCMA comprises: (i) a VH comprising a VH CDR1, a VH CDR2, and a VH CDR3 having an amino acid sequence of SEQ ID NOs:107, 108, and 109, respectively, and (ii) a VL comprising a VL CDR1, VL CDR2, and VL CDR3 having an amino acid sequence of SEQ ID NOs:110, 111, and 112, respectively. In some embodiments of the multispecific antibodies provided herein, the second binding domain that binds BCMA comprises: (i) a VH comprising a VH CDR1, a VH CDR2, and a VH CDR3 having an amino acid sequence of SEQ ID NOs:113, 114, and 115, respectively, and (ii) a VL comprising a VL CDR1, VL CDR2, and VL CDR3 having an amino acid sequence of SEQ ID NOs:116, 117, and 118, respectively. In some embodiments of the multispecific antibodies provided herein, the second binding domain that binds BCMA comprises: (i) a VH comprising a VH CDR1, a VH CDR2, and a VH CDR3 having an amino acid sequence of SEQ ID NOs:119, 120, and 121, respectively, and (ii) a VL comprising a VL CDR1, VL CDR2, and VL CDR3 having an amino acid sequence of SEQ ID NOs:122, 123, and 124, respectively. In some embodiments of the multispecific antibodies provided herein, the second binding domain that binds BCMA comprises: (i) a VH comprising a VH CDR1, a VH CDR2, and a VH CDR3 having an amino acid sequence of SEQ ID NOs:125, 126, and 127, respectively, and (ii) a VL comprising a VL CDR1, VL CDR2, and VL CDR3 having an amino acid sequence of SEQ ID NOs:128, 129, and 130, respectively. In some embodiments of the multispecific antibodies provided herein, the second binding domain that binds BCMA comprises a VH comprising a VH CDR1, a VH CDR2, and a VH CDR3 having an amino acid sequence of a VH CDR1, a VH CDR2, and a VH CDR3, respectively, of SEQ ID NO:99. In some embodiments of the multispecific antibodies provided herein, the second binding domain that binds BCMA comprises a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific antibodies provided herein, the second binding domain that binds BCMA comprises: (i) a VH comprising a VH CDR1, a VH CDR2, and a VH CDR3 having an amino acid sequence of a VH CDR1, a VH CDR2, and a VH CDR3, respectively, of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific antibodies provided herein, the second binding domain that binds BCMA comprises a VH having an amino acid sequence of SEQ ID NO:99. In some embodiments of the multispecific antibodies provided herein, the second binding domain that binds BCMA comprises a VL having an amino acid sequence of SEQ ID NO:100. In some embodiments of the multispecific antibodies provided herein, the second binding domain that binds BCMA comprises a VH having an amino acid sequence of SEQ ID NO:99, and a VL having an amino acid sequence of SEQ ID NO:100. In some embodiments of the multispecific antibodies provided herein, the second binding domain that binds BCMA comprises a VH comprising an amino acid sequence having at least 95% identity to the amino acid sequence of SEQ ID NO:99. In some embodiments of the multispecific antibodies provided herein, the second binding domain that binds BCMA comprises a VL comprising an amino acid sequence having at least 95% identity to the amino acid sequence of SEQ ID NO:100. In some embodiments of the multispecific antibodies provided herein, the second binding domain that binds BCMA comprises a VH comprising an amino acid sequence having at least 95% identity to the amino acid sequence of SEQ ID NO:99, and a VL comprising an amino acid sequence having at least 95% identity to the amino acid sequence of SEQ ID NO:100.

In another aspect, provided herein is a bispecific antibody comprising: (a) a first binding domain that binds to NKG2d, and (b) a second binding domain that binds to GPRC5d. In certain embodiments, the first binding domain of the bispecific antibody specifically binds NKG2d. In some embodiments, the NKG2d is present on the surface of an NK cell. In some embodiments, the GPRC5d is on the surface of a cell. In certain embodiments, the NKG2d is present on the surface of an NK cell, and the GPRC5d is on the surface of a cell. In some embodiments, the cell having the GPRC5d on the surface is killed when the bispecific antibody binds to the NKG2d on the surface of the NK cell and the GPRC5d on the surface of the cell. In some embodiments, the GPRC5d is on the surface of a cancer cell. In certain embodiments, the NKG2d is present on the surface of an NK cell, and the GPRC5d is on the surface of a cancer cell. In some embodiments, the cancer cell is killed when the bispecific antibody binds to the NKG2d on the surface of the NK cell and the GPRC5d on the surface of the cancer cell. Bispecific antibodies comprising any of the NKG2d antibodies provided herein as the first binding domain are contemplated, in certain embodiments. In addition, bispecific antibodies comprising any of the NKG2d antibodies provided herein as the first binding domain, and a second binding domain that binds to GPRC5d are also contemplated in certain embodiments.

In some embodiments, the multispecific antibodies provided herein, is a bispecific antibody comprising: (a) a first binding domain that binds to NKG2d, and (b) a second binding domain that binds to GPRC5d, wherein the second binding domain that binds GPRC5d comprises: (i) a VH comprising a VH CDR1, a VH CDR2, and a VH CDR3 having an amino acid sequence of SEQ ID NOs:133, 134, and 135, respectively, and (ii) a VL comprising a VL CDR1, VL CDR2, and VL CDR3 having an amino acid sequence of SEQ ID NOs:136, 137, and 138, respectively. In some embodiments of the multispecific antibodies provided herein, the second binding domain that binds GPRC5d comprises: (i) a VH comprising a VH CDR1, a VH CDR2, and a VH CDR3 having an amino acid sequence of SEQ ID NOs:139, 140, and 141, respectively, and (ii) a VL comprising a VL CDR1, VL CDR2, and VL CDR3 having an amino acid sequence of SEQ ID NOs:142, 143, and 144, respectively. In some embodiments of the multispecific antibodies provided herein, the second binding domain that binds GPRC5d comprises: (i) a VH comprising a VH CDR1, a VH CDR2, and a VH CDR3 having an amino acid sequence of SEQ ID NOs:145, 146, and 147, respectively, and (ii) a VL comprising a VL CDR1, VL CDR2, and VL CDR3 having an amino acid sequence of SEQ ID NOs:148, 149, and 150, respectively. In some embodiments of the multispecific antibodies provided herein, the second binding domain that binds GPRC5d comprises: (i) a VH comprising a VH CDR1, a VH CDR2, and a VH CDR3 having an amino acid sequence of SEQ ID NOs:151, 152, and 153, respectively, and (ii) a VL comprising a VL CDR1, VL CDR2, and VL CDR3 having an amino acid sequence of SEQ ID NOs:154, 155, and 156, respectively. In some embodiments of the multispecific antibodies provided herein, the second binding domain that binds GPRC5d comprises: (i) a VH comprising a VH CDR1, a VH CDR2, and a VH CDR3 having an amino acid sequence of SEQ ID NOs:157, 158, and 159, respectively, and (ii) a VL comprising a VL CDR1, VL CDR2, and VL CDR3 having an amino acid sequence of SEQ ID NOs:160, 161, and 162, respectively. In some embodiments of the multispecific antibodies provided herein, the second binding domain that binds GPRC5d comprises a VH comprising a VH CDR1, a VH CDR2, and a VH CDR3 having an amino acid sequence of a VH CDR1, a VH CDR2, and a VH CDR3, respectively, of SEQ ID NO:131. In some embodiments of the multispecific antibodies provided herein, the second binding domain that binds GPRC5d comprises a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific antibodies provided herein, the second binding domain that binds GPRC5d comprises: (i) a VH comprising a VH CDR1, a VH CDR2, and a VH CDR3 having an amino acid sequence of a VH CDR1, a VH CDR2, and a VH CDR3, respectively, of SEQ ID NO:131; and (ii) a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific antibodies provided herein, the second binding domain that binds GPRC5d comprises a VH having an amino acid sequence of SEQ ID NO:131. In some embodiments of the multispecific antibodies provided herein, the second binding domain that binds GPRC5d comprises a VL having an amino acid sequence of SEQ ID NO:132. In some embodiments of the multispecific antibodies provided herein, the second binding domain that binds GPRC5d comprises a VH having an amino acid sequence of SEQ ID NO:131, and a VL having an amino acid sequence of SEQ ID NO:132. In some embodiments of the multispecific antibodies provided herein, the second binding domain that binds GPRC5d comprises a VH comprising an amino acid sequence having at least 95% identity to the amino acid sequence of SEQ ID NO:131. In some embodiments of the multispecific antibodies provided herein, the second binding domain that binds GPRC5d comprises a VL comprising an amino acid sequence having at least 95% identity to the amino acid sequence of SEQ ID NO:132. In some embodiments of the multispecific antibodies provided herein, the second binding domain that binds GPRC5d comprises a VH comprising an amino acid sequence having at least 95% identity to the amino acid sequence of SEQ ID NO:131, and a VL comprising an amino acid sequence having at least 95% identity to the amino acid sequence of SEQ ID NO:132.

In specific embodiments, provided is a multispecific antibody comprising an NKG2d antibody provided herein in a knob-in-hole format. In specific embodiments, provided is a bispecific antibody comprising an NKG2d antibody provided herein in a knob-in-hole format. In specific embodiments, provided is a trispecific antibody comprising an NKG2d antibody provided herein in a knob-in-hole format. In specific embodiments, provided is a quadraspecific antibody comprising an NKG2d antibody provided herein in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, an NKG2d antibody provided herein is comprised in a bispecific antibody. In some embodiments, an NKG2d antibody provided herein is comprised in a trispecific antibody. In some embodiments, an NKG2d antibody provided herein is comprised in a quadraspecific antibody. In some embodiments, an NKG2d bispecific antibody provided herein is comprised in a multispecific antibody.

In certain embodiments, a multispecific antibody provided herein comprises a first binding domain comprising an NKG2d antibody provided herein that binds to a first NKG2d epitope, and a second binding domain that binds to a second epitope, wherein the first NKG2d epitope and the second epitope are not the same. In certain embodiments, a bispecific antibody provided herein comprises a first binding domain comprising an NKG2d antibody provided herein that binds to a first NKG2d epitope, and a second binding domain that binds to a second epitope, wherein the first NKG2d epitope and the second epitope are not the same. In certain embodiments, a trispecific antibody provided herein comprises a first binding domain comprising an NKG2d antibody provided herein that binds to a first NKG2d epitope, a second binding domain that binds to a second epitope, and a third binding domain that binds to a third epitope, wherein the first NKG2d epitope, the second epitope, and the third epitope are not the same. In certain embodiments, a quadraspecific antibody provided herein comprises a first binding domain comprising an NKG2d antibody provided herein that binds to a first NKG2d epitope, a second binding domain that binds to a second epitope, a third binding domain that binds to a third epitope, and a fourth binding domain that binds to a fourth epitope, wherein the first NKG2d epitope, the second epitope, the third epitope, and the fourth epitope are not the same. In certain embodiments, a multispecific antibody provided herein comprises a first binding domain comprising an NKG2d antibody provided herein that binds to a first NKG2d antigen, and a second binding domain that binds to a second antigen, wherein the first NKG2d antigen and the second antigen are not the same. In certain embodiments, a bispecific antibody provided herein comprises a first binding domain comprising an NKG2d antibody provided herein that binds to a first NKG2d antigen, and a second binding domain that binds to a second antigen, wherein the first NKG2d antigen and the second antigen are not the same. In certain embodiments, a trispecific antibody provided herein comprises a first binding domain comprising an NKG2d antibody provided herein that binds to a first NKG2d antigen, a second binding domain that binds to a second antigen, and a third binding domain that binds to a third antigen, wherein the first NKG2d antigen, the second antigen, and the third antigen are not the same. In certain embodiments, a quadraspecific antibody provided herein comprises a first binding domain comprising an NKG2d antibody provided herein that binds to a first NKG2d antigen, a second binding domain that binds to a second antigen, a third binding domain that binds to a third antigen, and a fourth binding domain that binds to a fourth antigen, wherein the first NKG2d antigen, the second antigen, the third antigen, and the fourth antigen are not the same. In a specific embodiment, an NKG2d antibody, or antigen binding fragment thereof, provided herein specifically binds to NKG2d.

In some embodiments, the multispecific antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the NKG2d antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the NKG2d multispecific antibodies or antigen binding fragments thereof bind to a first epitope located on NKG2d and a second epitope of a second target antigen. In some embodiments, provided herein is a multispecific antibody comprising: (a) a first binding domain that binds to an NKG2d antigen, and (b) a second binding domain that binds to a second target antigen. In some embodiments, provided herein is a multispecific antibody comprising: (a) a first binding domain that specifically binds to an NKG2d antigen, and (b) a second binding domain that specifically binds to a second target antigen. In some embodiments, provided herein is a multispecific antibody comprising: (a) a first binding domain that binds to a first epitope on an NKG2d antigen, and (b) a second binding domain that binds to a second epitope on a second target antigen. In some embodiments, provided herein is a multispecific antibody comprising: (a) a first binding domain that specifically binds to a first epitope on an NKG2d antigen, and (b) a second binding domain that specifically binds to a second epitope on a second target antigen.

In specific embodiments, the NKG2d antigen is on the surface of an NK cell. In certain embodiments, the second target antigen is not NKG2d. The binding of the NKG2d multispecific antibody to NKG2d present on the surface of the NK cell, and the binding of the second target antigen present on the surface of the second target cell can, for example, result in the killing of the second target cell. In other embodiment, the binding of the NKG2d multispecific antibody to NKG2d present on the surface of the NK cell, and the binding of a second target antigen can, for example, result in the activation of the NK cell.

In another aspect, provided herein is a multispecific antibody that comprises a first binding domain that binds to NKG2d and a second binding domain that binds to BCMA (“multispecific NKG2d/BCMA antibody”). In some embodiments, the multispecific NKG2d/BCMA antibody is a bispecific antibody. In some embodiments, the multispecific NKG2d/BCMA antibody is a trispecific antibody. In some embodiments, the multispecific NKG2d/BCMA antibody is a quadraspecific antibody.

In some embodiments, the multispecific NKG2d/BCMA antibody provided herein comprises: (a) a first binding domain that binds NKG2d, and (b) a second binding domain that binds to BCMA. In one embodiment, the multispecific NKG2d/BCMA antibody comprises: (a) a first binding domain that binds NKG2d, and (b) a second binding domain that binds to BCMA, and (c) a third binding domain that binds to a third target. In one embodiment, the multispecific NKG2d/BCMA antibody comprises: (a) a first binding domain that binds NKG2d, and (b) a second binding domain that binds to BCMA, (c) a third binding domain that binds to a third target, and (d) a fourth binding domain that binds to a fourth target.

In some embodiments of the multispecific NKG2d/BCMA antibodies provided herein, the first binding domain that binds NKG2d comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2. In some embodiments of the multispecific NKG2d/BCMA antibodies provided herein, the first binding domain that binds NKG2d comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3. In some embodiments of the multispecific NKG2d/BCMA antibodies provided herein, the first binding domain that binds NKG2d comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3. In some embodiments of the multispecific NKG2d/BCMA antibodies provided herein, the first binding domain that binds NKG2d comprises a VH having an amino acid sequence of SEQ ID NO:2. In some embodiments of the multispecific NKG2d/BCMA antibodies provided herein, the first binding domain that binds NKG2d comprises a VL having an amino acid sequence of SEQ ID NO:3. In some embodiments of the multispecific NKG2d/BCMA antibodies provided herein, the first binding domain that binds NKG2d comprises a VH having an amino acid sequence of SEQ ID NO:2, and a VL having an amino acid sequence of SEQ ID NO:3.

In some embodiments of the multispecific NKG2d/BCMA antibodies provided herein, the first binding domain that binds NKG2d comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34. In some embodiments of the multispecific NKG2d/BCMA antibodies provided herein, the first binding domain that binds NKG2d comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:35. In some embodiments of the multispecific NKG2d/BCMA antibodies provided herein, the first binding domain that binds NKG2d comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:35. In some embodiments of the multispecific NKG2d/BCMA antibodies provided herein, the first binding domain that binds NKG2d comprises a VH having an amino acid sequence of SEQ ID NO:34. In some embodiments of the multispecific NKG2d/BCMA antibodies provided herein, the first binding domain that binds NKG2d comprises a VL having an amino acid sequence of SEQ ID NO:35. In some embodiments of the multispecific NKG2d/BCMA antibodies provided herein, the first binding domain that binds NKG2d comprises a VH having an amino acid sequence of SEQ ID NO:34, and a VL having an amino acid sequence of SEQ ID NO:35.

In some embodiments of the multispecific NKG2d/BCMA antibodies provided herein, the VH CDR1, VH CDR2, VH CDR3, VL CDR1, VL CDR2, and VL CDR3 amino acid sequences of the first binding domain that binds NKG2d are according to the Kabat numbering system. In some embodiments of the multispecific NKG2d/BCMA antibodies provided herein, the VH CDR1, VH CDR2, VH CDR3, VL CDR1, VL CDR2, and VL CDR3 amino acid sequences of the first binding domain that binds NKG2d are according to the Chothia numbering system. In some embodiments of the multispecific NKG2d/BCMA antibodies provided herein, the VH CDR1, VH CDR2, VH CDR3, VL CDR1, VL CDR2, and VL CDR3 amino acid sequences of the first binding domain that binds NKG2d are according to the AbM numbering system. In some embodiments of the multispecific NKG2d/BCMA antibodies provided herein, the VH CDR1, VH CDR2, VH CDR3, VL CDR1, VL CDR2, and VL CDR3 amino acid sequences of the first binding domain that binds NKG2d are according to the Contact numbering system. In some embodiments of the multispecific NKG2d/BCMA antibodies provided herein, the VH CDR1, VH CDR2, VH CDR3, VL CDR1, VL CDR2, and VL CDR3 amino acid sequences of the first binding domain that binds NKG2d are according to the IMGT numbering system.

In some embodiments of the multispecific NKG2d/BCMA antibodies provided herein, the first binding domain binds an NKG2d antigen. In some embodiments of the multispecific NKG2d/BCMA antibodies provided herein, the first binding domain binds an NKG2d epitope. In some embodiments of the multispecific NKG2d/BCMA antibodies provided herein, the first binding domain specifically binds to NKG2d. In some embodiments of the multispecific NKG2d/BCMA antibodies provided herein, the VH CDR1, VH CDR2, VH CDR3, VL CDR1, VL CDR2 and VL CDR3 of the first binding domain form a binding site for an antigen of the NKG2d. In some embodiments of the multispecific NKG2d/BCMA antibodies provided herein, the VH CDR1, VH CDR2, VH CDR3, VL CDR1, VL CDR2 and VL CDR3 of the first binding domain form a binding site for an epitope of the NKG2d. In some embodiments of the multispecific NKG2d/BCMA antibodies provided herein, the NKG2d is present on the surface of an NK cell.

In some embodiments of the multispecific NKG2d/BCMA antibodies provided herein, the second binding domain that binds BCMA comprises a VH comprising a VH CDR1, a VH CDR2, and a VH CDR3 having an amino acid sequence of a VH CDR1, a VH CDR2, and a VH CDR3, respectively, of SEQ ID NO:99. In some embodiments of the multispecific NKG2d/BCMA antibodies provided herein, the second binding domain that binds BCMA comprises a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific NKG2d/BCMA antibodies provided herein, the second binding domain that binds BCMA comprises: (i) a VH comprising a VH CDR1, a VH CDR2, and a VH CDR3 having an amino acid sequence of a VH CDR1, a VH CDR2, and a VH CDR3, respectively, of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific NKG2d/BCMA antibodies provided herein, the second binding domain that binds BCMA comprises a VH having an amino acid sequence of SEQ ID NO:99. In some embodiments of the multispecific NKG2d/BCMA antibodies provided herein, the second binding domain that binds BCMA comprises a VL having an amino acid sequence of SEQ ID NO:100. In some embodiments of the multispecific NKG2d/BCMA antibodies provided herein, the second binding domain that binds BCMA comprises a VH having an amino acid sequence of SEQ ID NO:99, and a VL having an amino acid sequence of SEQ ID NO:100.

In some embodiments of the multispecific NKG2d/BCMA antibodies provided herein, the VH CDR1, VH CDR2, VH CDR3, VL CDR1, VL CDR2, and VL CDR3 amino acid sequences of the second binding domain that binds BCMA are according to the Kabat numbering system. In some embodiments of the multispecific NKG2d/BCMA antibodies provided herein, the VH CDR1, VH CDR2, VH CDR3, VL CDR1, VL CDR2, and VL CDR3 amino acid sequences of the second binding domain that binds BCMA are according to the Chothia numbering system. In some embodiments of the multispecific NKG2d/BCMA antibodies provided herein, the VH CDR1, VH CDR2, VH CDR3, VL CDR1, VL CDR2, and VL CDR3 amino acid sequences of the second binding domain that binds BCMA are according to the AbM numbering system. In some embodiments of the multispecific NKG2d/BCMA antibodies provided herein, the VH CDR1, VH CDR2, VH CDR3, VL CDR1, VL CDR2, and VL CDR3 amino acid sequences of the second binding domain that binds BCMA are according to the Contact numbering system. In some embodiments of the multispecific NKG2d/BCMA antibodies provided herein, the VH CDR1, VH CDR2, VH CDR3, VL CDR1, VL CDR2, and VL CDR3 amino acid sequences of the second binding domain that binds BCMA are according to the IMGT numbering system.

In some embodiments of the multispecific NKG2d/BCMA antibodies provided herein, the second binding domain binds a BCMA antigen. In some embodiments of the multispecific NKG2d/BCMA antibodies provided herein, the second binding domain binds a BCMA epitope. In some embodiments of the multispecific NKG2d/BCMA antibodies provided herein, the second binding domain specifically binds to BCMA. In some embodiments of the multispecific NKG2d/BCMA antibodies provided herein, the VH CDR1, VH CDR2, VH CDR3, VL CDR1, VL CDR2 and VL CDR3 of the second binding domain form a binding site for an antigen of the BCMA. In some embodiments, the VH CDR1, VH CDR2, VH CDR3, VL CDR1, VL CDR2 and VL CDR3 of the second binding domain form a binding site for an epitope of the BCMA. In some embodiments, the BCMA is present on the surface of a tumor cell.

In some embodiments of the multispecific NKG2d/BCMA antibodies provided herein, the third target is not an NKG2d antigen. In some embodiments of the multispecific NKG2d/BCMA antibodies provided herein, the fourth target is not an NKG2d antigen. In some embodiments of the multispecific NKG2d/BCMA antibodies provided herein, the third target is not an NKG2d antigen, and the fourth target is not an NKG2d antigen. In some embodiments of the multispecific NKG2d/BCMA antibodies provided herein, the third target is not a BCMA antigen. In some embodiments of the multispecific NKG2d/BCMA antibodies provided herein, the fourth target is not a BCMA antigen. In some embodiments of the multispecific NKG2d/BCMA antibodies provided herein, the third target is not a BCMA antigen, and the fourth target is not a BCMA antigen. In some embodiments of the multispecific NKG2d/BCMA antibodies provided herein, the third target is not an NKG2d epitope. In some embodiments of the multispecific NKG2d/BCMA antibodies provided herein, the fourth target is not an NKG2d epitope. In some embodiments of the multispecific NKG2d/BCMA antibodies provided herein, the third target is not an NKG2d epitope, and the fourth target is not an NKG2d epitope. In some embodiments of the multispecific NKG2d/BCMA antibodies provided herein, the third target is not a BCMA epitope. In some embodiments of the multispecific NKG2d/BCMA antibodies provided herein, the fourth target is not a BCMA epitope. In some embodiments of the multispecific NKG2d/BCMA antibodies provided herein, the third target is not a BCMA epitope, and the fourth target is not a BCMA epitope.

In a specific embodiment, the target is from a mammal. In a specific embodiment, the target is from a rat. In a specific embodiment, the target is from a mouse. In a specific embodiment, the target is from a primate. In a specific embodiment, the target is from a human.

In specific embodiments, provided is a multispecific NKG2d/BCMA antibody in a knob-in-hole format. In specific embodiments, provided is a bispecific NKG2d/BCMA antibody in a knob-in-hole format. In specific embodiments, provided is a trispecific antibody in a knob-in-hole format. In specific embodiments, provided is a quadraspecific antibody in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, an NKG2d/BCMA antibody provided herein is comprised in a bispecific antibody. In some embodiments, an NKG2d/BCMA antibody provided herein is comprised in a trispecific antibody. In some embodiments, an NKG2d/BCMA antibody provided herein is comprised in a quadraspecific antibody. In some embodiments, an NKG2d/BCMA bispecific antibody provided herein is comprised in a multispecific antibody.

In certain embodiments, a trispecific NKG2d/BCMA antibody provided herein comprises a first binding domain comprising an NKG2d antibody provided herein that binds to an NKG2d epitope, a second binding domain comprising a BCMA antibody provided herein that binds to a BCMA epitope, and a third binding domain that binds to a third epitope, wherein the NKG2d epitope, the BCMA epitope, and the third epitope are not the same. In certain embodiments, a quadraspecific antibody provided herein comprises a first binding domain comprising an NKG2d antibody provided herein that binds to an NKG2d epitope, a second binding domain comprising a BCMA antibody provided herein that binds to a BCMA epitope, a third binding domain that binds to a third epitope, and a fourth binding domain that binds to a fourth epitope, wherein the NKG2d epitope, the BCMA epitope, the third epitope, and the fourth epitope are not the same. In certain embodiments, a trispecific antibody provided herein comprises a first binding domain comprising an NKG2d antibody provided herein that binds to an NKG2d antigen, a second binding domain comprising a BCMA antibody provided herein that binds to a BCMA antigen, and a third binding domain that binds to a third antigen, wherein the NKG2d antigen, the BCMA antigen, and the third antigen are not the same. In certain embodiments, a quadraspecific antibody provided herein that binds to an NKG2d antigen, a second binding domain comprising a BCMA antibody provided herein that binds to a BCMA antigen, a third binding domain that binds to a third antigen, and a fourth binding domain that binds to a fourth antigen, wherein the NKG2d antigen, the BCMA antigen, the third antigen, and the fourth antigen are not the same. In certain embodiments of a multispecific NKG2d/BCMA antibody provided herein, the first binding domain that binds to NKG2d specifically binds to the NKG2d. In other embodiments of a multispecific NKG2d/BCMA antibody provided herein, the second binding domain that binds to BCMA specifically binds to the BCMA. In yet other embodiments of a multispecific NKG2d/BCMA antibody provided herein, the first binding domain that binds to NKG2d specifically binds to the NKG2d, and the second binding domain that binds to BCMA specifically binds to the BCMA.

In some embodiments, the multispecific NKG2d/BCMA antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the NKG2d antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the NKG2d/BCMA multispecific antibodies or antigen binding fragments thereof bind to a first epitope located on NKG2d and a second epitope of located on BCMA. In some embodiments, provided herein is a multispecific NKG2d/BCMA antibody comprising: (a) a first binding domain that binds to an NKG2d antigen, and (b) a second binding domain that binds to a BCMA antigen. In some embodiments, provided herein is a multispecific NKG2d/BCMA antibody comprising: (a) a first binding domain that specifically binds to an NKG2d antigen, and (b) a second binding domain that specifically binds to a BCMA antigen. In some embodiments, provided herein is a multispecific NKG2d/BCMA antibody comprising: (a) a first binding domain that binds to a first epitope on an NKG2d antigen, and (b) a second binding domain that binds to a second epitope on a BCMA antigen. In some embodiments, provided herein is a multispecific antibody comprising: (a) a first binding domain that specifically binds to a first epitope on an NKG2d antigen, and (b) a second binding domain that specifically binds to a second epitope on a BCMA antigen.

In specific embodiments, the NKG2d antigen is on the surface of an NK cell. In specific embodiments, the BCMA antigen is on the surface of a tumor cell. The binding of the NKG2d/BCMA multispecific antibody to NKG2d present on the surface of NK cells and BCMA present on the surface of tumor cells can, for example, result in the killing of the tumor cell. In other embodiments, the binding of the NKG2d/BCMA multispecific antibody to NKG2d present on the surface of NK cells and BCMA present on the surface of tumor cells can, for example, result in the activation of the NK cell.

In another aspect, provided herein is a multispecific antibody that comprises a first binding domain that binds to NKG2d and a second binding domain that binds to GPRC5d (“multispecific NKG2d/GPRC5d antibody”). In some embodiments, the multispecific NKG2d/GPRC5d antibody is a bispecific antibody. In some embodiments, the multispecific NKG2d/GPRC5d antibody is a trispecific antibody. In some embodiments, the multispecific NKG2d/GPRC5d antibody is a quadraspecific antibody.

In one embodiment, the multispecific NKG2d/GPRC5d antibody comprises: (a) a first binding domain that binds NKG2d, and (b) a second binding domain that binds to GPRC5d. In one embodiment, the multispecific NKG2d/GPRC5d antibody comprises: (a) a first binding domain that binds NKG2d, and (b) a second binding domain that binds to GPRC5d, and (c) a third binding domain that binds to a third target. In one embodiment, the multispecific NKG2d/GPRC5d antibody comprises: (a) a first binding domain that binds NKG2d, and (b) a second binding domain that binds to GPRC5d, (c) a third binding domain that binds to a third target, and (d) a fourth binding domain that binds to a fourth target.

In some embodiments of the multispecific NKG2d/GPRC5d antibodies provided herein, the first binding domain that binds NKG2d comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2. In some embodiments of the multispecific NKG2d/GPRC5d antibodies provided herein, the first binding domain that binds NKG2d comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3. In some embodiments of the multispecific NKG2d/GPRC5d antibodies provided herein, the first binding domain that binds NKG2d comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:2; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:3. In some embodiments of the multispecific NKG2d/GPRC5d antibodies provided herein, the first binding domain that binds NKG2d comprises a VH having an amino acid sequence of SEQ ID NO:2. In some embodiments of the multispecific NKG2d/GPRC5d antibodies provided herein, the first binding domain that binds NKG2d comprises a VL having an amino acid sequence of SEQ ID NO:3. In some embodiments of the multispecific NKG2d/GPRC5d antibodies provided herein, the first binding domain that binds NKG2d comprises a VH having an amino acid sequence of SEQ ID NO:2, and a VL having an amino acid sequence of SEQ ID NO:3.

In some embodiments of the multispecific NKG2d/GPRC5d antibodies provided herein, the first binding domain that binds NKG2d comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34. In some embodiments of the multispecific NKG2d/GPRC5d antibodies provided herein, the first binding domain that binds NKG2d comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:35. In some embodiments of the multispecific NKG2d/GPRC5d antibodies provided herein, the first binding domain that binds NKG2d comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:34; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:35. In some embodiments of the multispecific NKG2d/GPRC5d antibodies provided herein, the first binding domain that binds NKG2d comprises a VH having an amino acid sequence of SEQ ID NO:34. In some embodiments of the multispecific NKG2d/GPRC5d antibodies provided herein, the first binding domain that binds NKG2d comprises a VL having an amino acid sequence of SEQ ID NO:35. In some embodiments of the multispecific NKG2d/GPRC5d antibodies provided herein, the first binding domain that binds NKG2d comprises a VH having an amino acid sequence of SEQ ID NO:34, and a VL having an amino acid sequence of SEQ ID NO:35.

In some embodiments of the multispecific NKG2d/GPRC5d antibodies provided herein, the VH CDR1, VH CDR2, VH CDR3, VL CDR1, VL CDR2, and VL CDR3 amino acid sequences of the first binding domain that binds NKG2d are according to the Kabat numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the first binding domain that binds NKG2d are according to the Chothia numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the first binding domain that binds NKG2d are according to the AbM numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the first binding domain that binds NKG2d are according to the Contact numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the first binding domain that binds NKG2d are according to the IMGT numbering system.

In some embodiments of the multispecific NKG2d/GPRC5d antibodies provided herein, the first binding domain binds an NKG2d antigen. In some embodiments of the multispecific NKG2d/GPRC5d antibodies provided herein, the first binding domain binds an NKG2d epitope. In some embodiments of the multispecific NKG2d/GPRC5d antibodies provided herein, the first binding domain specifically binds to NKG2d. In some embodiments of the multispecific NKG2d/GPRC5d antibodies provided herein, the VH CDR1, VH CDR2, VH CDR3, VL CDR1, VL CDR2 and VL CDR3 of the first binding domain form a binding site for an antigen of the NKG2d. In some embodiments of the multispecific NKG2d/GPRC5d antibodies provided herein, the VH CDR1, VH CDR2, VH CDR3, VL CDR1, VL CDR2 and VL CDR3 of the first binding domain form a binding site for an epitope of the NKG2d. In some embodiments of the multispecific NKG2d/GPRC5d antibodies provided herein, the NKG2d is present on the surface of an NK cell.

In some embodiments of the multispecific NKG2d/GPRC5d antibodies provided herein, the second binding domain that binds GPRC5d comprises a VH comprising a VH CDR1, a VH CDR2, and a VH CDR3 having an amino acid sequence of a VH CDR1, a VH CDR2, and a VH CDR3, respectively, of SEQ ID NO:131. In some embodiments of the multispecific NKG2d/GPRC5d antibodies provided herein, the second binding domain that binds GPRC5d comprises a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific NKG2d/GPRC5d antibodies provided herein, the second binding domain that binds GPRC5d comprises: (i) a VH comprising a VH CDR1, a VH CDR2, and a VH CDR3 having an amino acid sequence of a VH CDR1, a VH CDR2, and a VH CDR3, respectively, of SEQ ID NO:131; and (ii) a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific NKG2d/GPRC5d antibodies provided herein, the second binding domain that binds GPRC5d comprises a VH having an amino acid sequence of SEQ ID NO:131. In some embodiments of the multispecific NKG2d/GPRC5d antibodies provided herein, the second binding domain that binds GPRC5d comprises a VL having an amino acid sequence of SEQ ID NO:132. In some embodiments of the multispecific NKG2d/GPRC5d antibodies provided herein, the second binding domain that binds GPRC5d comprises a VH having an amino acid sequence of SEQ ID NO:131, and a VL having an amino acid sequence of SEQ ID NO:132.

In some embodiments of the multispecific NKG2d/GPRC5d antibodies provided herein, the VH CDR1, VH CDR2, VH CDR3, VL CDR1, VL CDR2, and VL CDR3 amino acid sequences of the second binding domain that binds GPRC5d are according to the Kabat numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the second binding domain that binds GPRC5d are according to the Chothia numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the second binding domain that binds GPRC5d are according to the AbM numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the second binding domain that binds GPRC5d are according to the Contact numbering system. In some embodiments of the multispecific NKG2d/GPRC5d antibodies provided herein, the VH CDR1, VH CDR2, VH CDR3, VL CDR1, VL CDR2, and VL CDR3 amino acid sequences of the second binding domain that binds GPRC5d are according to the IMGT numbering system.

In some embodiments of the multispecific NKG2d/GPRC5d antibodies provided herein, the second binding domain binds a GPRC5d antigen. In some embodiments of the multispecific NKG2d/GPRC5d antibodies provided herein, the second binding domain binds a GPRC5d epitope. In some embodiments of the multispecific NKG2d/GPRC5d antibodies provided herein, the second binding domain specifically binds to GPRC5d. In some embodiments of the multispecific NKG2d/GPRC5d antibodies provided herein, the VH CDR1, VH CDR2, VH CDR3, VL CDR1, VL CDR2 and VL CDR3 of the second binding domain form a binding site for an antigen of the GPRC5d. In some embodiments, the VH CDR1, VH CDR2, VH CDR3, VL CDR1, VL CDR2 and VL CDR3 of the second binding domain form a binding site for an epitope of the GPRC5d. In some embodiments, the GPRC5d is present on the surface of a tumor cell.

In some embodiments of the multispecific NKG2d/GPRC5d antibodies provided herein, the third target is not an NKG2d antigen. In some embodiments of the multispecific NKG2d/GPRC5d antibodies provided herein, the fourth target is not an NKG2d antigen. In some embodiments of the multispecific NKG2d/GPRC5d antibodies provided herein, the third target is not an NKG2d antigen, and the fourth target is not an NKG2d antigen. In some embodiments of the multispecific NKG2d/GPRC5d antibodies provided herein, the third target is not a GPRC5d antigen. In some embodiments of the multispecific NKG2d/GPRC5d antibodies provided herein, the fourth target is not a GPRC5d antigen. In some embodiments of the multispecific NKG2d/GPRC5d antibodies provided herein, the third target is not a GPRC5d antigen, and the fourth target is not a GPRC5d antigen. In some embodiments of the multispecific NKG2d/GPRC5d antibodies provided herein, the third target is not an NKG2d epitope. In some embodiments of the multispecific NKG2d/GPRC5d antibodies provided herein, the fourth target is not an NKG2d epitope. In some embodiments of the multispecific NKG2d/GPRC5d antibodies provided herein, the third target is not an NKG2d epitope, and the fourth target is not an NKG2d epitope. In some embodiments of the multispecific NKG2d/GPRC5d antibodies provided herein, the third target is not a GPRC5d epitope. In some embodiments of the multispecific NKG2d/GPRC5d antibodies provided herein, the fourth target is not a GPRC5d epitope. In some embodiments of the multispecific NKG2d GPRC5d antibodies provided herein, the third target is not a GPRC5d epitope, and the fourth target is not a GPRC5d epitope.

In a specific embodiment, the target is from a mammal. In a specific embodiment, the target is from a rat. In a specific embodiment, the target is from a mouse. In a specific embodiment, the target is from a primate. In a specific embodiment, the target is from a human.

In specific embodiments, provided is a multispecific NKG2d/GPRC5d antibody in a knob-in-hole format. In specific embodiments, provided is a bispecific NKG2d/GPRC5d antibody in a knob-in-hole format. In specific embodiments, provided is a trispecific antibody in a knob-in-hole format. In specific embodiments, provided is a quadraspecific antibody in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, an NKG2d/GPRC5d antibody provided herein is comprised in a bispecific antibody. In some embodiments, an NKG2d/GPRC5d antibody provided herein is comprised in a trispecific antibody. In some embodiments, an NKG2d/GPRC5d antibody provided herein is comprised in a quadraspecific antibody. In some embodiments, an NKG2d/GPRC5d bispecific antibody provided herein is comprised in a multispecific antibody.

In certain embodiments, a trispecific NKG2d/GPRC5d antibody provided herein comprises a first binding domain comprising an NKG2d antibody provided herein that binds to an NKG2d epitope, a second binding domain comprising a GPRC5d antibody provided herein that binds to a GPRC5d epitope, and a third binding domain that binds to a third epitope, wherein the NKG2d epitope, the GPRC5d epitope, and the third epitope are not the same. In certain embodiments, a quadraspecific antibody provided herein comprises a first binding domain comprising an NKG2d antibody provided herein that binds to an NKG2d epitope, a second binding domain comprising a GPRC5d antibody provided herein that binds to a GPRC5d epitope, a third binding domain that binds to a third epitope, and a fourth binding domain that binds to a fourth epitope, wherein the NKG2d epitope, the GPRC5d epitope, the third epitope, and the fourth epitope are not the same. In certain embodiments, a trispecific antibody provided herein comprises a first binding domain comprising an NKG2d antibody provided herein that binds to an NKG2d antigen, a second binding domain comprising a GPRC5d antibody provided herein that binds to a GPRC5d antigen, and a third binding domain that binds to a third antigen, wherein the NKG2d antigen, the GPRC5d antigen, and the third antigen are not the same. In certain embodiments, a quadraspecific antibody provided herein that binds to an NKG2d antigen, a second binding domain comprising a GPRC5d antibody provided herein that binds to a GPRC5d antigen, a third binding domain that binds to a third antigen, and a fourth binding domain that binds to a fourth antigen, wherein the NKG2d antigen, the GPRC5d antigen, the third antigen, and the fourth antigen are not the same. In certain embodiments of a multispecific NKG2d/GPRC5d antibody provided herein, the first binding domain that binds to NKG2d specifically binds to the NKG2d. In other embodiments of a multispecific NKG2d/GPRC5d antibody provided herein, the second binding domain that binds to GPRC5d specifically binds to the GPRC5d. In yet other embodiments of a multispecific NKG2d/GPRC5d antibody provided herein, the first binding domain that binds to NKG2d specifically binds to the NKG2d, and the second binding domain that binds to GPRC5d specifically binds to the GPRC5d.

In some embodiments, the multispecific NKG2d/GPRC5d antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the NKG2d antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the NKG2d/GPRC5d multispecific antibodies or antigen binding fragments thereof bind to a first epitope located on NKG2d and a second epitope of located on GPRC5d. In some embodiments, provided herein is a multispecific NKG2d/GPRC5d antibody comprising: (a) a first binding domain that binds to an NKG2d antigen, and (b) a second binding domain that binds to a GPRC5d antigen. In some embodiments, provided herein is a multispecific NKG2d/GPRC5d antibody comprising: (a) a first binding domain that specifically binds to an NKG2d antigen, and (b) a second binding domain that specifically binds to a GPRC5d antigen. In some embodiments, provided herein is a multispecific NKG2d/GPRC5d antibody comprising: (a) a first binding domain that binds to a first epitope on an NKG2d antigen, and (b) a second binding domain that binds to a second epitope on a GPRC5d antigen. In some embodiments, provided herein is a multispecific antibody comprising: (a) a first binding domain that specifically binds to a first epitope on an NKG2d antigen, and (b) a second binding domain that specifically binds to a second epitope on a GPRC5d antigen.

In specific embodiments, the NKG2d antigen is on the surface of an NK cell. In specific embodiments, the GPRC5d antigen is on the surface of a tumor cell. The binding of the NKG2d/GPRC5d multispecific antibody to NKG2d present on the surface of NK cells and GPRC5d present on the surface of tumor cells can, for example, result in the killing of the tumor cell. In other embodiments, the binding of the NKG2d/GPRC5d multispecific antibody to NKG2d present on the surface of NK cells and GPRC5d present on the surface of tumor cells can, for example, result in the activation of the NK cell.

In specific embodiments, the NKp46 antibody comprises a VH region and a VL region. In some embodiments, the NKp46 antibody is a single chain antibody. In some embodiments, the NKp46 antibody is a single domain antibody. In some embodiments, the NKp46 antibody is a nanobody. In certain embodiments, the NKp46 antibody is a VHH antibody. In certain embodiments, the NKp46 antibody is a llama antibody. In some embodiments, the NKp46 antibody is not a single chain antibody. In some embodiments, the NKp46 antibody is not a single domain antibody. In some embodiments, the NKp46 antibody is not a nanobody. In certain embodiments, the NKp46 antibody is not a VHH antibody. In certain embodiments, the NKp46 antibody is not a llama antibody. In some embodiments, the NKp46 antibody is a multispecific antibody. In other embodiments, the NKp46 is a bispecific antibody. In certain embodiments, the multispecific antibody comprises an antigen binding fragment of an NKp46 antibody provided herein. In other embodiments, the bispecific antibody comprises an antigen binding fragment of an NKp46 antibody provided herein.

In specific embodiments, provided herein is a multispecific antibody that binds NKp46. In some embodiments, the multispecific antibody is a bispecific antibody. In some embodiments, the multispecific antibody is a trispecific antibody. In some embodiments, the multispecific antibody is a quadraspecific antibody. In one embodiment, the multispecific NKp46 antibody comprises: (a) a first binding domain that binds NKp46, and (b) a second binding domain that binds to a second target. In one embodiment, the multispecific NKp46 antibody comprises: (a) a first binding domain that binds NKp46, and (b) a second binding domain that binds to a second target, and (c) a third binding domain that binds to a third target. In one embodiment, the multispecific NKp46 antibody comprises: (a) a first binding domain that binds NKp46, and (b) a second binding domain that binds to a second target, (c) a third binding domain that binds to a third target, and (d) a fourth binding domain that binds to a fourth target.

In another aspect, provided herein is a bispecific antibody comprising: (a) a first binding domain that binds to NKp46, and (b) a second binding domain that binds to a second target that is not NKp46. In another aspect, provided herein is a bispecific antibody comprising: (a) a first binding domain that binds to NKp46, and (b) a second binding domain that binds to a second target that binds to an antigen expressed on a tumor cell. In some embodiments, the second binding domain binds to BCMA. In some embodiments, the second binding domain binds to GPRC5d.

In certain embodiments, provided herein is an anti-NKp46 bispecific antibody comprising a binding domain that binds to NKp46 having a VH region, VL region, VH CDR1, VH CDR2, VH CDR3, VL CDR1, VL CDR2, and/or VL CDR3 of any one of the antibodies described herein. In some embodiments, provided herein is an anti-NKp46 bispecific antibody comprising a binding domain that binds to NKp46 having a VH region of any one of the antibodies described herein. In some embodiments, provided herein is an anti-NKp46 bispecific antibody comprising a binding domain that binds to NKp46 having a VL region of any one of the antibodies described herein. In some embodiments, provided herein is an anti-NKp46 bispecific antibody comprising a binding domain that binds to NKp46 having a VH region of any one of the antibodies described herein, and a VL region of any one of the antibodies described herein. In some embodiments, provided herein is an anti-NKp46 bispecific antibody comprising a binding domain that binds to NKp46 having a VH CDR1, VH CDR2, and VH CDR3 of any one of the antibodies described. In some embodiments, provided herein is an anti-NKp46 bispecific antibody comprising a binding domain that binds to NKp46 having a VL CDR1, VL CDR2, and VL CDR3 of any one of the antibodies described herein. In some embodiments, provided herein is an anti-NKp46 bispecific antibody comprising a binding domain that binds to NKp46 having a VH CDR1, VH CDR2, and VH CDR3 of any one of the antibodies described herein; and a VL CDR1, VL CDR2, and VL CDR3 of any one of the antibodies described herein.

In certain embodiments, the anti-NKp46 antibody is a bispecific antibody. In some embodiments, the anti-NKp46 bispecific antibody further comprises a second binding domain that binds to BCMA having a VH region, VL region, VH CDR1, VH CDR2, VH CDR3, VL CDR1, VL CDR2, and/or VL CDR3 of an anti-BCMA antibody provided herein. In some embodiments, the anti-NKp46 bispecific antibody further comprises a second binding domain that binds to BCMA having a VH region of an anti-BCMA antibody provided herein. In some embodiments, the anti-NKp46 bispecific antibody further comprises a second binding domain that binds to BCMA having a VL region of an anti-BCMA antibody provided herein. In some embodiments, the anti-NKp46 bispecific antibody further comprises a second binding domain that binds to BCMA having a VH region of an anti-BCMA antibody provided herein, and a VL region of an anti-BCMA antibody provided herein. In some embodiments, the anti-NKp46 bispecific antibody further comprises a second binding domain that binds to BCMA having a VH CDR1, VH CDR2, and VH CDR3 of an anti-BCMA antibody provided herein. In some embodiments, the anti-NKp46 bispecific antibody further comprises a second binding domain that binds to BCMA having a VL CDR1, VL CDR2, and VL CDR3 of an anti-BCMA antibody provided herein. In some embodiments, the anti-NKp46 bispecific antibody further comprises a second binding domain that binds to BCMA having a VH CDR1, VH CDR2, and VH CDR3 of an anti-BCMA antibody provided herein, and a VL CDR1, VL CDR2, and VL CDR3 of an anti-BCMA antibody provided herein.

In certain embodiments, the anti-NKp46 antibody is a bispecific antibody. In some embodiments, the anti-NKp46 bispecific antibody further comprises a second binding domain that binds to GPRC5d having a VH region, VL region, VH CDR1, VH CDR2, VH CDR3, VL CDR1, VL CDR2, and/or VL CDR3 of an anti-GPRC5d antibody provided herein. In some embodiments, the anti-NKp46 bispecific antibody further comprises a second binding domain that binds to GPRC5d having a VH region of an anti-GPRC5d antibody provided herein. In some embodiments, the anti-NKp46 bispecific antibody further comprises a second binding domain that binds to GPRC5d having a VL region of an anti-GPRC5d antibody provided herein. In some embodiments, the anti-NKp46 bispecific antibody further comprises a second binding domain that binds to GPRC5d having a VH region of an anti-GPRC5d antibody provided herein, and a VL region of an anti-GPRC5d antibody provided herein. In some embodiments, the anti-NKp46 bispecific antibody further comprises a second binding domain that binds to GPRC5d having a VH CDR1, VH CDR2, and VH CDR3 of an anti-GPRC5d antibody provided herein. In some embodiments, the anti-NKp46 bispecific antibody further comprises a second binding domain that binds to GPRC5d having a VL CDR1, VL CDR2, and VL CDR3 of an anti-GPRC5d antibody provided herein. In some embodiments, the anti-NKp46 bispecific antibody further comprises a second binding domain that binds to GPRC5d having a VH CDR1, VH CDR2, and VH CDR3 of an anti-GPRC5d antibody provided herein, and a VL CDR1, VL CDR2, and VL CDR3 of an anti-GPRC5d antibody provided herein.

In some embodiments of the multispecific antibodies provided herein, the first binding domain that binds NKp46 is as described or derived from the antibodies described above. In some specific embodiments of the multispecific antibodies provided herein, the first binding domain that binds NKp46 comprising: (i) a VH comprising a VH CDR1, a VH CDR2, and a VH CDR3 having an amino acid sequence of SEQ ID NOs:69, 70, and 71, respectively, and (ii) a VL comprising a VL CDR1, VL CDR2, and VL CDR3 having an amino acid sequence of SEQ ID NOs:72, 73, and 74, respectively. In some embodiments of the multispecific antibodies provided herein, the first binding domain that binds NKp46 comprising: (i) a VH comprising a VH CDR1, a VH CDR2, and a VH CDR3 having an amino acid sequence of SEQ ID NOs:75, 76, and 77, respectively, and (ii) a VL comprising a VL CDR1, VL CDR2, and VL CDR3 having an amino acid sequence of SEQ ID NOs:78, 79, and 80, respectively. In some embodiments of the multispecific antibodies provided herein, the first binding domain that binds NKp46 comprising: (i) a VH comprising a VH CDR1, a VH CDR2, and a VH CDR3 having an amino acid sequence of SEQ ID NOs:81, 82, and 83, respectively, and (ii) a VL comprising a VL CDR1, VL CDR2, and VL CDR3 having an amino acid sequence of SEQ ID NOs:84, 85, and 86, respectively. In some embodiments of the multispecific antibodies provided herein, the first binding domain that binds NKp46 comprising: (i) a VH comprising a VH CDR1, a VH CDR2, and a VH CDR3 having an amino acid sequence of SEQ ID NOs:87, 88, and 89, respectively, and (ii) a VL comprising a VL CDR1, VL CDR2, and VL CDR3 having an amino acid sequence of SEQ ID NOs:90, 91, and 92, respectively. In some embodiments of the multispecific antibodies provided herein, the first binding domain that binds NKp46 comprising: (i) a VH comprising a VH CDR1, a VH CDR2, and a VH CDR3 having an amino acid sequence of SEQ ID NOs:93, 94, and 95, respectively, and (ii) a VL comprising a VL CDR1, VL CDR2, and VL CDR3 having an amino acid sequence of SEQ ID NOs:96, 97, and 98 respectively. In some embodiments of the multispecific antibodies provided herein, the first binding domain that binds NKp46 comprises a VH comprising a VH CDR1, a VH CDR2, and a VH CDR3 having an amino acid sequence of a VH CDR1, a VH CDR2, and a VH CDR3, respectively, of SEQ ID NO:67. In some embodiments of the multispecific antibodies provided herein, the first binding domain that binds NKp46 comprises a VL comprising a VL CDR1, a VL CDR2, and a VL CDR3 having an amino acid sequence of a VL CDR1, a VL CDR2, and a VL CDR3, respectively, of SEQ ID NO:68. In some embodiments of the multispecific antibodies provided herein, the first binding domain that binds NKp46 comprises: (i) a VH comprising a VH CDR1, a VH CDR2, and a VH CDR3 having an amino acid sequence of a VH CDR1, a VH CDR2, and a VH CDR3, respectively, of SEQ ID NO:67; and (ii) a VL comprising a VL CDR1, a VL CDR2, and a VL CDR3 having an amino acid sequence of a VL CDR1, a VL CDR2, and a VL CDR3, respectively, of SEQ ID NO:68. In some embodiments of the multispecific antibodies provided herein, the first binding domain that binds NKp46 comprises a VH having an amino acid sequence of SEQ ID NO:67. In some embodiments of the multispecific antibodies provided herein, the first binding domain that binds NKp46 comprises a VL having an amino acid sequence of SEQ ID NO:68. In some embodiments of the multispecific antibodies provided herein, the first binding domain that binds NKp46 comprises a VH having an amino acid sequence of SEQ ID NO:67, and a VL having an amino acid sequence of SEQ ID NO:68. In some embodiments of the multispecific antibodies provided herein, the first binding domain that binds NKp46 comprises a VH comprising an amino acid sequence having at least 95% identity to the amino acid sequence of SEQ ID NO:67. In some embodiments of the multispecific antibodies provided herein, the first binding domain that binds NKp46 comprises a VL comprising an amino acid sequence having at least 95% identity to the amino acid sequence of SEQ ID NO:68. In some embodiments of the multispecific antibodies provided herein, the first binding domain that binds NKp46 comprises a VH comprising an amino acid sequence having at least 95% identity to the amino acid sequence of SEQ ID NO:67, and a VL comprising an amino acid sequence having at least 95% identity to the amino acid sequence of SEQ ID NO:68.

In some embodiments, the VH CDR1, VH CDR2, VH CDR3, VL CDR1, VL CDR2, and VL CDR3 amino acid sequences of the first binding domain that binds NKp46 are according to the Kabat numbering system. In some embodiments, the VH CDR1, VH CDR2, VH CDR3, VL CDR1, VL CDR2, and VL CDR3 amino acid sequences of the first binding domain that binds NKp46 are according to the Chothia numbering system. In some embodiments, the VH CDR1, VH CDR2, VH CDR3, VL CDR1, VL CDR2, and VL CDR3 amino acid sequences of the first binding domain that binds NKp46 are according to the AbM numbering system. In some embodiments, the VH CDR1, VH CDR2, VH CDR3, VL CDR1, VL CDR2, and VL CDR3 amino acid sequences of the first binding domain that binds NKp46 are according to the Contact numbering system. In some embodiments, the VH CDR1, VH CDR2, VH CDR3, VL CDR1, VL CDR2, and VL CDR3 amino acid sequences of the first binding domain that binds NKp46 are according to the IMGT numbering system.

In some embodiments of the multispecific NKp46 antibodies provided herein, the first binding domain binds an NKp46 antigen. In some embodiments, the first binding domain binds an NKp46 epitope. In some embodiments, the first binding domain specifically binds to NKp46. In some embodiments, the VH CDR1, VH CDR2, VH CDR3, VL CDR1, VL CDR2 and VL CDR3 of the first binding domain form a binding site for an antigen of the NKp46. In some embodiments, the VH CDR1, VH CDR2, VH CDR3, VL CDR1, VL CDR2 and VL CDR3 of the first binding domain form a binding site for an epitope of the NKp46. In some embodiments, the NKp46 is present on the surface of an NK cell.

In another aspect, provided herein is a multispecific antibody that competes for binding to NKp46 with any of the NKp46 antibodies described herein. In another aspect, provided herein is a multispecific antibody that binds to the same epitope as any of the NKp46 antibodies described herein. In another aspect, provided is a multispecific NKp46 antibody that binds an epitope on NKp46 that overlaps with the epitope on NKp46 bound by an NKp46 antibody described herein.

In one aspect, provided is a multispecific antibody that competes for binding to NKp46 with an NKp46 reference antibody. In another aspect, provided is a multispecific NKp46 antibody that binds to the same NKp46 epitope as an NKp46 reference antibody. In another aspect, provided is a multispecific NKp46 antibody that binds an epitope on NKp46 that overlaps with the epitope on NKp46 bound by an NKp46 reference antibody.

In one embodiment, the NKp46 reference antibody comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68.

In some embodiments of the multispecific NKp46 antibodies provided herein, the second target is not an NKp46 antigen. In some embodiments of the multispecific NKp46 antibodies provided herein, the third target is not an NKp46 antigen. In some embodiments of the multispecific NKp46 antibodies provided herein, the fourth target is not an NKp46 antigen. In some embodiments of the multispecific NKp46 antibodies provided herein, the second target is not an NKp46 antigen, and the third target is not an NKp46 antigen. In some embodiments of the multispecific NKp46 antibodies provided herein, the second target is not an NKp46 antigen, and the fourth target is not an NKp46 antigen. In some embodiments of the multispecific NKp46 antibodies provided herein, the third target is not an NKp46 antigen, and the fourth target is not an NKp46 antigen. In some embodiments of the multispecific NKp46 antibodies provided herein, the second target is not an NKp46 antigen, the third target is not an NKp46 antigen, and the fourth target is not an NKp46 antigen. In some embodiments of the multispecific NKp46 antibodies provided herein, the second target is not an NKp46 epitope. In some embodiments of the multispecific NKp46 antibodies provided herein, the third target is not an NKp46 epitope. In some embodiments of the multispecific NKp46 antibodies provided herein, the fourth target is not an NKp46 epitope. In some embodiments of the multispecific NKp46 antibodies provided herein, the second target is not an NKp46 epitope, and the third target is not an NKp46 epitope. In some embodiments of the multispecific NKp46 antibodies provided herein, the second target is not an NKp46 epitope, and the fourth target is not an NKp46 epitope. In some embodiments of the multispecific NKp46 antibodies provided herein, the third target is not an NKp46 epitope, and the fourth target is not an NKp46 epitope. In some embodiments of the multispecific NKp46 antibodies provided herein, the second target is not an NKp46 epitope, the third target is not an NKp46 epitope, and the fourth target is not an NKp46 epitope. In some embodiments of the multispecific NKp46 antibodies provided herein, the second target is BCMA. In some embodiments of the multispecific NKp46 antibodies provided herein, the second target is GPRC5d.

The binding of the multispecific antibody provided herein to NKp46 present on the surface of the NK cell, and the binding of the second target antigen present on the surface of the second target cell can, for example, result in the killing of the second target cell. In other embodiment, the binding of the multispecific antibody provided herein to NKp46 present on the surface of the NK cell, and the binding of a second target antigen can, for example, result in the activation of the NK cell. In some embodiments, provided herein is a bispecific antibody comprising: (a) a first binding domain that binds to NKp46, and (b) a second binding domain that binds to a cancer antigen present on the surface of a cancer cell. In some embodiments, the antigen on the surface of the cancer cell is a tumor-specific antigen. In some embodiments, the antigen on the surface of the cancer cell is a tumor associated antigen. In some embodiments, the antigen on the surface of the cancer cell is a neoantigen. In certain embodiments, the first binding domain of the bispecific antibody specifically binds NKp46. In some embodiments, the NKp46 is present on the surface of an NK cell. In some embodiments, the cancer cell is killed when the bispecific antibody binds to the NKp46 on the surface of the NK cell and the antigen on the surface of the cancer cell.

The binding of the multispecific antibody provided herein to NKp46 present on the surface of the T cells, and the binding of the second target antigen present on the surface of the second target cell can, for example, result in the killing of the second target cell. In other embodiment, the binding of the multispecific antibody provided herein to NKp46 present on the surface of the T cells, and the binding of a second target antigen can, for example, result in the activation of the T cells. In some embodiments, provided herein is a bispecific antibody comprising: (a) a first binding domain that binds to NKp46, and (b) a second binding domain that binds to a cancer antigen present on the surface of a cancer cell. In some embodiments, the antigen on the surface of the cancer cell is a tumor-specific antigen. In some embodiments, the antigen on the surface of the cancer cell is a tumor associated antigen. In some embodiments, the antigen on the surface of the cancer cell is a neoantigen. In certain embodiments, the first binding domain of the bispecific antibody specifically binds NKp46. In some embodiments, the NKp46 is present on the surface of a T cells. In some embodiments, the cancer cell is killed when the bispecific antibody binds to the NKp46 on the surface of the T cells and the antigen on the surface of the cancer cell. In some embodiments, the T cells are gamma delta T cells. In some embodiments, the T cells are mucosal population of innate lymphoid cells.

In another aspect, provided herein is a bispecific antibody comprising: (a) a first binding domain that binds to NKp46, and (b) a second binding domain that binds to BCMA. In certain embodiments, the first binding domain of the bispecific antibody specifically binds NKp46. In some embodiments, the NKp46 is present on the surface of an NK cell. In some embodiments, the BCMA is on the surface of a cell. In certain embodiments, the NKp46 is present on the surface of an NK cell, and the BCMA is on the surface of a cell. In some embodiments, the cell having the BCMA on the surface is killed when the bispecific antibody binds to the NKp46 on the surface of the NK cell and the BCMA on the surface of the cell. In some embodiments, the BCMA is on the surface of a cancer cell. In certain embodiments, the NKp46 is present on the surface of an NK cell, and the BCMA is on the surface of a cancer cell. In some embodiments, the cancer cell is killed when the bispecific antibody binds to the NKp46 on the surface of the NK cell and the BCMA on the surface of the cell. Bispecific antibodies comprising any of the NKp46 antibodies provided herein as the first binding domain are contemplated, in certain embodiments. In addition, bispecific antibodies comprising any of the NKp46 antibodies provided herein as the first binding domain, and a second binding domain that binds to BCMA are also contemplated in certain embodiments.

In some embodiments, the multispecific antibodies provided herein, is a bispecific antibody comprising: (a) a first binding domain that binds to NKp46, and (b) a second binding domain that binds to BCMA, wherein the second binding domain that binds BCMA comprising: (i) a VH comprising a VH CDR1, a VH CDR2, and a VH CDR3 having an amino acid sequence of SEQ ID NOs:101, 102, and 103, respectively, and (ii) a VL comprising a VL CDR1, VL CDR2, and VL CDR3 having an amino acid sequence of SEQ ID NOs:104, 105, and 106, respectively. In some embodiments of the multispecific antibodies provided herein, the second binding domain that binds BCMA comprising: (i) a VH comprising a VH CDR1, a VH CDR2, and a VH CDR3 having an amino acid sequence of SEQ ID NOs:107, 108, and 109, respectively, and (ii) a VL comprising a VL CDR1, VL CDR2, and VL CDR3 having an amino acid sequence of SEQ ID NOs:110, 111, and 112, respectively. In some embodiments of the multispecific antibodies provided herein, the second binding domain that binds BCMA comprising: (i) a VH comprising a VH CDR1, a VH CDR2, and a VH CDR3 having an amino acid sequence of SEQ ID NOs:113, 114, and 115, respectively, and (ii) a VL comprising a VL CDR1, VL CDR2, and VL CDR3 having an amino acid sequence of SEQ ID NOs:116, 117, and 118, respectively. In some embodiments of the multispecific antibodies provided herein, the second binding domain that binds BCMA comprising: (i) a VH comprising a VH CDR1, a VH CDR2, and a VH CDR3 having an amino acid sequence of SEQ ID NOs:119, 120, and 121, respectively, and (ii) a VL comprising a VL CDR1, VL CDR2, and VL CDR3 having an amino acid sequence of SEQ ID NOs:122, 123, and 124, respectively. In some embodiments of the multispecific antibodies provided herein, the second binding domain that binds BCMA comprising: (i) a VH comprising a VH CDR1, a VH CDR2, and a VH CDR3 having an amino acid sequence of SEQ ID NOs:125, 126, and 127, respectively, and (ii) a VL comprising a VL CDR1, VL CDR2, and VL CDR3 having an amino acid sequence of SEQ ID NOs:128, 129, and 130, respectively. In some embodiments of the multispecific antibodies provided herein, the second binding domain that binds BCMA comprises a VH comprising a VH CDR1, a VH CDR2, and a VH CDR3 having an amino acid sequence of a VH CDR1, a VH CDR2, and a VH CDR3, respectively, of SEQ ID NO:99. In some embodiments of the multispecific antibodies provided herein, the second binding domain that binds BCMA comprises a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific antibodies provided herein, the second binding domain that binds BCMA comprises: (i) a VH comprising a VH CDR1, a VH CDR2, and a VH CDR3 having an amino acid sequence of a VH CDR1, a VH CDR2, and a VH CDR3, respectively, of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific antibodies provided herein, the second binding domain that binds BCMA comprises a VH having an amino acid sequence of SEQ ID NO:99. In some embodiments of the multispecific antibodies provided herein, the second binding domain that binds BCMA comprises a VL having an amino acid sequence of SEQ ID NO:100. In some embodiments of the multispecific antibodies provided herein, the second binding domain that binds BCMA comprises a VH having an amino acid sequence of SEQ ID NO:99, and a VL having an amino acid sequence of SEQ ID NO:100. In some embodiments of the multispecific antibodies provided herein, the second binding domain that binds BCMA comprises a VH comprising an amino acid sequence having at least 95% identity to the amino acid sequence of SEQ ID NO:99. In some embodiments of the multispecific antibodies provided herein, the second binding domain that binds BCMA comprises a VL comprising an amino acid sequence having at least 95% identity to the amino acid sequence of SEQ ID NO:100. In some embodiments of the multispecific antibodies provided herein, the second binding domain that binds BCMA comprises a VH comprising an amino acid sequence having at least 95% identity to the amino acid sequence of SEQ ID NO:99, and a VL comprising an amino acid sequence having at least 95% identity to the amino acid sequence of SEQ ID NO:100.

In another aspect, provided herein is a bispecific antibody comprising: (a) a first binding domain that binds to NKp46, and (b) a second binding domain that binds to GPRC5d. In certain embodiments, the first binding domain of the bispecific antibody specifically binds NKp46. In some embodiments, the NKp46 is present on the surface of an NK cell. In some embodiments, the GPRC5d is on the surface of a cell. In certain embodiments, the NKp46 is present on the surface of an NK cell, and the GPRC5d is on the surface of a cell. In some embodiments, the cell having the GPRC5d on the surface is killed when the bispecific antibody binds to the NKp46 on the surface of the NK cell and the GPRC5d on the surface of the cell. In some embodiments, the GPRC5d is on the surface of a cancer cell. In certain embodiments, the NKp46 is present on the surface of an NK cell, and the GPRC5d is on the surface of a cancer cell. In some embodiments, the cancer cell is killed when the bispecific antibody binds to the NKp46 on the surface of the NK cell and the GPRC5d on the surface of the cancer cell. Bispecific antibodies comprising any of the NKp46 antibodies provided herein as the first binding domain are contemplated, in certain embodiments. In addition, bispecific antibodies comprising any of the NKp46 antibodies provided herein as the first binding domain, and a second binding domain that binds to GPRC5d are also contemplated in certain embodiments.

In some embodiments, the multispecific antibodies provided herein is a bispecific antibody comprising: (a) a first binding domain that binds to NKp46, and (b) a second binding domain that binds to GPRC5d, wherein the second binding domain that binds GPRC5d comprising: (i) a VH comprising a VH CDR1, a VH CDR2, and a VH CDR3 having an amino acid sequence of SEQ ID NOs:133, 134, and 135, respectively, and (ii) a VL comprising a VL CDR1, VL CDR2, and VL CDR3 having an amino acid sequence of SEQ ID NOs:136, 137, and 138, respectively. In some embodiments of the multispecific antibodies provided herein, the second binding domain that binds GPRC5d comprising: (i) a VH comprising a VH CDR1, a VH CDR2, and a VH CDR3 having an amino acid sequence of SEQ ID NOs:139, 140, and 141, respectively, and (ii) a VL comprising a VL CDR1, VL CDR2, and VL CDR3 having an amino acid sequence of SEQ ID NOs:142, 143, and 144, respectively. In some embodiments of the multispecific antibodies provided herein, the second binding domain that binds GPRC5d comprising: (i) a VH comprising a VH CDR1, a VH CDR2, and a VH CDR3 having an amino acid sequence of SEQ ID NOs:145, 146, and 147, respectively, and (ii) a VL comprising a VL CDR1, VL CDR2, and VL CDR3 having an amino acid sequence of SEQ ID NOs:148, 149, and 150, respectively. In some embodiments of the multispecific antibodies provided herein, the second binding domain that binds GPRC5d comprising: (i) a VH comprising a VH CDR1, a VH CDR2, and a VH CDR3 having an amino acid sequence of SEQ ID NOs:151, 152, and 153, respectively, and (ii) a VL comprising a VL CDR1, VL CDR2, and VL CDR3 having an amino acid sequence of SEQ ID NOs:154, 155, and 156, respectively. In some embodiments of the multispecific antibodies provided herein, the second binding domain that binds GPRC5d comprising: (i) a VH comprising a VH CDR1, a VH CDR2, and a VH CDR3 having an amino acid sequence of SEQ ID NOs:157, 158, and 159, respectively, and (ii) a VL comprising a VL CDR1, VL CDR2, and VL CDR3 having an amino acid sequence of SEQ ID NOs:160, 161, and 162, respectively. In some embodiments of the multispecific antibodies provided herein, the second binding domain that binds GPRC5d comprises a VH comprising a VH CDR1, a VH CDR2, and a VH CDR3 having an amino acid sequence of a VH CDR1, a VH CDR2, and a VH CDR3, respectively, of SEQ ID NO:131. In some embodiments of the multispecific antibodies provided herein, the second binding domain that binds GPRC5d comprises a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific antibodies provided herein, the second binding domain that binds GPRC5d comprises: (i) a VH comprising a VH CDR1, a VH CDR2, and a VH CDR3 having an amino acid sequence of a VH CDR1, a VH CDR2, and a VH CDR3, respectively, of SEQ ID NO:131; and (ii) a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific antibodies provided herein, the second binding domain that binds GPRC5d comprises a VH having an amino acid sequence of SEQ ID NO:131. In some embodiments of the multispecific antibodies provided herein, the second binding domain that binds GPRC5d comprises a VL having an amino acid sequence of SEQ ID NO:132. In some embodiments of the multispecific antibodies provided herein, the second binding domain that binds GPRC5d comprises a VH having an amino acid sequence of SEQ ID NO:131, and a VL having an amino acid sequence of SEQ ID NO:132. In some embodiments of the multispecific antibodies provided herein, the second binding domain that binds GPRC5d comprises a VH comprising an amino acid sequence having at least 95% identity to the amino acid sequence of SEQ ID NO:131. In some embodiments of the multispecific antibodies provided herein, the second binding domain that binds GPRC5d comprises a VL comprising an amino acid sequence having at least 95% identity to the amino acid sequence of SEQ ID NO:132. In some embodiments of the multispecific antibodies provided herein, the second binding domain that binds GPRC5d comprises a VH comprising an amino acid sequence having at least 95% identity to the amino acid sequence of SEQ ID NO:131, and a VL comprising an amino acid sequence having at least 95% identity to the amino acid sequence of SEQ ID NO:132.

In specific embodiments, provided is a multispecific antibody comprising an NKp46 antibody provided herein in a knob-in-hole format. In specific embodiments, provided is a bispecific antibody comprising an NKp46 antibody provided herein in a knob-in-hole format. In specific embodiments, provided is a trispecific antibody comprising an NKp46 antibody provided herein in a knob-in-hole format. In specific embodiments, provided is a quadraspecific antibody comprising an NKp46 antibody provided herein in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, an NKp46 antibody provided herein is comprised in a bispecific antibody. In some embodiments, an NKp46 antibody provided herein is comprised in a trispecific antibody. In some embodiments, an NKp46 antibody provided herein is comprised in a quadraspecific antibody. In some embodiments, an NKp46 bispecific antibody provided herein is comprised in a multispecific antibody.

In certain embodiments, a multispecific antibody provided herein comprises a first binding domain comprising an NKp46 antibody provided herein that binds to a first NKp46 epitope, and a second binding domain that binds to a second epitope, wherein the first NKp46 epitope and the second epitope are not the same. In certain embodiments, a bispecific antibody provided herein comprises a first binding domain comprising an NKp46 antibody provided herein that binds to a first NKp46 epitope, and a second binding domain that binds to a second epitope, wherein the first NKp46 epitope and the second epitope are not the same. In certain embodiments, a trispecific antibody provided herein comprises a first binding domain comprising an NKp46 antibody provided herein that binds to a first NKp46 epitope, a second binding domain that binds to a second epitope, and a third binding domain that binds to a third epitope, wherein the first NKp46 epitope, the second epitope, and the third epitope are not the same. In certain embodiments, a quadraspecific antibody provided herein comprises a first binding domain comprising an NKp46 antibody provided herein that binds to a first NKp46 epitope, a second binding domain that binds to a second epitope, a third binding domain that binds to a third epitope, and a fourth binding domain that binds to a fourth epitope, wherein the first NKp46 epitope, the second epitope, the third epitope, and the fourth epitope are not the same. In certain embodiments, a multispecific antibody provided herein comprises a first binding domain comprising an NKp46 antibody provided herein that binds to a first NKp46 antigen, and a second binding domain that binds to a second antigen, wherein the first NKp46 antigen and the second antigen are not the same. In certain embodiments, a bispecific antibody provided herein comprises a first binding domain comprising an NKp46 antibody provided herein that binds to a first NKp46 antigen, and a second binding domain that binds to a second antigen, wherein the first NKp46 antigen and the second antigen are not the same. In certain embodiments, a trispecific antibody provided herein comprises a first binding domain comprising an NKp46 antibody provided herein that binds to a first NKp46 antigen, a second binding domain that binds to a second antigen, and a third binding domain that binds to a third antigen, wherein the first NKp46 antigen, the second antigen, and the third antigen are not the same. In certain embodiments, a quadraspecific antibody provided herein comprises a first binding domain comprising an NKp46 antibody provided herein that binds to a first NKp46 antigen, a second binding domain that binds to a second antigen, a third binding domain that binds to a third antigen, and a fourth binding domain that binds to a fourth antigen, wherein the first NKp46 antigen, the second antigen, the third antigen, and the fourth antigen are not the same. In a specific embodiment, an NKp46 antibody, or antigen binding fragment thereof, provided herein specifically binds to NKp46.

In some embodiments, the multispecific antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the NKp46 antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the NKp46 multispecific antibodies or antigen binding fragments thereof bind to a first epitope located on NKp46 and a second epitope of a second target antigen. In some embodiments, provided herein is a multispecific antibody comprising: (a) a first binding domain that binds to an NKp46 antigen, and (b) a second binding domain that binds to a second target antigen. In some embodiments, provided herein is a multispecific antibody comprising: (a) a first binding domain that specifically binds to an NKp46 antigen, and (b) a second binding domain that specifically binds to a second target antigen. In some embodiments, provided herein is a multispecific antibody comprising: (a) a first binding domain that binds to a first epitope on an NKp46 antigen, and (b) a second binding domain that binds to a second epitope on a second target antigen. In some embodiments, provided herein is a multispecific antibody comprising: (a) a first binding domain that specifically binds to a first epitope on an NKp46 antigen, and (b) a second binding domain that specifically binds to a second epitope on a second target antigen.

In specific embodiments, the NKp46 antigen is on the surface of an NK cell. In certain embodiments, the second target antigen is not NKp46. The binding of the NKp46 multispecific antibody to NKp46 present on the surface of the NK cell, and the binding of the second target antigen present on the surface of the second target cell can, for example, result in the killing of the second target cell. In other embodiment, the binding of the NKp46 multispecific antibody to NKp46 present on the surface of the NK cell, and the binding of a second target antigen can, for example, result in the activation of the NK cell.

In specific embodiments, the NKp46 antigen is on the surface of a T cell. In certain embodiments, the second target antigen is not NKp46. The binding of the NKp46 multispecific antibody to NKp46 present on the surface of the T cell, and the binding of the second target antigen present on the surface of the second target cell can, for example, result in the killing of the second target cell. In other embodiment, the binding of the NKp46 multispecific antibody to NKp46 present on the surface of the T cell, and the binding of a second target antigen can, for example, result in the activation of the T cell. In some embodiments, the T cell is a gamma delta T cell. In some embodiments, the T cell is an innate lymphoid cell.

In another aspect, provided herein is a multispecific antibody that comprises a first binding domain that binds to NKp46 and a second binding domain that binds to BCMA (“multispecific NKp46/BCMA antibody”). In some embodiments, the multispecific NKp46/BCMA antibody is a bispecific antibody. In some embodiments, the multispecific NKp46/BCMA antibody is a trispecific antibody. In some embodiments, the multispecific NKp46/BCMA antibody is a quadraspecific antibody.

In some embodiments, the multispecific NKp46/BCMA antibody provided herein, comprises: (a) a first binding domain that binds NKp46, and (b) a second binding domain that binds to BCMA. In one embodiment, the multispecific NKp46/BCMA antibody comprises: (a) a first binding domain that binds NKp46, and (b) a second binding domain that binds to BCMA, and (c) a third binding domain that binds to a third target. In one embodiment, the multispecific NKp46/BCMA antibody comprises: (a) a first binding domain that binds NKp46, and (b) a second binding domain that binds to BCMA, (c) a third binding domain that binds to a third target, and (d) a fourth binding domain that binds to a fourth target.

In some embodiments of the multispecific NKp46/BCMA antibodies provided herein, the first binding domain that binds NKp46 comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67. In some embodiments of the multispecific NKp46/BCMA antibodies provided herein, the first binding domain that binds NKp46 comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68. In some embodiments of the multispecific NKp46/BCMA antibodies provided herein, the first binding domain that binds NKp46 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68. In some embodiments of the multispecific NKp46/BCMA antibodies provided herein, the first binding domain that binds NKp46 comprises a VH having an amino acid sequence of SEQ ID NO:67. In some embodiments of the multispecific NKp46/BCMA antibodies provided herein, the first binding domain that binds NKp46 comprises a VL having an amino acid sequence of SEQ ID NO:68. In some embodiments of the multispecific NKp46/BCMA antibodies provided herein, the first binding domain that binds NKp46 comprises a VH having an amino acid sequence of SEQ ID NO:67, and a VL having an amino acid sequence of SEQ ID NO:68.

In some embodiments of the multispecific NKp46/BCMA antibodies provided herein, the VH CDR1, VH CDR2, VH CDR3, VL CDR1, VL CDR2, and VL CDR3 amino acid sequences of the first binding domain that binds NKp46 are according to the Kabat numbering system. In some embodiments of the multispecific NKp46/BCMA antibodies provided herein, the VH CDR1, VH CDR2, VH CDR3, VL CDR1, VL CDR2, and VL CDR3 amino acid sequences of the first binding domain that binds NKp46 are according to the Chothia numbering system. In some embodiments of the multispecific NKp46/BCMA antibodies provided herein, the VH CDR1, VH CDR2, VH CDR3, VL CDR1, VL CDR2, and VL CDR3 amino acid sequences of the first binding domain that binds NKp46 are according to the AbM numbering system. In some embodiments of the multispecific NKp46/BCMA antibodies provided herein, the VH CDR1, VH CDR2, VH CDR3, VL CDR1, VL CDR2, and VL CDR3 amino acid sequences of the first binding domain that binds NKp46 are according to the Contact numbering system. In some embodiments of the multispecific NKp46/BCMA antibodies provided herein, the VH CDR1, VH CDR2, VH CDR3, VL CDR1, VL CDR2, and VL CDR3 amino acid sequences of the first binding domain that binds NKp46 are according to the IMGT numbering system.

In some embodiments of the multispecific NKp46/BCMA antibodies provided herein, the first binding domain binds an NKp46 antigen. In some embodiments of the multispecific NKp46/BCMA antibodies provided herein, the first binding domain binds an NKp46 epitope. In some embodiments of the multispecific NKp46/BCMA antibodies provided herein, the first binding domain specifically binds to NKp46. In some embodiments of the multispecific NKp46/BCMA antibodies provided herein, the VH CDR1, VH CDR2, VH CDR3, VL CDR1, VL CDR2 and VL CDR3 of the first binding domain form a binding site for an antigen of the NKp46. In some embodiments of the multispecific NKp46/BCMA antibodies provided herein, the VH CDR1, VH CDR2, VH CDR3, VL CDR1, VL CDR2 and VL CDR3 of the first binding domain form a binding site for an epitope of the NKp46. In some embodiments of the multispecific NKp46/BCMA antibodies provided herein, the NKp46 is present on the surface of an NK cell.

In some embodiments of the multispecific NKp46/BCMA antibodies provided herein, the second binding domain that binds BCMA comprises a VH comprising a VH CDR1, a VH CDR2, and a VH CDR3 having an amino acid sequence of a VH CDR1, a VH CDR2, and a VH CDR3, respectively, of SEQ ID NO:99. In some embodiments of the multispecific NKp46/BCMA antibodies provided herein, the second binding domain that binds BCMA comprises a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific NKp46/BCMA antibodies provided herein, the second binding domain that binds BCMA comprises: (i) a VH comprising a VH CDR1, a VH CDR2, and a VH CDR3 having an amino acid sequence of a VH CDR1, a VH CDR2, and a VH CDR3, respectively, of SEQ ID NO:99; and (ii) a VL comprising a VL CDR1, a VL CDR2, and a VL CDR3 having an amino acid sequence of a VL CDR1, a VL CDR2, and a VL CDR3, respectively, of SEQ ID NO:100. In some embodiments of the multispecific NKp46/BCMA antibodies provided herein, the second binding domain that binds BCMA comprises a VH having an amino acid sequence of SEQ ID NO:99. In some embodiments of the multispecific NKp46/BCMA antibodies provided herein, the second binding domain that binds BCMA comprises a VL having an amino acid sequence of SEQ ID NO:100. In some embodiments of the multispecific NKp46/BCMA antibodies provided herein, the second binding domain that binds BCMA comprises a VH having an amino acid sequence of SEQ ID NO:99, and a VL having an amino acid sequence of SEQ ID NO:100.

In some embodiments of the multispecific NKp46/BCMA antibodies provided herein, the VH CDR1, VH CDR2, VH CDR3, VL CDR1, VL CDR2, and VL CDR3 amino acid sequences of the second binding domain that binds BCMA are according to the Kabat numbering system. In some embodiments of the multispecific NKp46/BCMA antibodies provided herein, the VH CDR1, VH CDR2, VH CDR3, VL CDR1, VL CDR2, and VL CDR3 amino acid sequences of the second binding domain that binds BCMA are according to the Chothia numbering system. In some embodiments of the multispecific NKp46/BCMA antibodies provided herein, the VH CDR1, VH CDR2, VH CDR3, VL CDR1, VL CDR2, and VL CDR3 amino acid sequences of the second binding domain that binds BCMA are according to the AbM numbering system. In some embodiments of the multispecific NKp46/BCMA antibodies provided herein, the VH CDR1, VH CDR2, VH CDR3, VL CDR1, VL CDR2, and VL CDR3 amino acid sequences of the second binding domain that binds BCMA are according to the Contact numbering system. In some embodiments of the multispecific NKp46/BCMA antibodies provided herein, the VH CDR1, VH CDR2, VH CDR3, VL CDR1, VL CDR2, and VL CDR3 amino acid sequences of the second binding domain that binds BCMA are according to the IMGT numbering system.

In some embodiments of the multispecific NKp46/BCMA antibodies provided herein, the second binding domain binds a BCMA antigen. In some embodiments of the multispecific NKp46/BCMA antibodies provided herein, the second binding domain binds a BCMA epitope. In some embodiments of the multispecific NKp46/BCMA antibodies provided herein, the second binding domain specifically binds to BCMA. In some embodiments of the multispecific NKp46/BCMA antibodies provided herein, the VH CDR1, VH CDR2, VH CDR3, VL CDR1, VL CDR2 and VL CDR3 of the second binding domain form a binding site for an antigen of the BCMA. In some embodiments, the VH CDR1, VH CDR2, VH CDR3, VL CDR1, VL CDR2 and VL CDR3 of the second binding domain form a binding site for an epitope of the BCMA. In some embodiments, the BCMA is present on the surface of a tumor cell.

In some embodiments of the multispecific NKp46/BCMA antibodies provided herein, the third target is not an NKp46 antigen. In some embodiments of the multispecific NKp46/BCMA antibodies provided herein, the fourth target is not an NKp46 antigen. In some embodiments of the multispecific NKp46/BCMA antibodies provided herein, the third target is not an NKp46 antigen, and the fourth target is not an NKp46 antigen. In some embodiments of the multispecific NKp46/BCMA antibodies provided herein, the third target is not a BCMA antigen. In some embodiments of the multispecific NKp46/BCMA antibodies provided herein, the fourth target is not a BCMA antigen. In some embodiments of the multispecific NKp46/BCMA antibodies provided herein, the third target is not a BCMA antigen, and the fourth target is not a BCMA antigen. In some embodiments of the multispecific NKp46/BCMA antibodies provided herein, the third target is not an NKp46 epitope. In some embodiments of the multispecific NKp46/BCMA antibodies provided herein, the fourth target is not an NKp46 epitope. In some embodiments of the multispecific NKp46/BCMA antibodies provided herein, the third target is not an NKp46 epitope, and the fourth target is not an NKp46 epitope. In some embodiments of the multispecific NKp46/BCMA antibodies provided herein, the third target is not a BCMA epitope. In some embodiments of the multispecific NKp46/BCMA antibodies provided herein, the fourth target is not a BCMA epitope. In some embodiments of the multispecific NKp46/BCMA antibodies provided herein, the third target is not a BCMA epitope, and the fourth target is not a BCMA epitope.

In a specific embodiment, the target is from a mammal. In a specific embodiment, the target is from a rat. In a specific embodiment, the target is from a mouse. In a specific embodiment, the target is from a primate. In a specific embodiment, the target is from a human.

In specific embodiments, provided is a multispecific NKp46/BCMA antibody in a knob-in-hole format. In specific embodiments, provided is a bispecific NKp46/BCMA antibody in a knob-in-hole format. In specific embodiments, provided is a trispecific antibody in a knob-in-hole format. In specific embodiments, provided is a quadraspecific antibody in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, an NKp46/BCMA antibody provided herein is comprised in a bispecific antibody. In some embodiments, an NKp46/BCMA antibody provided herein is comprised in a trispecific antibody. In some embodiments, an NKp46/BCMA antibody provided herein is comprised in a quadraspecific antibody. In some embodiments, an NKp46/BCMA bispecific antibody provided herein is comprised in an multispecific antibody.

In certain embodiments, a trispecific NKp46/BCMA antibody provided herein comprises a first binding domain comprising an NKp46 antibody provided herein that binds to an NKp46 epitope, a second binding domain comprising a BCMA antibody provided herein that binds to a BCMA epitope, and a third binding domain that binds to a third epitope, wherein the NKp46 epitope, the BCMA epitope, and the third epitope are not the same. In certain embodiments, a quadraspecific antibody provided herein comprises a first binding domain comprising an NKp46 antibody provided herein that binds to an NKp46 epitope, a second binding domain comprising a BCMA antibody provided herein that binds to a BCMA epitope, a third binding domain that binds to a third epitope, and a fourth binding domain that binds to a fourth epitope, wherein the NKp46 epitope, the BCMA epitope, the third epitope, and the fourth epitope are not the same. In certain embodiments, a trispecific antibody provided herein comprises a first binding domain comprising an NKp46 antibody provided herein that binds to an NKp46 antigen, a second binding domain comprising a BCMA antibody provided herein that binds to a BCMA antigen, and a third binding domain that binds to a third antigen, wherein the NKp46 antigen, the BCMA antigen, and the third antigen are not the same. In certain embodiments, a quadraspecific antibody provided herein that binds to an NKp46 antigen, a second binding domain comprising a BCMA antibody provided herein that binds to a BCMA antigen, a third binding domain that binds to a third antigen, and a fourth binding domain that binds to a fourth antigen, wherein the NKp46 antigen, the BCMA antigen, the third antigen, and the fourth antigen are not the same. In certain embodiments of a multispecific NKp46/BCMA antibody provided herein, the first binding domain that binds to NKp46 specifically binds to the NKp46. In other embodiments of a multispecific NKp46/BCMA antibody provided herein, the second binding domain that binds to BCMA specifically binds to the BCMA. In yet other embodiments of a multispecific NKp46/BCMA antibody provided herein, the first binding domain that binds to NKp46 specifically binds to the NKp46, and the second binding domain that binds to BCMA specifically binds to the BCMA.

In some embodiments, the multispecific NKp46/BCMA antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the NKp46 antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the NKp46/BCMA multispecific antibodies or antigen binding fragments thereof bind to a first epitope located on NKp46 and a second epitope of located on BCMA. In some embodiments, provided herein is a multispecific NKp46/BCMA antibody comprising: (a) a first binding domain that binds to an NKp46 antigen, and (b) a second binding domain that binds to a BCMA antigen. In some embodiments, provided herein is a multispecific NKp46/BCMA antibody comprising: (a) a first binding domain that specifically binds to an NKp46 antigen, and (b) a second binding domain that specifically binds to a BCMA antigen. In some embodiments, provided herein is a multispecific NKp46/BCMA antibody comprising: (a) a first binding domain that binds to a first epitope on an NKp46 antigen, and (b) a second binding domain that binds to a second epitope on a BCMA antigen. In some embodiments, provided herein is a multispecific antibody comprising: (a) a first binding domain that specifically binds to a first epitope on an NKp46 antigen, and (b) a second binding domain that specifically binds to a second epitope on a BCMA antigen.

In specific embodiments, the NKp46 antigen is on the surface of an NK cell. In specific embodiments, the BCMA antigen is on the surface of a tumor cell. The binding of the NKp46/BCMA multispecific antibody to NKp46 present on the surface of NK cells and BCMA present on the surface of tumor cells can, for example, result in the killing of the tumor cell. In other embodiments, the binding of the NKp46/BCMA multispecific antibody to NKp46 present on the surface of NK cells and BCMA present on the surface of tumor cells can, for example, result in the activation of the NK cell.

In specific embodiments, the NKp46 antigen is on the surface of a T cell. In certain embodiments, the BCMA antigen is on the surface of a tumor cell. The binding of the NKp46/BCMA multispecific antibody to NKp46 present on the surface of the T cell, and BCMA present on the surface of tumor cells can, for example, result in the killing of the tumor cells. In other embodiment, the binding of the NKp46/BCMA multispecific antibody to NKp46 present on the surface of the T cell, and BCMA present on the surface of tumor cells can, for example, result in the activation of the T cell. In some embodiments, the T cell is a gamma delta T cell. In some embodiments, the T cell is an innate lymphoid cell.

In another aspect, provided herein is a multispecific antibody that comprises a first binding domain that binds to NKp46 and a second binding domain that binds to GPRC5d (“multispecific NKp46/GPRC5d antibody”). In some embodiments, the multispecific NKp46/GPRC5d antibody is a bispecific antibody. In some embodiments, the multispecific NKp46/GPRC5d antibody is a trispecific antibody. In some embodiments, the multispecific NKp46/GPRC5d antibody is a quadraspecific antibody.

In some embodiments, the multispecific NKp46/GPRC5d antibody provided herein comprises: (a) a first binding domain that binds NKp46, and (b) a second binding domain that binds to GPRC5d. In one embodiment, the multispecific NKp46/GPRC5d antibody comprises: (a) a first binding domain that binds NKp46, and (b) a second binding domain that binds to GPRC5d, and (c) a third binding domain that binds to a third target. In one embodiment, the multispecific NKp46/GPRC5d antibody comprises: (a) a first binding domain that binds NKp46, and (b) a second binding domain that binds to GPRC5d, (c) a third binding domain that binds to a third target, and (d) a fourth binding domain that binds to a fourth target.

In some embodiments of the multispecific NKp46/GPRC5d antibodies provided herein, the first binding domain that binds NKp46 comprises a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67. In some embodiment of the multispecific NKp46/GPRC5d antibodies provided herein, the first binding domain that binds NKp46 comprises a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68. In some embodiment of the multispecific NKp46/GPRC5d antibodies provided herein, the first binding domain that binds NKp46 comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:67; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:68. In some embodiments of the multispecific NKp46/GPRC5d antibodies provided herein, the first binding domain that binds NKp46 comprises a VH having an amino acid sequence of SEQ ID NO:67. In some embodiments of the multispecific NKp46/GPRC5d antibodies provided herein, the first binding domain that binds NKp46 comprises a VL having an amino acid sequence of SEQ ID NO:68. In some embodiments of the multispecific NKp46/GPRC5d antibodies provided herein, the first binding domain that binds NKp46 comprises a VH having an amino acid sequence of SEQ ID NO:67, and a VL having an amino acid sequence of SEQ ID NO:68.

In some embodiments of the multispecific NKp46/GPRC5d antibodies provided herein, the VH CDR1, VH CDR2, VH CDR3, VL CDR1, VL CDR2, and VL CDR3 amino acid sequences of the first binding domain that binds NKp46 are according to the Kabat numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the first binding domain that binds NKp46 are according to the Chothia numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the first binding domain that binds NKp46 are according to the AbM numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the first binding domain that binds NKp46 are according to the Contact numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the first binding domain that binds NKp46 are according to the IMGT numbering system.

In some embodiments of the multispecific NKp46/GPRC5d antibodies provided herein, the first binding domain binds an NKp46 antigen. In some embodiments of the multispecific NKp46/GPRC5d antibodies provided herein, the first binding domain binds an NKp46 epitope. In some embodiments of the multispecific NKp46/GPRC5d antibodies provided herein, the first binding domain specifically binds to NKp46. In some embodiments of the multispecific NKp46/GPRC5d antibodies provided herein, the VH CDR1, VH CDR2, VH CDR3, VL CDR1, VL CDR2 and VL CDR3 of the first binding domain form a binding site for an antigen of the NKp46. In some embodiments of the multispecific NKp46/GPRC5d antibodies provided herein, the VH CDR1, VH CDR2, VH CDR3, VL CDR1, VL CDR2 and VL CDR3 of the first binding domain form a binding site for an epitope of the NKp46. In some embodiments of the multispecific NKp46/GPRC5d antibodies provided herein, the NKp46 is present on the surface of an NK cell.

In some embodiments of the multispecific NKp46/GPRC5d antibodies provided herein, the second binding domain that binds GPRC5d comprises a VH comprising a VH CDR1, a VH CDR2, and a VH CDR3 having an amino acid sequence of a VH CDR1, a VH CDR2, and a VH CDR3, respectively, of SEQ ID NO:131. In some embodiments of the multispecific NKp46/GPRC5d antibodies provided herein, the second binding domain that binds GPRC5d comprises a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific NKp46/GPRC5d antibodies provided herein, the second binding domain that binds GPRC5d comprises: (i) a VH comprising a VH CDR1, a VH CDR2, and a VH CDR3 having an amino acid sequence of a VH CDR1, a VH CDR2, and a VH CDR3, respectively, of SEQ ID NO:131; and (ii) a VL comprising a VL CDR1, a VL CDR2, and a VL CDR3 having an amino acid sequence of a VL CDR1, a VL CDR2, and a VL CDR3, respectively, of SEQ ID NO:132. In some embodiments of the multispecific NKp46/GPRC5d antibodies provided herein, the second binding domain that binds GPRC5d comprises a VH having an amino acid sequence of SEQ ID NO:131. In some embodiments of the multispecific NKp46/GPRC5d antibodies provided herein, the second binding domain that binds GPRC5d comprises a VL having an amino acid sequence of SEQ ID NO:132. In some embodiments of the multispecific NKp46/GPRC5d antibodies provided herein, the second binding domain that binds GPRC5d comprises a VH having an amino acid sequence of SEQ ID NO:131, and a VL having an amino acid sequence of SEQ ID NO:132.

In some embodiments of the multispecific NKp46/GPRC5d antibodies provided herein, the VH CDR1, VH CDR2, VH CDR3, VL CDR1, VL CDR2, and VL CDR3 amino acid sequences of the second binding domain that binds GPRC5d are according to the Kabat numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the second binding domain that binds GPRC5d are according to the Chothia numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the second binding domain that binds GPRC5d are according to the AbM numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the second binding domain that binds GPRC5d are according to the Contact numbering system. In some embodiments of the multispecific NKp46/GPRC5d antibodies provided herein, the VH CDR1, VH CDR2, VH CDR3, VL CDR1, VL CDR2, and VL CDR3 amino acid sequences of the second binding domain that binds GPRC5d are according to the IMGT numbering system.

In some embodiments of the multispecific NKp46/GPRC5d antibodies provided herein, the second binding domain binds a GPRC5d antigen. In some embodiments of the multispecific NKp46/GPRC5d antibodies provided herein, the second binding domain binds a GPRC5d epitope. In some embodiments of the multispecific NKp46/GPRC5d antibodies provided herein, the second binding domain specifically binds to GPRC5d. In some embodiments of the multispecific NKp46/GPRC5d antibodies provided herein, the VH CDR1, VH CDR2, VH CDR3, VL CDR1, VL CDR2 and VL CDR3 of the second binding domain form a binding site for an antigen of the GPRC5d. In some embodiments, the VH CDR1, VH CDR2, VH CDR3, VL CDR1, VL CDR2 and VL CDR3 of the second binding domain form a binding site for an epitope of the GPRC5d. In some embodiments, the GPRC5d is present on the surface of a tumor cell.

In some embodiments of the multispecific NKp46/GPRC5d antibodies provided herein, the third target is not an NKp46 antigen. In some embodiments of the multispecific NKp46/GPRC5d antibodies provided herein, the fourth target is not an NKp46 antigen. In some embodiments of the multispecific NKp46/GPRC5d antibodies provided herein, the third target is not an NKp46 antigen, and the fourth target is not an NKp46 antigen. In some embodiments of the multispecific NKp46/GPRC5d antibodies provided herein, the third target is not a GPRC5d antigen. In some embodiments of the multispecific NKp46/GPRC5d antibodies provided herein, the fourth target is not a GPRC5d antigen. In some embodiments of the multispecific NKp46/GPRC5d antibodies provided herein, the third target is not a GPRC5d antigen, and the fourth target is not a GPRC5d antigen. In some embodiments of the multispecific NKp46/GPRC5d antibodies provided herein, the third target is not an NKp46 epitope. In some embodiments of the multispecific NKp46/GPRC5d antibodies provided herein, the fourth target is not an NKp46 epitope. In some embodiments of the multispecific NKp46/GPRC5d antibodies provided herein, the third target is not an NKp46 epitope, and the fourth target is not an NKp46 epitope. In some embodiments of the multispecific NKp46/GPRC5d antibodies provided herein, the third target is not a GPRC5d epitope. In some embodiments of the multispecific NKp46/GPRC5d antibodies provided herein, the fourth target is not a GPRC5d epitope. In some embodiments of the multispecific NKp46/GPRC5d antibodies provided herein, the third target is not a GPRC5d epitope, and the fourth target is not a GPRC5d epitope.

In a specific embodiment, the target is from a mammal. In a specific embodiment, the target is from a rat. In a specific embodiment, the target is from a mouse. In a specific embodiment, the target is from a primate. In a specific embodiment, the target is from a human.

In specific embodiments, provided is a multispecific NKp46/GPRC5d antibody in a knob-in-hole format. In specific embodiments, provided is a bispecific NKp46/GPRC5d antibody in a knob-in-hole format. In specific embodiments, provided is a trispecific antibody in a knob-in-hole format. In specific embodiments, provided is a quadraspecific antibody in a knob-in-hole format. Other specificities can be added to an antibody in knob-in-hole format using methods well known in the art (e.g., adding an scFv to the N-terminus or C-terminus). In addition, other formats and methods of making multispecific antibodies are also known in the art and contemplated. In some embodiments, an NKp46/GPRC5d antibody provided herein is comprised in a bispecific antibody. In some embodiments, an NKp46/GPRC5d antibody provided herein is comprised in a trispecific antibody. In some embodiments, an NKp46/GPRC5d antibody provided herein is comprised in a quadraspecific antibody. In some embodiments, an NKp46/GPRC5d bispecific antibody provided herein is comprised in a multispecific antibody.

In certain embodiments, a trispecific NKp46/GPRC5d antibody provided herein comprises a first binding domain comprising an NKp46 antibody provided herein that binds to an NKp46 epitope, a second binding domain comprising a GPRC5d antibody provided herein that binds to a GPRC5d epitope, and a third binding domain that binds to a third epitope, wherein the NKp46 epitope, the GPRC5d epitope, and the third epitope are not the same. In certain embodiments, a quadraspecific antibody provided herein comprises a first binding domain comprising an NKp46 antibody provided herein that binds to an NKp46 epitope, a second binding domain comprising a GPRC5d antibody provided herein that binds to a GPRC5d epitope, a third binding domain that binds to a third epitope, and a fourth binding domain that binds to a fourth epitope, wherein the NKp46 epitope, the GPRC5d epitope, the third epitope, and the fourth epitope are not the same. In certain embodiments, a trispecific antibody provided herein comprises a first binding domain comprising an NKp46 antibody provided herein that binds to an NKp46 antigen, a second binding domain comprising a GPRC5d antibody provided herein that binds to a GPRC5d antigen, and a third binding domain that binds to a third antigen, wherein the NKp46 antigen, the GPRC5d antigen, and the third antigen are not the same. In certain embodiments, a quadraspecific antibody provided herein that binds to an NKp46 antigen, a second binding domain comprising a GPRC5d antibody provided herein that binds to a GPRC5d antigen, a third binding domain that binds to a third antigen, and a fourth binding domain that binds to a fourth antigen, wherein the NKp46 antigen, the GPRC5d antigen, the third antigen, and the fourth antigen are not the same. In certain embodiments of a multispecific NKp46/GPRC5d antibody provided herein, the first binding domain that binds to NKp46 specifically binds to the NKp46. In other embodiments of a multispecific NKp46/GPRC5d antibody provided herein, the second binding domain that binds to GPRC5d specifically binds to the GPRC5d. In yet other embodiments of a multispecific NKp46/GPRC5d antibody provided herein, the first binding domain that binds to NKp46 specifically binds to the NKp46, and the second binding domain that binds to GPRC5d specifically binds to the GPRC5d.

In some embodiments, the multispecific NKp46/GPRC5d antibody comprises heavy chain variable regions and light chain variable region. In some embodiments, the first binding domain comprises a heavy chain variable region and a light chain variable region. In some embodiments, the second binding domain comprises a heavy chain variable region and a light chain variable region. In some embodiments, the first binding domain comprises a heavy chain variable region and a light chain variable region, and the second binding domain comprises a heavy chain variable region and a light chain variable region. In some embodiments, the NKp46 antibody is not a single domain antibody or nanobody. In some embodiments, the third binding domain comprises a heavy chain variable region and a light chain variable region. In some embodiments, the fourth binding domain comprises a heavy chain variable region and a light chain variable region.

In certain embodiments, the NKp46/GPRC5d multispecific antibodies or antigen binding fragments thereof bind to a first epitope located on NKp46 and a second epitope of located on GPRC5d. In some embodiments, provided herein is a multispecific NKp46/GPRC5d antibody comprising: (a) a first binding domain that binds to an NKp46 antigen, and (b) a second binding domain that binds to a GPRC5d antigen. In some embodiments, provided herein is a multispecific NKp46/GPRC5d antibody comprising: (a) a first binding domain that specifically binds to an NKp46 antigen, and (b) a second binding domain that specifically binds to a GPRC5d antigen. In some embodiments, provided herein is a multispecific NKp46/GPRC5d antibody comprising: (a) a first binding domain that binds to a first epitope on an NKp46 antigen, and (b) a second binding domain that binds to a second epitope on a GPRC5d antigen. In some embodiments, provided herein is a multispecific antibody comprising: (a) a first binding domain that specifically binds to a first epitope on an NKp46 antigen, and (b) a second binding domain that specifically binds to a second epitope on a GPRC5d antigen.

In specific embodiments, the NKp46 antigen is on the surface of an NK cell. In specific embodiments, the GPRC5d antigen is on the surface of a tumor cell. The binding of the NKp46/GPRC5d multispecific antibody to NKp46 present on the surface of NK cells and GPRC5d present on the surface of tumor cells can, for example, result in the killing of the tumor cell. In other embodiments, the binding of the NKp46/GPRC5d multispecific antibody to NKp46 present on the surface of NK cells and GPRC5d present on the surface of tumor cells can, for example, result in the activation of the NK cell.

In specific embodiments, the NKp46 antigen is on the surface of a T cell. In certain embodiments, the GPRC5d antigen is on the surface of a tumor cell. The binding of the NKp46/GPRC5d multispecific antibody to NKp46 present on the surface of the T cell, and GPRC5d present on the surface of tumor cells can, for example, result in the killing of the tumor cells. In other embodiment, the binding of the NKp46/GPRC5d multispecific antibody to NKp46 present on the surface of the T cell, and GPRC5d present on the surface of tumor cells can, for example, result in the activation of the T cell. In some embodiments, the T cell is a gamma delta T cell. In some embodiments, the T cell is an innate lymphoid cell.

In some specific embodiments, provided herein is a bispecific antibody generated in Section 7 below, for example, as shown in Table 21 and Table 22 below.

In some embodiments, provided herein is a bispecific antibody comprising a first binding domain that binds to an antigen on NK cells comprising a first VH of SEQ ID NO:2 and a first VL of SEQ ID NO:3; and a second binding domain that binds to a tumor antigen comprising a second VH of SEQ ID NO:99 and a second VL of SEQ ID NO:100.

In some embodiments, provided herein is a bispecific antibody comprising a first binding domain that binds to an antigen on NK cells comprising a first VH of SEQ ID NO:2 and a first VL of SEQ ID NO:3; and a second binding domain that binds to a tumor antigen comprising a second VH of SEQ ID NO:131 and a second VL of SEQ ID NO:132.

In some embodiments, provided herein is a bispecific antibody comprising a first binding domain that binds to an antigen on NK cells comprising a first VH of SEQ ID NO:34 and a first VL of SEQ ID NO:35; and a second binding domain that binds to a tumor antigen comprising a second VH of SEQ ID NO:99 and a second VL of SEQ ID NO:100.

In some embodiments, provided herein is a bispecific antibody comprising a first binding domain that binds to an antigen on NK cells comprising a first VH of SEQ ID NO:34 and a first VL of SEQ ID NO:35; and a second binding domain that binds to a tumor antigen comprising a second VH of SEQ ID NO:131 and a second VL of SEQ ID NO:132.

In some embodiments, provided herein is a bispecific antibody comprising a first binding domain that binds to an antigen on NK cells comprising a first VH of SEQ ID NO:67 and a first VL of SEQ ID NO:68; and a second binding domain that binds to a tumor antigen comprising a second VH of SEQ ID NO:99 and a second VL of SEQ ID NO:100.

In some embodiments, provided herein is a bispecific antibody comprising a first binding domain that binds to an antigen on NK cells comprising a first VH of SEQ ID NO:67 and a first VL of SEQ ID NO:68; and a second binding domain that binds to a tumor antigen comprising a second VH of SEQ ID NO:131 and a second VL of SEQ ID NO:132.

In some embodiments, provided herein is a bispecific antibody comprising a first polypeptide of SEQ ID NO:164, a second polypeptide of SEQ ID NO:163, and third polypeptide of SEQ ID NO:165.

In some embodiments, provided herein is a bispecific antibody comprising a first polypeptide of SEQ ID NO:164, a second polypeptide of SEQ ID NO:163, and third polypeptide of SEQ ID NO:174.

In some embodiments, provided herein is a bispecific antibody comprising a first polypeptide of SEQ ID NO:168, a second polypeptide of SEQ ID NO:169, and third polypeptide of SEQ ID NO:165.

In some embodiments, provided herein is a bispecific antibody comprising a first polypeptide of SEQ ID NO:173, a second polypeptide of SEQ ID NO:170, and third polypeptide of SEQ ID NO:165.

In some embodiments, provided herein is a bispecific antibody comprising a first polypeptide of SEQ ID NO:168, a second polypeptide of SEQ ID NO:169, and third polypeptide of SEQ ID NO:174.

In some embodiments, provided herein is a bispecific antibody comprising a first polypeptide of SEQ ID NO:173, a second polypeptide of SEQ ID NO:170, and third polypeptide of SEQ ID NO:174.

In some embodiments, provided herein is a bispecific antibody comprising a first polypeptide of SEQ ID NO:177, a second polypeptide of SEQ ID NO:163, and third polypeptide of SEQ ID NO:180.

In some embodiments, provided herein is a bispecific antibody comprising a first polypeptide of SEQ ID NO:194, a second polypeptide of SEQ ID NO:163, and third polypeptide of SEQ ID NO:196.

In some embodiments, provided herein is a bispecific antibody comprising a first polypeptide of SEQ ID NO:166, and a second polypeptide of SEQ ID NO:163. In some embodiments, the bispecific antibody comprises two polypeptides each comprising SEQ ID NO:166, and two polypeptides each comprising SEQ ID NO:163.

In some embodiments, provided herein is a bispecific antibody comprising a first polypeptide of SEQ ID NO:167, and a second polypeptide of SEQ ID NO:163. In some embodiments, the bispecific antibody comprises two polypeptides each comprising SEQ ID NO:167, and two polypeptides each comprising SEQ ID NO:163.

In some embodiments, provided herein is a bispecific antibody comprising a first polypeptide of SEQ ID NO:171, and a second polypeptide of SEQ ID NO:170. In some embodiments, the bispecific antibody comprises two polypeptides each comprising SEQ ID NO:171, and two polypeptides each comprising SEQ ID NO:170.

In some embodiments, provided herein is a bispecific antibody comprising a first polypeptide of SEQ ID NO:172, and a second polypeptide of SEQ ID NO:169. In some embodiments, the bispecific antibody comprises two polypeptides each comprising SEQ ID NO:172, and two polypeptides each comprising SEQ ID NO:169.

In some embodiments, provided herein is a bispecific antibody comprising a first polypeptide of SEQ ID NO:175, and a second polypeptide of SEQ ID NO:170. In some embodiments, the bispecific antibody comprises two polypeptides each comprising SEQ ID NO:175, and two polypeptides each comprising SEQ ID NO:170.

In some embodiments, provided herein is a bispecific antibody comprising a first polypeptide of SEQ ID NO:176, and a second polypeptide of SEQ ID NO:169. In some embodiments, the bispecific antibody comprises two polypeptides each comprising SEQ ID NO:176, and two polypeptides each comprising SEQ ID NO:169.

The antibodies provided herein may be from any animal origin including birds and mammals (e.g., human, monkey, murine, donkey, sheep, rabbit, goat, guinea pig, camel, horse, or chicken). In certain embodiments, the antibodies provided herein are human or humanized monoclonal antibodies. As used herein, “human” antibodies include antibodies having the amino acid sequence of a human immunoglobulin and include antibodies isolated from human immunoglobulin libraries or from mice that express antibodies from human genes.

In certain embodiments, the antibodies are full mouse antibodies. In certain embodiments, the antibodies are mouse-human chimeric antibodies. In certain embodiments, the antibodies are humanized antibodies. In certain embodiments, the antibodies are fully human antibodies. In other embodiments, the antibodies provided herein are humanized antibodies (e.g., comprising human constant and framework regions). The antibodies provided herein may be bispecific, trispecific or of greater multispecificity.

In some embodiments, the antibody or antigen binding fragment provided herein binds NKG2d with a K_{D} of less than 1000 nM. In some embodiments, the antibody or antigen binding fragment provided herein binds NKG2d with a K_{D} of less than 100 nM. In some embodiments, the antibody or antigen binding fragment provided herein binds NKG2d with a K_{D} of less than 50 nM. In some embodiments, the antibody or antigen binding fragment provided herein binds NKG2d with a K_{D} of less than 40 nM. In some embodiments, the antibody or antigen binding fragment provided herein binds NKG2d with a K_{D} of less than 30 nM. In some embodiments, the antibody or antigen binding fragment provided herein binds NKG2d with a K_{D} of less than 20 nM. In some embodiments, the antibody or antigen binding fragment provided herein binds NKG2d with a K_{D} of less than 10 nM. In some embodiments, the antibody or antigen binding fragment provided herein binds NKG2d with a K_{D} of less than 9 nM. In some embodiments, the antibody or antigen binding fragment provided herein binds NKG2d with a K_{D} of less than 8 nM. In some embodiments, the antibody or antigen binding fragment provided herein binds NKG2d with a K_{D} of less than 7 nM. In some embodiments, the antibody or antigen binding fragment provided herein binds NKG2d with a K_{D} of less than 6 nM. In some embodiments, the antibody or antigen binding fragment provided herein binds NKG2d with a K_{D} of less than 5 nM. In some embodiments, the antibody or antigen binding fragment provided herein binds NKG2d with a K_{D} of less than 4 nM. In some embodiments, the antibody or antigen binding fragment provided herein binds NKG2d with a K_{D} of less than 3 nM. In some embodiments, the antibody or antigen binding fragment provided herein binds NKG2d with a K_{D} of less than 2 nM. In some embodiments, the antibody or antigen binding fragment provided herein binds NKG2d with a K_{D} of less than 1 nM. In some embodiments, the antibody or antigen binding fragment provided herein binds NKG2d with a K_{D} of less than 0.1 nM. In some embodiments, the antibody or antigen binding fragment provided herein binds NKG2d with a K_{D} of less than 0.01 nM. The K_{D} or K_{D} value may also be measured by any known methods in the art, for example, using biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system. In a specific embodiment, the K_{D} is determined by a Biacore® assay. In some embodiments, NKG2d is a human NKG2d. In some embodiments, NKG2d is a cynomolgus macaque NKG2d. In some embodiments, NKG2d is a rat NKG2d. In other embodiments, NKG2d is mouse NKG2d.

In other embodiments, the antibody or antigen binding fragment provided herein binds NKp46 with a K_{D} of less than 1000 nM. In some embodiments, the antibody or antigen binding fragment provided herein binds NKp46 with a K_{D} of less than 100 nM. In some embodiments, the antibody or antigen binding fragment provided herein binds NKp46 with a K_{D} of less than 50 nM. In some embodiments, the antibody or antigen binding fragment provided herein binds NKp46 with a K_{D} of less than 40 nM. In some embodiments, the antibody or antigen binding fragment provided herein binds NKp46 with a K_{D} of less than 30 nM. In some embodiments, the antibody or antigen binding fragment provided herein binds NKp46 with a K_{D} of less than 20 nM. In some embodiments, the antibody or antigen binding fragment provided herein binds NKp46 with a K_{D} of less than 10 nM. In some embodiments, the antibody or antigen binding fragment provided herein binds NKp46 with a K_{D} of less than 9 nM. In some embodiments, the antibody or antigen binding fragment provided herein binds NKp46 with a K_{D} of less than 8 nM. In some embodiments, the antibody or antigen binding fragment provided herein binds NKp46 with a K_{D} of less than 7 nM. In some embodiments, the antibody or antigen binding fragment provided herein binds NKp46 with a K_{D} of less than 6 nM. In some embodiments, the antibody or antigen binding fragment provided herein binds NKp46 with a K_{D} of less than 5 nM. In some embodiments, the antibody or antigen binding fragment provided herein binds NKp46 with a K_{D} of less than 4 nM. In some embodiments, the antibody or antigen binding fragment provided herein binds NKp46 with a K_{D} of less than 3 nM. In some embodiments, the antibody or antigen binding fragment provided herein binds NKp46 with a K_{D} of less than 2 nM. In some embodiments, the antibody or antigen binding fragment provided herein binds NKp46 with a K_{D} of less than 1 nM. In some embodiments, the antibody or antigen binding fragment provided herein binds NKp46 with a K_{D} of less than 0.1 nM. In some embodiments, the antibody or antigen binding fragment provided herein binds NKp46 with a K_{D} of less than 0.01 nM. The K_{D} or K_{D} value may also be measured by any known methods in the art, for example, using biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the Octet®Red96, the Biacore®TM-2000, or the Biacore®TM-3000 system. In a specific embodiment, the K_{D} is determined by a Biacore® assay. In some embodiments, NKp46 is a human NKp46. In some embodiments, NKp46 is a cynomolgus macaque NKp46. In some embodiments, NKp46 is a rat NKp46. In other embodiments, NKp46 is mouse NKp46.

Any multispecific antibody platform or formats known in the art can be used in the present disclosure, including any known bispecific antibody formats in the art.

In some embodiments, a multispecific antibody provided herein is a diabody, a cross-body, or a multispecific antibody obtained via a controlled Fab arm exchange as those described herein.

In some embodiments, the multispecific antibodies include IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual (ScFv)₂-Fab (National Research Center for Antibody Medicine—China).

In some embodiments, Fab fusion bispecific antibodies include F(ab)₂ (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies provided herein can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope. Other methods of making multispecific antibodies are known and contemplated.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/366A_K409F, or T350V_L351Y_F405A Y407V/T350V_T366L_K392L_T394W as described in U.S. Pat. Publ. No. US2012/0149876 or U.S. Pat. Publ. No. US2013/0195849.

In addition to methods described above, bispecific antibodies provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

In some embodiments, the multispecific antibody is a bispecific antibody, wherein the bispecific antibody is in a bipod-scaffold configuration. In some embodiments, the multispecific antibody is a bispecific antibody in a bipod-scaffold configuration, wherein the first binding domain is a Fab region, and the second binding domain is a scFv region. In some embodiments, the first binding domain binds to an antigen present on an NK cell. In some embodiments, the first binding domain binds to NKG2d. In some embodiments, the first binding domain binds to NKp46. In some embodiments, the second binding domain binds to an antigen present on a tumor cell. In some embodiments, the second binding domain binds to BCMA. In some embodiments, the second binding domain binds to GPRC5d.

In some embodiments, the multispecific antibody is a bispecific antibody, wherein the bispecific antibody is in a Morrison-scaffold configuration. In some embodiments, the multispecific antibody is a bispecific antibody in a Morrison-scaffold configuration, wherein the first binding domain comprises two Fab regions and the second binding domain comprises two scFv regions. In some embodiments, the first binding domain binds to an antigen present on an NK cell. In some embodiments, the first binding domain binds to NKG2d. In some embodiments, the first binding domain binds to NKp46. In some embodiments, the second binding domain binds to an antigen present on a tumor cell. In some embodiments, the second binding domain binds to BCMA. In some embodiments, the second binding domain binds to GPRC5d.

In some embodiments of the multispecific antibody provided herein, the Fc region comprises IgG1 silent mutations. In some embodiments, the Fc region comprise AAS mutation. In some embodiments of the multispecific antibody provided herein, the Fc region comprises mutations for enhancement of an effector function of the antibody. In some embodiments of the multispecific antibody provided herein, the Fc region comprises CDC enhancement mutations. In some embodiment, the Fc region comprises K248E and T437R mutations. In some embodiments of the multispecific antibody provided herein, the Fc region is afucosylated.

In some embodiments, provided herein are antibodies that specifically bind to NKG2d and can modulate NK cell activity. In some embodiments, provided herein are antibodies that specifically bind to NKp46 and can modulate NK cell activity.

In some embodiments, the multispecific antibody described herein activate an NK cell. In certain embodiments, the multispecific antibody described herein contact or direct NK cells to a target cell. In certain embodiments, the multispecific antibody described herein contact or direct NK cells to a target cell, wherein the multispecific antibody comprises a first binding domain that binds to a first antigen on an NK cell and a second binding domain that binds to a second antigen on a target cell. In specific embodiments, the target cells are tumor cells. In certain embodiments, the multispecific antibody described herein induces NK cell dependent cytotoxicity of the tumor cell, wherein the multispecific antibody comprises a first binding domain that binds to a first antigen on an NK cell and a second binding domain that binds to a second antigen on a target cell. In specific embodiments, the NK cells are human NK cells.

In some embodiments, the multispecific antibody that bind to NKG2d described herein activate an NK cell. In certain embodiments, the multispecific antibody that bind to NKG2d described herein contact or direct NK cells to a target cell. In certain embodiments, the multispecific antibody that bind to NKG2d described herein contact or direct NK cells to a target cell, wherein the multispecific antibody comprises a first binding domain that binds to NKG2d on an NK cell and a second binding domain that binds to a second antigen on a target cell. In specific embodiments, the target cells are tumor cells. In certain embodiments, the multispecific antibody described herein induces NK cell dependent cytotoxicity of the tumor cell, wherein the multispecific antibody comprises a first binding domain that binds to NKG2d on an NK cell and a second binding domain that binds to a second antigen on a tumor cell. In specific embodiments, the NK cells are human NK cells.

In some embodiments, the multispecific antibody that bind to NKp46 described herein activate an NK cell. In certain embodiments, the multispecific antibody that bind to NKp46 described herein contact or direct NK cells to a target cell. In certain embodiments, the multispecific antibody that bind to NKp46 described herein contact or direct NK cells to a target cell, wherein the multispecific antibody comprises a first binding domain that binds to NKp46 on an NK cell and a second binding domain that binds to a second antigen on a target cell. In specific embodiments, the target cells are tumor cells. In certain embodiments, the multispecific antibody described herein induces NK cell dependent cytotoxicity of the tumor cell, wherein the multispecific antibody comprises a first binding domain that binds to NKp46 on an NK cell and a second binding domain that binds to a second antigen on a tumor cell. In specific embodiments, the NK cells are human NK cells.

In some embodiments, the multispecific antibody described herein binds to NKp46 expressed on NKp46-expressing immune cells. In some embodiments, the NKp46-expressing immune cells are T cells. In some embodiments, the T cell are gamma delta T cells. In some embodiments, the T cell are mucosal population of innate lymphoid cells.

In some embodiments, the multispecific antibodies that bind to NKp46 described herein can activate an NKp46-expressing immune cell. In certain embodiments, the multispecific antibodies that bind to NKp46 described herein contact or direct NKp46-expressing immune cell to a target cell. In certain embodiments, the multispecific antibody that bind to NKp46 described herein contact or direct NKp46-expressing immune cell to a target cell, wherein the multispecific antibody comprises a first binding domain that binds to NKp46 on an NKp46-expressing immune cell and a second binding domain that binds to a second antigen on a target cell. In specific embodiments, the target cells are tumor cells. In certain embodiments, the multispecific antibody described herein induces NKp46-expressing immune cell dependent cytotoxicity of the tumor cell, wherein the multispecific antibody comprises a first binding domain that binds to NKp46 on an NKp46-expressing immune cell and a second binding domain that binds to a second antigen on a tumor cell. In specific embodiments, the NK cells are human NK cells.

In some embodiments, the multispecific antibody described herein can activate NK cells by functioning through both activating NK cell receptor and Fc receptor. In certain embodiments, the multispecific antibody described herein contact or direct NK cells to a tumor cell. In certain embodiments, the multispecific antibody described herein induces NK cell dependent cytotoxicity of the tumor cell by functioning through both activating NK cell receptor and Fc receptor. In certain embodiments, the multispecific antibody described herein induces NK cell dependent cytotoxicity of the tumor cell, even if the Fc region of the multispecific antibody comprises IgG1 silent mutations.

In some embodiments, the multispecific antibody described herein can activate an NK cell in immune suppressive tumor environment. In certain embodiments, the multispecific antibody described herein contact or direct NK cells to a tumor cell in immune suppressive tumor environment. In certain embodiments, the multispecific antibody described herein induces NK cell dependent cytotoxicity of the tumor cell in immune suppressive tumor environment. In certain embodiments, the multispecific antibody described herein induces NK cell dependent cytotoxicity of the tumor cell in immune suppressive tumor environment, even if the Fc region of the multispecific antibody comprises IgG1 silent mutations.

In some embodiments, the multispecific antibodies described herein do not exert detrimental effects to NK cells. In some embodiments, the multispecific antibodies described herein are lack of anti-NK cell cytotoxicity. In some embodiments, the multispecific antibodies described herein with WT IgG1 backbone do not cause CDC killing of NK cells. In some embodiments, the multispecific antibodies described herein with a CDC-enhancing set of mutations (e.g., K248E/T437R) do not cause CDC killing of NK cells. In some embodiments, the multispecific antibody is NKp46 x BCMA bispecific antibody. In some embodiments, the multispecific antibody is NKG2d x BCMA bispecific antibody. In some embodiments, the multispecific antibody is NKp46 x GPRC5d bispecific antibody. In some embodiments, the multispecific antibody is NKG2d x GPRC5d bispecific antibody.

In some embodiments, the multispecific antibodies described herein induce NK cell lysis less than 25%. In some embodiments, the multispecific antibodies described herein induce NK cell lysis less than 20%. In some embodiments, the multispecific antibodies described herein induce NK cell lysis less than 15%. In some embodiments, the multispecific antibodies described herein induce NK cell lysis less than 10%. In some embodiments, the multispecific antibodies described herein induce NK cell lysis less than 5%. In some embodiments, the multispecific antibodies described herein induce NK cell lysis less than 3%. In some embodiments, the multispecific antibodies described herein induce NK cell lysis less than 2%. In some embodiments, the multispecific antibodies described herein induce NK cell lysis less than 1%.

In some embodiments, the multispecific antibodies described herein activate an NK cell. In some embodiments, the multispecific antibodies described herein activate an NK cell activity by at least about 10%. In some embodiments, the multispecific antibodies described herein activate an NK cell activity by at least about 20%. In some embodiments, the multispecific antibodies described herein activate an NK cell activity by at least about 30%. In some embodiments, the multispecific antibodies described herein activate an NK cell activity by at least about 40%. In some embodiments, the multispecific antibodies described herein activate an NK cell activity by at least about 50%. In some embodiments, the multispecific antibodies described herein activate an NK cell activity by at least about 60%. In some embodiments, the multispecific antibodies described herein activate an NK cell activity by at least about 70%. In some embodiments, the multispecific antibodies described herein activate an NK cell activity by at least about 80%. In some embodiments, the multispecific antibodies described herein activate an NK cell activity by at least about 90%. In some embodiments, the multispecific antibodies described herein activate an NK cell activity by at least about 95%. In some embodiments, the multispecific antibodies described herein activate an NK cell activity by at least about 95%. In certain embodiments, the multispecific antibodies described herein activate an NK cell activity by at least about 15% to about 65%. In certain embodiments, the multispecific antibodies described herein activate an NK cell activity by at least about 20% to about 65%. In certain embodiments, the multispecific antibodies described herein activate an NK cell activity by at least about 30% to about 65%. In specific embodiments, the NK cells are human NK cells.

In some embodiments, the multispecific antibodies described herein promote IFNg production by NK cells. In some embodiments, the multispecific antibodies described herein promote IFNg production by NK cells by at least 10%. In some embodiments, the multispecific antibodies described herein promote IFNg production by NK cells by at least 20%. In some embodiments, the multispecific antibodies described herein promote IFNg production by NK cells by at least 30%. In some embodiments, the multispecific antibodies described herein promote IFNg production by NK cells by at least 40%. In some embodiments, the multispecific antibodies described herein promote IFNg production by NK cells by at least 50%. In some embodiments, the multispecific antibodies described herein promote IFNg production by NK cells by at least 60%. In some embodiments, the multispecific antibodies described herein promote IFNg production by NK cells by at least 70%. In some embodiments, the multispecific antibodies described herein promote IFNg production by NK cells by at least 80%. In some embodiments, the multispecific antibodies described herein promote IFNg production by NK cells by at least 90%. In some embodiments, the multispecific antibodies described herein promote IFNg production by NK cells by at least 95%. In certain embodiments, the multispecific antibodies described herein promote IFNg production by NK cells by at least about 15% to about 65%. In certain embodiments, the multispecific antibodies described herein promote IFNg production by NK cells by at least about 20% to about 65%. In certain embodiments, the multispecific antibodies described herein promote IFNg production by NK cells by at least about 30% to about 65%. In specific embodiments, the NK cells are human NK cells.

In some embodiments of the multispecific antibody provided herein, the multispecific antibody induces NK cell dependent cytotoxicity of the tumor cell by at least 10%. In some embodiments of the multispecific antibody provided herein, the multispecific antibody induces NK cell dependent cytotoxicity of the tumor cell by at least 20%. In some embodiments of the multispecific antibody provided herein, the multispecific antibody induces NK cell dependent cytotoxicity of the tumor cell by at least 30%. In some embodiments of the multispecific antibody provided herein, the multispecific antibody induces NK cell dependent cytotoxicity of the tumor cell by at least 40%. In some embodiments of the multispecific antibody provided herein, the multispecific antibody induces NK cell dependent cytotoxicity of the tumor cell by at least 50%. In some embodiments of the multispecific antibody provided herein, the multispecific antibody induces NK cell dependent cytotoxicity of the tumor cell by at least 60%. In some embodiments of the multispecific antibody provided herein, the multispecific antibody induces NK cell dependent cytotoxicity of the tumor cell by at least 70%. In some embodiments of the multispecific antibody provided herein, the multispecific antibody induces NK cell dependent cytotoxicity of the tumor cell by at least 80%. In some embodiments of the multispecific antibody provided herein, the multispecific antibody induces NK cell dependent cytotoxicity of the tumor cell by at least 90%. In some embodiments of the multispecific antibody provided herein, the multispecific antibody induces NK cell dependent cytotoxicity of the tumor cell by at least 95%. In certain embodiments of the multispecific antibody provided herein, the multispecific antibody induces NK cell dependent cytotoxicity of the tumor cell by at least about 15% to about 65%. In certain embodiments of the multispecific antibody provided herein, the multispecific antibody induces NK cell dependent cytotoxicity of the tumor cell by at least about 20% to about 65%. In certain embodiments of the multispecific antibody provided herein, the multispecific antibody induces NK cell dependent cytotoxicity of the tumor cell by at least about 30% to about 65%.

In some embodiments of the multispecific antibody provided herein, the multispecific antibody induces NK cell dependent cytotoxicity of the tumor cell in vitro with an IC₅₀ of less than about 500 pM. In some embodiments, the multispecific antibody induces NK cell dependent cytotoxicity of the tumor cell in vitro with an IC₅₀ of less than about 300 pM. In some embodiments, the multispecific antibody induces NK cell dependent cytotoxicity of the tumor cell in vitro with an IC₅₀ of less than about 100 pM. In some embodiments, the multispecific antibody induces NK cell dependent cytotoxicity of the tumor cell in vitro with an IC₅₀ of less than about 50 pM. In some embodiments, the multispecific antibody induces NK cell dependent cytotoxicity of the tumor cell in vitro with an IC₅₀ of less than about 20 pM. In some embodiments, the multispecific antibody induces NK cell dependent cytotoxicity of the tumor cell in vitro with an IC₅₀ of less than about 15 pM. In some embodiments, the multispecific antibody induces NK cell dependent cytotoxicity of the tumor cell in vitro with an IC₅₀ of less than about 10 pM.

In some embodiments of the multispecific antibody provided herein, the IC₅₀ is assessed with a mixture of NK effector cells and target cells expressing the second antigen.

In some embodiments of the multispecific antibody provided herein, the multispecific antibody induces NK cell dependent cytotoxicity of the tumor cell in vitro with an EC₅₀ of less than about 2000 pM. In some embodiments of the multispecific antibody provided herein, the multispecific antibody induces NK cell dependent cytotoxicity of the tumor cell in vitro with an EC₅₀ of less than about 1000 pM. In some embodiments of the multispecific antibody provided herein, the multispecific antibody induces NK cell dependent cytotoxicity of the tumor cell in vitro with an EC₅₀ of less than about 500 pM. In some embodiments, the multispecific antibody induces NK cell dependent cytotoxicity of the tumor cell in vitro with an EC₅₀ of less than about 300 pM. In some embodiments, the multispecific antibody induces NK cell dependent cytotoxicity of the tumor cell in vitro with an EC₅₀ of less than about 100 pM. In some embodiments, the multispecific antibody induces NK cell dependent cytotoxicity of the tumor cell in vitro with an EC₅₀ of less than about 50 pM. In some embodiments, the multispecific antibody induces NK cell dependent cytotoxicity of the tumor cell in vitro with an EC₅₀ of less than about 20 pM. In some embodiments, the multispecific antibody induces NK cell dependent cytotoxicity of the tumor cell in vitro with an EC₅₀ of less than about 15 pM. In some embodiments, the multispecific antibody induces NK cell dependent cytotoxicity of the tumor cell in vitro with an EC₅₀ of less than about 10 pM.

In some embodiments of the multispecific antibody provided herein, the EC₅₀ is assessed with a mixture of NK effector cells and target cells expressing the second antigen.

In some embodiments of the multispecific antibody provided herein, the effector cell to target cell ratio is about 0.01 to 1 to about 10 to 1. In some embodiments of the multispecific antibody provided herein, the effector cell to target cell ratio is about 0.01 to 1 to about 5 to 1. In some embodiments, the effector cell to target cell ratio is about 0.1 to 1 to about 2 to 1. In some embodiments, the effector cell to target cell ratio is about 1:1. In certain embodiments, the effector to target cell ratio can, for example, be 0.01:1, 0.02:1, 0.03:1, 0.04:1, 0.05:1, 0.06:1, 0.07:1, 0.08:1, 0.09:1, 1:1, 2:1, 3:1, 4:1, 5:1, 6:1, 7:1, 8:1, 9:1, or 10:1. In certain embodiments, the concentration of the multispecific antibody or antigen-binding fragment thereof is about 0.000005 ng/mL, about 0.00005 ng/mL, about 0.0005, about 0.005 ng/mL, about 0.01 ng/mL, about 0.02 ng/mL, about 0.03 ng/mL, about 0.04 ng/mL, about 0.05 ng/mL, about 0.06 ng/mL, about 0.07 ng/mL, about 0.08 ng/mL, about 0.09 ng/mL, about 0.1 ng/mL, about 0.5 ng/mL, about 1.0 ng/mL, about 10 ng/mL, about 20 ng/mL about, about 30 ng/mL about 40 ng/mL, about 50 ng/mL, about 60 ng/mL, about 70 ng/mL, about 80 ng/mL, about 90 ng/mL, about 100 ng/mL, or about 1000 ng/mL.

### 5.2.3. Monoclonal Antibodies

The antibodies of the present disclosure can be or derived from monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium, which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., 1987, Monoclonal Antibody Production Techniques and Applications 51-63).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Plückthun, 1992, Immunol. Revs. 130:151-88.

In a further embodiment, monoclonal antibodies or antibody fragments can be isolated from antibody phage libraries generated using the techniques described in, for example, Antibody Phage Display: Methods and Protocols (O'Brien and Aitken eds., 2002). In phage display methods, functional antibody domains are displayed on the surface of phage particles which carry the polynucleotide sequences encoding them. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, NKG2d or NKp46 (e.g., an NKG2d or NKp46 polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Antibodies can be obtained by designing a suitable antigen screening procedure to select for the phage clone of interest followed by construction of a full length antibody clone using VH and/or VL sequences (e.g., the Fv sequences), or various CDR sequences from VH and VL sequences, from the phage clone of interest and suitable constant region (e.g., Fc) sequences described in Kabat et al., supra.

Antibodies described herein can also, for example, include chimeric antibodies. A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

Antibodies or antigen binding fragments produced using techniques such as those described herein can be isolated using standard, well known techniques. For example, antibodies or antigen binding fragments can be suitably separated from, e.g., culture medium, ascites fluid, serum, cell lysate, synthesis reaction material or the like by conventional immunoglobulin purification procedures such as, for example, protein A-Sepharose, hydroxylapatite chromatography, gel electrophoresis, dialysis, or affinity chromatography. As used herein, an “isolated” or “purified” antibody is substantially free of cellular material or other proteins from the cell or tissue source from which the antibody is derived, or substantially free of chemical precursors or other chemicals when chemically synthesized.

### 5.2.4. Antibody Fragments

The present disclosure provides antibodies (e.g., multispecific antibodies) comprising antibody fragments that bind to, e.g., NKG2d, and NKp46.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)2 fragments can be isolated directly from recombinant host cell culture. Fab and F(ab′)2 fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to, e.g., an NKG2d, or an NKp46 epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule. In some embodiments, the antibody is an IgG antibody. In some embodiments, the IgG antibody is an IgG1 antibody. In some embodiments, the IgG antibody is an IgG2, IgG3, or IgG4 antibody.

Variants and derivatives of antibodies include antibody functional fragments that retain the ability to bind to, e.g., NKG2d, or NKp46 epitope. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)2 (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes); and a tetrabody (e.g., a tetramerized scFv, formed in a manner similar to a diabody, but in which four antigen-binding domains are created in a single complex; the four antigen binding domains may be directed towards the same or different epitopes).

In some embodiments, the multispecific antibody is a bispecific antibody.

In some embodiments, the multispecific antibody is a bispecific antibody, wherein the bispecific antibody is in a bipod-scaffold configuration. In some embodiments, the multispecific antibody is a bispecific antibody in a bipod-scaffold configuration, wherein the first binding domain is a Fab region, and the second binding domain is a scFv region.

In some embodiments, the multispecific antibody is a bispecific antibody, wherein the bispecific antibody is in a Morrison-scaffold configuration. In some embodiments, the multispecific antibody is a bispecific antibody in a Morrison-scaffold configuration, wherein the first binding domain comprises two Fab regions and the second binding domain comprises two scFv regions.

### 5.2.5. Humanized Antibodies

The antibodies (e.g., multispecific antibodies) described herein can, for example, include humanized antibodies, e.g., deimmunized or composite human antibodies.

A humanized antibody can comprise human framework region and human constant region sequences. For example, a humanized antibody can comprise human constant region sequences. In certain embodiments, a humanized antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). In certain embodiments, a humanized antibody can comprise kappa or lambda light chain constant sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

In some embodiments, antibodies provided herein can be humanized antibodies that bind NKG2d, or NKp46, including human, cynomolgus macaque, rat and mouse NKG2d, or NKp46. For example, humanized antibodies of the present disclosure may comprise one or more CDRs as shown in the Sequence Listing provided herein. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

A deimmunized antibody is an antibody in which T-cell epitopes have been removed. Methods for making deimmunized antibodies have been described. See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

### 5.2.6. Human Antibodies

In specific embodiments, the multispecific antibody provided herein comprises a fully human anti-human antibody or fragment thereof. Fully human antibodies may be produced by any method known in the art. Human antibodies provided herein can be constructed by combining Fv clone variable domain sequence(s) selected from human-derived phage display libraries with known human constant domain sequences(s). Alternatively, human monoclonal antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Brüggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In some instances, however, VH CDR3 and VL CDR3, as well as VH CDR2, VL CDR2, and VL CDR1 of the non-human antibody may be retained.

### 5.2.7. Fc Engineering

It may be desirable to modify an antibody provided herein by Fc engineering. In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. In some embodiment, the Fc region comprises IgG1 silent mutations. In some embodiments, the Fc region comprise AAS mutation.

In certain embodiments, the modification to the Fc region of the antibody results in the enhancement of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. In certain embodiments, the Fc region comprises CDC enhancement mutation. In some embodiment, the Fc region comprises K248E and T437R mutations.

In certain embodiments of the antibody provided herein, the Fc region is afucosylated.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### 5.2.8. Alternative Binding Agents

The present disclosure encompasses non-immunoglobulin binding agents that specifically bind to the same epitope as an antibody disclosed herein. In some embodiments, a non-immunoglobulin binding agent is identified as an agent that displaces or is displaced by an antibody of the present disclosure in a competitive binding assay. These alternative binding agents may include, for example, any of the engineered protein scaffolds known in the art. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352: 95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### 5.2.9. Antibody Variants

In some embodiments, amino acid sequence modification(s) of the antibodies or antigen binding fragments that bind to, e.g., NKG2d, NKp46, provided herein are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art would appreciate that amino acid changes may alter post-translational processes of the antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites. Accordingly, in one embodiment, an antibody or antigen binding fragment thereof that binds to an NKG2d epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein. Accordingly, in one embodiment, an antibody or antigen binding fragment thereof that binds to an NKp46 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein.

The variations can be made using methods known in the art such as oligonucleotide-mediated (site-directed) mutagenesis, alanine scanning, and PCR mutagenesis. Site-directed mutagenesis (see. e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-NKG2d or anti-NKp46 antibody variant DNA.

Any cysteine residue not involved in maintaining the proper conformation of the antibody provided herein also may be substituted, for example, with another amino acid, such as alanine or serine, to improve the oxidative stability of the molecule and to prevent aberrant crosslinking. Conversely, cysteine bond(s) may be added to the antibody to improve its stability (e.g., where the antibody is an antibody fragment such as an Fv fragment).

In some embodiments, an antibody molecule of the present disclosure is a “de-immunized” antibody. A “de-immunized” antibody is an antibody derived from a humanized or chimeric antibody, which has one or more alterations in its amino acid sequence resulting in a reduction of immunogenicity of the antibody, compared to the respective original non-de-immunized antibody. One of the procedures for generating such antibody mutants involves the identification and removal of T-cell epitopes of the antibody molecule. In a first step, the immunogenicity of the antibody molecule can be determined by several methods, for example, by in vitro determination of T-cell epitopes or in silico prediction of such epitopes, as known in the art. Once the critical residues for T-cell epitope function have been identified, mutations can be made to remove immunogenicity and retain antibody activity. For review, see, for example, Jones et al., 2009, Methods in Molecular Biology 525:405-23.

### 5.2.10. In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, the antibodies can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 5.2.11. Antibody Modifications

Covalent modifications of the antibodies binding to, e.g., NKG2d and NKp46, provided herein are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see. e.g., Creighton, Proteins: Structure and Molecular Properties 79-86 (1983)), acetylation of the N-terminal amine, and amidation of any C-terminal carboxyl group.

Other types of covalent modification of the antibody provided herein included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An antibody of the present disclosure may also be modified to form chimeric molecules comprising the antibody fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are fusion proteins comprising an antibody provided herein that binds to, e.g., NKG2d, NKp46, and a heterologous polypeptide.

Also provided herein are panels of antibodies that bind to an NKG2d, NKp46 antigen. In specific embodiments, the panels of antibodies have different association rates, different dissociation rates, different affinities for an NKG2d, NKp46 antigen, and/or different specificities for an NKG2d, NKp46 antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96-well or 384-well plates, for assays such as ELISAs.

### 5.2.12. Immunoconjugates

The present disclosure also provides conjugates comprising any one of the antibodies of the present disclosure covalently bound by a synthetic linker to one or more non-antibody agents.

In some embodiments, antibodies provided herein are conjugated or recombinantly fused, e.g., to a therapeutic agent (e.g., a cytotoxic agent) or a diagnostic or detectable molecule. The conjugated or recombinantly fused antibodies can be useful, for example, for treating or preventing a disease or disorder. The conjugated or recombinantly fused antibodies can be useful, for example, for monitoring or prognosing the onset, development, progression, and/or severity of a disease or disorder.

Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (131I, 125I, 123I, and 121I,), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 11 7Sn; positron emitting metals using various positron emission tomographies; and non-radioactive paramagnetic metal ions.

Also provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising an antigen-binding fragment of an antibody provided herein (e.g., CDR1, CDR2, and/or CDR3) and a heterologous protein, polypeptide, or peptide. In one embodiment, the heterologous protein, polypeptide, or peptide that the antibody is fused to is useful for targeting the antibody to a particular cell type.

Moreover, antibodies provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to antibodies are known (see, e.g., Amon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described, for example, in U.S. Pat. No. 4,676,980.

Antibodies as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37).

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

### 5.3. Polynucleotides

In certain embodiments, the disclosure encompasses polynucleotides that encode the antibodies described herein. The term “polynucleotides that encode a polypeptide” encompasses a polynucleotide that includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, a polynucleotide comprises the coding sequence for a polypeptide fused in the same reading frame to a polynucleotide which aids, for example, in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In certain embodiments, a polynucleotide comprises the coding sequence for a polypeptide fused in the same reading frame to a marker or tag sequence. For example, in some embodiments, a marker sequence is a hexa-histidine tag supplied by a vector that allows efficient purification of the polypeptide fused to the marker in the case of a bacterial host. In some embodiments, a marker is used in conjunction with other affinity tags.

The present disclosure further relates to variants of the polynucleotides described herein, wherein the variant encodes, for example, fragments, analogs, and/or derivatives of a polypeptide. In certain embodiments, the present disclosure provides a polynucleotide comprising a polynucleotide having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising an antibody or antigen binding fragment thereof described herein.

As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations that produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, the present disclosure provides a polynucleotide comprising a nucleotide sequence at least about 80% identical, at least about 85% identical, at least about 90% identical, at least about 95% identical, and in some embodiments, at least about 96%, 97%, 98% or 99% identical to a polynucleotide listed in the Sequence Listing provided herein.

In certain embodiments, the present disclosure provides a polynucleotide comprising a nucleotide sequence at least about 80% identical, at least about 85% identical, at least about 90% identical, at least about 95% identical, and in some embodiments, at least about 96%, 97%, 98% or 99% identical to a polynucleotide selected from the polynucleotides provided herein.

In certain embodiments, a polynucleotide is isolated. In certain embodiments, a polynucleotide is substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises one or more expression vectors comprising polynucleotide molecules. In some embodiments, a host cell comprises a polynucleotide molecule. In some embodiments, a host cell comprises one or more polynucleotide molecules.

### 5.4. Methods or Processes of Making the Antibodies

In yet another aspect, provided herein are methods or processes for making the various molecules provided herein. In some embodiments, provided herein is a process for making a molecule that binds to more than one target molecule, comprising: a step for performing a function of obtaining a binding domain capable of binding to a first antigen on the surface of an NK cell; a step for performing a function of obtaining a binding domain capable of binding to a second antigen; and a step for performing a function of providing a molecule capable of binding to the first antigen and the second antigen.

Recombinant expression of an antibody provided herein requires construction of an expression vector containing a polynucleotide that encodes the antibody or antigen binding fragment thereof. Once a polynucleotide encoding an antibody molecule, heavy or light chain of an antibody, or fragment thereof (such as, but not necessarily, containing the heavy and/or light chain variable domain) provided herein has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule provided herein, a heavy or light chain of an antibody, a heavy or light chain variable domain of an antibody or a fragment thereof, or a heavy or light chain CDR, operably linked to a promoter. Such vectors may include the nucleotide sequence encoding the constant region of the antibody molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody provided herein. Thus, also provided herein are host cells containing a polynucleotide encoding an antibody provided herein or fragments thereof, or a heavy or light chain thereof, or fragment thereof, or a single chain antibody provided herein, operably linked to a heterologous promoter. In certain embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules provided herein (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule provided herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In some embodiments, antibodies provided herein are produced in CHO cells. In a specific embodiment, the expression of nucleotide sequences encoding antibodies provided herein which immunospecifically bind to NKG2d antigen is regulated by a constitutive promoter, inducible promoter or tissue specific promoter. In a specific embodiment, the expression of nucleotide sequences encoding antibodies provided herein which immunospecifically bind to NKp46 antigen is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells. In some embodiments, fully human monoclonal antibodies provided herein are produced in mammalian cells, such as CHO cells.

For long-term, high-yield production of recombinant proteins, stable expression can be utilized. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics. John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two or more expression vectors provided herein. The two or more vectors may contain identical selectable markers which enable equal expression of, e.g., heavy and light chain polypeptides. Alternatively, a single vector may be used which encodes, and is capable of expressing different component polypeptides of the present antibodies, e.g., both heavy and light chain polypeptides. The coding sequences may comprise cDNA or genomic DNA.

Once an antibody molecule provided herein has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies provided herein can be fused to heterologous polypeptide sequences described herein or otherwise known in the art to facilitate purification.

### 5.5. Pharmaceutical Compositions

In one aspect, the present disclosure further provides pharmaceutical compositions comprising at least one antibody or antigen binding fragment thereof of the present disclosure. In some embodiments, a pharmaceutical composition comprises therapeutically effective amount of an antibody or antigen binding fragment thereof provided herein and a pharmaceutically acceptable excipient. Also provided is a method of producing the pharmaceutical composition, comprising combining the antibody or antigen binding fragment thereof provided herein with a pharmaceutically acceptable carrier to obtain the pharmaceutical composition.

In another general aspect, provided is a pharmaceutical composition comprising a multispecific antibody provided herein and a pharmaceutically acceptable carrier. In certain embodiments, the multispecific antibody is isolated. Also provided is a method of producing the pharmaceutical composition, comprising combining the multispecific antibody with a pharmaceutically acceptable carrier to obtain the pharmaceutical composition. In another aspect, provided herein is a pharmaceutical composition comprising a comprising: (a) a first binding domain that binds to NKG2d, and (b) a second binding domain that binds to a second target, and a pharmaceutically acceptable carrier. In another aspect, provided herein is a pharmaceutical composition comprising a comprising: (a) a first binding domain that binds to NKp46, and (b) a second binding domain that binds to a second target, and a pharmaceutically acceptable carrier. Any of the multispecific antibodies provided herein are contemplated in the pharmaceutical compositions. In certain embodiments, the second binding domain binds to BCMA. In certain embodiments, the second binding domain binds to GPRC5d. Any of the antibodies provided herein are contemplated in the pharmaceutical compositions.

Pharmaceutical compositions comprising an antibody or antigen binding fragment thereof are prepared for storage by mixing the protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody or antigen binding fragment thereof of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof as described herein, including, but not limited to, encapsulation in liposomes, microparticles, microcapsules, recombinant cells capable of expressing the antibody or antigen binding fragment thereof, receptor-mediated endocytosis (see. e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6. Methods of Using

In yet another aspect, provided herein is a method of enriching, isolating, separating, purifying, sorting, selecting, capturing, detecting or depleting cells expressing NKG2d, comprising providing a sample comprising the cells expressing NKG2d; contacting the sample with the NKG2d antibody provided herein; and enriching, isolating, separating, purifying, sorting, selecting, capturing, detecting or depleting the cells expressing NKG2d and bound to the NKG2d antibody.

In yet another aspect, provided herein is use of an NKG2d antibody provided herein for enriching, isolating, separating, purifying, sorting, selecting, capturing, detecting or depleting cells expressing NKG2d, comprising providing a sample comprising the cells expressing NKG2d; contacting the sample with the NKG2d antibody provided herein; and enriching, isolating, separating, purifying, sorting, selecting, capturing, detecting or depleting the cells expressing NKG2d and bound to the NKG2d antibody.

In another aspect, provided herein is a method of enriching, isolating, separating, purifying, sorting, selecting, capturing, detecting or depleting cells expressing NKp46, comprising providing a sample comprising the cells expressing NKp46; contacting the sample with the NKp46 antibody provided herein; and enriching, isolating, separating, purifying, sorting, selecting, capturing, detecting or depleting the cells expressing NKp46 and bound to the NKp46 antibody.

In another aspect, provided herein is use of an NKp46 antibody provided herein for enriching, isolating, separating, purifying, sorting, selecting, capturing, detecting or depleting cells expressing NKp46, comprising providing a sample comprising the cells expressing NKp46; contacting the sample with the NKp46 antibody provided herein; and enriching, isolating, separating, purifying, sorting, selecting, capturing, detecting or depleting the cells expressing NKp46 and bound to the NKp46 antibody.

In some embodiments, the cells are NK cells. In some embodiments, the sample is a blood sample. In other embodiments, the sample is a tissue sample.

In yet another aspect, provided herein is a method of directing an NK cell to a target cell, comprising contacting the NK cell with a multispecific antibody provided herein, thereby directing the NK cell to the target cell, wherein the multispecific antibody comprises a first binding domain that binds to a first antigen on an NK cell and a second binding domain that binds to a second antigen on a target cell. In some embodiments, the target cell is a tumor cell. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d.

In yet another aspect, provided herein is use of a multispecific antibody provided herein for directing an NK cell to a target cell, comprising contacting the NK cell with the multispecific antibody provided herein, thereby directing the NK cell to the target cell, wherein the multispecific antibody comprises a first binding domain that binds to a first antigen on an NK cell and a second binding domain that binds to a second antigen on a target cell. In some embodiments, the target cell is a tumor cell. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d.

In yet another aspect, provided herein is a method of activating an NK cell, comprising contacting the NK cell with a multispecific antibody provided herein, wherein the multispecific antibody comprises a first binding domain that binds to a first antigen on the NK cell and a second binding domain that binds to a second antigen on a target cell. In some embodiments, the target cell is a tumor cell. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d.

In yet another aspect, provided herein is use of a multispecific antibody provided herein for activating an NK cell, comprising contacting the NK cell with the multispecific antibody provided herein, wherein the multispecific antibody comprises a first binding domain that binds to a first antigen on the NK cell and a second binding domain that binds to a second antigen on a target cell. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d.

In yet another aspect, provided herein is a method of inhibiting growth or proliferation of target cells expressing a second antigen on the cell surface, the method comprising contacting the target cells with a multispecific antibody provided herein, wherein the multispecific antibody comprises a first binding domain that binds to a first antigen on an NK cell and a second binding domain that binds to the second antigen. In some embodiments, the second antigen express on a tumor cell. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d.

In yet another aspect, provided herein is use of a multispecific antibody provided herein for inhibiting growth or proliferation of target cells expressing a second antigen on the cell surface, the use comprising contacting the target cells with the multispecific antibody provided herein, wherein the multispecific antibody comprises a first binding domain that binds to a first antigen on an NK cell and a second binding domain that binds to the second antigen. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d

In yet another aspect, provided herein is a method for eliminating target cells expressing a second antigen or treating a disease or disorder caused all or in part by target cells expressing the second antigen in a subject, comprising administering an effective amount of a multispecific antibody provided herein to the subject, wherein the multispecific antibody comprises a first binding domain that binds to a first antigen on an NK cell and a second binding domain that binds to the second antigen. In some embodiments, the second antigen express on a tumor cell. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d.

In yet another aspect, provided herein is use of a multispecific antibody provided herein for eliminating target cells expressing a second antigen or treating a disease or disorder caused all or in part by target cells expressing the second antigen in a subject, comprising administering an effective amount of the multispecific antibody provided herein to the subject, wherein the multispecific antibody comprises a first binding domain that binds to a first antigen on an NK cell and a second binding domain that binds to the second antigen. In certain embodiments, the multispecific antibody is a multispecific NKG2d/BCMA antibody, wherein the first target is NKG2d and the second target is BCMA. In certain embodiments, the multispecific antibody is a multispecific NKG2d/GPRC5d antibody, wherein the first target is NKG2d and the second target is GPRC5d. In certain embodiments, the multispecific antibody is a multispecific NKp46/BCMA antibody, wherein the first target is NKp46 and the second target is BCMA. In certain embodiments, the multispecific antibody is a multispecific NKp46/GPRC5d antibody, wherein the first target is NKp46 and the second target is GPRC5d.

In yet another aspect, provided herein is a method of inhibiting or depleting cancer cells in a subject having cancer, comprising administering to the subject the multispecific antibody provided herein.

In yet another aspect, provided herein is use of a multispecific antibody provided herein for inhibiting or depleting cancer cells in a subject having cancer, comprising administering to the subject the multispecific antibody provided herein.

In yet another aspect, provided herein is a method of treating cancer in a subject, comprising administering to the subject the multispecific antibody provided herein. Also provided herein is a multispecific antibody as described herein for use in the treatment of cancer in a subject. In some embodiments, the cancer is a solid tumor cancer. In other embodiments, the cancer is a blood cancer. In yet another aspect, provided herein is a multispecific antibody as described herein for use as a medicament.

In another aspect, provided herein is a method of treating a disease or disorder in a subject comprising administering to the subject an effective amount of an antibody or antigen binding fragment thereof provided herein. Also provided herein is an antibody or antigen binding fragment thereof as described herein for use in the treatment of a disease or disorder.

In another aspect, provided herein is a method of treating a disease or disorder in a subject comprising administering to the subject an effective amount of the multispecific antibody provided herein. Also provided herein is a multispecific antibody as described herein for use in the treatment of a disease or disorder.

Also provided herein is a method of treatment of a disease or disorder, wherein the subject is administered one or more therapeutic agents in combination with the antibody or antigen-binding fragment thereof provided herein.

In another aspect, provided herein is the use of the antibody or antigen binding fragment thereof provided herein in the manufacture of a medicament for treating a disease or disorder in a subject. In another aspect, provided herein is the use of the multispecific antibody provided herein in the manufacture of a medicament for treating a disease or disorder in a subject.

In another aspect, provided herein is the use of a pharmaceutical composition provided herein in the manufacture of a medicament for treating a disease or disorder in a subject.

In a specific embodiment, provided herein is a composition for use as a medicament, wherein the composition comprises an antibody or antigen binding fragment thereof and a pharmaceutically-acceptable excipient/carrier. In a specific embodiment, provided herein is a composition for use in the prevention and/or treatment of a disease or condition, wherein the composition comprises an antibody or antigen binding fragment thereof and a pharmaceutically-acceptable excipient/carrier. In one embodiment, provided herein is a composition for use in the prevention of a disease or condition, wherein the composition comprises an antibody or antigen binding fragment thereof and a pharmaceutically-acceptable excipient/carrier. In one embodiment, provided herein is a composition for use in the treatment of a disease or condition, wherein the composition comprises an antibody or antigen binding fragment thereof and a pharmaceutically-acceptable excipient/carrier. In one embodiment, provided herein is a composition for use in the treatment of cancer, wherein the composition comprises an antibody or antigen-binding fragment thereof and a pharmaceutically-acceptable excipient/carrier. In certain embodiments, the subject is a subject in need thereof. In some embodiments, the subject has the disease or condition. In other embodiments, the subject is at risk of having the disease or condition. In some embodiments, the administration results in the prevention, management, treatment or amelioration of the disease or condition.

In one embodiment, provided herein is a composition for use in the prevention and/or treatment of a symptom of a disease or condition, wherein the composition comprises an antibody or antigen binding fragment thereof provided and a pharmaceutically-acceptable excipient/carrier. In one embodiment, provided herein is a composition for use in the prevention of a symptom of a disease or condition, wherein the composition comprises an antibody or antigen binding fragment thereof and a pharmaceutically-acceptable excipient/carrier. In one embodiment, provided herein is a composition for use in the treatment of a symptom of a disease or condition, wherein the composition comprises an antibody or antigen binding fragment thereof and a pharmaceutically-acceptable excipient/carrier. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of an antibody or antigen binding fragment thereof provided herein. In one embodiment, provided herein is a method of preventing a disease or condition in a subject, comprising administering an effective amount of an antibody or antigen binding fragment thereof provided herein. In one embodiment, provided herein is a method of treating a disease or condition in a subject, comprising administering an effective amount of an antibody or antigen binding fragment thereof provided herein. In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of an antibody or antigen binding fragment thereof provided herein. In one embodiment, provided herein is a method of preventing a symptom of a disease or condition in a subject, comprising administering an effective amount of an antibody or antigen binding fragment thereof provided herein. In one embodiment, provided herein is a method of treating a symptom of a disease or condition in a subject, comprising administering an effective amount of an antibody or antigen binding fragment thereof provided herein. In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

Also provided herein are methods of preventing and/or treating a disease or condition by administrating to a subject of an effective amount of an antibody or antigen binding fragment thereof provided herein, or pharmaceutical composition comprising an antibody or antigen binding fragment thereof provided herein. In one aspect, the antibody or antigen binding fragment thereof is substantially purified (i.e., substantially free from substances that limit its effect or produce undesired side-effects). The subject administered a therapy can be a mammal such as non-primate or a primate (e.g., a human). In a one embodiment, the subject is a human. In another embodiment, the subject is a human with a disease or condition.

Various delivery systems are known and can be used to administer a prophylactic or therapeutic agent (e.g., an antibody or antigen binding fragment thereof provided herein), including, but not limited to, encapsulation in liposomes, microparticles, microcapsules, recombinant cells capable of expressing the antibody or antigen binding fragment thereof, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody or antigen binding fragment thereof provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody or antigen binding fragment thereof provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering an antibody or antigen binding fragment thereof provided herein, care must be taken to use materials to which the antibody or antigen binding fragment thereof does not absorb.

In another embodiment, a prophylactic or therapeutic agent, or a composition provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more antibody or antigen binding fragment thereof provided herein. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition provided herein is a nucleic acid encoding a prophylactic or therapeutic agent (e.g., an antibody or antigen binding fragment thereof provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition provided herein comprises one, two or more antibodies or antigen binding fragments thereof provided herein. In another embodiment, a composition provided herein comprises one, two or more antibodies or antigen binding fragments thereof provided herein and a prophylactic or therapeutic agent other than an antibody or antigen binding fragment thereof provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antibody or antigen binding fragment thereof provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the antibody or antigen binding fragment thereof provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antibody or antigen binding fragment thereof provided herein can be packaged in a hermetically sealed container such as an ampoule or sachette indicating the quantity of antibody. In one embodiment, the antibody or antigen binding fragment thereof is supplied as a dry sterilized lyophilized powder or water free concentrate in a hermetically sealed container and can be reconstituted, e.g., with water or saline to the appropriate concentration for administration to a subject. The lyophilized antibody or antigen binding fragment thereof can be stored at between 2 and 8° C. in its original container and the antibody or antigen binding fragment thereof can be administered within 12 hours, such as within 6 hours, within 5 hours, within 3 hours, or within 1 hour after being reconstituted. In an alternative embodiment, an antibody or antigen binding fragment thereof provided herein is supplied in liquid form in a hermetically sealed container indicating the quantity and concentration of the antibody.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antibody or antigen binding fragment thereof provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In certain embodiments, the route of administration for a dose of an antibody or antigen binding fragment thereof provided herein to a patient is intranasal, intramuscular, intravenous, subcutaneous, or a combination thereof, but other routes described herein are also acceptable. Each dose may or may not be administered by an identical route of administration. In some embodiments, an antibody or antigen binding fragment thereof provided herein may be administered via multiple routes of administration simultaneously or subsequently to other doses of the same or a different antibody or antigen binding fragment thereof provided herein.

In certain embodiments, the antibody or antigen binding fragment thereof provided herein are administered prophylactically or therapeutically to a subject. The antibody or antigen binding fragment thereof provided herein can be prophylactically or therapeutically administered to a subject so as to prevent, lessen or ameliorate a disease or symptom thereof.

### 5.7. Gene Therapy

In a specific embodiment, nucleic acids comprising sequences encoding antibodies or functional derivatives thereof, are administered to a subject for use in a method provided herein, for example, to prevent, manage, treat and/or ameliorate a disease, disorder or condition, by way of gene therapy. Such therapy encompasses that performed by the administration to a subject of an expressed or expressible nucleic acid. In an embodiment, the nucleic acids produce their encoded antibody, and the antibody mediates a prophylactic or therapeutic effect. Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-5%; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, a composition comprises nucleic acids encoding an antibody provided herein, the nucleic acids being part of an expression vector that expresses the antibody or chimeric proteins or heavy or light chains thereof in a suitable host. In particular, such nucleic acids have promoters, such as heterologous promoters, operably linked to the antibody coding region, the promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In some embodiments, the expressed antibody molecule is a single chain antibody; alternatively, the nucleic acid sequences include sequences encoding both the heavy and light chains, or fragments thereof, of the antibody.

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an antibody as provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for gene therapy is autologous to the subject.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.8. Diagnostic Assays and Methods

Labeled antibodies and derivatives and analogs thereof, which immunospecifically bind to an antigen provided herein can be used for diagnostic purposes to detect, diagnose, or monitor a disease or disorder.

Antibodies provided herein can be used to assay an antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (121In), and technetium (99Tc); luminescent labels, such as luminol; and fluorescent labels, such as fluorescein and rhodamine, and biotin. One aspect provided herein is the detection and diagnosis of a disease or disorder in a human.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of a disease or disorder is carried out by repeating the method for diagnosing the disease or disorder, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.9. Kits

Also provided herein are kits comprising an antibody (e.g., an anti-NKG2d multispecific antibody or anti-NKp46 multispecific antibody) provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to an antigen, e.g., NKG2d or NKp46. In specific embodiments, provided herein are panels of antibodies having different association rate constants different dissociation rate constants, different affinities for an antigen, and/or different specificities for an antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. EMBODIMENTS

This invention provides the following non-limiting embodiments.

In one set of embodiments (embodiment set A), provided are:
A1. A multispecific antibody comprising:
   (a) a first binding domain that binds to a first antigen expressed on a Natural Killer (NK) cell, and
   (b) a second binding domain that binds to a second antigen.
A2. The multispecific antibody of embodiment A1, wherein the first antigen is an NK cell activating receptor.
A3. The multispecific antibody of embodiment A2, wherein the first antigen is NKG2d.
A4. The multispecific antibody of embodiment A3, wherein the first binding domain comprises:
   (i) a heavy chain variable region (VH) comprising:
      (a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6;
      (b) a VH complementarity determining region (CDR) 1 having an amino acid sequence of SEQ ID NO:10, a VH CDR2 having an amino acid sequence of SEQ ID NO:11, and a VH CDR3 having an amino acid sequence of SEQ ID NO:12;
      (c) a VH complementarity determining region (CDR) 1 having an amino acid sequence of SEQ ID NO:16, a VH CDR2 having an amino acid sequence of SEQ ID NO:17, and a VH CDR3 having an amino acid sequence of SEQ ID NO:18;
      (d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or
      (e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30;
      and
      a light chain variable region (VL) comprising:
      (a) a VL CDR1 having an amino acid sequence of SEQ ID NO:7, a VL CDR2 having an amino acid sequence of SEQ ID NO:8, and a VL CDR3 having an amino acid sequence of SEQ ID NO:9;
      (b) a VL CDR1 having an amino acid sequence of SEQ ID NO:13, a VL CDR2 having an amino acid sequence of SEQ ID NO:14, and a VL CDR3 having an amino acid sequence of SEQ ID NO:15;
      (c) a VL CDR1 having an amino acid sequence of SEQ ID NO:19, a VL CDR2 having an amino acid sequence of SEQ ID NO:20, and a VL CDR3 having an amino acid sequence of SEQ ID NO:21;
      (d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or
      (e) a VL CDR1 having an amino acid sequence of SEQ ID NO:31, a VL CDR2 having an amino acid sequence of SEQ ID NO:32, and a VL CDR3 having an amino acid sequence of SEQ ID NO:33;
   or
   (ii) a heavy chain variable region (VH) comprising:
      (a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38;
      (b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44;
      (c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50;
      (d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or
      (e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62;
      and
      a light chain variable region (VL) comprising:
      (a) a VL CDR1 having an amino acid sequence of SEQ ID NO:39, a VL CDR2 having an amino acid sequence of SEQ ID NO:40, and a VL CDR3 having an amino acid sequence of SEQ ID NO:41;
      (b) a VL CDR1 having an amino acid sequence of SEQ ID NO:45, a VL CDR2 having an amino acid sequence of SEQ ID NO:46, and a VL CDR3 having an amino acid sequence of SEQ ID NO:47;
      (c) a VL CDR1 having an amino acid sequence of SEQ ID NO:51, a VL CDR2 having an amino acid sequence of SEQ ID NO:52, and a VL CDR3 having an amino acid sequence of SEQ ID NO:53;
      (d) a VL CDR1 having an amino acid sequence of SEQ ID NO:57, a VL CDR2 having an amino acid sequence of SEQ ID NO:58, and a VL CDR3 having an amino acid sequence of SEQ ID NO:59; or
      (e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.
A5. The multispecific antibody of embodiment A4, wherein the first binding domain comprises a VH having an amino acid sequence of SEQ ID NO:2, and a VL having an amino acid sequence of SEQ ID NO:3, or wherein the first binding domain comprises a VH having an amino acid sequence of SEQ ID NO:34, and a VL having an amino acid sequence of SEQ ID NO:35.
A6. The multispecific antibody of embodiment A2, wherein the first antigen is NKp46.
A7. The multispecific antibody of embodiment A6, wherein the first binding domain comprises:
   (i) a heavy chain variable region (VH) comprising:
      (a) a VH complementarity determining region (CDR) 1 having an amino acid sequence of SEQ ID NO:69, a VH CDR2 having an amino acid sequence of SEQ ID NO:70, and a VH CDR3 having an amino acid sequence of SEQ ID NO:71;
      (b) a VH complementarity determining region (CDR) 1 having an amino acid sequence of SEQ ID NO:75, a VH CDR2 having an amino acid sequence of SEQ ID NO:76, and a VH CDR3 having an amino acid sequence of SEQ ID NO:77;
      (c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83;
      (d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or
      (e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95;
   and
   (ii) a light chain variable region (VL) comprising:
      (a) a VL CDR1 having an amino acid sequence of SEQ ID NO:72, a VL CDR2 having an amino acid sequence of SEQ ID NO:73, and a VL CDR3 having an amino acid sequence of SEQ ID NO:74;
      (b) a VL CDR1 having an amino acid sequence of SEQ ID NO:78, a VL CDR2 having an amino acid sequence of SEQ ID NO:79, and a VL CDR3 having an amino acid sequence of SEQ ID NO:80;
      (c) a VL CDR1 having an amino acid sequence of SEQ ID NO:84, a VL CDR2 having an amino acid sequence of SEQ ID NO:85, and a VL CDR3 having an amino acid sequence of SEQ ID NO:86;
      (d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or
      (e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.
A8. The multispecific antibody of embodiment A7, wherein the first binding domain comprises: a VH having an amino acid sequence of SEQ ID NO:67, and a VL having an amino acid sequence of SEQ ID NO:68.
A9. The multispecific antibody of any one of embodiments A1 to A8, wherein the second antigen is on a cell surface.
A10. The multispecific antibody of any one of embodiments A1 to A8, wherein the second antigen is expressed on a tumor cell.
A11. The multispecific antibody of embodiment A10, wherein the second antigen is a tumor specific antigen (TSA) or a tumor associated antigen (TAA).
A12. The multispecific antibody of embodiment A10, wherein the second antigen is BCMA.
A13. The multispecific antibody of embodiment A10, wherein the second antigen is GPRC5d.
A14. The multispecific antibody of any one of embodiments A1 to A13, wherein the first binding domain is humanized, the second binding domain is humanized, or both the first binding domain and the second binding domain are humanized.
A15. The multispecific antibody of any one of embodiments A1 to A14, wherein the multispecific antibody is an IgG antibody.
A16. The multispecific antibody of embodiment A15, wherein the IgG antibody is an IgG1, IgG2, IgG3, or IgG4 antibody.
A17. The multispecific antibody of embodiment A16, wherein the IgG antibody is an IgG1 antibody.
A18. The multispecific antibody of any one of embodiments A1 to A17, wherein the multispecific antibody is a bispecific antibody.
A19. The multispecific antibody of embodiment A18, wherein the bispecific antibody is in a bipod-scaffold configuration.
A20. The multispecific antibody of embodiment A19, wherein the first binding domain is a Fab region, and the second binding domain is a scFv region.
A21. The multispecific antibody of embodiment A18, wherein the bispecific antibody is in a Morrison-scaffold configuration.
A22. The multispecific antibody of embodiment A21, wherein the first binding domain comprises two Fab regions and the second binding domain comprises two scFv regions.
A23. The multispecific antibody of any one of embodiments A10 to A22, wherein the multispecific antibody induces NK cell dependent cytotoxicity of the tumor cell in vitro with an IC₅₀ of less than about 500 pM.
A24. The multispecific antibody of any one of embodiments A10 to A22, wherein the multispecific antibody induces NK cell dependent cytotoxicity of the tumor cell in vitro with an IC₅₀ of less than about 300 pM.
A25. The multispecific antibody of any one of embodiments A10 to A22, wherein the multispecific antibody induces NK cell dependent cytotoxicity of the tumor cell in vitro with an IC₅₀ of less than about 100 pM.
A26. The multispecific antibody of any one of embodiments A10 to A22, wherein the multispecific antibody induces NK cell dependent cytotoxicity of the tumor cell in vitro with an IC₅₀ of less than about 50 pM.
A27. The multispecific antibody of any one of embodiments A10 to A22, wherein the multispecific antibody induces NK cell dependent cytotoxicity of the tumor cell in vitro with an IC₅₀ of less than about 20 pM.
A28. The multispecific antibody of any one of embodiments A10 to A22, wherein the multispecific antibody induces NK cell dependent cytotoxicity of the tumor cell in vitro with an IC₅₀ of less than about 15 pM.
A29. The multispecific antibody of any one of embodiments A10 to A22, wherein the multispecific antibody induces NK cell dependent cytotoxicity of the tumor cell in vitro with an IC₅₀ of less than about 10 pM.
A30. The multispecific antibody of any one of embodiments A23 to A29, wherein the IC₅₀ is assessed with a mixture of NK effector cells and target cells expressing the second antigen.
A31. The multispecific antibody of embodiment A30, wherein the effector cell to target cell ratio is about 0.01 to 1 to about 5 to 1.
A32. The multispecific antibody of embodiment A30, wherein the effector cell to target cell ratio is about 0.1 to 1 to about 2 to 1.
A33. The multispecific antibody of embodiment A30, wherein the effector cell to target cell ratio is about 1:1.
A34. A nucleic acid encoding the multispecific antibody of any one of embodiments A1 to A33.
A35. A vector comprising the nucleic acid of embodiment A34.
A36. A host cell comprising the vector of embodiment A35.
A37. A kit comprising the vector of embodiment A35 and packaging for the same.
A38. An antibody that binds NKG2d, comprising:
   (i) a heavy chain variable region (VH) comprising:
      (a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6;
      (b) a VH complementarity determining region (CDR) 1 having an amino acid sequence of SEQ ID NO:10, a VH CDR2 having an amino acid sequence of SEQ ID NO:11, and a VH CDR3 having an amino acid sequence of SEQ ID NO:12;
      (c) a VH complementarity determining region (CDR) 1 having an amino acid sequence of SEQ ID NO:16, a VH CDR2 having an amino acid sequence of SEQ ID NO:17, and a VH CDR3 having an amino acid sequence of SEQ ID NO:18;
      (d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or
      (e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30;
      and
      a light chain variable region (VL) comprising:
      (a) a VL CDR1 having an amino acid sequence of SEQ ID NO:7, a VL CDR2 having an amino acid sequence of SEQ ID NO:8, and a VL CDR3 having an amino acid sequence of SEQ ID NO:9;
      (b) a VL CDR1 having an amino acid sequence of SEQ ID NO:13, a VL CDR2 having an amino acid sequence of SEQ ID NO:14, and a VL CDR3 having an amino acid sequence of SEQ ID NO:15;
      (c) a VL CDR1 having an amino acid sequence of SEQ ID NO:19, a VL CDR2 having an amino acid sequence of SEQ ID NO:20, and a VL CDR3 having an amino acid sequence of SEQ ID NO:21;
      (d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or
      (e) a VL CDR1 having an amino acid sequence of SEQ ID NO:31, a VL CDR2 having an amino acid sequence of SEQ ID NO:32, and a VL CDR3 having an amino acid sequence of SEQ ID NO:33;
   or
   (ii) a heavy chain variable region (VH) comprising:
      (a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38;
      (b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44;
      (c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50;
      (d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or
      (e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62;
      and
      a light chain variable region (VL) comprising:
      (a) a VL CDR1 having an amino acid sequence of SEQ ID NO:39, a VL CDR2 having an amino acid sequence of SEQ ID NO:40, and a VL CDR3 having an amino acid sequence of SEQ ID NO:41;
      (b) a VL CDR1 having an amino acid sequence of SEQ ID NO:45, a VL CDR2 having an amino acid sequence of SEQ ID NO:46, and a VL CDR3 having an amino acid sequence of SEQ ID NO:47;
      (c) a VL CDR1 having an amino acid sequence of SEQ ID NO:51, a VL CDR2 having an amino acid sequence of SEQ ID NO:52, and a VL CDR3 having an amino acid sequence of SEQ ID NO:53;
      (d) a VL CDR1 having an amino acid sequence of SEQ ID NO:57, a VL CDR2 having an amino acid sequence of SEQ ID NO:58, and a VL CDR3 having an amino acid sequence of SEQ ID NO:59; or
      (e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.
A39. The antibody of embodiment A38, wherein the first binding domain comprises a VH having an amino acid sequence of SEQ ID NO:2, and a VL having an amino acid sequence of SEQ ID NO:3, or wherein the first binding domain comprises a VH having an amino acid sequence of SEQ ID NO:34, and a VL having an amino acid sequence of SEQ ID NO:35.
A40. An antibody that binds NKp46, comprising:
   (i) a heavy chain variable region (VH) comprising
      (a) a VH complementarity determining region (CDR) 1 having an amino acid sequence of SEQ ID NO:69, a VH CDR2 having an amino acid sequence of SEQ ID NO:70, and a VH CDR3 having an amino acid sequence of SEQ ID NO:71;
      (b) a VH complementarity determining region (CDR) 1 having an amino acid sequence of SEQ ID NO:75, a VH CDR2 having an amino acid sequence of SEQ ID NO:76, and a VH CDR3 having an amino acid sequence of SEQ ID NO:77;
      (c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83;
      (d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or
      (e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95;
   and
   (ii) a light chain variable region (VL) comprising
      (a) a VL CDR1 having an amino acid sequence of SEQ ID NO:72, a VL CDR2 having an amino acid sequence of SEQ ID NO:73, and a VL CDR3 having an amino acid sequence of SEQ ID NO:74;
      (b) a VL CDR1 having an amino acid sequence of SEQ ID NO:78, a VL CDR2 having an amino acid sequence of SEQ ID NO:79, and a VL CDR3 having an amino acid sequence of SEQ ID NO:80;
      (c) a VL CDR1 having an amino acid sequence of SEQ ID NO:84, a VL CDR2 having an amino acid sequence of SEQ ID NO:85, and a VL CDR3 having an amino acid sequence of SEQ ID NO:86;
      (d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or
      (e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.
A41. The antibody of embodiment A40, wherein the first binding domain comprises a VH having an amino acid sequence of SEQ ID NO:67, and a VL having an amino acid sequence of SEQ ID NO:68.
A42. A nucleic acid encoding the antibody of any one of embodiments A38 to A41.
A43. A vector comprising the nucleic acid of embodiment A42.
A44. A host cell comprising the vector of embodiment A43.
A45. A kit comprising the vector of embodiment A43 and packaging for the same.
In one set of embodiments (embodiment set B), provided are:
B1. A pharmaceutical composition comprising a multispecific antibody, and a pharmaceutically acceptable carrier, wherein the multispecific antibody comprises:
   (a) a first binding domain that binds to a first antigen expressed on a Natural Killer (NK) cell, and
   (b) a second binding domain that binds to a second antigen.
B2. The pharmaceutical composition of embodiment B1, wherein the first antigen is an NK cell activating receptor.
B3. The pharmaceutical composition of embodiment B2, wherein the first antigen is NKG2d.
B4. The pharmaceutical composition of embodiment B3, wherein the first binding domain comprises:
   (i) a heavy chain variable region (VH) comprising:
      (a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6;
      (b) a VH complementarity determining region (CDR) 1 having an amino acid sequence of SEQ ID NO:10, a VH CDR2 having an amino acid sequence of SEQ ID NO:11, and a VH CDR3 having an amino acid sequence of SEQ ID NO:12;
      (c) a VH complementarity determining region (CDR) 1 having an amino acid sequence of SEQ ID NO:16, a VH CDR2 having an amino acid sequence of SEQ ID NO:17, and a VH CDR3 having an amino acid sequence of SEQ ID NO:18;
      (d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or
      (e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30;
      and
      a light chain variable region (VL) comprising:
      (a) a VL CDR1 having an amino acid sequence of SEQ ID NO:7, a VL CDR2 having an amino acid sequence of SEQ ID NO:8, and a VL CDR3 having an amino acid sequence of SEQ ID NO:9;
      (b) a VL CDR1 having an amino acid sequence of SEQ ID NO:13, a VL CDR2 having an amino acid sequence of SEQ ID NO:14, and a VL CDR3 having an amino acid sequence of SEQ ID NO:15;
      (c) a VL CDR1 having an amino acid sequence of SEQ ID NO:19, a VL CDR2 having an amino acid sequence of SEQ ID NO:20, and a VL CDR3 having an amino acid sequence of SEQ ID NO:21;
      (d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or
      (e) a VL CDR1 having an amino acid sequence of SEQ ID NO:31, a VL CDR2 having an amino acid sequence of SEQ ID NO:32, and a VL CDR3 having an amino acid sequence of SEQ ID NO:33;
      or
   (ii) a heavy chain variable region (VH) comprising:
      (a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38;
      (b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44;
      (c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50;
      (d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or
      (e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62;
      and
      a light chain variable region (VL) comprising:
      (a) a VL CDR1 having an amino acid sequence of SEQ ID NO:39, a VL CDR2 having an amino acid sequence of SEQ ID NO:40, and a VL CDR3 having an amino acid sequence of SEQ ID NO:41;
      (b) a VL CDR1 having an amino acid sequence of SEQ ID NO:45, a VL CDR2 having an amino acid sequence of SEQ ID NO:46, and a VL CDR3 having an amino acid sequence of SEQ ID NO:47;
      (c) a VL CDR1 having an amino acid sequence of SEQ ID NO:51, a VL CDR2 having an amino acid sequence of SEQ ID NO:52, and a VL CDR3 having an amino acid sequence of SEQ ID NO:53;
      (d) a VL CDR1 having an amino acid sequence of SEQ ID NO:57, a VL CDR2 having an amino acid sequence of SEQ ID NO:58, and a VL CDR3 having an amino acid sequence of SEQ ID NO:59; or
      (e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.
B5. The pharmaceutical composition of embodiment B4, wherein the first binding domain comprises a VH having an amino acid sequence of SEQ ID NO:2, and a VL having an amino acid sequence of SEQ ID NO:3, or wherein the first binding domain comprises: a VH having an amino acid sequence of SEQ ID NO:34, and a VL having an amino acid sequence of SEQ ID NO:35.
B6. The pharmaceutical composition of embodiment B2, wherein the first antigen is NKp46.
B7. The pharmaceutical composition of embodiment B6, wherein the first binding domain comprises:
   (i) a heavy chain variable region (VH) comprising
      (a) a VH complementarity determining region (CDR) 1 having an amino acid sequence of SEQ ID NO:69, a VH CDR2 having an amino acid sequence of SEQ ID NO:70, and a VH CDR3 having an amino acid sequence of SEQ ID NO:71;
      (b) a VH complementarity determining region (CDR) 1 having an amino acid sequence of SEQ ID NO:75, a VH CDR2 having an amino acid sequence of SEQ ID NO:76, and a VH CDR3 having an amino acid sequence of SEQ ID NO:77;
      (c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83;
      (d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or
      (e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95;
   and
   (ii) a light chain variable region (VL) comprising
      (a) a VL CDR1 having an amino acid sequence of SEQ ID NO:72, a VL CDR2 having an amino acid sequence of SEQ ID NO:73, and a VL CDR3 having an amino acid sequence of SEQ ID NO:74;
      (b) a VL CDR1 having an amino acid sequence of SEQ ID NO:78, a VL CDR2 having an amino acid sequence of SEQ ID NO:79, and a VL CDR3 having an amino acid sequence of SEQ ID NO:80;
      (c) a VL CDR1 having an amino acid sequence of SEQ ID NO:84, a VL CDR2 having an amino acid sequence of SEQ ID NO:85, and a VL CDR3 having an amino acid sequence of SEQ ID NO:86;
      (d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or
      (e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.
B8. The pharmaceutical composition of embodiment B7, wherein the first binding domain comprises a VH having an amino acid sequence of SEQ ID NO:67, and a VL having an amino acid sequence of SEQ ID NO:68.
B9. The pharmaceutical composition of any one of embodiments B1 to B8, wherein the second antigen is on a cell surface.
B10. The pharmaceutical composition of any one of embodiments B1 to B8, wherein the second antigen is expressed on a tumor cell.
B11. The pharmaceutical composition of embodiment B10, wherein the second antigen is a tumor specific antigen (TSA) or a tumor associated antigen (TAA).
B12. The pharmaceutical composition of embodiment B10, wherein the second antigen is BCMA.
B13. The pharmaceutical composition of embodiment B10, wherein the second antigen is GPRC5d.
B14. The pharmaceutical composition of any one of embodiments B1 to B13, wherein the first binding domain is humanized, the second binding domain is humanized, or both the first binding domain and the second binding domain are humanized.
B15. The pharmaceutical composition of any one of embodiments B1 to B14, wherein the multispecific antibody is an IgG antibody.
B16. The pharmaceutical composition of embodiment B15, wherein the IgG antibody is an IgG1, IgG2, IgG3, or IgG4 antibody.
B17. The pharmaceutical composition of embodiment B16, wherein the IgG antibody is an IgG1 antibody.
B18. The pharmaceutical composition of any one of embodiments B1 to B17, wherein the multispecific antibody is a bispecific antibody.
B19. The pharmaceutical composition of embodiment B18, wherein the bispecific antibody is in a bipod-scaffold configuration.
B20. The pharmaceutical composition of embodiment B19, wherein the first binding domain is a Fab region, and the second binding domain is a scFv region.
B21. The pharmaceutical composition of embodiment B18, wherein the bispecific antibody is in a Morrison-scaffold configuration.
B22. The pharmaceutical composition of embodiment B21, wherein the first binding domain comprises two Fab regions and the second binding domain comprises two scFv regions.
B23. The pharmaceutical composition of any one of embodiments B10 to B22, wherein the multispecific antibody induces NK cell dependent cytotoxicity of the tumor cell in vitro with an IC₅₀ of less than about 500 pM.
B24. The pharmaceutical composition of any one of embodiments B10 to B22, wherein the multispecific antibody induces NK cell dependent cytotoxicity of the tumor cell in vitro with an IC₅₀ of less than about 300 pM.
B25. The pharmaceutical composition of any one of embodiments B10 to B22, wherein the multispecific antibody induces NK cell dependent cytotoxicity of the tumor cell in vitro with an IC₅₀ of less than about 100 pM.
B26. The pharmaceutical composition of any one of embodiments B10 to B22, wherein the multispecific antibody induces NK cell dependent cytotoxicity of the tumor cell in vitro with an IC₅₀ of less than about 50 pM.
B27. The pharmaceutical composition of any one of embodiments B10 to B22, wherein the multispecific antibody induces NK cell dependent cytotoxicity of the tumor cell in vitro with an IC₅₀ of less than about 20 pM.
B28. The pharmaceutical composition of any one of embodiments B10 to B22, wherein the multispecific antibody induces NK cell dependent cytotoxicity of the tumor cell in vitro with an IC₅₀ of less than about 15 pM.
B29. The pharmaceutical composition of any one of embodiments B10 to B22, wherein the multispecific antibody induces NK cell dependent cytotoxicity of the tumor cell in vitro with an IC₅₀ of less than about 10 pM.
B30. The pharmaceutical composition of any one of embodiments B23 to B29, wherein the IC₅₀ is assessed with a mixture of NK effector cells and target cells expressing the second antigen.
B31. The pharmaceutical composition of embodiment B30, wherein the effector cell to target cell ratio is about 0.01 to 1 to about 5 to 1.
B32. The pharmaceutical composition of embodiment B30, wherein the effector cell to target cell ratio is about 0.1 to 1 to about 2 to 1.
B33. The pharmaceutical composition of embodiment B30, wherein the effector cell to target cell ratio is about 1:1.
In one set of embodiments (embodiment set C), provided are:
C1. A process for making a multispecific antibody comprising introducing into a host cell one or more nucleic acids encoding a first binding domain that binds to a first antigen expressed on a Natural Killer (NK) cell, and a second binding domain that binds to a second antigen.
C2. The process of embodiment C1, wherein the first antigen is an NK cell activating receptor.
C3. The process of embodiment C2, wherein the first antigen is NKG2d.
C4. The process of embodiment C3, wherein the first binding domain comprises:
   (i) a heavy chain variable region (VH) comprising
      (a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6;
      (b) a VH complementarity determining region (CDR) 1 having an amino acid sequence of SEQ ID NO:10, a VH CDR2 having an amino acid sequence of SEQ ID NO:11, and a VH CDR3 having an amino acid sequence of SEQ ID NO:12;
      (c) a VH complementarity determining region (CDR) 1 having an amino acid sequence of SEQ ID NO:16, a VH CDR2 having an amino acid sequence of SEQ ID NO:17, and a VH CDR3 having an amino acid sequence of SEQ ID NO:18;
      (d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or
      (e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30;
      and
      a light chain variable region (VL) comprising
      (a) a VL CDR1 having an amino acid sequence of SEQ ID NO:7, a VL CDR2 having an amino acid sequence of SEQ ID NO:8, and a VL CDR3 having an amino acid sequence of SEQ ID NO:9;
      (b) a VL CDR1 having an amino acid sequence of SEQ ID NO:13, a VL CDR2 having an amino acid sequence of SEQ ID NO:14, and a VL CDR3 having an amino acid sequence of SEQ ID NO:15;
      (c) a VL CDR1 having an amino acid sequence of SEQ ID NO:19, a VL CDR2 having an amino acid sequence of SEQ ID NO:20, and a VL CDR3 having an amino acid sequence of SEQ ID NO:21;
      (d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or
      (e) a VL CDR1 having an amino acid sequence of SEQ ID NO:31, a VL CDR2 having an amino acid sequence of SEQ ID NO:32, and a VL CDR3 having an amino acid sequence of SEQ ID NO:33;
      or
   (ii) a heavy chain variable region (VH) comprising
      (a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38;
      (b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44;
      (c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50;
      (d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or
      (e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62;
      and
      a light chain variable region (VL) comprising
      (a) a VL CDR1 having an amino acid sequence of SEQ ID NO:39, a VL CDR2 having an amino acid sequence of SEQ ID NO:40, and a VL CDR3 having an amino acid sequence of SEQ ID NO:41;
      (b) a VL CDR1 having an amino acid sequence of SEQ ID NO:45, a VL CDR2 having an amino acid sequence of SEQ ID NO:46, and a VL CDR3 having an amino acid sequence of SEQ ID NO:47;
      (c) a VL CDR1 having an amino acid sequence of SEQ ID NO:51, a VL CDR2 having an amino acid sequence of SEQ ID NO:52, and a VL CDR3 having an amino acid sequence of SEQ ID NO:53;
      (d) a VL CDR1 having an amino acid sequence of SEQ ID NO:57, a VL CDR2 having an amino acid sequence of SEQ ID NO:58, and a VL CDR3 having an amino acid sequence of SEQ ID NO:59; or
      (e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.
C5. The process of embodiment C4, wherein the first binding domain comprises a VH having an amino acid sequence of SEQ ID NO:2, and a VL having an amino acid sequence of SEQ ID NO:3, or wherein the first binding domain comprises: a VH having an amino acid sequence of SEQ ID NO:34, and a VL having an amino acid sequence of SEQ ID NO:35.
C6. The process of embodiment C2, wherein the first antigen is NKp46.
C7. The process of embodiment C6, wherein the first binding domain comprises:
   (i) a heavy chain variable region (VH) comprising
      (a) a VH complementarity determining region (CDR) 1 having an amino acid sequence of SEQ ID NO:69, a VH CDR2 having an amino acid sequence of SEQ ID NO:70, and a VH CDR3 having an amino acid sequence of SEQ ID NO:71;
      (b) a VH complementarity determining region (CDR) 1 having an amino acid sequence of SEQ ID NO:75, a VH CDR2 having an amino acid sequence of SEQ ID NO:76, and a VH CDR3 having an amino acid sequence of SEQ ID NO:77;
      (c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83;
      (d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or
      (e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95;
      and
   (ii) a light chain variable region (VL) comprising
      (a) a VL CDR1 having an amino acid sequence of SEQ ID NO:72, a VL CDR2 having an amino acid sequence of SEQ ID NO:73, and a VL CDR3 having an amino acid sequence of SEQ ID NO:74;
      (b) a VL CDR1 having an amino acid sequence of SEQ ID NO:78, a VL CDR2 having an amino acid sequence of SEQ ID NO:79, and a VL CDR3 having an amino acid sequence of SEQ ID NO:80;
      (c) a VL CDR1 having an amino acid sequence of SEQ ID NO:84, a VL CDR2 having an amino acid sequence of SEQ ID NO:85, and a VL CDR3 having an amino acid sequence of SEQ ID NO:86;
      (d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or
      (e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.
C8. The process of embodiment C7, wherein the first binding domain comprises a VH having an amino acid sequence of SEQ ID NO:67, and a VL having an amino acid sequence of SEQ ID NO:68.
C9. The process of any one of embodiments C1 to C8, wherein the second antigen is on a cell surface.
C10. The process of any one of embodiments C1 to C8, wherein the second antigen is expressed on a tumor cell.
C11. The process of embodiment C10, wherein the second antigen is a tumor specific antigen (TSA) or a tumor associated antigen (TAA).
C12. The process of embodiment C10, wherein the second antigen is BCMA.
C13. The process of embodiment C10, wherein the second antigen is GPRC5d.
C14. The process of any one of embodiments C1 to C13, wherein the first binding domain is humanized, the second binding domain is humanized, or both the first binding domain and the second binding domain are humanized.
C15. The process of any one of embodiments C1 to C14, wherein the multispecific antibody is an IgG antibody.
C16. The process of embodiment C15, wherein the IgG antibody is an IgG1, IgG2, IgG3, or IgG4 antibody.
C17. The process of embodiment C16, wherein the IgG antibody is an IgG1 antibody.
C18. The process of any one of embodiments C1 to C17, wherein the multispecific antibody is a bispecific antibody.
C19. The process of embodiment C18, wherein the bispecific antibody is in a bipod-scaffold configuration.
C20. The process of embodiment C19, wherein the first binding domain is a Fab region, and the second binding domain is a scFv region.
C21. The process of embodiment C18, wherein the bispecific antibody is in a Morrison-scaffold configuration.
C22. The process of embodiment C21, wherein the first binding domain comprises two Fab regions and the second binding domain comprises two scFv regions.
C23. The process of any one of embodiments C10 to C22, wherein the multispecific antibody induces NK cell dependent cytotoxicity of the tumor cell in vitro with an IC₅₀ of less than about 500 pM.
C24. The process of any one of embodiments C10 to C22, wherein the multispecific antibody induces NK cell dependent cytotoxicity of the tumor cell in vitro with an IC₅₀ of less than about 300 pM.
C25. The process of any one of embodiments C1O to C22, wherein the multispecific antibody induces NK cell dependent cytotoxicity of the tumor cell in vitro with an IC₅₀ of less than about 100 pM.
C26. The process of any one of embodiments C10 to C22, wherein the multispecific antibody induces NK cell dependent cytotoxicity of the tumor cell in vitro with an IC₅₀ of less than about 50 pM.
C27. The process of any one of embodiments C1O to C22, wherein the multispecific antibody induces NK cell dependent cytotoxicity of the tumor cell in vitro with an IC₅₀ of less than about 20 pM.
C28. The process of any one of embodiments C1O to C22, wherein the multispecific antibody induces NK cell dependent cytotoxicity of the tumor cell in vitro with an IC₅₀ of less than about 15 pM.
C29. The process of any one of embodiments C10 to C22, wherein the multispecific antibody induces NK cell dependent cytotoxicity of the tumor cell in vitro with an IC₅₀ of less than about 10 pM.
C30. The process of any one of embodiments C23 to C29, wherein the IC₅₀ is assessed with a mixture of NK effector cells and target cells expressing the second antigen.
C31. The process of embodiment C30, wherein the effector cell to target cell ratio is about 0.01 to 1 to about 5 to 1.
C32. The process of embodiment C30, wherein the effector cell to target cell ratio is about 0.1 to 1 to about 2 to 1.
C33. The process of embodiment C30, wherein the effector cell to target cell ratio is about 1:1.
In one set of embodiments (embodiment set D), provided are:
D1. A method of directing an NK cell to a target cell, comprising contacting the NK cell with a multispecific antibody, thereby directing the NK cell to the target cell, wherein the multispecific antibody comprises a first binding domain that binds to a first antigen on an NK cell and a second binding domain that binds to a second antigen on a target cell.
D2. A method of activating an NK cell, comprising contacting the NK with a multispecific antibody, wherein the multispecific antibody comprises a first binding domain that binds to a first antigen on the NK cell and a second binding domain that binds to a second antigen on a target cell.
D3. A method of inhibiting growth or proliferation of target cells expressing a second antigen on the cell surface, the method comprising contacting the target cells with a multispecific antibody, wherein the multispecific antibody comprises a first binding domain that binds to a first antigen on an NK cell and a second binding domain that binds to the second antigen.
D4. A method for eliminating target cells expressing a second antigen or treating a disease or disorder caused all or in part by target cells expressing the second antigen in a subject, comprising administering an effective amount of a multispecific antibody to the subject, wherein the multispecific antibody comprises a first binding domain that binds to a first antigen on an NK cell and a second binding domain that binds to the second antigen.
D5. The method of embodiment D4, wherein the subject is a subject in need thereof.
D6. The method of embodiment D4 or embodiment D5, wherein the subject is a human.
D7. The method of any one of embodiments D4 to D6, wherein the disease or disorder is cancer.
D8. The method of embodiment D7, wherein the cancer is a blood cancer.
D9. The method of embodiment D7, wherein the cancer is a solid tumor cancer.
D10. The method of any one of embodiments D1 to D9, wherein the first antigen is an NK cell activating receptor.
D11. The method of embodiment D10, wherein the first antigen is NKG2d.
D12. The method of embodiment D11, wherein the first binding domain comprises:
   (i) a heavy chain variable region (VH) comprising
      (a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6;
      (b) a VH complementarity determining region (CDR) 1 having an amino acid sequence of SEQ ID NO:10, a VH CDR2 having an amino acid sequence of SEQ ID NO:11, and a VH CDR3 having an amino acid sequence of SEQ ID NO:12;
      (c) a VH complementarity determining region (CDR) 1 having an amino acid sequence of SEQ ID NO:16, a VH CDR2 having an amino acid sequence of SEQ ID NO:17, and a VH CDR3 having an amino acid sequence of SEQ ID NO:18;
      (d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or
      (e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30;
      and
      a light chain variable region (VL) comprising
      (a) a VL CDR1 having an amino acid sequence of SEQ ID NO:7, a VL CDR2 having an amino acid sequence of SEQ ID NO:8, and a VL CDR3 having an amino acid sequence of SEQ ID NO:9;
      (b) a VL CDR1 having an amino acid sequence of SEQ ID NO:13, a VL CDR2 having an amino acid sequence of SEQ ID NO:14, and a VL CDR3 having an amino acid sequence of SEQ ID NO:15;
      (c) a VL CDR1 having an amino acid sequence of SEQ ID NO:19, a VL CDR2 having an amino acid sequence of SEQ ID NO:20, and a VL CDR3 having an amino acid sequence of SEQ ID NO:21;
      (d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or
      (e) a VL CDR1 having an amino acid sequence of SEQ ID NO:31, a VL CDR2 having an amino acid sequence of SEQ ID NO:32, and a VL CDR3 having an amino acid sequence of SEQ ID NO:33;
      or
   (ii) a heavy chain variable region (VH) comprising
      (a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38;
      (b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44;
      (c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50;
      (d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or
      (e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62;
      and
      a light chain variable region (VL) comprising
      (a) a VL CDR1 having an amino acid sequence of SEQ ID NO:39, a VL CDR2 having an amino acid sequence of SEQ ID NO:40, and a VL CDR3 having an amino acid sequence of SEQ ID NO:41;
      (b) a VL CDR1 having an amino acid sequence of SEQ ID NO:45, a VL CDR2 having an amino acid sequence of SEQ ID NO:46, and a VL CDR3 having an amino acid sequence of SEQ ID NO:47;
      (c) a VL CDR1 having an amino acid sequence of SEQ ID NO:51, a VL CDR2 having an amino acid sequence of SEQ ID NO:52, and a VL CDR3 having an amino acid sequence of SEQ ID NO:53;
      (d) a VL CDR1 having an amino acid sequence of SEQ ID NO:57, a VL CDR2 having an amino acid sequence of SEQ ID NO:58, and a VL CDR3 having an amino acid sequence of SEQ ID NO:59; or
      (e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.
D13. The method of embodiment D12, wherein the first binding domain comprises a VH having an amino acid sequence of SEQ ID NO:2, and a VL having an amino acid sequence of SEQ ID NO:3, or wherein the first binding domain comprises a VH having an amino acid sequence of SEQ ID NO:34, and a VL having an amino acid sequence of SEQ ID NO:35.
D14. The method of embodiment D10, wherein the first antigen is NKp46.
D15. The method of embodiment D14, wherein the first binding domain comprises:
   (i) a heavy chain variable region (VH) comprising
      (a) a VH complementarity determining region (CDR) 1 having an amino acid sequence of SEQ ID NO:69, a VH CDR2 having an amino acid sequence of SEQ ID NO:70, and a VH CDR3 having an amino acid sequence of SEQ ID NO:71;
      (b) a VH complementarity determining region (CDR) 1 having an amino acid sequence of SEQ ID NO:75, a VH CDR2 having an amino acid sequence of SEQ ID NO:76, and a VH CDR3 having an amino acid sequence of SEQ ID NO:77;
      (c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83;
      (d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or
      (e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95;
      and
   (ii) a light chain variable region (VL) comprising
      (a) a VL CDR1 having an amino acid sequence of SEQ ID NO:72, a VL CDR2 having an amino acid sequence of SEQ ID NO:73, and a VL CDR3 having an amino acid sequence of SEQ ID NO:74;
      (b) a VL CDR1 having an amino acid sequence of SEQ ID NO:78, a VL CDR2 having an amino acid sequence of SEQ ID NO:79, and a VL CDR3 having an amino acid sequence of SEQ ID NO:80;
      (c) a VL CDR1 having an amino acid sequence of SEQ ID NO:84, a VL CDR2 having an amino acid sequence of SEQ ID NO:85, and a VL CDR3 having an amino acid sequence of SEQ ID NO:86;
      (d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or
      (e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.
D16. The method of embodiment D15, wherein the first binding domain comprises a VH having an amino acid sequence of SEQ ID NO:67, and a VL having an amino acid sequence of SEQ ID NO:68.
D17. The method of any one of embodiments D1 to D16, wherein the second antigen is on a cell surface.
D18. The method of any one of embodiments D1 to D17, wherein the second antigen is expressed on a tumor cell.
D19. The method of embodiment D18, wherein the second antigen is a tumor specific antigen (TSA) or a tumor associated antigen (TAA).
D20. The method of embodiment D19, wherein the second antigen is BCMA.
D21. The method of embodiment D19, wherein the second antigen is GPRC5d.
D22. The method of any one of embodiments D1 to D21, wherein the first binding domain is humanized, the second binding domain is humanized, or both the first binding domain and the second binding domain are humanized.
D23. The method of any one of embodiments D1 to D21, wherein the multispecific antibody is an IgG antibody.
D24. The method of embodiment D23, wherein the IgG antibody is an IgG1, IgG2, IgG3, or IgG4 antibody.
D25. The method of embodiment D24, wherein the IgG antibody is an IgG1 antibody.
D26. The method of any one of embodiments D1 to D25, wherein the multispecific antibody is a bispecific antibody.
D27. The method of embodiment D26, wherein the bispecific antibody is in a bipod-scaffold configuration.
D28. The method of embodiment D27, wherein the first binding domain is a Fab region, and the second binding domain is a scFv region.
D29. The method of embodiment D26, wherein the bispecific antibody is in a Morrison-scaffold configuration.
D30. The method of embodiment D29, wherein the first binding domain comprises two Fab regions and the second binding domain comprises two scFv regions.
D31. The method of any one of embodiments D18 to D30, wherein the multispecific antibody induces NK cell dependent cytotoxicity of the tumor cell in vitro with an IC₅₀ of less than about 500 pM.
D32. The method of any one of embodiments D18 to D30, wherein the multispecific antibody induces NK cell dependent cytotoxicity of the tumor cell in vitro with an IC₅₀ of less than about 300 pM.
D33. The method of any one of embodiments D18 to D30, wherein the multispecific antibody induces NK cell dependent cytotoxicity of the tumor cell in vitro with an IC₅₀ of less than about 100 pM.
D34. The method of any one of embodiments D18 to D30, wherein the multispecific antibody induces NK cell dependent cytotoxicity of the tumor cell in vitro with an IC₅₀ of less than about 50 pM.
D35. The method of any one of embodiments D18 to D30, wherein the multispecific antibody induces NK cell dependent cytotoxicity of the tumor cell in vitro with an IC₅₀ of less than about 20 pM.
D36. The method of any one of embodiments D18 to D30, wherein the multispecific antibody induces NK cell dependent cytotoxicity of the tumor cell in vitro with an IC₅₀ of less than about 15 pM.
D37. The method of any one of embodiments D18 to D30, wherein the multispecific antibody induces NK cell dependent cytotoxicity of the tumor cell in vitro with an IC₅₀ of less than about 10 pM.
D38. The method of any one of embodiments D31 to D37, wherein the IC₅₀ is assessed with a mixture of NK effector cells and target cells expressing the second antigen.
D39. The method of embodiment D38, wherein the effector cell to target cell ratio is about 0.01 to 1 to about 5 to 1.
D40. The method of embodiments D38, wherein the effector cell to target cell ratio is about 0.1 to 1 to about 2 to 1.
D41. The method of embodiments D38, wherein the effector cell to target cell ratio is about 1:1.
In one set of embodiments (embodiment set E), provided are:
E1. A molecule comprising a first means for engaging or activating a Natural Killer (NK) cell, and a second means for binding a tumor cell, wherein the molecule is capable of inducing NK cell dependent cytotoxicity against the tumor cell.
E2. The molecule of embodiment E1, wherein the first means comprises a first binding domain that binds to a first antigen expressed on the NK cell, and the second means comprises a second binding domain that binds to a second antigen expressed on the tumor cell.
E3. The molecule of embodiment E2, wherein the first antigen is an NK cell activating receptor.
E4. The molecule of embodiment E2, wherein the first antigen is NKG2d.
E5. The molecule of embodiment E2, wherein the first antigen is NKp46.
E6. The molecule of embodiment E2, wherein the second antigen is a tumor specific antigen (TSA) or a tumor associated antigen (TAA).
E7. The molecule of embodiment E6, wherein the second antigen is BCMA.
E8. The molecule of embodiment E6, wherein the second antigen is GPRC5d.
E9. A process for making a molecule that binds to more than one target molecule, comprising: a step for performing a function of obtaining a binding domain capable of binding to a first antigen on an NK cell; a step for performing a function of obtaining a binding domain capable of binding to a second antigen on a tumor cell; and a step for performing a function of providing a molecule capable of binding to the first antigen and the second antigen.
E10. A method of directing an NK cell to a target cell, comprising contacting the NK cell with a molecule of any one of embodiments E1 to E8.
E11. A method of activating an NK cell, comprising contacting the NK with a molecule of any one of embodiments E1 to E8.
E12. A method of inhibiting growth or proliferation of target cells, the method comprising contacting the target cells with molecule of any one of embodiments E1 to E8.
E13. A method for eliminating target cells expressing a second antigen or treating a disease or disorder caused all or in part by target cells expressing the second antigen in a subject, comprising administering an effective amount of molecule of any one of embodiments E1 to E8 to the subject.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### 7. EXAMPLES

### 7.1 Example 1: Production of Anti-NKG2D Antiboby that Bind NK Cells

### 7.1.1. Production of Anti-NKG2D Antibody (NKGB125) by Immunization

To obtain binders for human NKG2d, antibody discovery was conducted by immunization with NKGW1 (NKG2d80-216 extracellular domain). OmniRats were immunized with human NKGW1 (Table 1) and boosted by weekly immunizations over 7 weeks after which serum was collected from euthanized animals.

TABLE 1
Antigen used for immunization

| Antigen | Description | Sequence |
|---|---|---|
| NKGW1 | Human | HHHHHHNSLFNQEVQIPLTESYCGPC |
| | NKG2d₈₀₋₂₁₆ | PKNWICYKNNCYQFFDESKNWYESQ |
| | extracellular | ASCMSQNASLLKVYSKEDQDLLKLV |
| | region with | KSYHWMGLVHIPTNGSWQWEDGSIL |
| | N-term | SPNLLTIIEMQKGDCALYASSFKGY |
| | hexahistidine | IENCSTPNTYICMQRTV |
| | tag | (SEQ ID NO: 1) |

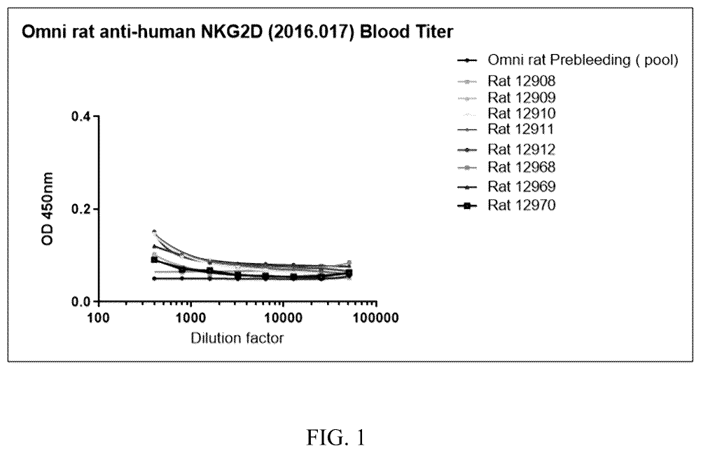

Inguinal and Popliteal lymph nodes were aseptically harvested and pooled. Mandibular lymph nodes from all 8 animals were also harvested. Whole blood was collected into RNA tubes from 2 animals. Bone marrow from the femurs of these 2 animals was also collected into cold sterile 1×PBS (HYB:212, Jen Pitcher ELN: NKG2d-00011). Sera titers were measured to determine immune response to immunizations (FIG. 1).

The total lymphocytes from immunized rats were combined into two groups, and viable cells counts were measured as about 60% (Hai Sheng, ELN: NKG2d-00023). Cells were collected by centrifugation. FO cells for 1:1 fusion ratio were collected. Briefly, a cell bank of the non-secreting Balb/c mouse myeloma fusion partner, FO cell was obtained from ATCC (cat #CRL-1646) banked through Janssen's Cell Biology Services (CBS). One frozen vial was received and placed into culture using DMEM+glutamax (Invitrogen cat. #10569 lot #1676884)/10% FBS (Invitrogen 16140 Lot 1671884). Cells were kept in log phase splitting every few days at 1:2 to 1:10. Cells were collected by centrifugation, washed once in 1×PBS and counted, FO at 5×10⁷ cells at 96.4% viable were used for each group fusion. Lymphocytes and FO cells were added together and washed in 1×PBS and supernatant was discarded and the cell pellet was resuspended by flicking. To each mixed cell population, 1 mL of 37° C. PEG 4000 (2 g PEG (EMD cat #9727.2), 2 mL DMEM (Invitrogen cat. #11995 lot #1676884), 400 μL of DMSO (sigma D2650) were added per 10⁸ cells (1 mL max). The cell mixtures were swirled in a 37° C. water bath for one minute. 37° C. DMEM+Glutamax (Invitrogen cat #11995 lot #1676884) (40 mL) was added over one minute to stop the reaction. Cells were rested for 5 minutes at room temperature prior to centrifugation to collect cell pellet. Cells were resuspended in MediumE (StemCell Technologies cat #15A60952 lot #15F64268)+HAT (Gibco cat #21060-017) and then plated at 200 μL/well, resulting in 1.13×10⁴ lymphocytes/well. Cells were incubated for 7 days at 37° C., 5% CO2. Cells were then re-fed with 200 μL fresh Medium (StemCell Technologies cat #03805 lot #15A60952).

ELISA-based screening assays were run on supernatants (Mike Miller, ELN: NKG2d Oncology-00001) using immobilized human and cynomolgus monkey NKG2d. For the immobilized antigen format, briefly, plates were coated with 50 pg/well of NKG2d at 1 μg/mL. Plates were blocked by the addition of 200 μL/well of 0.4% BSA-PBS and incubated overnight at 4° C., then washed three times using 300 μL/well of 1×PBS+0.02% Tween 20. 50 μL of test supernatant and media control were then added to wells, incubated at room temperature for 1 hour. 50 μL/well of a mixture sera from AFP2016.017.JP (1:2000 dilution in blocking buffer) were added as positive control. Background control wells were added FO cultured media (50 μL/well). Wells were washed 3 times in 1×PBS+0.02% Tween 20, then added 50 μL/well of goat anti-Rat IgG Fc-HRP (Jackson cat #112-036-071 diluted 1:10K in blocking buffer), incubated 30 minutes at room temperature, washed 3 times in 1×PBS+0.02% Tween 20, added 50 μL/well of TMB substrate buffer (Thermo cat #34022) and incubated in the dark for about 10 minutes, then reactions were stopped by adding 25 μL/well of 4N H2SO4 to all wells. Plates were read at 450 nm using Biotek (Gen5 software). Hits which had OD value greater than 2× Average of media background control were selected for binding confirmation to human NKG2d.

In summary, culture supernatants from 95 fusion plates were screened for antibody binding to human NKG2d. Supernatants were screened in two ELISA formats; directly coated NKG2d and biotinylated NKG2d. R analysis was used to analyze ELISA data for both primary screens and generated separate platemaps for each format. Duplicate hits were removed between the two hit platemaps to generate a final compiled platemap, consisting of 259 hits for binding confirmation. Duplicate hits were removed between the two lists to generate a final list of 168 confirmed human NKG2d-specific hits (Table 2). Hits were characterized for cross-reactive binding to cyno NKG2d (after MPB hand-off). Antigens were screened in both formats: directly coated and biotinylated. Positive binding was determined as OD value greater than 2× Average background control. 163 of the 168 hits were cross-reactive to cyno NKG2d protein in at least one of the two assay formats (directly coated antigen and/or biotinylated antigen).

TABLE 2
Summary of hybridoma hits against human NKG2d

| | # Confirmed hits for | # Confirmed hits for | Consolidated |
|---|---|---|---|
| Fusion | directly coated NKG2d | biotinylated NKG2d | list |
| Group#1 | 97 | 82 | 100 |
| Group#2 | 65 | 52 | 68 |
| Total | 162 | 134 | 168 |

RNA was isolated from NKGY1 fusion hybridomas derived from OmniRats immunized with human NKG2d (Maria MacWilliams, ELN: Biologics Research Requests—2016-00387). The RNA was used as the template to prepare cDNA in a reverse transcriptase reaction and then the cDNA was used as the template for PCR amplification of the Ig variable regions. Briefly, the plates were first centrifuged at 500×g for 5 minutes. The medium was flicked off and the plate gently placed in contact with a clean area to remove additional medium. These steps were repeated to remove residual medium from the wells. 140 μL RLT+143 mM 2-mercaptoethanol was added to each well. The plates were rapidly moved back and forth on the bench surface for 10 seconds, then turned 90 degrees and shaken again for 10 seconds. The RNA was isolated on the Qiagen Biorobot 8000 using RNeasy 96 protocol (For procedure information, see RNeasy 96 Biorobot 8000 Kit in the Metadata tab). The automated procedure began just after the RLT+BME cell lysis step. Final Elution Volume was 100 μL. The BioMek robot transferred 8 μL of each RNA sample to the microtiter plate for cDNA synthesis and 80 μL to a microtiter plate. The 8 μL aliquots were immediately used as template for cDNA synthesis. The 80 μL plates were each covered with sealing film and then wrapped in foil. These plates are stored at −80° C. To prepare cDNA, the Invitrogen Superscript III First Strand Synthesis System (catalog #18080-051) was used according to the manufacturer's instructions. Briefly, 8 μL of RNA was used in a 20 μL reaction. Gene-specific primers (one each for vH, vK, and vL mRNAs) targeted the antibody variable regions. In order to detect antibody gene mRNA and to determine the sequences of the variable regions present on the antibody chains being expressed, PCR were performed. For each hybridoma, two separate reactions were run: one for the IgG Heavy chain and one for the Lambda Light chain. The primer sequences for reactions are listed in a sub-tab of the RT-PCR tab. Platinum Pfx polymerase (Invitrogen catalog #11708-021) was used in a procedure adapted from the manufacturer. The immunized animals only produced Lambda light chains and thus the Kappa reactions were not run. Agarose Gel Analysis was performed to confirm the presence of PCR products. Clone NKGY1_045_F04 was selected based on its specific binding to both human and cyno NKG2d.

In summary, of the 128 hybridomas, about 125 had visible vH and vL PCR products. Samples were sequenced by Sanger sequencing to obtain v-region sequences and then v-regions were cloned into human IgG1 sigma and lambda constant regions. Clone NKGY1_045_F04 thus obtained the mAb identifier NKGB125. Variable region sequences of NKGB125 are provided below in Table 3. The CDRs sequences of NKGB125 are provided in Table 4. The antibody panel was expressed at 2 mL scale, and NKGB125 gave a final antibody of 0.37 mg, which fell within the top ˜6% of antibody yields (Mike Diem, ELN: Biologics Research Requests—2016-00739) and was ˜97% monomer (Ed Swift, ELN: Biologics Research Requests—2016-00796).

TABLE 3
V-regions of NKGB125

| mAb | V-region | VH | VL |
|---|---|---|---|
| NKGB | VR000 | EVQLLESGGGLVQPG | SYVLTQPPSVSVAPG |
| 125 | 2381 | GSLRLSCAASGFTFS | QTARITCGGNNIGSK |
| | | IYAMTWVRQAPGKGL | SVHWYQQKAGQAPVL |
| | | EWVSIISGSGDHTFY | VVYDDSDRPSGIPER |
| | | ADSVKGRFTISRDNS | FSGSNSGNTATLTIS |
| | | RNTLYLQMDSLRAED | RVEAGDEADYYCQVW |
| | | TAVYYCAKEGKWVQL | DGRSDHVVFGGGTKL |
| | | SHFANWGQGTLVTVS | TVL |
| | | S | (SEQ ID NO: 3) |
| | | (SEQ ID NO: 2) | |

TABLE 4
CDR Amino Acid Sequences of NKGB125

| | HC | HC | HC | LC | LC | LC |
|---|---|---|---|---|---|---|
| System | CDR1 | CDR2 | CDR3 | CDR1 | CDR2 | CDR3 |
| AbM | GFTFSIY | IISGSGD | EGKWV | GGNNIG | DDSDRP | QVWDG |
| | AMT | HTF | QLSHFA | SKSVH | S (SEQ | RSDHVV |
| | (SEQ ID | (SEQ ID | N (SEQ | (SEQ ID | ID | (SEQ ID |
| | NO: 4) | NO: 5) | ID NO: 6) | NO: 7) | NO: 8) | NO: 9) |
| Kabat | IY AMT | IISGSGD | EGKWV | GGNNIG | DDSDRP | QVWDG |
| | (SEQ ID | HTFYAD | QLSHFA | SKSVH | S (SEQ | RSDHVV |
| | NO: 10) | SVKG | N (SEQ | (SEQ ID | ID NO: | (SEQ ID |
| | | (SEQ ID | ID NO: | NO: 13) | 14) | NO: 15) |
| | | NO: 11) | 12) | | | |
| Chothia | GFTFSIY | SGSGDH | EGKWV | NNIGSK | DDS | WDGRS |
| | (SEQ ID | (SEQ ID | QLSHFA | S (SEQ | (SEQ ID | DHV |
| | NO: 16) | NO: 17) | (SEQ ID | ID NO: | NO: 20) | (SEQ ID |
| | | | NO: 18) | 19) | | NO: 21) |
| Contact | SIYAMT | WVSIISG | AKEGK | SKSVHW | LVVYDD | QVWDG |
| | (SEQ ID | SGDHTF | WVQLS | Y (SEQ | SDRP | RSDHV |
| | NO: 22) | (SEQ ID | HFA | ID NO: | (SEQ ID | (SEQ ID |
| | | NO: 23) | (SEQ ID | 25) | NO: 26) | NO: 27) |
| | | | NO: 24) | | | |
| IMGT | GFTFSIY | ISGSGD | AKEGK | NIGSKS | DDS | QVWDG |
| | A (SEQ | HT (SEQ | WVQLS | (SEQ ID | (SEQ ID | RSDHVV |
| | ID NO: | ID NO: | HFAN | NO: 31) | NO: 32) | (SEQ ID |
| | 28) | 29) | (SEQ ID | | | NO: 33) |
| | | | NO: 30) | | | |

### 7.1.2. Assays for Binding Activity of Anti-NKG2D Antibodies Obtained by Immunication on NKL Cell Lines

The panel of antibodies was screened for their abilities to bind NKL cells which express NKG2d (Lamar Blackwell, ELN: NKG2d-00048). Briefly, cells were washed and resuspended in PBS˜1×10⁶ cells/ml to which 1 μL of green live-dead stain (L-23101 Thermo) were added and cells were plated at 100 μL/well for 30 minutes at 4° C. Cells were resuspended in 200 μL staining buffer, spin 400 g for 5 minutes, and treated with 50 μL of either NKG2d antibody or Isotype control, both diluted in PBS at 60, 6, and 0.6 nM. Plates were incubated at 4° C. for 1 hour. Cells were then resuspended in 150 μL staining buffer, spin 400 g for 5 minutes, flick, resuspended in 200 μL staining buffer, spin 400 g for 5 minutes, and Goat anti hu-Fc AF647 (Jackson 109-606-098 lot 122473) in PBS was added to 2 pg/ml and add 50 μL/well for 30 minutes at 4° C. Samples were acquired on HyperCyt® Autosampler (by Intellicyte). Data was analyzed using ForeCyt™ screening software. Geometric mean fluorescent intensities (geomeans) values from the ForCyte were used for analysis (Table 5). No antibodies displayed significant binding to HEK293 cells, which do not express NKG2d.

TABLE 5
Analysis of the abilities of anti-NKG2d antibodies to bind NKL cells.
(Lamar Blackwell, ELN: NKG2d-00048)

| | [Antibody] (nM) | | | | [Antibody ] (nM) | | |
|---|---|---|---|---|---|---|---|
| Antibody | 60 | 6 | 0.6 | Antibody | 60 | 6 | 0.6 |
| NKGB118 | 71.4 | 169.3 | 502.9 | NKGB174 | 1197.2 | 95.6 | 97.3 |
| NKGB126 | 61.8 | 132.5 | 124 | NKGB181 | 197.7 | 74.9 | 84.5 |
| NKGB134 | 59.2 | 118.7 | 97.2 | NKGB189 | 112.2 | 88.5 | 357.5 |
| NKGB142 | 109 | 189.5 | 234.2 | NKGB198 | 982.1 | 94.7 | 94.2 |
| NKGB150 | 59.5 | 149.1 | 90.9 | NKGB205 | 132.7 | 93.2 | 113.9 |
| NKGB158 | 84.7 | 157.3 | 91.5 | NKGB213 | 1674.6 | 102.6 | 125.1 |
| NKGB166 | 65.1 | 108.5 | 130.2 | NKGB221 | 41809.7 | 23164.9 | 5322.3 |
| isotype | 76.5 | 105.4 | 108.7 | NKGB173 | 1664.1 | 96.5 | 89.2 |
| NKGB119 | 82 | 418.8 | 366.6 | NKGB182 | 194.5 | 98 | 85 |
| NKGB128 | 36.1 | 113.1 | 103.6 | NKGB190 | 140.3 | 89.1 | 84.4 |
| NKGB135 | 76.6 | 103.2 | 87.1 | NKGB197 | 1076.7 | 101.4 | 108.9 |
| NKGB143 | 65.9 | 187.4 | 232.2 | NKGB206 | 39749.6 | 31376.9 | 7966.9 |
| NKGB151 | 72.8 | 125.2 | 161 | NKGB214 | 122.9 | 93.4 | 84.8 |
| NKGB159 | 93.4 | 123.5 | 88.1 | NKGB222 | 115.1 | 110.4 | 91.1 |
| NKGB167 | 86.9 | 116 | 111.5 | NKGB175 | 117.9 | 90.4 | 88.2 |
| isotype | 64.9 | 138 | 121.6 | NKGB183 | 102.8 | 82.3 | 88.1 |
| NKGB120 | 64.5 | 116.3 | 85.8 | NKGB191 | 111.7 | 80.6 | 103.2 |
| NKGB127 | 60.4 | 94 | 95.7 | NKGB199 | 153.5 | 88.8 | 101.6 |
| NKGB136 | 75 | 112 | 92.8 | NKGB207 | 155.4 | 107.1 | 80.8 |
| NKGB144 | 73.1 | 112.5 | 96.2 | NKGB215 | 110.6 | 92.7 | 91.8 |
| NKGB152 | 79 | 117.5 | 77.1 | isotype | 119.6 | 75.7 | 121.4 |
| NKGB160 | 66 | 103.7 | 96.2 | NKGB176 | 108.9 | 78.9 | 90.2 |
| NKGB168 | 74.4 | 124.2 | 105.7 | NKGB184 | 104.3 | 108.3 | 89.3 |
| isotype | 91.2 | 192.2 | 99.8 | NKGB192 | 111.8 | 87.4 | 86.8 |
| NKGB121 | 58.3 | 95.5 | 89.9 | NKGB200 | 25974.2 | 85.3 | 131.6 |
| NKGB129 | 10004.7 | 560.1 | 133.6 | NKGB208 | 1786.9 | 166 | 183.7 |
| NKGB137 | 417.6 | 137.9 | 91.3 | NKGB216 | 140.5 | 132.8 | 120.9 |
| NKGB145 | 65.3 | 144.4 | 84 | isotype | 124 | 104.3 | 108.9 |
| NKGB153 | 99.4 | 129.5 | 112.1 | NKGB177 | 92 | 88.2 | 82.7 |
| NKGB161 | 385.6 | 124.8 | 89 | NKGB185 | 118.7 | 82.3 | 93.6 |
| NKGB169 | 70.2 | 144.4 | 103.9 | NKGB194 | 108.9 | 102 | 127.4 |
| isotype | 70.5 | 117.4 | 87.6 | NKGB201 | 100.5 | 127.4 | 88.4 |
| NKGB122 | 60.7 | 180.7 | 92.5 | NKGB209 | 68.1 | 90.3 | 107.7 |
| NKGB130 | 43603.4 | 36221.2 | 9031.2 | NKGB217 | 183.8 | 115.5 | 130.8 |
| NKGB138 | 21084.9 | 474.3 | 132.9 | isotype | 196.4 | 97.9 | 136.9 |
| NKGB146 | 52.5 | 141.6 | 97.2 | NKGB178 | 109.3 | 73.9 | 73.1 |
| NKGB154 | 86.2 | 96.5 | 97.5 | NKGB186 | 120.7 | 96.5 | 97.5 |
| NKGB162 | 72.1 | 126.2 | 108.1 | NKGB193 | 1317.8 | 109 | 109.4 |
| NKGB170 | 74.9 | 137.6 | 117.2 | NKGB202 | 14597.9 | 796.4 | 111.5 |
| isotype | 83 | 122.1 | 119.6 | NKGB210 | 121.8 | 95.9 | 143.1 |
| NKGB123 | 24.9 | 138.9 | 74.9 | NKGB218 | 334.5 | 97.4 | 120.3 |
| NKGB131 | 59.5 | 132.4 | 102.2 | isotype | 138.2 | 111.4 | 121.1 |
| NKGB139 | 61.8 | 92.6 | 95.4 | NKGB179 | 104.9 | 96.3 | 72 |
| NKGB147 | 49.9 | 109.5 | 97.7 | NKGB187 | 113.9 | 89.7 | 198.7 |
| NKGB155 | 87 | 103.7 | 155.6 | NKGB195 | 119.9 | 88.3 | 90.4 |
| NKGB163 | 79.7 | 105.9 | 107.3 | NKGB203 | 45180.9 | 26710.6 | 4601 |
| NKGB171 | 81.9 | 95.3 | 99.8 | NKGB211 | 6859.8 | 273.4 | 131.9 |
| isotype | 87.9 | 130.2 | 125.3 | NKGB219 | 42256.1 | 30438.7 | 5129.4 |
| NKGB124 | 69.9 | 119.2 | 85 | isotype | 393 | 116.8 | 95.1 |
| NKGB132 | 45.2 | 124.3 | 92.5 | NKGB180 | 90.9 | 86.1 | 65.4 |
| NKGB140 | 69.4 | 106.8 | 76.5 | | | | |
| NKGB148 | 157.2 | 119.9 | 120 | | | | |
| NKGB156 | 60 | 125 | 99.4 | | | | |
| NKGB164 | 109.5 | 107.7 | 111.9 | | | | |
| NKGB172 | 78.4 | 113 | 125.8 | | | | |
| isotype | 61.8 | 110.3 | 111.1 | | | | |
| NKGB125 | 36401.8 | 24367.3 | 2693.3 | | | | |
| NKGB133 | 65.6 | 95.6 | 95.7 | | | | |
| NKGB141 | 78.9 | 118.2 | 87.1 | | | | |
| NKGB149 | 84.7 | 329.1 | 111.3 | | | | |
| NKGB157 | 72.2 | 107 | 89.8 | | | | |
| NKGB165 | 61.9 | 93.3 | 97.7 | | | | |
| isotype | 59.8 | 77 | 111.6 | | | | |

In summary, 10 antibodies displayed dose-dependent, NKG2d-specific binding to NKL cells: NKGB129, NKGB130, NKGB138, NKGB125, NKGB221, NKGB206, NKGB200, NKGB202, NKGB203, and NKGB219 (Table 5).

### 7.1.3. Production of Anti-NKG2D Antibody (NKGB83) by Screening Phage Display Libraries

To obtain binders for human NKG2d, antibody discovery was conducted by screening phage display libraries (Ramia Reddy, ELN: Biologics Research Requests—2016-00137). Libraries used were: De novo Fab-pIX phage libraries (WO 2009/085462 A1). De novo Fab-pIX phage libraries (WO 2009/085462 A1):
V2.1—Heavy chains (1-69, 3-23, 5-51); germline light chains (A27, B3, L6, 012)
V3.0—Heavy chains (1-69, 3-23, 5-51); diversified light chains (A27, B3, L6, 012) Pool 100 uL of each library by heavy chain, eg {1-69+(A27, B3, L6, 012)} to generate 6 library pools
V5.0—Cloning Heavy chains (1-69, 3-23, 5-51); germline light chains (A27, B3, L6, 012) (ELN: DeNovo 2010 phage libray SRI-005, DeNovo 2010 phage library SRI-006 & DeNovo 2010 phage library SRI-007)
   Libraries were pooled 10 uL from each H3-length for each Hc/Lc pair, resulting in 110 μL of library phage for the panning. Each HC pair was then pooled, resulting in 3 libraries for panning.

Briefly, antibody libraries were displayed on the PIX phage surface and colonies were selected by ELISA-based binding to immobilized human biotinylated-NKGW1.

Phages were amplified in E. coli and sequenced by v-regions using Sanger methods. Overall, 45 unique antibody v-regions were identified as capable of binding NKG2d. V-regions were then cloned into silent human IgG1/kappa constant regions and expressed at 2 mL scale.

### 7.1.4. Assays for Binding Activity of Anti-NKG2D Antibodies Obtained by Screening Phage Display Libraries on NKL Cell Lines

The antibodies were tested for their abilities to bind NKL cells (Lamar Blackwell, ELN: NKG2d-00035) in the same way as for the antibodies in Example 1.2 (Table 6).

TABLE 6
Analysis of the abilities of anti-NKG2d antibodies to
bind NKL cells. (Lamar Blackwell, ELN: NKG2d-00048)

| Antibody | 60 nM | 6 nM | 0.6 nM |
|---|---|---|---|
| NKGB108.001 | 7583.2 | 1574.0 | 164.5 |
| NKGB116.001 | 9039.5 | 281.4 | 174.7 |
| NKGB73.001 | 84.3 | 899.6 | 63.5 |
| NKGB83.001 | 5082.3 | 343.3 | 90.1 |
| NKGB89.001 | 2016.2 | 199.3 | 72.4 |
| NKGB90.001 | 1306.2 | 4202.7 | 73.6 |
| NKGB95.001 | 18517.1 | 5109.6 | 275.4 |
| NKGB99.001 | 12200.0 | 0.0 | 540.5 |
| NKGB100.001 | 10586.3 | 2744.3 | 252.5 |
| NKGB101.001 | 133.9 | 92.4 | 59.4 |
| NKGB104.001 | 92.4 | 106.4 | 59.9 |
| NKGB105.001 | 33.5 | 72.4 | 58.6 |
| NKGB107.001 | 47.4 | 64.4 | 65.4 |
| NKGB110.001 | 52.6 | 63.7 | 62.8 |
| NKGB113.001 | 45.2 | 76.5 | 64.0 |
| NKGB117.001 | 53.8 | 110.4 | 59.1 |
| NKGB74.001 | 259.7 | 82.8 | 70.1 |
| NKGB78.001 | 94.6 | 92.4 | 65.2 |
| NKGB84.001 | 183.9 | 134.1 | 74.9 |
| NKGB87.001 | 53.4 | 71.7 | 68.5 |
| NKGB88.001 | 6259.3 | 2194.4 | 236.8 |
| NKGB92.001 | 112.1 | 74.9 | 62.7 |
| NKGB93.001 | 11337.0 | 1970.1 | 182.4 |
| NKGB97.001 | 62.2 | 75.8 | 67.1 |
| NKGB103.001 | 100.1 | 65.7 | 65.6 |
| NKGB111.001 | 82.8 | 70.8 | 71.6 |
| NKGB112.001 | 403.9 | 165.0 | 72.8 |
| NKGB77.001 | 60.9 | 85.6 | 64.1 |
| NKGB85.001 | 87.2 | 104.8 | 69.2 |
| NKGB86.001 | 215.0 | 165.2 | 71.2 |
| NKGB106.001 | 465.2 | 122.7 | 64.5 |
| NKGB109.001 | 295.2 | 109.8 | 74.3 |
| NKGB114.001 | 418.6 | 129.6 | 79.3 |
| NKGB82.001 | 293.3 | 120.8 | 96.5 |
| NKGB94.001 | 1464.8 | 271.7 | 87.4 |
| NKGB96.001 | 431.2 | 132.9 | 72.0 |
| NKGB75.001 | 12352.7 | 9388.0 | 5186.8 |
| NKGB76.001 | 90.1 | 94.8 | 74.4 |
| NKGB79.001 | 219.4 | 155.2 | 85.3 |
| NKGB80.001 | 234.1 | 117.1 | 80.2 |
| NKGB81.001 | 29.6 | 88.6 | 80.2 |
| NKGB91.001 | 1421.5 | 287.9 | 114.4 |
| NKGB98.001 | 25567.7 | 22713.6 | 9497.4 |
| NKGB102.001 | 13446.3 | 3883.4 | 314.6 |
| NKGB115.001 | 105.6 | 98.1 | 88.3 |
| iso | 63.9 | 93.0 | 87.0 |
| 2nd only | 63.8 | 114.7 | 92.6 |

In summary, 11 antibodies showed significant, specific binding to NKL cells: NKGB108, NKGB116, NKGB83, NKGB95, NKGB99, NKGB100, NKGB88, NKGB93, NKGB75, NKGB98, and NKGB102.

Variable region sequences of NKGB83 are provided below in Table 7. The CDRs sequences of NKGB83 are provided in Table 8S.

TABLE 7
V-regions of NKGB83

| mAb | V-region | VH | VL |
|---|---|---|---|
| NKGB | VR00004 | EVQLVQSGAEVKKPG | DIVMTQSPDSLAVSL |
| 83 | 4290 | ESLKISCKGSGYSFT | GERATINCKSSQSVL |
| | | SYWIGWVRQMPGKGL | YSSNNKNYLAWYQQK |
| | | EWMGIIYPGDSYTRY | PGQPPKLLIYWASTR |
| | | SPSFQGQVTISADKS | ESGVPDRFSGSGSGT |
| | | ISTAYLQWSSLKASD | DFTLTISSLQAEDVA |
| | | TAMYYCARGGVSASG | VYYCQQYYSTPLTFG |
| | | NYRALDYWGQGTLVT | QGTKVEIK |
| | | VSS | (SEQ ID NO: 35) |
| | | (SEQ ID NO: 34) | |

TABLE 8
CDR Amino Acid Sequences of NKGB83

| | HC | HC | HC | LC | LC | LC |
|---|---|---|---|---|---|---|
| System | CDR1 | CDR2 | CDR3 | CDR1 | CDR2 | CDR3 |
| AbM | GYSFTS | IIYPGDS | GGVSAS | KSSQSV | WASTRE | QQYYST |
| | YWIG | YTR | GNYRAL | LYSSNN | S (SEQ | PLT |
| | (SEQ ID | (SEQ ID | DY (SEQ | KNYLA | ID NO: | (SEQ ID |
| | NO: 36) | NO: 37) | ID NO: | (SEQ ID | 40) | NO: 41) |
| | | | 38) | NO: 39) | | |
| Kabat | SYWIG | IIYPGDS | GGVSAS | KSSQSV | WASTRE | QQYYST |
| | (SEQ ID | YTRYSP | GNYRAL | LYSSNN | S (SEQ | PLT |
| | NO: 42) | SFQG | DY (SEQ | KNYLA | ID NO: | (SEQ ID |
| | | (SEQ ID | ID NO: | (SEQ ID | 46) | NO: 47) |
| | | NO: 43) | 44) | NO: 45) | | |
| Chothia | GYSFTS | YPGDSY | GGVSAS | SQSVLY | WAS | YYSTPL |
| | Y(SEQ | (SEQ ID | GNYRAL | SSNNKN | (SEQ ID | (SEQ ID |
| | ID NO: | NO: 49) | D (SEQ | Y (SEQ | NO: 52) | NO: 53) |
| | 48) | | ID NO: | ID NO: | | |
| | | | 50) | 51) | | |
| Contact | TSYWIG | WMGIIY | ARGGVS | LYSSNN | LLIYWA | QQYYST |
| | (SEQ ID | PGDSYT | ASGNYR | KNYLA | STRE | PL (SEQ |
| | NO: 54) | R (SEQ | ALD | WY (SEQ | (SEQ ID | ID NO: |
| | | ID NO: | (SEQ ID | ID NO: | NO: 58) | 59) |
| | | 55) | NO: 56) | 57) | | |
| IMGT | GYSFTS | IYPGDS | ARGGVS | QSVLYS | WAS | QQYYST |
| | YW (SEQ | YT (SEQ | ASGNYR | SNNKNY | (SEQ ID | PLT |
| | ID NO: | ID NO: | ALDY | (SEQ ID | NO: 64) | (SEQ ID |
| | 60) | 61) | (SEQ ID | NO: 63) | | NO: 65) |
| | | | NO: 62) | | | |

### 7.1.5. Assays for Binding Activity of Anti-NKG2D Antibodies Obtained by Immunization and Screening Phage Display Libraries Against NKG2D

Antibodies targeting NKG2d obtained by immunization (HYB:212) and by phage display (APD182) were then combined into a single panel and were then tested on the binding affinity against human and cyno NKG2d which was measured by Surface plasmon resonance (Joseph Bourghol, ELN: NKG2d-00074) (Table 9).

TABLE 9
Binding affinities of anti-NKG2d antibodies to human and cyno NKG2d

| | human NKG2d (NKGW1) | | | Cyno NKG2d (NKGW2.001) | | |
|---|---|---|---|---|---|---|
| Protein | | kd | | | kd | |
| ID | ka (1/Ms) | (1/s) | KD (nM) | ka (1/Ms) | (1/s) | KD (nM) |
| NKGB100 | 1.42E+05 | 1.17E−03 | 8.3 | 1.06E+05 | 1.92E−03 | 18.1 |
| NKGB102 | 1.41E+05 | 6.45E−04 | 4.6 | 1.33E+05 | 1.17E−03 | 8.8 |
| NKGB108 | 1.11E+05 | 1.32E−03 | 11.9 | 1.09E+05 | 1.59E−03 | 14.6 |
| NKGB116 | 1.48E+05 | 9.21E−04 | 6.2 | 1.28E+05 | 2.34E−03 | 18.2 |
| NKGB118 | 9.11E+03 | 2.11E−04 | 23.2 | 1.07E+04 | 1.45E−04 | 13.5 |
| NKGB125 | 7.51E+05 | 3.85E−05 | 0.05 | 5.71E+05 | 5.84E−05 | 0.10 |
| NKGB129 | 1.35E+04 | 2.52E−04 | 18.7 | 1.12E+04 | 1.93E−04 | 17.1 |
| NKGB130 | 9.26E+05 | 9.68E−05 | 0.11 | 5.83E+05 | 1.43E−04 | 0.25 |
| NKGB137 | 2.35E+04 | 3.63E−04 | 15.5 | 1.95E+04 | 3.29E−04 | 16.9 |
| NKGB138 | No/low binding response | | | No/low binding response | | |
| NKGB148 | No/low binding response | | | No/low binding response | | |
| NKGB149 | 4.72E+03 | 1.96E−04 | 41.5 | 1.23E+04 | 2.22E−04 | 17.8 |
| NKGB173 | No/low binding response | | | 3.85E+03 | 1.63E−04 | 42.3 |
| NKGB174 | 3.71E+03 | 1.37E−04 | 36.8 | 1.76E+04 | 2.52E−04 | 16.2 |
| NKGB193 | No/low binding response | | | 5.57E+03 | 2.64E−04 | 47.5 |
| NKGB196 | 4.22E+03 | 2.15E−04 | 50.8 | 1.87E+04 | 2.56E−04 | 15.1 |
| NKGB197 | 2.44E+04 | 2.15E−04 | 8.8 | 1.92E+04 | 1.54E−04 | 8.0 |
| NKGB198 | No/low binding response | | | No/low binding response | | |
| NKGB200 | Binding confirmed, fit not | | | Binding confirmed, fit not valid | | |
| | valid | | | | | |
| NKGB202 | Binding confirmed, fit not | | | No/low binding response | | |
| | valid | | | | | |
| NKGB203 | 2.79E+06 | 1.12E−04 | 0.04 | 1.88E+06 | 2.28E−04 | 0.12 |
| NKGB204 | 3.72E+06 | 2.65E−04 | 0.07 | 3.14E+06 | 7.91E−04 | 0.25 |
| NKGB206 | 3.49E+06 | 5.80E−04 | 0.17 | 3.01E+06 | 9.89E−04 | 0.33 |
| NKGB208 | 4.28E+04 | 8.08E−04 | 18.9 | 7.01E+03 | 8.38E−04 | 120.0 |
| NKGB211 | No/low binding response | | | No/low binding response | | |
| NKGB213 | No/low binding response | | | 1.36E+04 | 2.70E−04 | 19.8 |
| NKGB219 | 1.53E+06 | 3.70E−05 | 0.02 | 1.36E+06 | 2.52E−05 | 0.02 |
| NKGB220 | 2.56E+04 | 8.00E−04 | 31.3 | 1.37E+04 | 5.60E−04 | 41.0 |
| NKGB221 | 6.05E+05 | 1.02E−04 | 0.17 | 4.94E+05 | 9.85E−05 | 0.20 |
| NKGB222 | Insufficient mAb Capture | | | Insufficient mAb Capture | | |
| NKGB54 | 8.41E+04 | 7.62E−04 | 9.1 | 6.85E+04 | 9.41E−04 | 13.7 |
| NKGB56 | 2.77E+05 | 9.32E−04 | 3.4 | 2.27E+05 | 1.30E−03 | 5.7 |
| NKGB63 | 2.10E+05 | 5.56E−04 | 2.7 | 1.68E+05 | 6.55E−04 | 3.9 |
| NKGB65 | 2.38E+05 | 5.97E− | 2.5 | 1.97E+05 | 8.61E− | 4.4 |
| | | 04 | | | 04 | |
| NKGB75 | Binding confirmed, fit not | | | 5.08E+04 | 9.01E−03 | 177.0 |
| | valid | | | | | |
| NKGB83 | 1.14E+05 | 9.41E−04 | 8.2 | 7.76E+04 | 2.12E−03 | 27.4 |
| NKGB88 | Binding confirmed, fit not | | | No/low binding response | | |
| | valid | | | | | |
| NKGB89 | Binding confirmed, fit not | | | 1.78E+05 | 3.68E−03 | 20.7 |
| | valid | | | | | |
| NKGB90 | Binding confirmed, fit not | | | 1.30E+05 | 3.32E−03 | 25.4 |
| | valid | | | | | |
| NKGB93 | 1.80E+05 | 5.29E−04 | 2.9 | 1.22E+05 | 7.42E−04 | 6.1 |
| NKGB95 | 9.87E+04 | 1.05E−03 | 10.7 | 9.82E+04 | 2.18E−03 | 22.3 |

| NKGB98 | Binding confirmed, fit not | No/low binding response | | |
|---|---|---|---|---|
| | valid | | | |
| NKGB99 | Binding confirmed, fit not | 1.29E+05 | 4.73E−03 | 36.6 |
| | valid | | | |

In summary, 7 antibodies displayed affinity for human and cyno NKG2d tighter than 1 nM: NKGB125, NKGB130, NKGB203, NKGB204, NKGB206, NKGB219, and NKGB221. NKGB83 displayed weaker affinity (K_{D}˜8 nM).

### 7.2 Example 2: Functional Assay of Anti-NKG2D Antiboby

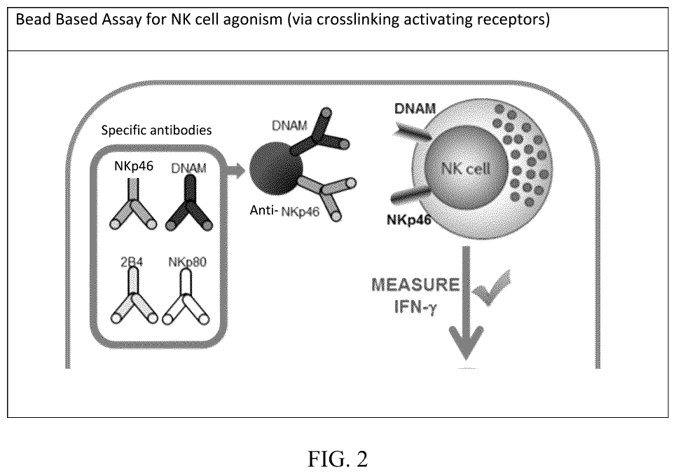

Antibodies targeting NKG2d obtained immunization (HYB:212) and by phage display (APD182) were combined into a single panel and were then tested for their abilities to activate NK cells by measuring IFNg production. FIG. 2 illustrates the Bead Based Assay for NK cell agonism via crosslinking activating receptors.

2000 μLs of beads were washed in 2-5 mL PBS/2% FBS using magnet two times and then resuspended in 2000 μL PBS. 1000 μL of washed beads were then aliquoted in falcon tube and diluted 1:10. Total volume was 10 mL. Then 10 μL of diluted beads were aliquoted into assay plates. 100 μL of antibodies were added into assay plate containing 10 pL of beads. Resulting complexes were incubated for 120 minutes at 4° C. with rocking. Beads were washed in 200 μL PBS using magnet three times then resuspended in assay media. 10 μL of beads in assay media were aliquoted to assigned wells in 96-well and 1×10e5 NK cells were added per well. Total volume per well were 200 μL. The plates were incubated for 16-24 hours at 37° C./5% CO2 and then spinned for 3 minutes at 1300 RPMI. Supernatants were collected for IFNg production measurement.

In summary, based on induction of IFNg production (Table 10), 11 antibodies in total (4 from OMT rats, 7 from phage display) displayed the ability to activate NK cells: NKGB125, NKGB130, NKGB208, NKGB202 were discovered using OMT rats, and NKGB116, NKGB83, NKGB89, NKGB90, NKGB99, NKGB63, NKGB65 were discovered using phage display.

TABLE 10
Analysis of the abilities of anti-NKG2d antibodies to activate NK cells

| Plate 1 | Antibody (ug) | | | Plate 2 | Antibody (ug) | | |
|---|---|---|---|---|---|---|---|
| Antibody | 1 | 0.1 | 0.01 | Antibody | 1 | 0.1 | 0.01 |
| NKGB118.002 | 174.8 | 214.9 | 173.7 | NKGB208.002 | 3850.4 | 788.2 | 226.8 |
| NKGB125.002 | 2773.9 | 2588.2 | 291.2 | NKGB211.002 | 330.1 | 134.1 | 181.6 |
| NKGB129.002 | 259.7 | 151.1 | 153.1 | NKGB213.002 | 205.1 | 135.5 | 177.8 |
| NKGB130.002 | 2546.7 | 1091.1 | 152.3 | NKGB219.002 | 1576.6 | 1108.7 | 309.4 |
| NKGB137.002 | 139.0 | 151.6 | 143.7 | NKGB220.002 | 139.5 | 123.4 | 178.9 |
| NKGB138.002 | 322.7 | 141.9 | 140.3 | NKGB221.002 | 1544.4 | 847.4 | 220.8 |
| NKGB148.002 | 138.9 | 137.1 | 145.3 | NKGB222.002 | 234.3 | 127.0 | 180.5 |
| NKGB149.002 | 155.3 | 147.6 | 160.8 | NKGB116.002 | 4434.5 | 637.8 | 204.5 |
| NKGB174.002 | 151.9 | 190.7 | 169.3 | NKGB83.002 | 4299.1 | 1863.4 | 208.0 |
| NKGB173.002 | 130.8 | 152.2 | 148.7 | NKGB89.002 | 3216.1 | 1281.5 | 184.7 |
| NKGB193.002 | 135.0 | 149.0 | 148.1 | NKGB90.002 | 2679.2 | 1041.5 | 177.0 |
| NKGB196.002 | 145.7 | 140.7 | 142.7 | NKGB99.002 | 3154.9 | 956.4 | 185.3 |
| NKGB198.002 | 135.2 | 138.9 | 141.4 | NKGB94.002 | 674.0 | 118.6 | 170.2 |
| NKGB197.002 | 140.2 | 130.6 | 136.7 | NKGB91.002 | 930.5 | 124.2 | 170.7 |
| NKGB200.002 | 1277.0 | 277.1 | 141.5 | NKGB54.002 | 1897.1 | 503.8 | 180.2 |
| NKGB202.002 | 3706.1 | 482.7 | 160.8 | NKGB63.002 | 3568.1 | 374.2 | 183.8 |
| NKGB203.002 | 1604.5 | 2779.2 | 601.3 | NKGB65.002 | 2746.4 | 1254.3 | 201.7 |
| NKGB204.002 | 1673.4 | 1809.4 | 276.7 | NKGB58.002 | 153.6 | 126.1 | 182.8 |
| NKGB206.002 | 1267.6 | 1800.6 | 204.7 | NKGB56.002 | 1981.6 | 1152.8 | 183.3 |
| NKp46 only | 140.8 | 145.9 | 141.4 | NKp46 only | 117.1 | 127.4 | 180.9 |
| IgG1/NKp46 | 160.1 | 150.9 | 152.0 | IgG1/NKp46 | 143.9 | 130.5 | 176.3 |
| Commerical | 1238.4 | 909.9 | 236.5 | PC | 1291.2 | 817.6 | 317.0 |
| NKp46/NKG2d | | | | | | | |
| NKG2d only | 192.2 | 198.0 | 168.7 | NKG2d only | 186.3 | 167.2 | 182.9 |
| Beads/cells | 122.7 | 120.1 | 148.0 | Beads/cells | 109.1 | 122.9 | 182.9 |
| cells only | | | 169.0 | | | | |

Of these, NKGB125 and NKGB83 displayed high levels of activation. Although some antibodies displayed higher induction, these two antibodies also displayed higher levels of cell binding, suggesting they would be more amenable to cell binding in vivo.

### 7.3 Example 3: Production of Anti-NKP46 Antiboby that Bind NK Cells

### 7.3.1. Production of Anti-NKp46 Antibody(N46B105) by Immunization

To obtain binders for human NKp46, antibody discovery was conducted by immunization. Human NKp46 for immunization was 5T4 was purchased from R&D systems (cat. #1850-N).

Omnirats were immunized with human NKp46 (Table 11, R&D systems cat. #1850-NK) and boosted by twice-weekly immunizations over 8 weeks after which sera was collected from euthanized animals (Jen Pitcher, ELN: Oncology Target Discovery-00192).

TABLE 11
Antigens used for immunizations

| Antigen | Description | Sequence |
|---|---|---|
| NKp4622-254 | NKp4622- | QQQTLPKPFIWAEPHF |
| (Accession # | 254-Fc | MVPKEKQVTICCQGN |
| AAH64806, | | YGAVEYQLHFEGSLF |
| R&D systems | | AVDRPKPPERINKVK |
| cat # | | FYIPDMNSRMAGQYS |
| 1850-NK | | CIYRVGELWSEPSNL |
| | | LDLVVTEMYDTPTLS |
| | | VHPGPEVISGEKVTF |
| | | YCRLDTATSMFLLLK |
| | | EGRSSHVQRGYGKVQ |
| | | AEFPLGPVTTAHRGT |
| | | YRCFGSYNNHAWSFP |
| | | SEPVKLLVTGDIENT |
| | | SLAPEDPTFPDTWGT |
| | | YLLTTETGLQKDHAL |
| | | WDHTAQNIEGRMDPS |
| | | CDKTHTCPPCPAPEL |
| | | LGGPSVFLFPPKPKD |
| | | TLMISRTPEVTCVVV |
| | | DVSHEDPEVKFNWYV |
| | | DGVEVHNAKTKPREE |
| | | QYNSTYRVVSVLTVL |
| | | HQDWLNGKEYKCKVS |
| | | NKALPAPIEKTISKA |
| | | KGQPREPQVYTLPPS |
| | | REEMTKNQVSLTCLV |
| | | KGFYPSDIAEWESNG |
| | | QPENNYKTTPPVLDS |
| | | DGSFFLYSKLTVDKS |
| | | RWQQGNVFSCSVMHE |
| | | ALHNHYTQKLSLSPG |
| | | K |
| | | (SEQ ID NO: 66) |

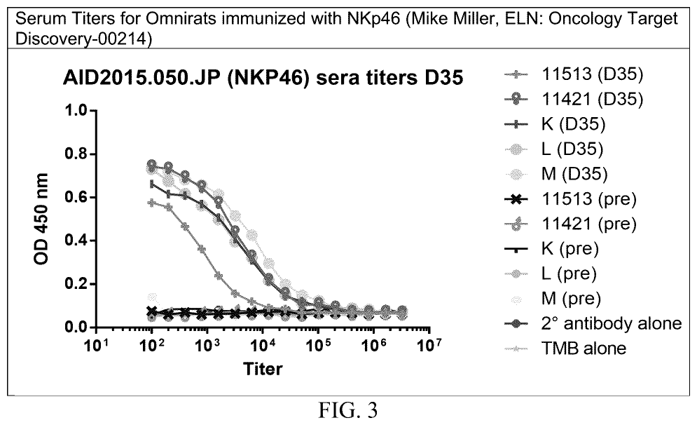

Lymph nodes were obtained as described above. Sera titers were measured to determine immune response to immunizations (FIG. 3).

Lymph nodes and whole blood were harvested and hybridomas were generated as described above (Mike Miller, ELN: Oncology Target Discovery-00217). Briefly, lymphocytes were extracted from lymph nodes and fused to FO cells for hybridoma generation. 100 fusion plates were generated. Average fusion efficiency was 150%. Lymphocytes were also obtained from whole blood and fused to FO cells for hybridoma generation. 10 fusion plates were generated. Average fusion efficiency was 78%. Culture supernatants from 110 fusion plates were screened for antibody binding to NKp46. R analysis was used to analyze ELISA data and yielded 797 hits based on combined hit list (using both R median polish hits and 2× background). R analysis generated a plate map of 264 hits for binding confirmation. Overall, 264 hits from primary screen were re-screened for binding confirmation to NKp46 and cross-screened against B7-H6/Fc. 258 hits (97%) were confirmed as positive binders to the immunogen, NKp46. 117 (out of 258; 45%) confirmed binders bound specifically to NKp46, with no cross-reactivity to Fc portion of B7-H6/Fc. Due to high number of HitGroup Ranking “1, an additional 158 hits from primary screen were re-screened for binding confirmation to NKp46 and cross-screened against B7-H6/Fc. 153 hits (97%) were confirmed as positive binders to the immunogen, NKp46. 91 (out of 153; 59%) confirmed binders bound specifically to NKp46, with no cross-reactivity to Fc portion of GITR/Fc. In total, 208 confirmed specific binders to NKp46, with no cross-reactivity to Fc portion of either B7-H6/Fc or GITR/Fc. Following expansion of hits to 48-well plates, 169 confluent hybridomas were identified by visual inspection and were handed off for v-region cloning and sequencing. Security freezes were prepared. Slow-growing hybridomas were removed from the initial panel and allowed to achieve higher confluency prior to hand-off. Following another round of binding confirmation, 35 additional hits were provided in a second round of v-region cloning. Security freezes were prepared. In summary, a total of 204 specific binders against NKp46 were handed off for v-region sequencing (Req8788 and Req8799). In total, 168 antibody sequences were recovered (Lauren Peters, ELN: Biologics Research Requests—2015-00839) and expressed at 2 mL scale.

Antibodies were tested for their abilities to bind recombinant NKp46 by ELISA (Lamar Blackwell, ELN: ADIPOR-00126). Briefly, recombinant NKp46 was diluted to 2 pg/ml in PBS and added 50 μL/well overnight at 4° C. Plates were washed with PBS, 50 IL of block buffer (PBS 0.4% BSA) were added. Plates were shaked for 30 minutes at room temperature. Plates were washed with PBS again, and 50 μL of 15 ug/ml NKp46 antibodies were added to wells. Plate were shaked for 30 minutes at room temperature. Plates were washed with PBS, and 50 μL of Goat Anti Human Kappa and lambda 2nd antibodies were added. Plate were shaked for 30 minutes at room temperature. Plates were washed with PBS and 50 μL of Sigma Substrate were added, incubated in room temperature for 2-4 minutes. Then 50 μL of stop solution were added. Plates were read on the Envision at 450 nm for ELISA. In total, 28 antibodies displayed significant binding.

### 7.3.2. Assays for Binding Activity of Anti-NKp46 Antibodies Obtained by Immunization on NKL Cell Lines

Antibodies were then assessed for their abilities to bind NKL cells as described above (Lamar Blackwell, ELN: NKG2d-00022). Overall, 104 antibodies displayed significant binding to NKL cells (Table 12).

TABLE 12
NKL cell binding by anti-NKp46 antibodies

| Plate 1 | 10 ug ml | 1 ug ml | 0.1 ug ml |
|---|---|---|---|
| N46B5 | 858 | 269 | 113 |
| N46B6 | 220 | 68 | 54 |
| N46B7 | 70 | 59 | 92 |
| N46B8 | 76 | 52 | 75 |
| N46B9 | 256 | 52 | 83 |
| N46B10 | 189 | 66 | 96 |
| N46B11 | 202 | 59 | 95 |
| N46B12 | 418 | 55 | 88 |
| N46B13 | 270 | 56 | 97 |
| N46B14 | 860 | 216 | 108 |
| N46B15 | 516 | 64 | 102 |
| N46B16 | 2876 | 1512 | 325 |
| N46B17 | 2263 | 381 | 93 |
| N46B18 | 1070 | 95 | 87 |
| N46B19 | 405 | 62 | 112 |
| N46B20 | 630 | 87 | 128 |
| N46B21 | 59 | 50 | 100 |
| N46B22 | 629 | 58 | 121 |
| N46B23 | 250 | 71 | 115 |
| N46B24 | 1892 | 94 | 121 |
| N46B25 | 1601 | 65 | 117 |
| N46B26 | 1588 | 232 | 139 |
| N46B27 | 1689 | 254 | 128 |
| N46B28 | 1668 | 257 | 119 |
| N46B29 | 479 | 195 | 138 |
| N46B30 | 666 | 236 | 119 |
| N46B31 | 852 | 253 | 139 |
| N46B32 | 1443 | 509 | 184 |
| N46B33 | 1584 | 814 | 197 |
| N46B34 | 1115 | 411 | 235 |
| N46B35 | 2971 | 2021 | 1739 |
| N46B36 | 2402 | 87 | 108 |
| N46B37 | 2425 | 1393 | 864 |
| N46B38 | 67 | 100 | 99 |
| N46B39 | 242 | 80 | 113 |
| N46B40 | 2788 | 1414 | 267 |
| N46B41 | 74 | 78 | 106 |
| N46B42 | 646 | 216 | 95 |
| N46B43 | 172 | 71 | 106 |
| N46B44 | 75 | 78 | 97 |
| N46B45 | 1857 | 296 | 138 |
| N46B46 | 70 | 82 | 96 |
| N46B47 | 2674 | 1314 | 228 |
| N46B48 | 75 | 93 | 106 |
| N46B49 | 74 | 79 | 109 |
| N46B50 | 80 | 85 | 98 |
| N46B51 | 9296 | 331 | 130 |
| N46B52 | 87 | 94 | 98 |
| N46B53 | 1000 | 453 | 157 |
| N46B54 | 723 | 236 | 106 |
| N46B55 | 1883 | 728 | 153 |
| N46B56 | 942 | 331 | 166 |
| N46B57 | 2701 | 2100 | 1083 |
| N46B58 | 2875 | 1902 | 530 |
| N46B59 | 788 | 242 | 107 |
| N46B60 | 348 | 144 | 99 |
| N46B61 | 1219 | 415 | 172 |
| N46B62 | 2630 | 1693 | 336 |
| N46B63 | 2842 | 1776 | 260 |
| N46B64 | 240 | 120 | 101 |
| N46B65 | 2521 | 1476 | 245 |
| N46B66 | 2921 | 1290 | 194 |
| N46B67 | 104 | 114 | 80 |
| N46B68 | 2393 | 507 | 160 |
| N46B69 | 2869 | 1565 | 313 |
| N46B70 | 104 | 112 | 90 |
| N46B71 | 5404 | 2080 | 1628 |
| N46B72 | 2792 | 1637 | 294 |
| N46B73 | 2034 | 1299 | 237 |
| N46B74 | 2155 | 1375 | 380 |
| N46B75 | 91 | 103 | 106 |
| N46B76 | 2343 | 1724 | 451 |
| N46B77 | 3045 | 1427 | 231 |
| N46B78 | 2138 | 1155 | 316 |
| N46B79 | 3701 | 1591 | 284 |
| N46B80 | 3282 | 2253 | 563 |
| N46B81 | 2387 | 1155 | 147 |
| N46B82 | 853 | 338 | 95 |
| N46B83 | 1965 | 1085 | 419 |
| N46B84 | 2235 | 1680 | 418 |
| N46B85 | 3241 | 1892 | 393 |
| N46B86 | 3177 | 1846 | 416 |
| N46B87 | 1923 | 897 | 192 |
| N46B88 | 2682 | 2055 | 664 |
| N46B89 | 2620 | 2057 | 633 |
| N46B90 | 2912 | 2371 | 532 |
| N46B91 | 139 | 130 | 98 |
| N46B92 | 7118 | 872 | 127 |
| N46B93 | 2209 | 221 | 98 |
| N46B94 | 2371 | 573 | 111 |
| N46B95 | 2209 | 1454 | 277 |
| N46B96 | 2387 | 1673 | 655 |
| N46B97 | 361 | 98 | 62 |
| N46B98 | 1252 | 359 | 92 |
| N46B99 | 68 | 211 | 79 |
| N46B100 | 1518 | 216 | 85 |
| N46B101 | 1257 | 291 | 82 |
| N46B102 | 1256 | 207 | 76 |
| N46B103 | 1803 | 154 | 59 |
| N46B104 | 662 | 426 | 166 |
| N46B105 | 1457 | 215 | 95 |
| N46B106 | 700 | 183 | 119 |
| N46B107 | 2939 | 2161 | 317 |
| N46B108 | 2808 | 1957 | 453 |
| N46B109 | 1548 | 996 | 92 |
| N46B110 | 1283 | 526 | 113 |
| N46B111 | 2381 | 1460 | 291 |
| N46B112 | 799 | 509 | 120 |
| N46B113 | 50 | 81 | 65 |
| N46B114 | 2342 | 869 | 91 |
| N46B115 | 2482 | 1383 | 135 |
| N46B116 | 1323 | 599 | 88 |
| N46B117 | 1248 | 279 | 85 |
| N46B118 | 1557 | 322 | 100 |
| N46B119 | 1614 | 314 | 87 |
| N46B120 | 1712 | 305 | 89 |
| N46B121 | 1850 | 346 | 100 |
| N46B122 | 1671 | 298 | 130 |
| N46B123 | 2140 | 601 | 89 |
| N46B124 | 2634 | 1712 | 269 |
| N46B125 | 2610 | 1832 | 347 |
| N46B126 | 1724 | 351 | 83 |
| N46B127 | 86 | 74 | 74 |
| N46B128 | 85 | 86 | 80 |
| N46B129 | 1966 | 1531 | 485 |
| N46B130 | 2787 | 1525 | 251 |
| N46B131 | 2862 | 1824 | 310 |
| N46B132 | 156 | 88 | 78 |
| N46B133 | 2279 | 1653 | 378 |
| N46B134 | 2544 | 1648 | 201 |
| N46B135 | 2814 | 1574 | 214 |
| N46B136 | 2905 | 2023 | 416 |
| N46B137 | 2840 | 1885 | 423 |
| N46B138 | 2331 | 1340 | 340 |
| N46B139 | 1721 | 1077 | 242 |
| N46B140 | 2303 | 1910 | 446 |
| N46B141 | 1918 | 1452 | 302 |
| N46B142 | 2202 | 1479 | 208 |
| N46B143 | 2074 | 1315 | 207 |
| N46B144 | 1864 | 1244 | 300 |
| N46B145 | 1839 | 1096 | 174 |
| N46B146 | 2296 | 1600 | 329 |
| N46B147 | 1800 | 1016 | 249 |
| N46B148 | 2269 | 1661 | 410 |
| N46B149 | 481 | 194 | 123 |
| N46B150 | 1380 | 370 | 92 |
| N46B151 | 1449 | 255 | 110 |
| N46B152 | 1305 | 265 | 181 |
| N46B153 | 2041 | 267 | 110 |
| N46B154 | 91 | 132 | 80 |
| N46B155 | 1823 | 1189 | 420 |
| N46B156 | 2104 | 1356 | 275 |
| N46B157 | 3233 | 2262 | 1402 |
| N46B158 | 71 | 100 | 133 |
| N46B159 | 2760 | 1663 | 704 |
| N46B160 | 414 | 118 | 156 |
| N46B161 | 87 | 113 | 176 |
| N46B162 | 548 | 242 | 130 |
| N46B163 | 82 | 154 | 95 |
| N46B164 | 98 | 110 | 105 |
| N46B165 | 67 | 99 | 108 |
| N46B166 | 100 | 136 | 113 |
| N46B167 | 2524 | 1770 | 850 |
| N46B168 | 3124 | 1977 | 1084 |
| N46B169 | 141 | 148 | 118 |
| N46B170 | 108 | 98 | 125 |
| N46B171 | 77 | 136 | 108 |
| N46B172 | 215 | 132 | 130 |
| 2nd only | 87 | 115 | 239 |
| 2nd only | 114 | 114 | 80 |
| 2nd only | 96 | 138 | 101 |
| 2nd only | 126 | 136 | 161 |
| iso | 129 | 133 | 86 |
| iso | 118 | 137 | 83 |
| iso | 131 | 91 | 108 |
| iso | 99 | 137 | 132 |

### 7.3.3. Assays for Binding Activity of Anti-NKP46 Antibodies Against N46W3 (NKP46 D1)

Antibodies were additionally tested for their abilities to bind recombinant full length NKp46: N46W1 and NKp46 D1: N46W3 (Table 13) (Sanjib Dutta, ELN: NKG2d-00026). N46B105 and N46B76 displayed specific binding to N46W3, which contained only domain 1 of NKp46 with K_{D} values of 3.5 and 70 nM, respectively (Table 13). Table 13. Binding affinities of anti-NKp46 antibodies to N46W3

| protein AA ID | Antigen | ka | kd | KD (M) |
|---|---|---|---|---|
| N46B101 | W1 | 4.6E+03 | 1.2E−04 | 2.7E−08 |
| | W3 | 5.1E+04 | 3.8E−04 | 7.4E−09 |
| N46B110 | W1 | 2.0E+04 | 2.7E−03 | 1.3E−07 |
| | W3 | 2.6E+04 | 3.4E−03 | 1.3E−07 |
| N46B120 | W1 | 1.2E+04 | 1.0E−04 | 8.5E−09 |
| | W3 | N/A | N/A | N/A |
| N46B133 | W1 | 2.9E+04 | 1.2E−03 | 4.1E−08 |
| | W3 | 3.5E+04 | 1.5E−03 | 4.3E−08 |
| N46B143 | W1 | 2.8E+04 | 1.5E−03 | 5.2E−08 |
| | W3 | 3.4E+04 | 1.9E−03 | 5.6E−08 |
| N46B155 | W1 | 5.1E+04 | 1.2E−03 | 2.4E−08 |
| | W3 | N/A | N/A | N/A |
| N46B18 | W1 | N/A | N/A | N/A |
| | W3 | N/A | N/A | N/A |
| N46B24 | W1 | N/A | N/A | N/A |
| | W3 | N/A | N/A | N/A |
| N46B102 | W1 | 5.6E+03 | 1.4E−04 | 2.6E−08 |
| | W3 | 4.3E+04 | 9.2E−04 | 2.1E−08 |
| N46B111 | W1 | 4.3E+04 | 8.7E−04 | 2.0E−08 |
| | W3 | N/A | N/A | N/A |
| N46B122 | W1 | 1.2E+04 | 1.0E−04 | 8.6E−09 |
| | W3 | N/A | N/A | N/A |
| N46B135 | W1 | 1.8E+04 | 3.5E−04 | 1.9E−08 |
| | W3 | 2.2E+04 | 4.1E−04 | 1.8E−08 |
| N46B144 | W1 | 2.5E+04 | 1.6E−03 | 6.2E−08 |
| | W3 | 3.1E+04 | 2.0E−03 | 6.4E−08 |
| N46B156 | W1 | 2.0E+04 | 1.6E−03 | 8.1E−08 |
| | W3 | 2.7E+04 | 3.3E−03 | 1.3E−07 |
| N46B25 | W1 | N/A | N/A | N/A |
| | W3 | N/A | N/A | N/A |
| N46B103 | W1 | 3.0E+03 | 1.0E−04 | 3.9E−09 |
| | W3 | 3.1E+03 | 1.0E−04 | 3.2E−08 |
| N46B114 | W1 | 1.9E+04 | 6.0E−04 | 3.2E−08 |
| | W3 | 2.1E+04 | 7.4E−04 | 3.6E−08 |
| N46B124 | W1 | 2.1E+04 | 4.9E−04 | 2.3E−08 |
| | W3 | 2.6E+04 | 7.7E−04 | 3.0E−08 |
| N46B136 | W1 | 2.9E+04 | 1.5E−04 | 5.2E−09 |
| | W3 | 2.9E+04 | 2.1E−04 | 7.4E−09 |
| N46B145 | W1 | 2.5E+04 | 1.6E−03 | 6.3E−08 |
| | W3 | 3.1E+04 | 1.9E−03 | 6.3E−08 |
| N46B157 | W1 | N/A | N/A | N/A |
| | W3 | N/A | N/A | N/A |
| N46B105 | W1 | N/A | N/A | N/A |
| | W3 | 2.9E+04 | 1.0E−04 | 3.5E−09 |
| N46B115 | W1 | 2.3E+04 | 1.0E−04 | 4.3E−09 |
| | W3 | N/A | N/A | N/A |
| N46B126 | W1 | 6.0E+03 | 1.0E−04 | 1.6E−08 |
| | W3 | 3.6E+04 | 3.4E−04 | 9.4E−09 |
| N46B137 | W1 | 2.8E+04 | 3.7E−04 | 1.3E−08 |
| | W3 | 3.2E+04 | 4.6E−04 | 1.5E−08 |
| N46B146 | W1 | 2.3E+04 | 9.6E−04 | 4.2E−08 |
| | W3 | 2.6E+04 | 1.2E−03 | 4.5E−08 |
| N46B159 | W1 | 2.7E+04 | 7.1E−04 | 2.6E−08 |
| | W3 | N/A | N/A | N/A |
| N46B106 | W1 | 3.8E+03 | 1.2E−04 | 3.1E−08 |
| | W3 | N/A | N/A | N/A |
| N46B116 | W1 | 3.8E+03 | 2.4E−04 | 6.3E−08 |
| | W3 | 4.8E+04 | 3.6E−04 | 7.6E−09 |
| N46B127 | W1 | N/A | N/A | N/A |
| | W3 | N/A | N/A | N/A |
| N46B138 | W1 | 3.0E+04 | 1.0E−03 | 3.4E−08 |
| | W3 | 3.4E+04 | 1.3E−03 | 3.8E−08 |
| N46B147 | W1 | 3.0E+04 | 1.9E−03 | 6.2E−08 |
| | W3 | 3.7E+04 | 2.5E−03 | 6.8E−08 |
| N46B16 | W1 | 2.8E+04 | 5.2E−04 | 1.9E−08 |
| | W3 | 3.1E+04 | 6.1E−04 | 2.0E−08 |
| N46B36 | W1 | N/A | N/A | N/A |
| | W3 | N/A | N/A | N/A |
| N46B107 | W1 | 3.1E+04 | 1.5E−04 | 4.7E−09 |
| | W3 | 3.8E+04 | 1.7E−04 | 4.5E−09 |
| N46B117 | W1 | 3.8E+03 | 1.0E−04 | 2.4E−08 |
| | W3 | 4.4E+04 | 1.0E−04 | 2.1E−09 |
| N46B128 | W1 | N/A | N/A | N/A |
| | W3 | N/A | N/A | N/A |
| N46B139 | W1 | 2.9E+04 | 2.0E−03 | 6.6E−08 |
| | W3 | 3.5E+04 | 2.6E−03 | 7.3E−08 |
| N46B150 | W1 | 3.2E+03 | 1.0E−04 | 3.1E−08 |
| | W3 | 6.9E+04 | 1.0E−04 | 1.5E−09 |
| N46B160 | W1 | N/A | N/A | N/A |
| | W3 | N/A | N/A | N/A |
| N46B37 | W1 | 5.0E+04 | 3.3E−04 | 6.6E−09 |
| | W3 | N/A | N/A | N/A |
| N46B108 | W1 | 1.3E+05 | 1.0E−04 | 7.6E−10 |
| | W3 | 4.4E+04 | 1.0E−04 | 9.7E−10 |
| N46B118 | W1 | 4.5E+03 | 2.9E−04 | 6.4E−08 |
| | W3 | 5.9E+04 | 7.7E−04 | 1.3E−08 |
| N46B130 | W1 | 2.8E+04 | 5.8E−04 | 2.1E−08 |
| | W3 | 3.1E+04 | 6.8E−04 | 2.2E−08 |
| N46B140 | W1 | 3.3E+04 | 8.1E−04 | 2.5E−08 |
| | W3 | 3.8E+04 | 1.0E−03 | 2.7E−08 |
| N46B151 | W1 | 6.6E+03 | 1.0E−04 | 1.5E−08 |
| | W3 | 6.3E+04 | 1.0E−04 | 1.6E−09 |
| N46B163 | W1 | N/A | N/A | N/A |
| | W3 | N/A | N/A | N/A |
| N46B109 | W1 | 2.0E+03 | 2.7E−04 | 1.4E−07 |
| | W3 | 5.1E+04 | 5.5E−04 | 1.1E−08 |
| N46B119 | W1 | 5.0E+03 | 3.8E−04 | 7.6E−08 |
| | W3 | 5.8E+04 | 1.0E−03 | 1.8E−08 |
| N46B131 | W1 | N/A | N/A | N/A |
| | W3 | N/A | N/A | N/A |
| N46B142 | W1 | 3.1E+04 | 1.4E−03 | 4.4E−08 |
| | W3 | 3.7E+04 | 1.8E−03 | 5.0E−08 |
| N46B152 | W1 | 6.7E+03 | 1.0E−04 | 1.5E−08 |
| | W3 | 7.1E+04 | 1.0E−04 | 1.4E−09 |
| N46B45 | W1 | 3.2E+04 | 1.7E−04 | 5.1E−09 |
| | W3 | 5.7E+04 | 2.3E−03 | 4.0E−08 |
| N46B69 | W1 | 2.4E+04 | 1.5E−04 | 6.4E−09 |
| | W3 | 2.2E+04 | 2.8E−04 | 1.3E−08 |
| N46B73 | W1 | 2.1E+04 | 1.2E−03 | 5.9E−08 |
| | W3 | 5.9E+04 | 1.0E−03 | 1.7E−08 |
| N46B74 | W1 | 7.2E+04 | 1.1E−03 | 1.6E−08 |
| | W3 | 6.6E+04 | 2.0E−03 | 3.0E−08 |
| N46B47 | W1 | 3.4E+04 | 1.0E−04 | 2.7E−09 |
| | W3 | 2.3E+04 | 1.8E−03 | 8.0E−08 |
| N46B5 | W1 | 2.5E+04 | 8.9E−04 | 3.5E−08 |
| | W3 | 1.7E+04 | 1.3E−03 | 7.3E−08 |
| N46B51 | W1 | N/A | N/A | N/A |
| | W3 | N/A | N/A | N/A |
| N46B53 | W1 | 2.2E+04 | 1.4E−03 | 6.6E−08 |
| | W3 | 2.5E+04 | 3.2E−03 | 1.3E−07 |
| N46B58 | WI | 1.2E+05 | 1.0E−04 | 8.4E−10 |
| | W3 | 4.2E+04 | 1.4E−04 | 3.4E−09 |
| N46B63 | W1 | 1.8E+04 | 2.1E−04 | 1.2E−08 |
| | W3 | 1.9E+04 | 2.5E−04 | 1.3E−08 |
| N46B65 | W1 | 1.4E+04 | 2.4E−04 | 1.7E−08 |
| | W3 | 1.2E+04 | 9.3E−04 | 8.0E−08 |
| N46B68 | W1 | 1.2E+04 | 1.0E−04 | 7.9E−09 |
| | W3 | 4.7E+04 | 2.7E−04 | 5.8E−09 |
| N46B77 | W1 | 3.1E+04 | 3.6E−04 | 1.2E−08 |
| | W3 | 2.9E+04 | 6.2E−04 | 2.1E−08 |
| N46B88 | W1 | 4.2E+04 | 8.2E−04 | 2.0E−08 |
| | W3 | 4.8E+04 | 1.1E−03 | 2.3E−08 |
| N46B89 | W1 | 3.7E+04 | 6.6E−04 | 1.8E−08 |
| | W3 | 4.3E+04 | 8.6E−04 | 2.0E−08 |
| N46B90 | W1 | 4.5E+04 | 3.4E−04 | 7.4E−09 |
| | W3 | 3.9E+04 | 5.6E−04 | 1.5E−08 |
| N46B78 | W1 | 3.9E+04 | 2.4E−03 | 6.2E−08 |
| | W3 | 4.8E+04 | 3.6E−03 | 7.4E−08 |
| N46B79 | W1 | 2.2E+04 | 2.3E−04 | 1.0E−08 |
| | W3 | 2.5E+04 | 2.7E−04 | 1.1E−08 |
| N46B80 | W1 | 3.9E+04 | 1.4E−04 | 3.6E−09 |
| | W3 | 2.8E+04 | 2.5E−04 | 8.8E−09 |
| N46B81 | W1 | 1.8E+04 | 4.4E−04 | 2.5E−08 |
| | W3 | N/A | N/A | N/A |
| N46B82 | W1 | 2.3E+04 | 2.7E−03 | 1.2E−07 |
| | W3 | 2.4E+04 | 5.6E−03 | 2.3E−07 |
| N46B84 | W1 | 3.1E+04 | 1.3E−03 | 4.2E−08 |
| | W3 | 3.7E+04 | 1.7E−03 | 4.6E−08 |
| N46B85 | W1 | 2.9E+04 | 1.0E−04 | 2.2E−09 |
| | W3 | 3.3E+04 | 1.0E−04 | 2.9E−09 |
| N46B87 | W1 | 1.8E+04 | 2.0E−03 | 1.1E−07 |
| | W3 | 2.4E+04 | 2.5E−03 | 1.1E−07 |
| N46B95 | W1 | 3.7E+04 | 1.5E−03 | 4.1E−08 |
| | W3 | 4.3E+04 | 2.1E−03 | 4.9E−08 |
| N46B148 | W1 | 2.0E+04 | 1.1E−03 | 5.3E−08 |
| | W3 | 2.3E+04 | 1.5E−03 | 6.2E−08 |
| N46B153 | W1 | N/A | N/A | N/A |
| | W3 | N/A | N/A | N/A |
| N46B96 | W1 | 6.7E+04 | 1.7E−03 | 2.6E−08 |
| | W3 | 8.1E+04 | 2.5E−03 | 3.1E−08 |
| N46B100 | W1 | 2.0E+06 | 1.0E−04 | 4.9E−11 |
| | W3 | N/A | N/A | N/A |
| N46B121 | W1 | N/A | N/A | N/A |
| | W3 | N/A | N/A | N/A |
| N46B123 | W1 | N/A | N/A | N/A |
| | W3 | N/A | N/A | N/A |
| N46B125 | W1 | N/A | N/A | N/A |
| | W3 | N/A | N/A | N/A |
| N46B129 | W1 | 2.3E+04 | 9.3E−04 | 4.0E−08 |
| | W3 | 2.7E+04 | 1.3E−03 | 4.8E−08 |
| N46B134 | W1 | 1.6E+04 | 1.1E−03 | 6.8E−08 |
| | W3 | 2.0E+04 | 1.4E−03 | 6.7E−08 |
| N46B141 | W1 | N/A | N/A | N/A |
| | W3 | N/A | N/A | N/A |
| N46B167 | W1 | N/A | N/A | N/A |
| | W3 | N/A | N/A | N/A |
| N46B32 | W1 | N/A | N/A | N/A |
| | W3 | N/A | N/A | N/A |
| N46B33 | W1 | N/A | N/A | N/A |
| | W3 | N/A | N/A | N/A |
| N46B168 | W1 | N/A | N/A | N/A |
| | W3 | N/A | N/A | N/A |
| N46B17 | W1 | N/A | N/A | N/A |
| | W3 | N/A | N/A | N/A |
| N46B216 | W1 | 3.0E+02 | 4.6E−04 | 1.4E−06 |
| | W3 | N/A | N/A | N/A |
| N46B217 | W1 | N/A | N/A | N/A |
| | W3 | N/A | N/A | N/A |
| N46B218 | W1 | 1.7E+04 | 1.4E−04 | 8.0E−09 |
| | W3 | N/A | N/A | N/A |
| N46B26 | W1 | N/A | N/A | N/A |
| | W3 | N/A | N/A | N/A |
| N46B27 | W1 | N/A | N/A | N/A |
| | W3 | N/A | N/A | N/A |
| N46B28 | W1 | N/A | N/A | N/A |
| | W3 | N/A | N/A | N/A |
| N46B55 | W1 | N/A | N/A | N/A |
| | W3 | N/A | N/A | N/A |
| N46B86 | W1 | N/A | N/A | N/A |
| | W3 | N/A | N/A | N/A |
| N46B92 | W1 | N/A | N/A | N/A |
| | W3 | N/A | N/A | N/A |
| N46B93 | W1 | N/A | N/A | N/A |
| | W3 | N/A | N/A | N/A |
| N46B57 | W1 | N/A | N/A | N/A |
| | W3 | N/A | N/A | N/A |
| N46B61 | W1 | 3.4E+04 | 2.8E−03 | 8.0E−08 |
| | W3 | 4.8E+04 | 3.3E−03 | 7.0E−08 |
| N46B62 | W1 | 1.5E+04 | 2.9E−04 | 1.9E−08 |
| | W3 | 2.5E+04 | 1.5E−03 | 5.9E−08 |
| N46B66 | W1 | 2.0E+04 | 4.2E−04 | 2.1E−08 |
| | W3 | 2.5E+04 | 7.7E−04 | 3.1E−08 |
| N46B71 | W1 | N/A | N/A | N/A |
| | W3 | N/A | N/A | N/A |
| N46B72 | W1 | 1.8E+04 | 4.2E−04 | 2.3E−08 |
| | W3 | 2.6E+04 | 6.3E−04 | 2.5E−08 |
| N46B76 | W1 | N/A | N/A | N/A |
| | W3 | 3.9E+04 | 2.7E−03 | 7.0E−08 |
| N46B83 | W1 | N/A | N/A | N/A |
| | W3 | N/A | N/A | N/A |
| N46B94 | W1 | 1.2E+04 | 7.2E−04 | 5.9E−08 |
| | W3 | 8.7E+03 | 9.1E−04 | 1.1E−07 |
| N46B208_p2 | W1 | 1.7E+04 | 1.2E−04 | 7.0E−09 |
| | W3 | 1.9E+04 | 1.6E−04 | 8.4E−09 |
| | W1 | 3.6E+04 | 1.4E−04 | 3.8E−09 |
| | W3 | 3.0E+04 | 2.3E−04 | 7.8E−09 |
| | W1 | 1.0E+05 | 1.6E−04 | 1.6E−09 |
| | W3 | 1.0E+05 | 1.8E−04 | 1.8E−09 |
| N46B98 | W1 | N/A | N/A | N/A |
| | W3 | N/A | N/A | N/A |
| N46B157_p2 | W1 | N/A | N/A | N/A |
| | W3 | N/A | N/A | N/A |
| N46B225_p2 | W1 | 4.2E+03 | 1.0E−04 | 2.8E−09 |
| | W3 | N/A | N/A | N/A |
| N46B220_p2 | W1 | 4.2E+03 | 1.0E−04 | 2.4E−08 |
| | W3 | N/A | N/A | N/A |
| N46B183_p2 | W1 | 1.5E+04 | 1.0E−04 | 4.5E−09 |
| | W3 | N/A | N/A | N/A |
| N46B131_p2 | W1 | 3.8E+04 | 1.5E−03 | 3.9E−08 |
| | W3 | N/A | N/A | N/A |
| N46B34 | W1 | 3.9E+04 | 2.3E−03 | 6.0E−08 |
| | W3 | 4.9E+04 | 5.6E−03 | 1.1E−07 |
| N46B35 | W1 | 7.9E+04 | 4.2E−04 | 5.3E−09 |
| | W3 | 9.3E+04 | 5.5E−04 | 5.9E−09 |

Variable region sequences of N46B 105 are provided below in Table 14. The CDRs sequences of N46B105 are provided in Table 15.

TABLE 14
V-regions of N46B105

| mAb | V-region | VH | VL |
|---|---|---|---|
| N46B1 | VR00004 | QLQLQESGPGPVRPS | DIQLTQSPSFLSASV |
| 5 | 4289 | ETLSLTCTVSGDSIR | GDRVTITCRASQGIS |
| | | SSSYYWGWIRQPPGK | SYLAWYQQKPGKAPK |
| | | GLDWIGSIYYTGSTY | LLIYVASTLQSGVPS |
| | | YNPSLMSRVTISVDT | RFSGSGSGTEFTLTI |
| | | SKNQFSLKLSSVTAA | SSLQPEDFATYYCQQ |
| | | DTAVYYCASPGYSSG | LNSYPRMTFGGGTKV |
| | | WSIDYWGQGTLVTVS | EIK |
| | | S | (SEQ ID NO: 68) |
| | | (SEQ ID NO: 67) | |

TABLE 15
CDR Amino Acid Sequences of N46B105

| | HC | HC | HC | LC | LC | LC |
|---|---|---|---|---|---|---|
| System | CDR1 | CDR2 | CDR3 | CDR1 | CDR2 | CDR3 |
| AbM | GDSIRSS | SIYYTGS | PGYSSG | RASQGI | VASTLQ | QQLNSY |
| | SYYWG | TY (SEQ | WSIDY | SSYLA | S (SEQ | PRMT |
| | (SEQ ID | ID NO: | (SEQ ID | (SEQ ID | ID NO: | (SEQ ID |
| | NO: 69) | 70) | NO: 71) | NO: 72) | 73) | NO: 74) |
| Kabat | SSSYYW | SIYYTGS | PGYSSG | RASQGI | VASTLQ | QQLNSY |
| | G (SEQ | TYYNPS | WSIDY | SSYLA | S (SEQ | PRMT |
| | ID NO: | LMS | (SEQ ID | (SEQ ID | ID NO: | (SEQ ID |
| | 75) | (SEQ ID | NO: 77) | NO: 78) | 79) | NO: 80) |
| | | NO: 76) | | | | |
| Chothia | GDSIRSS | YYTGS | PGYSSG | SQGISSY | VAS | LNSYPR |
| | SY (SEQ | (SEQ ID | WSID | (SEQ ID | (SEQ ID | M (SEQ |
| | ID NO: | NO: 82) | (SEQ ID | NO: 84) | NO: 85) | ID NO: |
| | 81) | | NO: 83) | | | 86) |
| Contact | RSSSYY | WIGSIY | ASPGYS | SSYLAW | LLIYVA | QQLNSY |
| | WG (SEQ | YTGSTY | SGWSID | Y (SEQ | STLQ | PRM |
| | ID NO: | (SEQ ID | (SEQ ID | ID NO: | (SEQ ID | (SEQ ID |
| | 87) | NO: 88) | NO: 89) | 90) | NO: 91) | NO: 92) |
| IMGT | GDSIRSS | IYYTGS | ASPGYS | QGISSY | VAS | QQLNSY |
| | SYY | T (SEQ | SGWSID | (SEQ ID | (SEQ ID | PRMT |
| | (SEQ ID | ID NO: | Y (SEQ | NO: 96) | NO: 97) | (SEQ ID |
| | NO: 93) | 94) | ID NO: | | | NO: 98) |
| | | | 95) | | | |

### 7.4 Example 4: Production of Bispecific Antiboby

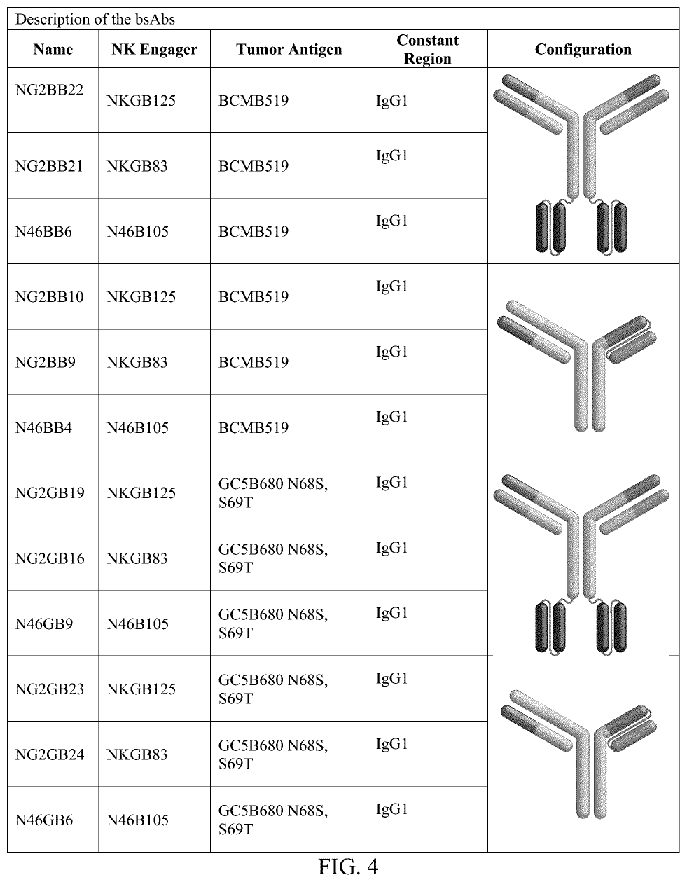

BsAbs were generated using either an anti-BCMA or an anti-GPRC5d scFv (Table 16). The molecules were formatted as Morrison-scaffold antibodies, harboring two sets of identical NK cell-binding Fab regions and C-terminal tumor-targeting scFv moieties. The molecules were also formatted as bipod scaffold antibodies, comprising a tumor-targeting scFv and an NK cell-binding Fab region. All molecules were generated using a normal IgG1, The configurations of the antibodies are shown in FIG. 4.

TABLE 16
Description of the bsAbs

| Name | NK Engager | Tumor Antigen | Constant Region |
|---|---|---|---|
| NG2BB22 | NKGB125 | BCMB519 | IgG1 |
| NG2BB21 | NKGB83 | BCMB519 | IgG1 |
| N46BB6 | N46B105 | BCMB519 | IgG1 |
| NG2BB10 | NKGB125 | BCMB519 | IgG1 |
| NG2BB9 | NKGB83 | BCMB519 | IgG1 |
| N46BB4 | N46B105 | BCMB519 | IgG1 |
| NG2GB19 | NKGB125 | GC5B680 N68S, | IgG1 |
| NG2GB16 | NKGB83 | GC5B680 N68S, | IgG1 |
| N46GB9 | N46B105 | GC5B680 N68S, | IgG1 |
| NG2GB23 | NKGB125 | GC5B680 N68S, | IgG1 |
| NG2GB24 | NKGB83 | GC5B680 N68S, | IgG1 |
| N46GB6 | N46B105 | GC5B680 N68S, | IgG1 |

Variable region sequences of anti-BCMA antibody are provided below in Table 17. The CDRs sequences of anti-BCMA antibody are provided in Table 18.

TABLE 17
VH and VL Amino Acid Sequences
of anti-BCMA Antibodies

| mAb | VH | VL |
|---|---|---|
| anti- | EVQLLESGGGLVQPG | EIVLTQSPGTLSLSP |
| BCMA | GSLRLSCAASGFTFS | GERATLSCRASQSIS |
| | SYAMSWVRQAPGKGL | SSFLTWYQQKPGQAP |
| | EWVSAISGSGGSTYY | RLLIYGASSRATGIP |
| | ADSVKGRFTISRDNS | DRFSGGGSGTDFTLT |
| | KNTLYLQMNSLRAED | ISRLEPEDFAVYYCQ |
| | TAVYYCAKDEGYSSG | HYGSSPMYTFGQGTK |
| | HYYGMDVWGQGTTVT | LEIK |
| | VSS | (SEQ ID NO: 100) |
| | (SEQ ID NO: 99) | |

TABLE 18
CDR Amino Acid Sequences of anti-BCMA Antibodies

| | HC | HC | HC | LC | LC | LC |
|---|---|---|---|---|---|---|
| System | CDR1 | CDR2 | CDR3 | CDR1 | CDR2 | CDR3 |
| AbM | GFTFSS | AISGSG | DEGYSS | RASQSIS | GASSRA | QHYGSS |
| | YAMS | GSTY | GHYYG | SSFLT | T (SEQ | PMYT |
| | (SEQ ID | (SEQ ID | MDV | (SEQ ID | ID NO: | (SEQ ID |
| | NO: 101) | NO: 102) | (SEQ ID | NO: 104) | 105) | NO: 106) |
| | | | NO: 103) | | | |
| Kabat | SYAMS | AISGSG | DEGYSS | RASQSIS | GASSRA | QHYGSS |
| | (SEQ ID | GSTYYA | GHYYG | SSFLT | T (SEQ | PMYT |
| | NO: 107) | DSVKG | MDV | (SEQ ID | ID NO: | (SEQ ID |
| | | (SEQ ID | (SEQ ID | NO: 110) | 111) | NO: 112) |
| | | NO: 108) | NO: 109) | | | |
| Chothia | GFTFSS | SGSGGS | DEGYSS | SQSISSS | GAS | YGSSPM |
| | Y (SEQ | (SEQ ID | GHYYG | F (SEQ | (SEQ ID | Y (SEQ |
| | ID NO: | NO: 114) | MD (SEQ | ID NO: | NO: 117) | ID NO: |
| | 113) | | ID NO: | 116) | | 118) |
| | | | 115) | | | |
| Contact | SSYAMS | WVSAIS | AKDEGY | SSSFLT | LLIYGA | QHYGSS |
| | (SEQ ID | GSGGST | SSGHYY | WY (SEQ | SSRA | PMY |
| | NO: 119) | Y (SEQ | GMD | ID NO: | (SEQ ID | (SEQ ID |
| | | ID NO: | (SEQ ID | 122) | NO: 123) | NO: 124) |
| | | 120) | NO: 121) | | | |
| IMGT | GFTFSS | ISGSGGS | AKDEGY | QSISSSF | GAS | QHYGSS |
| | YA (SEQ | T (SEQ | SSGHYY | (SEQ ID | (SEQ ID | PMYT |
| | ID NO: | ID NO: | GMDV | NO: 128) | NO: 129) | (SEQ ID |
| | 125) | 126) | (SEQ ID | | | NO: 130) |
| | | | NO: 127) | | | |

Variable region sequences of anti-GPRC5d antibody are provided below in Table 19. The CDRs sequences of anti-GPRC5d antibody are provided in Table 20.

TABLE 19
VH and VL Amino Acid Sequences
of anti-GPRC5d Antibodies

| mAb | VH | VL |
|---|---|---|
| anti- | QVTLKESGPVLVKPT | DIVMTQTPLSSPVTL |
| GPRC | ETLTLTCTVSGFSLT | GQPASISCRSSQSLV |
| 5d | NIRMSVSWIRQPPGK | HSDGNTYLSWLQQRP |
| | ALEWLAHIFSNDEKS | GQPPRLLIYKISNRF |
| | YSTSLKSRLTISRDT | FGVPDRFSGSGAGTD |
| | SKSQVVLTLTNVDPV | FTLKISRVEAEDVGV |
| | DTATYYCARMRLPYG | YYCMQATQFPHTFGQ |
| | MDVWGQGTTVTVSS | GTKLEIK |
| | (SEQ ID NO: | (SEQ ID NO: 132) |
| | 131) | |

TABLE 20
CDR Amino Acid Sequences of anti-GPRC5d Antibodies

| | HC | HC | HC | LC | LC | LC |
|---|---|---|---|---|---|---|
| System | CDR1 | CDR2 | CDR3 | CDR1 | CDR2 | CDR3 |
| AbM | GFSLTNI | HIFSNDE | MRLPYG | RSSQSL | KISNRFF | MQATQF |
| | RMSVS | KS (SEQ | MDV | VHSDGN | (SEQ ID | PHT |
| | (SEQ ID | ID NO: | (SEQ ID | TYLS | NO: 137) | (SEQ ID |
| | NO: 133) | 134) | NO: 135) | (SEQ ID | | NO: 138) |
| | | | | NO: 136) | | |
| Kabat | NIRMSV | HIFSNDE | MRLPYG | RSSQSL | KISNRFF | MQATQF |
| | S (SEQ | KSYSTS | MDV | VHSDGN | (SEQ ID | PHT |
| | ID NO: | LKS | (SEQ ID | TYLS | NO: 143) | (SEQ ID |
| | 139) | (SEQ ID | NO: 141) | (SEQ ID | | NO: 144) |
| | | NO: 140) | | NO: 142) | | |
| Chothia | GFSLTNI | FSNDE | MRLPYG | SQSLVH | KIS (SEQ | ATQFPH |
| | RM (SEQ | (SEQ ID | MD (SEQ | SDGNTY | ID NO: | (SEQ ID |
| | ID NO: | NO: 146) | ID NO: | (SEQ ID | 149) | NO: 150) |
| | 145) | | 147) | NO: 148) | | |
| Contact | TNIRMS | WLAHIF | ARMRLP | VHSDGN | LLIYKIS | MQATQF |
| | VS (SEQ | SNDEKS | YGMD | TYLSWL | NRF | PH (SEQ |
| | ID NO: | (SEQ ID | (SEQ ID | (SEQ ID | (SEQ ID | ID NO: |
| | 151) | NO: 152) | NO: 153) | NO: 154) | NO: 155) | 156) |
| IMGT | GFSLTNI | IFSNDEK | ARMRLP | QSL VHS | KIS (SEQ | MQATQF |
| | RMS | (SEQ ID | YGMDV | DGNTY | ID NO: | PHT |
| | (SEQ ID | NO: 158) | (SEQ ID | (SEQ ID | 161) | (SEQ ID |
| | NO: 157) | | NO: 159) | NO: 160) | | NO: 162) |

The sequences of exemplary bispecific antibodies (including those in FIG. 4) are provided below in Table 21 and Table 22.

TABLE 21
VH and VL Sequences of the bsAbs

| Name | VH1 | VL1 | VH2 | VL2 |
|---|---|---|---|---|
| N46BB4 | SEQ ID NO: 67 | SEQ ID NO: 68 | SEQ ID NO: 99 | SEQ ID NO: 100 |
| N46BB6 | SEQ ID NO: 67 | SEQ ID NO: 68 | SEQ ID NO: 99 | SEQ ID NO: 100 |
| N46GB6 | SEQ ID NO: 67 | SEQ ID NO: 68 | SEQ ID NO: 131 | SEQ ID NO: 132 |
| N46GB9 | SEQ ID NO: 67 | SEQ ID NO: 68 | SEQ ID NO: 131 | SEQ ID NO: 132 |
| NG2BB10 | SEQ ID NO: 2 | SEQ ID NO: 3 | SEQ ID NO: 99 | SEQ ID NO: 100 |
| NG2BB21 | SEQ ID NO: 34 | SEQ ID NO: 35 | SEQ ID NO: 99 | SEQ ID NO: 100 |
| NG2BB22 | SEQ ID NO: 2 | SEQ ID NO: 3 | SEQ ID NO: 99 | SEQ ID NO: 100 |
| NG2BB9 | SEQ ID NO: 34 | SEQ ID NO: 35 | SEQ ID NO: 99 | SEQ ID NO: 100 |
| NG2GB16 | SEQ ID NO: 34 | SEQ ID NO: 35 | SEQ ID NO: 131 | SEQ ID NO: 132 |
| NG2GB19 | SEQ ID NO: 2 | SEQ ID NO: 3 | SEQ ID NO: 131 | SEQ ID NO: 132 |
| NG2GB23 | SEQ ID NO: 2 | SEQ ID NO: 3 | SEQ ID NO: 131 | SEQ ID NO: 132 |
| NG2GB24 | SEQ ID NO: 34 | SEQ ID NO: 35 | SEQ ID NO: 131 | SEQ ID NO: 132 |

TABLE 22
Heavy Chain and Light Chain Sequences of the bsAbs

| Name | HC1 | LC1 | HC2 |
|---|---|---|---|
| N46BB4 | QLQLQESGPGPVRPS | DIQLTQSPSFLSASV | EIVLTQSPGTLSLSP |
| | ETLSLTCTVSGDSIR | GDRVTITCRASQGIS | GERATLSCRASQSIS |
| | SSSYYWGWIRQPPGK | SYLAWYQQKPGKAPK | SSFLTWYQQKPGQAP |
| | GLDWIGSIYYTGSTY | LLIYVASTLQSGVPS | RLLIYGASSRATGIP |
| | YNPSLMSRVTISVDT | RFSGSGSGTEFTLTI | DRFSGGGSGTDFTLT |
| | SKNQFSLKLSSVTAA | SSLQPEDFATYYCQQ | ISRLEPEDFAVYYCQ |
| | DTAVYYCASPGYSSG | LNSYPRMTFGGGTKV | HYGSSPMYTFGQGTK |
| | WSIDYWGQGTLVTVS | EIKRTVAAPSVFIFP | LEIKGGSEGKSSGSG |
| | SASTKGPSVFPLAPS | PSDEQLKSGTASVVC | SESKSTGGSEVQLLE |
| | SKSTSGGTAALGCLV | LLNNFYPREAKVQWK | SGGGLVQPGGSLRLS |
| | KDYFPEPVTVSWNSG | VDNALQSGNSQESVT | CAASGFTFSSYAMSW |
| | ALTSGVHTFPAVLQS | EQDSKDSTYSLSSTL | VRQAPGKGLEWVSAI |
| | SGLYSLSSVVTVPSS | TLSKADYEKHKVYAC | SGSGGSTYYADSVKG |
| | SLGTQTYICNVNHKP | EVTHQGLSSPVTKSF | RFTISRDNSKNTLYL |
| | SNTKVDKKVEPKSCD | NRGEC | QMNSLRAEDTAVYYC |
| | KTHTCPPCPAPELLG | (SEQ ID NO: 163) | AKDEGYSSGHYYGMD |
| | GPSVFLFPPKPKDTL | | VWGQGTTVTVSSEPK |
| | MISRTPEVTCVVVDV | | SSDKTHTCPPCPAPE |
| | SHEDPEVKFNWYVDG | | LLGGPSVFLFPPKPK |
| | VEVHNAKTKPREEQY | | DTLMISRTPEVTCVV |
| | NSTYRVVSVLTVLHQ | | VDVSHEDPEVKFNWY |
| | DWLNGKEYKCKVSNK | | VDGVEVHNAKTKPRE |
| | ALPAPIEKTISKAKG | | EQYNSTYRVVSVLTV |
| | QPREPQVYVYPPSRE | | LHQDWLNGKEYKCKV |
| | EMTKNQVSLTCLVKG | | SNKALPAPIEKTISK |
| | FYPSDIAVEWESNGQ | | AKGQPREPQVYVLPP |
| | PENNYKTTPPVLDSD | | SREEMTKNQVSLLCL |
| | GSFALVSKLTVDKSR | | VKGFYPSDIAVEWES |
| | WQQGNVFSCSVMHEA | | NGQPENNYLTWPPVL |
| | LHNHYTQKSLSLSPG | | DSDGSFFLYSKLTVD |
| | K | | KSRWQQGNVFSCSVM |
| | (SEQ ID NO: 164) | | HEALHNHYTQKSLSL |
| | | | SPGK |
| | | | (SEQ ID NO: 165) |
| N46BB6 | QLQLQESGPGPVRPS | DIQLTQSPSFLSASV | NA |
| | ETLSLTCTVSGDSIR | GDRVTITCRASQGIS | |
| | SSSYYWGWIRQPPGK | SYLAWYQQKPGKAPK | |
| | GLDWIGSIYYTGSTY | LLIYVASTLQSGVPS | |
| | YNPSLMSRVTISVDT | RFSGSGSGTEFTLTI | |
| | SKNQFSLKLSSVTAA | SSLQPEDFATYYCQQ | |
| | DTAVYYCASPGYSSG | LNSYPRMTFGGGTKV | |
| | WSIDYWGQGTLVTVS | EIKRTVAAPSVFIFP | |
| | SASTKGPSVFPLAPS | PSDEQLKSGTASVVC | |
| | SKSTSGGTAALGCLV | LLNNFYPREAKVQWK | |
| | KDYFPEPVTVSWNSG | VDNALQSGNSQESVT | |
| | ALTSGVHTFPAVLQS | EQDSKDSTYSLSSTL | |
| | SGLYSLSSVVTVPSS | TLSKADYEKHKVYAC | |
| | SLGTQTYICNVNHKP | EVTHQGLSSPVTKSF | |
| | SNTKVDKKVEPKSCD | NRGEC | |
| | KTHTCPPCPAPELLG | (SEQ ID NO: 163) | |
| | GPSVFLFPPKPKDTL | | |
| | MISRTPEVTCVVVDV | | |
| | SHEDPEVKFNWYVDG | | |
| | VEVHNAKTKPREEQY | | |
| | NSTYRVVSVLTVLHQ | | |
| | DWLNGKEYKCKVSNK | | |
| | ALPAPIEKTISKAKG | | |
| | QPREPQVYTLPPSRE | | |
| | EMTKNQVSLTCLVKG | | |
| | FYPSDIAVEWESNGQ | | |
| | PENNYKTTPPVLDSD | | |
| | GSFFLYSKLTVDKSR | | |
| | WQQGNVFSCSVMHEA | | |
| | LHNHYTQKSLSLSPG | | |
| | GGSEGKSSGSGSESK | | |
| | STGGSEIVLTQSPGT | | |
| | LSLSPGERATLSCRA | | |
| | SQSISSSFLTWYQQK | | |
| | PGQAPRLLIYGASSR | | |
| | ATGIPDRFSGGGSGT | | |
| | DFTLTISRLEPEDFA | | |
| | VYYCQHYGSSPMYTF | | |
| | GQGTKLEIKGGSEGK | | |
| | SSGSGSESKSTGGSE | | |
| | VQLLESGGGLVQPGG | | |
| | SLRLSCAASGFTFSS | | |
| | YAMSWVRQAPGKGLE | | |
| | WVSAISGSGGSTYYA | | |
| | DSVKGRFTISRDNSK | | |
| | NTLYLQMNSLRAEDT | | |
| | AVYYCAKDEGYSSGH | | |
| | YYGMDVWGQGTTVTV | | |
| | SS | | |
| | (SEQ ID NO: 166) | | |
| N46GB6 | QLQLQESGPGPVRPS | DIQLTQSPSFLSASV | DIVMTQTPLSSPVTL |
| | ETLSLTCTVSGDSIR | GDRVTITCRASQGIS | GQPASISCRSSQSLV |
| | SSSYYWGWIRQPPGK | SYLAWYQQKPGKAPK | HSDGNTYLSWLQQRP |
| | GLDWIGSIYYTGSTY | LLIYVASTLQSGVPS | GQPPRLLIYKISNRF |
| | YNPSLMSRVTISVDT | RFSGSGSGTEFTLTI | FGVPDRFSGSGAGTD |
| | SKNQFSLKLSSVTAA | SSLQPEDFATYYCQQ | FTLKISRVEAEDVGV |
| | DTAVYYCASPGYSSG | LNSYPRMTFGGGTKV | YYCMQATQFPHTFGQ |
| | WSIDYWGQGTLVTVS | EIKRTVAAPSVFIFP | GTKLEIKGGSEGKSS |
| | SASTKGPSVFPLAPS | PSDEQLKSGTASVVC | GSGSESKSTGGSQVT |
| | SKSTSGGTAALGCLV | LLNNFYPREAKVQWK | LKESGPVLVKPTETL |
| | KDYFPEPVTVSWNSG | VDNALQSGNSQESVT | TLTCTVSGFSLTNIR |
| | ALTSGVHTFPAVLQS | EQDSKDSTYSLSSTL | MSVSWIRQPPGKALE |
| | SGLYSLSSVVTVPSS | TLSKADYEKHKVYAC | WLAHIFSNDEKSYST |
| | SLGTQTYICNVNHKP | EVTHQGLSSPVTKSF | SLKSRLTISRDTSKS |
| | SNTKVDKKVEPKSCD | NRGEC | QVVLTLTNVDPVDTA |
| | KTHTCPPCPAPELLG | (SEQ ID NO: 163) | TYYCARMRLPYGMDV |
| | GPSVFLFPPKPKDTL | | WGQGTTVTVSSEPKS |
| | MISRTPEVTCVVVDV | | SDKTHTCPPCPAPEL |
| | SHEDPEVKFNWYVDG | | LGGPSVFLFPPKPKD |
| | VEVHNAKTKPREEQY | | TLMISRTPEVTCVVV |
| | NSTYRVVSVLTVLHQ | | DVSHEDPEVKFNWYV |
| | DWLNGKEYKCKVSNK | | DGVEVHNAKTKPREE |
| | ALPAPIEKTISKAKG | | QYNSTYRVVSVLTVL |
| | QPREPQVYVYPPSRE | | HQDWLNGKEYKCKVS |
| | EMTKNQVSLTCLVKG | | NKALPAPIEKTISKA |
| | FYPSDIAVEWESNGQ | | KGQPREPQVYVLPPS |
| | PENNYKTTPPVLDSD | | REEMTKNQVSLLCLV |
| | GSFALVSKLTVDKSR | | KGFYPSDIAVEWESN |
| | WQQGNVFSCSVMHEA | | GQPENNYLTWPPVLD |
| | LHNHYTQKSLSLSPG | | SDGSFFLYSKLTVDK |
| | K | | SRWQQGNVFSCSVMH |
| | (SEQ ID NO: 164) | | EALHNHYTQKSLSLS |
| | | | PGK |
| | | | (SEQ ID NO: 174) |
| N46GB9 | QLQLQESGPGPVRPS | DIQLTQSPSFLSASV | NA |
| | ETLSLTCTVSGDSIR | GDRVTITCRASQGIS | |
| | SSSYYWGWIRQPPGK | SYLAWYQQKPGKAPK | |
| | GLDWIGSIYYTGSTY | LLIYVASTLQSGVPS | |
| | YNPSLMSRVTISVDT | RFSGSGSGTEFTLTI | |
| | SKNQFSLKLSSVTAA | SSLQPEDFATYYCQQ | |
| | DTAVYYCASPGYSSG | LNSYPRMTFGGGTKV | |
| | WSIDYWGQGTLVTVS | EIKRTVAAPSVFIFP | |
| | SASTKGPSVFPLAPS | PSDEQLKSGTASVVC | |
| | SKSTSGGTAALGCLV | LLNNFYPREAKVQWK | |
| | KDYFPEPVTVSWNSG | VDNALQSGNSQESVT | |
| | ALTSGVHTFPAVLQS | EQDSKDSTYSLSSTL | |
| | SGLYSLSSVVTVPSS | TLSKADYEKHKVYAC | |
| | SLGTQTYICNVNHKP | EVTHQGLSSPVTKSF | |
| | SNTKVDKKVEPKSCD | NRGEC | |
| | KTHTCPPCPAPELLG | (SEQ ID NO: 163) | |
| | GPSVFLFPPKPKDTL | | |
| | MISRTPEVTCVVVDV | | |
| | SHEDPEVKFNWYVDG | | |
| | VEVHNAKTKPREEQY | | |
| | NSTYRVVSVLTVLHQ | | |
| | DWLNGKEYKCKVSNK | | |
| | ALPAPIEKTISKAKG | | |
| | QPREPQVYTLPPSRE | | |
| | EMTKNQVSLTCLVKG | | |
| | FYPSDIAVEWESNGQ | | |
| | PENNYKTTPPVLDSD | | |
| | GSFFLYSKLTVDKSR | | |
| | WQQGNVFSCSVMHEA | | |
| | LHNHYTQKSLSLSPG | | |
| | GGSEGKSSGSGSESK | | |
| | STGGSDIVMTQTPLS | | |
| | SPVTLGQPASISCRS | | |
| | SQSLVHSDGNTYLSW | | |
| | LQQRPGQPPRLLIYK | | |
| | ISNRFFGVPDRFSGS | | |
| | GAGTDFTLKISRVEA | | |
| | EDVGVYYCMQATQFP | | |
| | HTFGQGTKLEIKGGS | | |
| | EGKSSGSGSESKSTG | | |
| | GSQVTLKESGPVLVK | | |
| | PTETLTLTCTVSGFS | | |
| | LTNIRMSVSWIRQPP | | |
| | GKALEWLAHIFSNDE | | |
| | KSYSTSLKSRLTISR | | |
| | DTSKSQVVLTLTNVD | | |
| | PVDTATYYCARMRLP | | |
| | YGMDVWGQGTTVTVS | | |
| | S | | |
| | (SEQ ID NO: 167) | | |
| NG2BB10 | EVQLLESGGGLVQPG | SYVLTQPPSVSVAPG | EIVLTQSPGTLSLSP |
| | GSLRLSCAASGFTFS | QTARITCGGNNIGSK | GERATLSCRASQSIS |
| | IYAMTWVRQAPGKGL | SVHWYQQKAGQAPVL | SSFLTWYQQKPGQAP |
| | EWVSIISGSGDHTFY | VVYDDSDRPSGIPER | RLLIYGASSRATGIP |
| | ADSVKGRFTISRDNS | FSGSNSGNTATLTIS | DRFSGGGSGTDFTLT |
| | RNTLYLQMDSLRAED | RVEAGDEADYYCQVW | ISRLEPEDFAVYYCQ |
| | TAVYYCAKEGKWVQL | DGRSDHVVFGGGTKL | HYGSSPMYTFGQGTK |
| | SHFANWGQGTLVTVS | TVLGQPKAAPSVTLF | LEIKGGSEGKSSGSG |
| | SASTKGPSVFPLAPS | PPSSEELQANKATLV | SESKSTGGSEVQLLE |
| | SKSTSGGTAALGCLV | CLISDFYPGAVTVAW | SGGGLVQPGGSLRLS |
| | KDYFPEPVTVSWNSG | KADSSPVKAGVETTT | CAASGFTFSSYAMSW |
| | ALTSGVHTFPAVLQS | PSKQSNNKYAASSYL | VRQAPGKGLEWVSAI |
| | SGLYSLSSVVTVPSS | SLTPEQWKSHRSYSC | SGSGGSTYYADSVKG |
| | SLGTQTYICNVNHKP | QVTHEGSTVEKTVAP | RFTISRDNSKNTLYL |
| | SNTKVDKKVEPKSCD | TECS | QMNSLRAEDTAVYYC |
| | KTHTCPPCPAPELLG | (SEQ ID NO: 169) | AKDEGYSSGHYYGMD |
| | GPSVFLFPPKPKDTL | | VWGQGTTVTVSSEPK |
| | MISRTPEVTCVVVDV | | SSDKTHTCPPCPAPE |
| | SHEDPEVKFNWYVDG | | LLGGPSVFLFPPKPK |
| | VEVHNAKTKPREEQY | | DTLMISRTPEVTCVV |
| | NSTYRVVSVLTVLHQ | | VDVSHEDPEVKFNWY |
| | DWLNGKEYKCKVSNK | | VDGVEVHNAKTKPRE |
| | ALPAPIEKTISKAKG | | EQYNSTYRVVSVLTV |
| | QPREPQVYVYPPSRE | | LHQDWLNGKEYKCKV |
| | EMTKNQVSLTCLVKG | | SNKALPAPIEKTISK |
| | FYPSDIAVEWESNGQ | | AKGQPR |
| | PENNYKTTPPVLDSD | | |
| | GSFALVSKLTVDKSR | | |
| | WQQGNVFSCSVMHEA | | |
| | LHNHYTQKSLSLSPG | | |
| | K | | |
| | (SEQ ID NO: 168) | | |
| | | | EPQVYVLPPSREEMT |
| | | | KNQVSLLCLVKGFYP |
| | | | SDIAVEWESNGQPEN |
| | | | NYLTWPPVLDSDGSF |
| | | | FLYSKLTVDKSRWQQ |
| | | | GNVFSCSVMHEALHN |
| | | | HYTQKSLSLSPGK |
| | | | (SEQ ID NO: 165) |
| NG2BB21 | EVQLVQSGAEVKKPG | DIVMTQSPDSLAVSL | NA |
| | ESLKISCKGSGYSFT | GERATINCKSSQSVL | |
| | SYWIGWVRQMPGKGL | YSSNNKNYLAWYQQK | |
| | EWMGIIYPGDSYTRY | PGQPPKLLIYWASTR | |
| | SPSFQGQVTISADKS | ESGVPDRFSGSGSGT | |
| | ISTAYLQWSSLKASD | DFTLTISSLQAEDVA | |
| | TAMYYCARGGVSASG | VYYCQQYYSTPLTFG | |
| | NYRALDYWGQGTLVT | QGTKVEIKRTVAAPS | |
| | VSSASTKGPSVFPLA | VFIFPPSDEQLKSGT | |
| | PSSKSTSGGTAALGC | ASVVCLLNNFYPREA | |
| | LVKDYFPEPVTVSWN | KVQWKVDNALQSGNS | |
| | SGALTSGVHTFPAVL | QESVTEQDSKDSTYS | |
| | QSSGLYSLSSVVTVP | LSSTLTLSKADYEKH | |
| | SSSLGTQTYICNVNH | KVYACEVTHQGLSSP | |
| | KPSNTKVDKKVEPKS | VTKSFNRGEC | |
| | CDKTHTCPPCPAPEL | (SEQ ID NO: 170) | |
| | LGGPSVFLFPPKPKD | | |
| | TLMISRTPEVTCVVV | | |
| | DVSHEDPEVKFNWYV | | |
| | DGVEVHNAKTKPREE | | |
| | QYNSTYRVVSVLTVL | | |
| | HQDWLNGKEYKCKVS | | |
| | NKALPAPIEKTISKA | | |
| | KGQPREPQVYTLPPS | | |
| | REEMTKNQVSLTCLV | | |
| | KGFYPSDIAVEWESN | | |
| | GQPENNYKTTPPVLD | | |
| | SDGSFFLYSKLTVDK | | |
| | SRWQQGNVFSCSVMH | | |
| | EALHNHYTQKSLSLS | | |
| | PGGGSEGKSSGSGSE | | |
| | SKSTGGSEIVLTQSP | | |
| | GTLSLSPGERATLSC | | |
| | RASQSISSSFLTWYQ | | |
| | QKPGQAPRLLIYGAS | | |
| | SRATGIPDRFSGGGS | | |
| | GTDFTLTISRLEPED | | |
| | FAVYYCQHYGSSPMY | | |
| | TFGQGTKLEIKGGSE | | |
| | GKSSGSGSESKSTGG | | |
| | SEVQLLESGGGLVQP | | |
| | GGSLRLSCAASGFTF | | |
| | SSYAMSWVRQAPGKG | | |
| | LEWVSAISGSGGSTY | | |
| | YADSVKGRFTISRDN | | |
| | SKNTLYLQMNSLRAE | | |
| | DTAVYYCAKDEGYSS | | |
| | GHYYGMDVWGQGTTV | | |
| | TVSS | | |
| | (SEQ ID NO: 171) | | |
| NG2BB22 | EVQLLESGGGLVQPG | SYVLTQPPSVSVAPG | NA |
| | GSLRLSCAASGFTFS | QTARITCGGNNIGSK | |
| | IYAMTWVRQAPGKGL | SVHWYQQKAGQAPVL | |
| | EWVSIISGSGDHTFY | VVYDDSDRPSGIPER | |
| | ADSVKGRFTISRDNS | FSGSNSGNTATLTIS | |
| | RNTLYLQMDSLRAED | RVEAGDEADYYCQVW | |
| | TAVYYCAKEGKWVQL | DGRSDHVVFGGGTKL | |
| | SHFANWGQGTLVTVS | TVLGQPKAAPSVTLF | |
| | SASTKGPSVFPLAPS | PPSSEELQANKATLV | |
| | SKSTSGGTAALGCLV | CLISDFYPGAVTVAW | |
| | KDYFPEPVTVSWNSG | KADSSPVKAGVETTT | |
| | ALTSGVHTFPAVLQS | PSKQSNNKYAASSYL | |
| | SGLYSLSSVVTVPSS | SLTPEQWKSHRSYSC | |
| | SLGTQTYICNVNHKP | QVTHEGSTVEKTVAP | |
| | SNTKVDKKVEPKSCD | TECS | |
| | KTHTCPPCPAPELLG | (SEQ ID NO: 169) | |
| | GPSVFLFPPKPKDTL | | |
| | MISRTPEVTCVVVDV | | |
| | SHEDPEVKFNWYVDG | | |
| | VEVHNAKTKPREEQY | | |
| | NSTYRVVSVLTVLHQ | | |
| | DWLNGKEYKCKVSNK | | |
| | ALPAPIEKTISKAKG | | |
| | QPREPQVYTLPPSRE | | |
| | EMTKNQVSLTCLVKG | | |
| | FYPSDIAVEWESNGQ | | |
| | PENNYKTTPPVLDSD | | |
| | GSFFLYSKLTVDKSR | | |
| | WQQGNVFSCSVMHEA | | |
| | LHNHYTQKSLSLSPG | | |
| | GGSEGKSSGSGSESK | | |
| | STGGSEIVLTQSPGT | | |
| | LSLSPGERATLSCRA | | |
| | SQSISSSFLTWYQQK | | |
| | PGQAPRLLIYGASSR | | |
| | ATGIPDRFSGGGSGT | | |
| | DFTLTISRLEPEDFA | | |
| | VYYCQHYGSSPMYTF | | |
| | GQGTKLEIKGGSEGK | | |
| | SSGSGSESKSTGGSE | | |
| | VQLLESGGGLVQPGG | | |
| | SLRLSCAASGFTFSS | | |
| | YAMSWVRQAPGKGLE | | |
| | WVSAISGSGGSTYYA | | |
| | DSVKGRFTISRDNSK | | |
| | NTLYLQMNSLRAEDT | | |
| | AVYYCAKDEGYSSGH | | |
| | YYGMDVWGQGTTVTV | | |
| | SS | | |
| | (SEQ ID NO: 172) | | |
| NG2BB9 | EVQLVQSGAEVKKPG | DIVMTQSPDSLAVSL | EIVLTQSPGTLSLSP |
| | ESLKISCKGSGYSFT | GERATINCKSSQSVL | GERATLSCRASQSIS |
| | SYWIGWVRQMPGKGL | YSSNNKNYLAWYQQK | SSFLTWYQQKPGQAP |
| | EWMGIIYPGDSYTRY | PGQPPKLLIYWASTR | RLLIYGASSRATGIP |
| | SPSFQGQVTISADKS | ESGVPDRFSGSGSGT | DRFSGGGSGTDFTLT |
| | ISTAYLQWSSLKASD | DFTLTISSLQAEDVA | ISRLEPEDFAVYYCQ |
| | TAMYYCARGGVSASG | VYYCQQYYSTPLTFG | HYGSSPMYTFGQGTK |
| | NYRALDYWGQGTLVT | QGTKVEIKRTVAAPS | LEIKGGSEGKSSGSG |
| | VSSASTKGPSVFPLA | VFIFPPSDEQLKSGT | SESKSTGGSEVQLLE |
| | PSSKSTSGGTAALGC | ASVVCLLNNFYPREA | SGGGLVQPGGSLRLS |
| | LVKDYFPEPVTVSWN | KVQWKVDNALQSGNS | CAASGFTFSSYAMSW |
| | SGALTSGVHTFPAVL | QESVTEQDSKDSTYS | VRQAPGKGLEWVSAI |
| | QSSGLYSLSSVVTVP | LSSTLTLSKADYEKH | SGSGGSTYYADSVKG |
| | SSSLGTQTYICNVNH | KVYACEVTHQGLSSP | RFTISRDNSKNTLYL |
| | KPSNTKVDKKVEPKS | VTKSFNRGEC | QMNSLRAEDTAVYYC |
| | CDKTHTCPPCPAPEL | (SEQ ID NO: 170) | AKDEGYSSGHYYGMD |
| | LGGPSVFLFPPKPKD | | VWGQGTTVTVSSEPK |
| | TLMISRTPEVTCVVV | | SSDKTHTCPPCPAPE |
| | DVSHEDPEVKFNWYV | | LLGGPSVFLFPPKPK |
| | DGVEVHNAKTKPREE | | DTLMISRTPEVTCVV |
| | QYNSTYRVVSVLTVL | | VDVSHEDPEVKFNWY |
| | HQDWLNGKEYKCKVS | | VDGVEVHNAKTKPRE |
| | NKALPAPIEKTISKA | | EQYNSTYRVVSVLTV |
| | KGQPREPQVYVYPPS | | LHQDWLNGKEYKCKV |
| | REEMTKNQVSLTCLV | | SNKALPAPIEKTISK |
| | KGFYPSDIAVEWESN | | AKGQPREPQVYVLPP |
| | GQPENNYKTTPPVLD | | SREEMTKNQVSLLCL |
| | SDGSFALVSKLTVDK | | VKGFYPSDIAVEWES |
| | SRWQQGNVFSCSVMH | | NGQPENNYLTWPPVL |
| | EALHNHYTQKSLSLS | | DSDGSFFLYSKLTVD |
| | PGK | | KSRWQQGNVFSCSVM |
| | (SEQ ID NO: 173) | | HEALHNHYTQKSLSL |
| | | | SPGK |
| | | | (SEQ ID NO: 165) |
| NG2GB16 | EVQLVQSGAEVKKPG | DIVMTQSPDSLAVSL | NA |
| | ESLKISCKGSGYSFT | GERATINCKSSQSVL | |
| | SYWIGWVRQMPGKGL | YSSNNKNYLAWYQQK | |
| | EWMGIIYPGDSYTRY | PGQPPKLLIYWASTR | |
| | SPSFQGQVTISADKS | ESGVPDRFSGSGSGT | |
| | ISTAYLQWSSLKASD | DFTLTISSLQAEDVA | |
| | TAMYYCARGGVSASG | VYYCQQYYSTPLTFG | |
| | NYRALDYWGQGTLVT | QGTKVEIKRTVAAPS | |
| | VSSASTKGPSVFPLA | VFIFPPSDEQLKSGT | |
| | PSSKSTSGGTAALGC | ASVVCLLNNFYPREA | |
| | LVKDYFPEPVTVSWN | KVQWKVDNALQSGNS | |
| | SGALTSGVHTFPAVL | QESVTEQDSKDSTYS | |
| | QSSGLYSLSSVVTVP | LSSTLTLSKADYEKH | |
| | SSSLGTQTYICNVNH | KVYACEVTHQGLSSP | |
| | KPSNTKVDKKVEPKS | VTKSFNRGEC | |
| | CDKTHTCPPCPAPEL | (SEQ ID NO: 170) | |
| | LGGPSVFLFPPKPKD | | |
| | TLMISRTPEVTCVVV | | |
| | DVSHEDPEVKFNWYV | | |
| | DGVEVHNAKTKPREE | | |
| | QYNSTYRVVSVLTVL | | |
| | HQDWLNGKEYKCKVS | | |
| | NKALPAPIEKTISKA | | |
| | KGQPREPQVYTLPPS | | |
| | REEMTKNQVSLTCLV | | |
| | KGFYPSDIAVEWESN | | |
| | GQPENNYKTTPPVLD | | |
| | SDGSFFLYSKLTVDK | | |
| | SRWQQGNVFSCSVMH | | |
| | EALHNHYTQKSLSLS | | |
| | PGGGSEGKSSGSGSE | | |
| | SKSTGGSDIVMTQTP | | |
| | LSSPVTLGQPASISC | | |
| | RSSQSLVHSDGNTYL | | |
| | SWLQQRPGQPPRLLI | | |
| | YKISNRFFGVPDRFS | | |
| | GSGAGTDFTLKISRV | | |
| | EAEDVGVYYCMQATQ | | |
| | FPHTFGQGTKLEIKG | | |
| | GSEGKSSGSGSESKS | | |
| | TGGSQVTLKESGPVL | | |
| | VKPTETLTLTCTVSG | | |
| | FSLTNIRMSVSWIRQ | | |
| | PPGKALEWLAHIFSN | | |
| | DEKSYSTSLKSRLTI | | |
| | SRDTSKSQVVLTLTN | | |
| | VDPVDTATYYCARMR | | |
| | LPYGMDVWGQGTTVT | | |
| | VSS | | |
| | (SEQ ID NO: 175) | | |
| NG2GB19 | EVQLLESGGGLVQPG | SYVLTQPPSVSVAPG | NA |
| | GSLRLSCAASGFTFS | QTARITCGGNNIGSK | |
| | IYAMTWVRQAPGKGL | SVHWYQQKAGQAPVL | |
| | EWVSIISGSGDHTFY | VVYDDSDRPSGIPER | |
| | ADSVKGRFTISRDNS | FSGSNSGNTATLTIS | |
| | RNTLYLQMDSLRAED | RVEAGDEADYYCQVW | |
| | TAVYYCAKEGKWVQL | DGRSDHVVFGGGTKL | |
| | SHFANWGQGTLVTVS | TVLGQPKAAPSVTLF | |
| | SASTKGPSVFPLAPS | PPSSEELQANKATLV | |
| | SKSTSGGTAALGCLV | CLISDFYPGAVTVAW | |
| | KDYFPEPVTVSWNSG | KADSSPVKAGVETTT | |
| | ALTSGVHTFPAVLQS | PSKQSNNKYAASSYL | |
| | SGLYSLSSVVTVPSS | SLTPEQWKSHRSYSC | |
| | SLGTQTYICNVNHKP | QVTHEGSTVEKTVAP | |
| | SNTKVDKKVEPKSCD | TECS | |
| | KTHTCPPCPAPELLG | (SEQ ID NO: 169) | |
| | GPSVFLFPPKPKDTL | | |
| | MISRTPEVTCVVVDV | | |
| | SHEDPEVKFNWYVDG | | |
| | VEVHNAKTKPREEQY | | |
| | NSTYRVVSVLTVLHQ | | |
| | DWLNGKEYKCKVSNK | | |
| | ALPAPIEKTISKAKG | | |
| | QPREPQVYTLPPSRE | | |
| | EMTKNQVSLTCLVKG | | |
| | FYPSDIAVEWESNGQ | | |
| | PENNYKTTPPVLDSD | | |
| | GSFFLYSKLTVDKSR | | |
| | WQQGNVFSCSVMHEA | | |
| | LHNHYTQKSLSLSPG | | |
| | GGSEGKSSGSGSESK | | |
| | STGGSDIVMTQTPLS | | |
| | SPVTLGQPASISCRS | | |
| | SQSLVHSDGNTYLSW | | |
| | LQQRPGQPPRLLIYK | | |
| | ISNRFFGVPDRFSGS | | |
| | GAGTDFTLKISRVEA | | |
| | EDVGVYYCMQATQFP | | |
| | HTFGQGTKLEIKGGS | | |
| | EGKSSGSGSESKSTG | | |
| | GSQVTLKESGPVLVK | | |
| | PTETLTLTCTVSGFS | | |
| | LTNIRMSVSWIRQPP | | |
| | GKALEWLAHIFSNDE | | |
| | KSYSTSLKSRLTISR | | |
| | DTSKSQVVLTLTNVD | | |
| | PVDTATYYCARMRLP | | |
| | YGMDVWGQGTTVTVS | | |
| | S | | |
| | (SEQ ID NO: 176) | | |
| NG2GB2 | EVQLLESGGGLVQPG | SYVLTQPPSVSVAPG | DIVMTQTPLSSPVTL |
| 3 | GSLRLSCAASGFTFS | QTARITCGGNNIGSK | GQPASISCRSSQSLV |
| | IYAMTWVRQAPGKGL | SVHWYQQKAGQAPVL | HSDGNTYLSWLQQRP |
| | EWVSIISGSGDHTFY | VVYDDSDRPSGIPER | GQPPRLLIYKISNRF |
| | ADSVKGRFTISRDNS | FSGSNSGNTATLTIS | FGVPDRFSGSGAGTD |
| | RNTLYLQMDSLRAED | RVEAGDEADYYCQVW | FTLKISRVEAEDVGV |
| | TAVYYCAKEGKWVQL | DGRSDHVVFGGGTKL | YYCMQATQFPHTFGQ |
| | SHFANWGQGTLVTVS | TVLGQPKAAPSVTLF | GTKLEIKGGSEGKSS |
| | SASTKGPSVFPLAPS | PPSSEELQANKATLV | GSGSESKSTGGSQVT |
| | SKSTSGGTAALGCLV | CLISDFYPGAVTVAW | LKESGPVLVKPTETL |
| | KDYFPEPVTVSWNSG | KADSSPVKAGVETTT | TLTCTVSGFSLTNIR |
| | ALTSGVHTFPAVLQS | PSKQSNNKYAASSYL | MSVSWIRQPPGKALE |
| | SGLYSLSSVVTVPSS | SLTPEQWKSHRSYSC | WLAHIFSNDEKSYST |
| | SLGTQTYICNVNHKP | QVTHEGSTVEKTVAP | SLKSRLTISRDTSKS |
| | SNTKVDKKVEPKSCD | TECS | QVVLTLTNVDPVDTA |
| | KTHTCPPCPAPELLG | (SEQ ID NO: 169) | TYYCARMRLPYGMDV |
| | GPSVFLFPPKPKDTL | | WGQGTTVTVSSEPKS |
| | MISRTPEVTCVVVDV | | SDKTHTCPPCPAPEL |
| | SHEDPEVKFNWYVDG | | LGGPSVFLFPPKPKD |
| | VEVHNAKTKPREEQY | | TLMISRTPEVTCVVV |
| | NSTYRVVSVLTVLHQ | | DVSHEDPEVKFNWYV |
| | DWLNGKEYKCKVSNK | | DGVEVHNAKTKPREE |
| | ALPAPIEKTISKAKG | | QYNSTYRVVSVLTVL |
| | QPREPQVYVYPPSRE | | HQDWLNGKEYKCKVS |
| | EMTKNQVSLTCLVKG | | NKALPAPIEKTISKA |
| | FYPSDIAVEWESNGQ | | KGQPREPQVYVLPPS |
| | PENNYKTTPPVLDSD | | REEMTKNQVSLLCLV |
| | GSFALVSKLTVDKSR | | KGFYPSDIAVEWESN |
| | WQQGNVFSCSVMHEA | | GQPENNYLTWPPVLD |
| | LHNHYTQKSLSLSPG | | SDGSFFLYSKLTVDK |
| | K | | SRWQQGNVFSCSVMH |
| | (SEQ ID NO: 168) | | EALHNHYTQKSLSLS |
| | | | PGK |
| | | | (SEQ ID NO: 174) |
| NG2GB24 | EVQLVQSGAEVKKPG | DIVMTQSPDSLAVSL | DIVMTQTPLSSPVTL |
| | ESLKISCKGSGYSFT | GERATINCKSSQSVL | GQPASISCRSSQSLV |
| | SYWIGWVRQMPGKGL | YSSNNKNYLAWYQQK | HSDGNTYLSWLQQRP |
| | EWMGIIYPGDSYTRY | PGQPPKLLIYWASTR | GQPPRLLIYKISNRF |
| | SPSFQGQVTISADKS | ESGVPDRFSGSGSGT | FGVPDRFSGSGAGTD |
| | ISTAYLQWSSLKASD | DFTLTISSLQAEDVA | FTLKISRVEAEDVGV |
| | TAMYYCARGGVSASG | VYYCQQYYSTPLTFG | YYCMQATQFPHTFGQ |
| | NYRALDYWGQGTLVT | QGTKVEIKRTVAAPS | GTKLEIKGGSEGKSS |
| | VSSASTKGPSVFPLA | VFIFPPSDEQLKSGT | GSGSESKSTGGSQVT |
| | PSSKSTSGGTAALGC | ASVVCLLNNFYPREA | LKESGPVLVKPTETL |
| | LVKDYFPEPVTVSWN | KVQWKVDNALQSGNS | TLTCTVSGFSLTNIR |
| | SGALTSGVHTFPAVL | QESVTEQDSKDSTYS | MSVSWIRQPPGKALE |
| | QSSGLYSLSSVVTVP | LSSTLTLSKADYEKH | WLAHIFSNDEKSYST |
| | SSSLGTQTYICNVNH | KVYACEVTHQGLSSP | SLKSRLTISRDTSKS |
| | KPSNTKVDKKVEPKS | VTKSFNRGEC | QVVLTLTNVDPVDTA |
| | CDKTHTCPPCPAPEL | (SEQ ID NO: 170) | TYYCARMRLPYGMDV |
| | LGGPSVFLFPPKPKD | | WGQGTTVTVSSEPKS |
| | TLMISRTPEVTCVVV | | SDKTHTCPPCPAPEL |
| | DVSHEDPEVKFNWYV | | LGGPSVFLFPPKPKD |
| | DGVEVHNAKTKPREE | | TLMISRTPEVTCVVV |
| | QYNSTYRVVSVLTVL | | DVSHEDPEVKFNWYV |
| | HQDWLNGKEYKCKVS | | DGVEVHNAKTKPREE |
| | NKALPAPIEKTISKA | | QYNSTYRVVSVLTVL |
| | KGQPREPQVYVYPPS | | HQDWLNGKEYKCKVS |
| | REEMTKNQVSLTCLV | | NKALPAPIEKTISKA |
| | KGFYPSDIAVEWESN | | KGQPREPQVYVLPPS |
| | GQPENNYKTTPPVLD | | REEMTKNQVSLLCLV |
| | SDGSFALVSKLTVDK | | KGFYPSDIAVEWESN |
| | SRWQQGNVFSCSVMH | | GQPENNYLTWPPVLD |
| | EALHNHYTQKSLSLS | | SDGSFFLYSKLTVDK |
| | PGK | | SRWQQGNVFSCSVMH |
| | (SEQ ID NO: 173) | | EALHNHYTQKSLSLS |
| | | | PGK |
| | | | (SEQ ID NO: 174) |

### 7.5 Example 5: Evaluation of Cytotoxic Properties of the Bispecific Antibody

The bsAbs were further evaluated for their cytotoxic properties. Briefly, H929/GFP cells that endogenously express BCMA and GPRC5d were used as target cells and human PBMC (Hemcare, PBO09C-50, Lot #19054456) were used as effector cells. Target cells, effector cells and antibody treatments were prepared and added to wells in clear bottom plates (PerkinElmer #6057300), with 6.6 to 1 effector to target ratio. Real-time live-cell imaging system Incucyte (Sartarious) was used to image the cells every hour and total GFP integrated signal per well was quantified. Average and standard deviation was calculated for replicates. Time points data are normalized to time 0 when treatments were added. Endpoint (47-hour) dose response curves were plotted for these molecules and four-parameter non-linear regression was performed to obtain IC50 by Graphpad Prism.

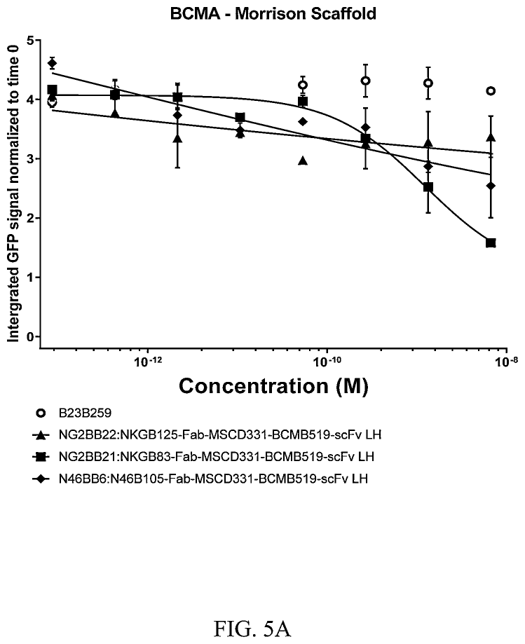

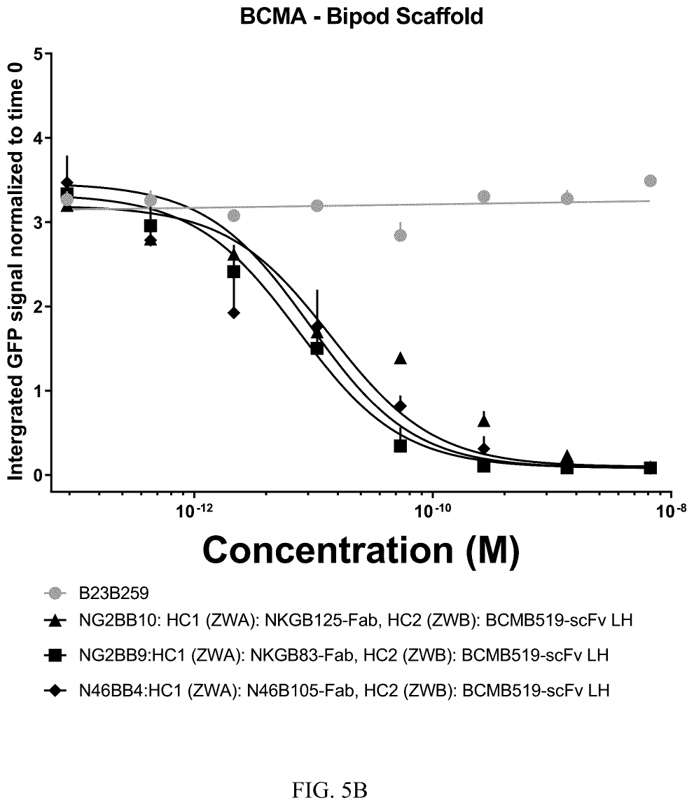

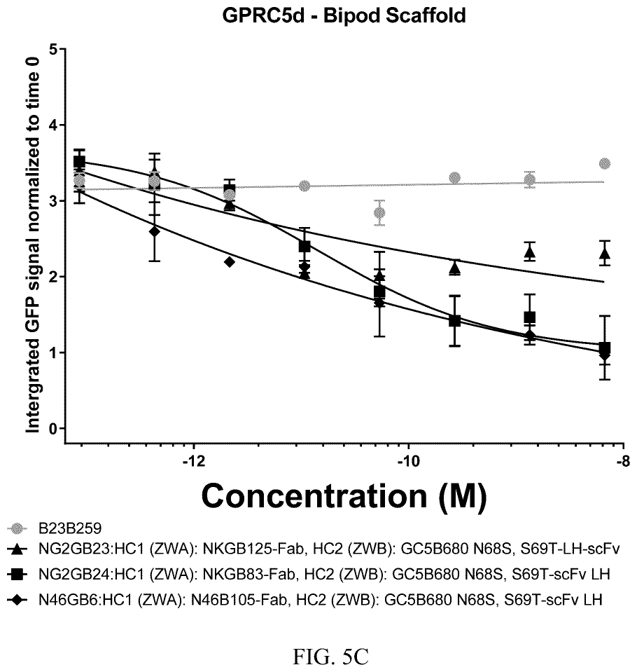

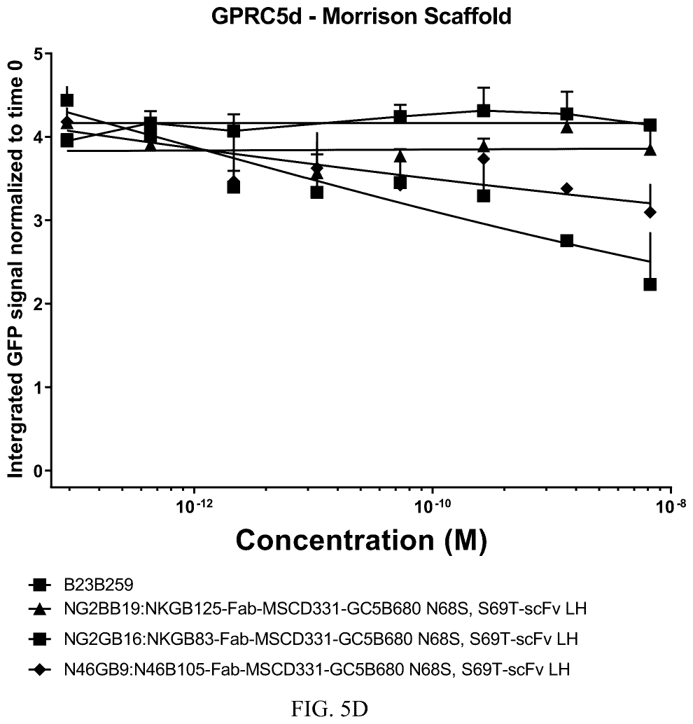

Overall, the Morrison-scaffold bsAbs displayed weak activity, likely due to weaker binding to target cells. In this format, only NG2BB21 and NG2GB26 mediated NK cell-based cytotoxicity, having IC50 values in the nanomolar range (FIG. 5A-D, Table 23).

TABLE 23
IC50 values for BsAb cytotoxicity

| Name | IC50 (nM) | Name | IC50 (nM) |
|---|---|---|---|
| NG2BB22 | too weak | NG2GB19 | 1.99E−11 |
| NG2BB21 | 1.236E−09 | NG2GB16 | too weak |
| N46BB6 | too weak | N46GB9 | too weak |
| NG2BB10 | 2.095E−11 | NG2GB23 | 3.37E−12 |
| NG2BB9 | 7.224E−12 | NG2GB24 | 1.44E−11 |
| N46BB4 | 1.534E−12 | N46GB6 | too weak |

Bipod-scaffold molecules, although characterized by monovalency, displayed more potent activity. In this format, the all BCMA-based bsAbs had activity NG2BB10 (NKGB125-based), NG2BB9 (NKGB83-based), and N46BB4 (N46B105-based) all featured a normal IgG1 constant region but a different NK cell engager. These molecules all had almost identical activity, with IC₅₀ values˜10 pM. This trend was true of the GPRC5d-targeting bipod bsAbs as well, wherein the identity of the constant region impacted the IC50 more prominently than the identity of the NK cell engager.

The results illustrate that, first, the three NK cell engagers: NKGB125, NKGB83, and N46B105 were all competent to mediate NK cell redirection similar activity. Second, the bipod-based configuration was more amenable to NK cell redirection compared to the Morrison-scaffold bsAb configuration.

### 7.6 Example 6: Bispecific NK Engagers have Superior Cytotoxic Properties by Functioning Through Both Activating NK Receptor and Fc Receptor

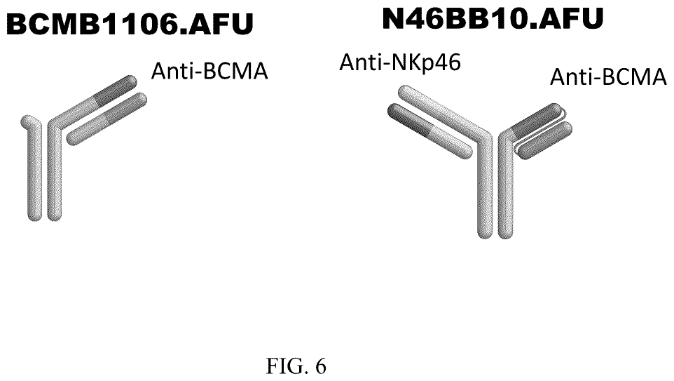

To further investigate the cytotoxic properties of the bispecific NK engagers, one NKp46 binder, N46B105, was paired with a BCMA binder with either silent or wild type Fc made in regular and afucosylated cells (FIG. 6). ADCC was run on these molecules with NK cells or PMBCs directly or in conditions expected to mimicking immune suppressive tumor environment such as in the presence of TGFβ or under hypoxia.

In the TGFβ study, PBMC was revived and rested overnight. Upon NK cells isolation (CD16⁺/CD56⁺) from PBMC, they were treated with different concentrations of TGFβ or incubated in media without TGFβ for 72 hours and ADCC assay using DELFIA-EuTDA time-resolved fluorescence cytotoxicity kit (PerkinElmer) was performed after 4 hours of addition with TGFβ-treated or none-treated NK cells.

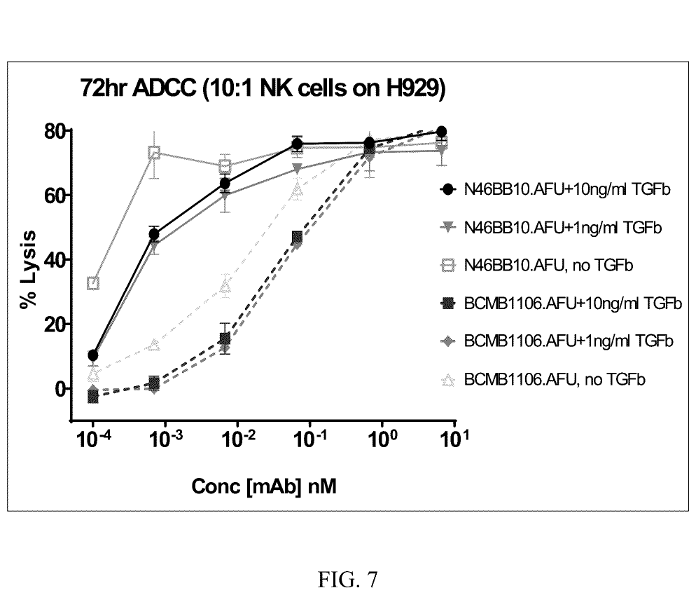

FIG. 7 shows ADCC activities with NK cells on BCMA-endogenous expressing H929 cells. Without preconditioning effector cells with TGFβ, the bispecific NK engager N46BB10. AFU outperformed the corresponding antibody BCMB1106. AFU, indicating the NKp46 arm brings in further cytotoxic effects by NK cells in addition to the effect induced by Fc receptors, in particular, CD16. After the effector cells being preconditioned in TGFβ, the cytotoxicity effect decreased due to the immune-suppressing property of TGFβ. However, the bispecific NK engager was still more potent than the corresponding antibody lacking NKp46 binder, suggesting this benefit of having the NKp46 translates into immune-suppressing environment.

In the hypoxia study, PBMC (Hemacare PB009C-3 Lot 19055785) was thawed, responded and incubated in Avatar Hipoxia chamber (Xcellbio) under the condition of 37° C., 5% CO₂, 2% O₂, 3.0 PSI for four days. Then MM1R/GFP cells, effector cells and antibody treatments were prepared and added to wells in clear bottom plates (PerkinElmer #6057300), with 10 to 1 PBMC to target ratio. Real-time live-cell imaging system Incucyte (Sartarious) was used to image the cells every hour and total GFP integrated signal per well was quantified. Average and standard deviation was calculated for replicates. Time points data are normalized to time 0 when treatments were added. Endpoint (48-hour) dose response curves were plotted for these molecules and four-parameter non-linear regression was performed to obtain IC50 by Graphpad Prism.

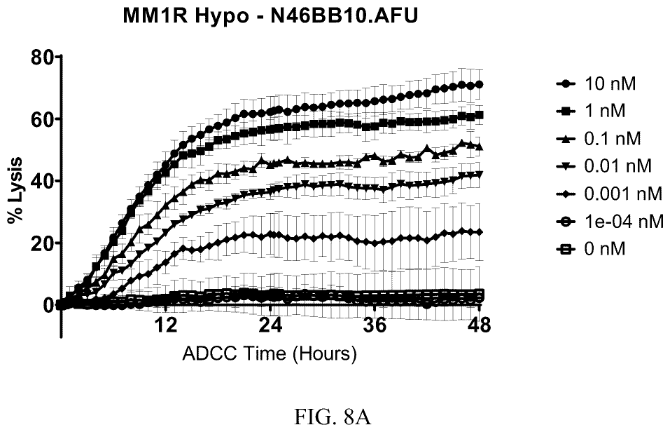

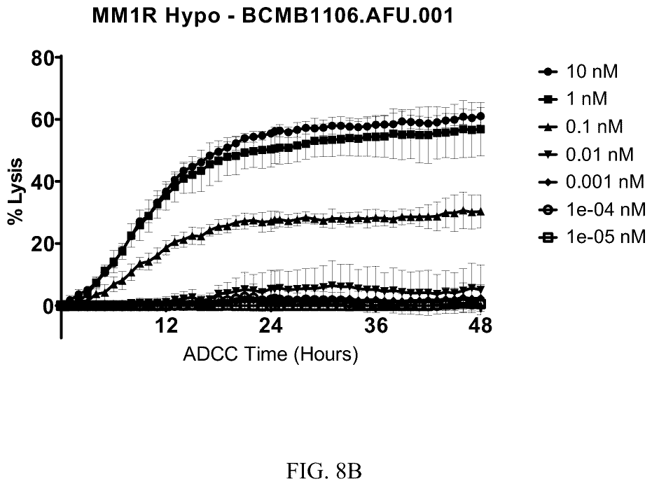

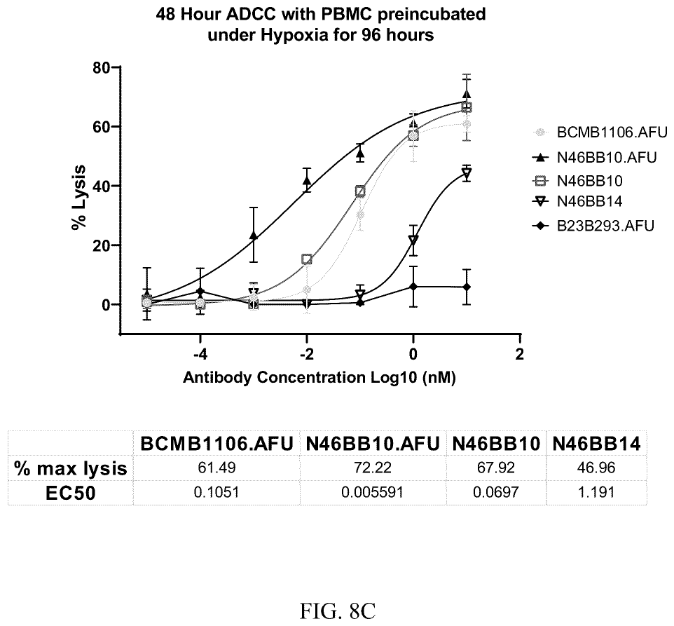

The hypoxia cytotoxicity kinetics and the endpoint dose-response are shown in FIGS. 8A, 8B, and 8C. ADCC activity can be observed within hours after antibodies and effector cells were added and plated after a day for both bispecific NK engager N46BB10.AFU and the corresponding antibody BCMB1106.AFU (FIGS. 8A and 81D). The NK engager variant with IgG1 silent mutations (N46BB14) alone is able to induce ˜47% cytotoxicity with EC50 of ˜1.191 nM (FIG. 8C), representing the contribution of cytotoxicity effect of NKp46 redirection alone. Adding the Fc receptor-induced cytotoxicity through an active wild type Fc (N46BB10) or through an afucosylated Fc (N46BB10.AFU) increased the max percent lysis to approximately 68% and 72%, respectively, while decreased the EC50 values to −0.07 and 0.006 nM, respectively. Comparing the afucosylated bispecific NK engager (N46BB1.AFU) that functions through both NKp46 as well as Fc receptor to the corresponding antibody (BCMB1106.AFU) that functions through Fc receptor alone, there is an increase in both % max lysis as well as potency, demonstrating the potential superiority in efficacy of the bi-specific and bi-functional NK engagers as therapeutics.

The amino acid sequence information of N46BB10 is listed as below in Table 24.

TABLE 24
Amino acid sequence of N46BB10
NKp46 Heavy Chain 1

| | |
|---|---|
| Full AA | QLQLQESGPGPVRPSETLSLTCTVSGDSIRSSSYYWGWIRQPPGKGLD |
| sequence | WIGSIYYTGSTYYNPSLMSRVTISVDTSKNQFSLKLSSVTAADTAVYY |
| | CASPGYSSGWSIDYWGQGTLVTVSSASTKGPSVFPLAPSSKSTSGGTA |
| | ALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELL |
| | GGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKAL |
| | PAPIEKTISKAKGQPREPQVYTLPPSREEMTKNQVSLWCLVKGFYPSDI |
| | AVEWESNGQPENNYKTTPPVLDSDGSFFLYSKLTVDKSRWQQGNVFS |
| | CSVMHEALHNRFTQKSLSLSPGK (SEQ ID NO: 177) |
| VH | QLQLQESGPGPVRPSETLSLTCTVSGDSIRSSSYYWGWIRQPPGKGLD |
| | WIGSIYYTGSTYYNPSLMSRVTISVDTSKNQFSLKLSSVTAADTAVYY |
| | CASPGYSSGWSIDYWGQGTLVTVSS (SEQ ID NO: 67) |
| Constant | ASTKGPSVFPLAPSSKSTSGGTAALGCLVKDYFPEPVTVSWNSGALTS |
| Region | GVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRE |
| | EMTKNQVSLWCLVKGFYPSDIAVEWESNGQPENNYKTTPPVLDSDGS |
| | FFLYSKLTVDKSRWQQGNVFSCSVMHEALHNRFTQKSLSLSPGK |
| | (SEQ ID NO: 178) |

NKp46 Light Chain 1

| | |
|---|---|
| Full AA | DIQLTQSPSFLSASVGDRVTITCRASQGISSYLAWYQQKPGKAPKLLIY |
| sequence | VASTLQSGVPSRFSGSGSGTEFTLTISSLQPEDFATYYCQQLNSYPRMT |
| | FGGGTKVEIKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC (SEQ ID NO: 163) |
| VL | DIQLTQSPSFLSASVGDRVTITCRASQGISSYLAWYQQKPGKAPKLLIY |
| | VASTLQSGVPSRFSGSGSGTEFTLTISSLQPEDFATYYCQQLNSYPRMT |
| | FGGGTKVEIK (SEQ ID NO: 68) |
| Constant | RTVAAPSVFIFPPSDEQLKSGTASVVCLLNNFYPREAKVQWKVDNAL |
| Region | QSGNSQESVTEQDSKDSTYSLSSTLTLSKADYEKHKVYACEVTHQGL |
| | SSPVTKSFNRGEC (SEQ ID NO: 179) |

BCMA Heavy Chain 2

| | |
|---|---|
| Full AA | EIVLTQSPGTLSLSPGERATLSCRASQSISSSFLTWYQQKPGQAPRLLIY |
| sequence | GASSRATGIPDRFSGGGSGTDFTLTISRLEPEDFAVYYCQHYGSSPMYT |
| | FGQGTKLEIKGGSEGKSSGSGSESKSTGGSEVQLLESGGGLVQPGGSL |
| | RLSCAASGFTFSSY AMSWVRQAPGKGLEWVSAISGSGGSTYYADSVK |
| | GRFTISRDNSKNTLYLQMNSLRAEDTAVYYCAKDEGYSSGHYYGMD |
| | VWGQGTTVTVSSEPKSSDKTHTCPPCPAPELLGGPSVFLFPPKPKDTL |
| | MISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPR |
| | EPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQ |
| | KSLSLSPGK (SEQ ID NO: 180) |
| VH | EVQLLESGGGLVQPGGSLRLSCAASGFTFSSYAMSWVRQAPGKGLE |
| | WVSAISGSGGSTYYADSVKGRFTISRDNSKNTLYLQMNSLRAEDTAV |
| | YYCAKDEGYSSGHYYGMDVWGQGTTVTVSS (SEQ ID NO: 99) |
| Linker | GGSEGKSSGSGSESKSTGGS (SEQ ID NO: 181) |
| VL | EIVLTQSPGTLSLSPGERATLSCRASQSISSSFLTWYQQKPGQAPRLLIY |
| | GASSRATGIPDRFSGGGSGTDFTLTISRLEPEDFAVYYCQHYGSSPMYT |
| | FGQGTKLEIK (SEQ ID NO: 100) |
| Constant | EPKSSDKTHTCPPCPAPELLGGPSVFLFPPKPKDTLMISRTPEVTCVVV |
| Region | DVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREE |
| | MTKNQVSLSCAVKGFYPSDIAVEWESNGQPENNYKTTPPVLDSDGSF |
| | FLVSKLTVDKSRWQQGNVFSCSVMHEALHNHYTQKSLSLSPGK (SEQ |
| | ID NO: 182) |

The nucleic acid sequence information of N46BB10 is listed as below in Table 25.

TABLE 25
Nucleic acid sequence of N46BB10
NKp46 Heavy Chain 1

| | |
|---|---|
| Full DNA | CAGCTCCAGTTGCAGGAATCAGGTCCCGGTCCCGTCCGCCCCTCTG |
| sequence | AGACCCTTTCTTTGACCTGCACAGTATCCGGCGACTCTATCCGCAG |
| | CTCATCATACTACTGGGGTTGGATTCGGCAGCCTCCAGGAAAAGGT |
| | CTCGACTGGATCGGATCTATTTACTATACTGGCTCTACCTACTACA |
| | ATCCTAGCTTGATGAGCAGGGTTACCATTTCTGTCGACACCTCAAA |
| | AAATCAGTTCAGTCTTAAACTGTCCAGCGTCACTGCTGCAGACACT |
| | GCAGTGTATTATTGTGCCTCACCCGGATATTCATCCGGGTGGAGCA |
| | TTGATTATTGGGGCCAAGGTACACTCGTAACAGTCTCTTCAGCCTC |
| | CACCAAGGGCCCATCGGTCTTCCCCCTGGCACCCTCCTCCAAGAGC |
| | ACCTCTGGGGGCACAGCGGCCCTGGGCTGCCTGGTCAAGGACTAC |
| | TTCCCCGAACCGGTGACGGTGTCGTGGAACTCAGGCGCCCTGACC |
| | AGCGGCGTGCACACCTTCCCGGCTGTCCTACAGTCCTCAGGACTCT |
| | ACTCCCTCAGCAGCGTGGTGACCGTGCCCTCCAGCAGCTTGGGCAC |
| | CCAGACCTACATCTGCAACGTGAATCACAAGCCCAGCAACACCAA |
| | GGTGGACAAGAAAGTTGAGCCCAAATCTTGTGACAAAACTCACAC |
| | ATGCCCACCGTGCCCAGCACCTGAACTCCTGGGGGGACCGTCAGT |
| | CTTCCTCTTCCCCCCAAAACCCAAGGACACCCTCATGATCTCCCGG |
| | ACCCCTGAGGTCACATGCGTGGTGGTGGACGTGAGCCACGAAGAC |
| | CCTGAGGTCAAGTTCAACTGGTACGTGGACGGCGTGGAGGTGCAT |
| | AATGCCAAGACAAAGCCGCGGGAGGAGCAGTACAACAGCACGTA |
| | CCGTGTGGTCAGCGTCCTCACCGTCCTGCACCAGGACTGGCTGAAT |
| | GGCAAGGAGTACAAGTGCAAGGTCTCCAACAAAGCCCTCCCAGCC |
| | CCCATCGAGAAAACCATCTCCAAAGCCAAAGGGCAGCCCCGAGAA |
| | CCACAGGTGTACACCCTGCCCCCATCCCGGGAGGAGATGACCAAG |
| | AACCAGGTCAGCCTGTGGTGCCTGGTCAAAGGCTTCTATCCCAGCG |
| | ACATCGCCGTGGAGTGGGAGAGCAATGGGCAGCCGGAGAACAACT |
| | ACAAGACCACGCCTCCCGTGCTGGACTCCGACGGCTCCTTCTTCCT |
| | CTACAGCAAGCTCACCGTGGACAAGAGCAGGTGGCAGCAGGGGA |
| | ACGTCTTCTCATGCTCCGTGATGCATGAGGCTCTGCACAACCGGTT |
| | CACGCAGAAGAGCCTCTCCCTGTCTCCGGGTAAA (SEQ ID NO: 183) |
| VH DNA | CAGCTCCAGTTGCAGGAATCAGGTCCCGGTCCCGTCCGCCCCTCTG |
| | AGACCCTTTCTTTGACCTGCACAGTATCCGGCGACTCTATCCGCAG |
| | CTCATCATACTACTGGGGTTGGATTCGGCAGCCTCCAGGAAAAGGT |
| | CTCGACTGGATCGGATCTATTTACTATACTGGCTCTACCTACTACA |
| | ATCCTAGCTTGATGAGCAGGGTTACCATTTCTGTCGACACCTCAAA |
| | AAATCAGTTCAGTCTTAAACTGTCCAGCGTCACTGCTGCAGACACT |
| | GCAGTGTATTATTGTGCCTCACCCGGATATTCATCCGGGTGGAGCA |
| | TTGATTATTGGGGCCAAGGTACACTCGTAACAGTCTCTTCA (SEQ |
| | ID NO: 184) |
| Constant | GCCTCCACCAAGGGCCCATCGGTCTTCCCCCTGGCACCCTCCTCCA |
| Region | AGAGCACCTCTGGGGGCACAGCGGCCCTGGGCTGCCTGGTCAAGG |
| DNA | ACTACTTCCCCGAACCGGTGACGGTGTCGTGGAACTCAGGCGCCCT |
| | GACCAGCGGCGTGCACACCTTCCCGGCTGTCCTACAGTCCTCAGGA |
| | CTCTACTCCCTCAGCAGCGTGGTGACCGTGCCCTCCAGCAGCTTGG |
| | GCACCCAGACCTACATCTGCAACGTGAATCACAAGCCCAGCAACA |
| | CCAAGGTGGACAAGAAAGTTGAGCCCAAATCTTGTGACAAAACTC |
| | ACACATGCCCACCGTGCCCAGCACCTGAACTCCTGGGGGGACCGT |
| | CAGTCTTCCTCTTCCCCCCAAAACCCAAGGACACCCTCATGATCTC |
| | CCGGACCCCTGAGGTCACATGCGTGGTGGTGGACGTGAGCCACGA |
| | AGACCCTGAGGTCAAGTTCAACTGGTACGTGGACGGCGTGGAGGT |
| | GCATAATGCCAAGACAAAGCCGCGGGAGGAGCAGTACAACAGCA |
| | CGTACCGTGTGGTCAGCGTCCTCACCGTCCTGCACCAGGACTGGCT |
| | GAATGGCAAGGAGTACAAGTGCAAGGTCTCCAACAAAGCCCTCCC |
| | AGCCCCCATCGAGAAAACCATCTCCAAAGCCAAAGGGCAGCCCCG |
| | AGAACCACAGGTGTACACCCTGCCCCCATCCCGGGAGGAGATGAC |
| | CAAGAACCAGGTCAGCCTGTGGTGCCTGGTCAAAGGCTTCTATCCC |
| | AGCGACATCGCCGTGGAGTGGGAGAGCAATGGGCAGCCGGAGAA |
| | CAACTACAAGACCACGCCTCCCGTGCTGGACTCCGACGGCTCCTTC |
| | TTCCTCTACAGCAAGCTCACCGTGGACAAGAGCAGGTGGCAGCAG |
| | GGGAACGTCTTCTCATGCTCCGTGATGCATGAGGCTCTGCACAACC |
| | GGTTCACGCAGAAGAGCCTCTCCCTGTCTCCGGGTAAA (SEQ ID |
| | NO: 185) |

NKp46 Light Chain 1

| | |
|---|---|
| Full DNA | GACATCCAGCTGACCCAGAGCCCCAGCTTTCTGAGCGCCAGCGTG |
| sequence | GGCGACAGGGTGACCATCACCTGCAGGGCCAGCCAGGGCATCAGC |
| | AGCTACCTGGCCTGGTACCAGCAGAAGCCCGGCAAAGCCCCCAAG |
| | CTGCTGATCTACGTGGCCAGCACACTGCAGAGCGGCGTGCCTAGC |
| | AGATTTAGCGGCAGCGGCAGCGGCACAGAGTTCACCCTGACCATC |
| | TCCAGCCTGCAGCCCGAGGACTTCGCCACCTACTACTGCCAGCAGC |
| | TGAACAGCTACCCCAGGATGACCTTCGGCGGCGGCACCAAGGTGG |
| | AGATCAAGCGTACGGTGGCTGCACCATCTGTCTTCATCTTCCCGCC |
| | ATCTGATGAGCAGTTGAAATCTGGAACTGCCTCTGTTGTGTGCCTG |
| | CTGAATAACTTCTATCCCAGAGAGGCCAAAGTACAGTGGAAGGTG |
| | GATAACGCCCTCCAATCGGGTAACTCCCAGGAGAGTGTCACAGAG |
| | CAGGACAGCAAGGACAGCACCTACAGCCTCAGCAGCACCCTGACG |
| | CTGAGCAAAGCAGACTACGAGAAACACAAAGTCTACGCCTGCGAA |
| | GTCACCCATCAGGGCCTGAGCTCGCCCGTCACAAAGAGCTTCAAC |
| | AGGGGAGAGTGT (SEQ ID NO: 186) |
| VL DNA | GACATCCAGCTGACCCAGAGCCCCAGCTTTCTGAGCGCCAGCGTG |
| | GGCGACAGGGTGACCATCACCTGCAGGGCCAGCCAGGGCATCAGC |
| | AGCTACCTGGCCTGGTACCAGCAGAAGCCCGGCAAAGCCCCCAAG |
| | CTGCTGATCTACGTGGCCAGCACACTGCAGAGCGGCGTGCCTAGC |
| | AGATTTAGCGGCAGCGGCAGCGGCACAGAGTTCACCCTGACCATC |
| | TCCAGCCTGCAGCCCGAGGACTTCGCCACCTACTACTGCCAGCAGC |
| | TGAACAGCTACCCCAGGATGACCTTCGGCGGCGGCACCAAGGTGG |
| | AGATCAAG (SEQ ID NO: 187) |
| Constant | CGTACGGTGGCTGCACCATCTGTCTTCATCTTCCCGCCATCTGATG |
| Region | AGCAGTTGAAATCTGGAACTGCCTCTGTTGTGTGCCTGCTGAATAA |
| DNA | CTTCTATCCCAGAGAGGCCAAAGTACAGTGGAAGGTGGATAACGC |
| | CCTCCAATCGGGTAACTCCCAGGAGAGTGTCACAGAGCAGGACAG |
| | CAAGGACAGCACCTACAGCCTCAGCAGCACCCTGACGCTGAGCAA |
| | AGCAGACTACGAGAAACACAAAGTCTACGCCTGCGAAGTCACCCA |
| | TCAGGGCCTGAGCTCGCCCGTCACAAAGAGCTTCAACAGGGGAGA |
| | GTGT (SEQ ID NO: 188) |

BCMA Heavy Chain 2

| | |
|---|---|
| Full DNA | GAGATCGTGCTGACTCAGAGCCCTGGCACTCTGAGCCTGAGCCCTG |
| Sequence | GCGAGAGGGCCACTCTGAGCTGTAGGGCCAGCCAGAGCATCAGCA |
| | GCAGCTTCCTGACTTGGTACCAGCAGAAGCCTGGCCAGGCCCCTA |
| | GGCTGCTGATCTACGGCGCCAGCAGCAGGGCCACTGGCATCCCTG |
| | ATAGGTTCAGCGGCGGCGGCAGCGGCACTGATTTCACTCTGACTAT |
| | CAGCAGGCTGGAGCCTGAGGATTTCGCCGTGTACTACTGTCAGCAC |
| | TACGGCAGCAGCCCTATGTACACTTTCGGCCAGGGCACTAAGCTG |
| | GAGATCAAGGGCGGCTCCGAGGGCAAGAGCAGCGGCAGCGGCAG |
| | CGAGAGCAAGAGCACCGGCGGCAGCGAAGTGCAGCTGCTGGAGTC |
| | AGGGGGCGGTCTGGTACAACCGGGCGGCAGCCTGAGGCTGAGCTG |
| | TGCCGCCAGCGGCTTCACTTTCAGCAGCTACGCCATGAGCTGGGTG |
| | AGGCAGGCCCCTGGCAAGGGCCTGGAGTGGGTGAGCGCCATCAGC |
| | GGCAGCGGCGGCAGCACTTACTACGCCGATAGCGTGAAGGGCAGG |
| | TTCACTATCAGCAGGGATAATAGCAAGAATACTCTGTACCTGCAG |
| | ATGAATAGCCTGAGGGCCGAGGATACTGCCGTGTACTACTGTGCC |
| | AAGGATGAGGGCTACAGCAGCGGCCACTACTACGGCATGGATGTG |
| | TGGGGCCAGGGCACTACTGTGACTGTGAGCAGCGAGCCCAAATCT |
| | AGCGACAAAACTCACACATGTCCACCGTGCCCAGCACCTGAACTC |
| | CTGGGGGGACCGTCAGTCTTCCTCTTCCCCCCAAAACCCAAGGACA |
| | CCCTCATGATCTCCCGGACCCCTGAGGTCACATGCGTGGTGGTGGA |
| | CGTGAGCCACGAAGACCCTGAGGTCAAGTTCAACTGGTACGTGGA |
| | CGGCGTGGAGGTGCATAATGCCAAGACAAAGCCGCGGGAGGAGC |
| | AGTACAACAGCACGTACCGTGTGGTCAGCGTCCTCACCGTCCTGCA |
| | CCAGGACTGGCTGAATGGCAAGGAGTACAAGTGCAAGGTCTCCAA |
| | CAAAGCCCTCCCAGCCCCCATCGAGAAAACCATCTCCAAAGCCAA |
| | AGGGCAGCCCCGAGAACCACAGGTGTACACCCTGCCCCCATCCCG |
| | GGAGGAGATGACCAAGAACCAGGTCAGCCTGTCCTGCGCCGTCAA |
| | AGGCTTCTATCCCAGCGACATCGCCGTGGAGTGGGAGAGCAATGG |
| | GCAGCCGGAGAACAACTACAAGACCACGCCTCCCGTGCTGGACTC |
| | CGACGGCTCCTTCTTCCTCGTGAGCAAGCTCACCGTGGACAAGTCT |
| | AGATGGCAGCAGGGGAACGTCTTCTCATGCTCCGTGATGCATGAG |
| | GCTCTGCACAACCACTACACGCAGAAGAGCCTCTCCCTGTCTCCGG |
| | GTAAA (SEQ ID NO: 189) |
| VH DNA | GAAGTGCAGCTGCTGGAGTCAGGGGGCGGTCTGGTACAACCGGGC |
| | GGCAGCCTGAGGCTGAGCTGTGCCGCCAGCGGCTTCACTTTCAGCA |
| | GCTACGCCATGAGCTGGGTGAGGCAGGCCCCTGGCAAGGGCCTGG |
| | AGTGGGTGAGCGCCATCAGCGGCAGCGGCGGCAGCACTTACTACG |
| | CCGATAGCGTGAAGGGCAGGTTCACTATCAGCAGGGATAATAGCA |
| | AGAATACTCTGTACCTGCAGATGAATAGCCTGAGGGCCGAGGATA |
| | CTGCCGTGTACTACTGTGCCAAGGATGAGGGCTACAGCAGCGGCC |
| | ACTACTACGGCATGGATGTGTGGGGCCAGGGCACTACTGTGACTG |
| | TGAGCAGC (SEQ ID NO: 190) |
| Linker | GGCGGCTCCGAGGGCAAGAGCAGCGGCAGCGGCAGCGAGAGCAA |
| DNA | GAGCACCGGCGGCAGC (SEQ ID NO: 191) |
| VL DNA | GAGATCGTGCTGACTCAGAGCCCTGGCACTCTGAGCCTGAGCCCTG |
| | GCGAGAGGGCCACTCTGAGCTGTAGGGCCAGCCAGAGCATCAGCA |
| | GCAGCTTCCTGACTTGGTACCAGCAGAAGCCTGGCCAGGCCCCTA |
| | GGCTGCTGATCTACGGCGCCAGCAGCAGGGCCACTGGCATCCCTG |
| | ATAGGTTCAGCGGCGGCGGCAGCGGCACTGATTTCACTCTGACTAT |
| | CAGCAGGCTGGAGCCTGAGGATTTCGCCGTGTACTACTGTCAGCAC |
| | TACGGCAGCAGCCCTATGTACACTTTCGGCCAGGGCACTAAGCTG |
| | GAGATCAAG (SEQ ID NO: 192) |
| Constant | GAGCCCAAATCTAGCGACAAAACTCACACATGTCCACCGTGCCCA |
| Region | GCACCTGAACTCCTGGGGGGACCGTCAGTCTTCCTCTTCCCCCCAA |
| DNA | AACCCAAGGACACCCTCATGATCTCCCGGACCCCTGAGGTCACAT |
| | GCGTGGTGGTGGACGTGAGCCACGAAGACCCTGAGGTCAAGTTCA |
| | ACTGGTACGTGGACGGCGTGGAGGTGCATAATGCCAAGACAAAGC |
| | CGCGGGAGGAGCAGTACAACAGCACGTACCGTGTGGTCAGCGTCC |
| | TCACCGTCCTGCACCAGGACTGGCTGAATGGCAAGGAGTACAAGT |
| | GCAAGGTCTCCAACAAAGCCCTCCCAGCCCCCATCGAGAAAACCA |
| | TCTCCAAAGCCAAAGGGCAGCCCCGAGAACCACAGGTGTACACCC |
| | TGCCCCCATCCCGGGAGGAGATGACCAAGAACCAGGTCAGCCTGT |
| | CCTGCGCCGTCAAAGGCTTCTATCCCAGCGACATCGCCGTGGAGTG |
| | GGAGAGCAATGGGCAGCCGGAGAACAACTACAAGACCACGCCTCC |
| | CGTGCTGGACTCCGACGGCTCCTTCTTCCTCGTGAGCAAGCTCACC |
| | GTGGACAAGTCTAGATGGCAGCAGGGGAACGTCTTCTCATGCTCC |
| | GTGATGCATGAGGCTCTGCACAACCACTACACGCAGAAGAGCCTC |
| | TCCCTGTCTCCGGGTAAA (SEQ ID NO: 193) |

The amino acid sequence information of N46BB14 is listed as below in Table 26.

TABLE 26
Amino acid sequence of N46BB14
NKp46 Heavy Chain 1

| | |
|---|---|
| Full AA | QLQLQESGPGPVRPSETLSLTCTVSGDSIRSSSYYWGWIRQPPGKGLD |
| sequence | WIGSIYYTGSTYYNPSLMSRVTISVDTSKNQFSLKLSSVTAADTAVYY |
| | CASPGYSSGWSIDYWGQGTLVTVSSASTKGPSVFPLAPSSKSTSGGTA |
| | ALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPEAA |
| | GGPSVFLFPPKPKDTLMISRTPEVTCVVVSVSHEDPEVKFNWYVDGVE |
| | VHNAKTKPREEQYNSTYRVVSVLTVLHQDWLNGKEYKCKVSNKALP |
| | APIEKTISKAKGQPREPQVYTLPPSREEMTKNQVSLWCLVKGFYPSDI |
| | AVEWESNGQPENNYKTTPPVLDSDGSFFLYSKLTVDKSRWQQGNVFS |
| | CSVMHEALHNRFTQKSLSLSPGK (SEQ ID NO: 194) |
| VH | QLQLQESGPGPVRPSETLSLTCTVSGDSIRSSSYYWGWIRQPPGKGLD |
| | WIGSIYYTGSTYYNPSLMSRVTISVDTSKNQFSLKLSSVTAADTAVYY |
| | CASPGYSSGWSIDYWGQGTLVTVSS (SEQ ID NO: 67) |
| Constant | ASTKGPSVFPLAPSSKSTSGGTAALGCLVKDYFPEPVTVSWNSGALTS |
| Region | GVHTFPAVLQSSGLYSLSSVVTVPSSSLGTQTYICNVNHKPSNTKVDK |
| | KVEPKSCDKTHTCPPCPAPEAAGGPSVFLFPPKPKDTLMISRTPEVTCV |
| | VVSVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRE |
| | EMTKNQVSLWCLVKGFYPSDIAVEWESNGQPENNYKTTPPVLDSDGS |
| | FFLYSKLTVDKSRWQQGNVFSCSVMHEALHNRFTQKSLSLSPGK |
| | (SEQ ID NO: 195) |

NKp46 Light Chain 1

| | |
|---|---|
| Full AA | DIQLTQSPSFLSASVGDRVTITCRASQGISSYLAWYQQKPGKAPKLLIY |
| sequence | VASTLQSGVPSRFSGSGSGTEFTLTISSLQPEDFATYYCQQLNSYPRMT |
| | FGGGTKVEIKRTVAAPSVFIFPPSDEQLKSGTASVVCLLNNFYPREAK |
| | VQWKVDNALQSGNSQESVTEQDSKDSTYSLSSTLTLSKADYEKHKV |
| | YACEVTHQGLSSPVTKSFNRGEC (SEQ ID NO: 163) |
| VL | DIQLTQSPSFLSASVGDRVTITCRASQGISSYLAWYQQKPGKAPKLLIY |
| | VASTLQSGVPSRFSGSGSGTEFTLTISSLQPEDFATYYCQQLNSYPRMT |
| | FGGGTKVEIK (SEQ ID NO: 68) |
| Constant | RTVAAPSVFIFPPSDEQLKSGTASVVCLLNNFYPREAKVQWKVDNAL |
| Region | QSGNSQESVTEQDSKDSTYSLSSTLTLSKADYEKHKVYACEVTHQGL |
| | SSPVTKSFNRGEC (SEQ ID NO: 179) |

BCMA Heavy Chain 2

| | |
|---|---|
| Full AA | EIVLTQSPGTLSLSPGERATLSCRASQSISSSFLTWYQQKPGQAPRLLIY |
| sequence | GASSRATGIPDRFSGGGSGTDFTLTISRLEPEDFAVYYCQHYGSSPMYT |
| | FGQGTKLEIKGGSEGKSSGSGSESKSTGGSEVQLLESGGGLVQPGGSL |
| | RLSCAASGFTFSSYAMSWVRQAPGKGLEWVSAISGSGGSTYYADSVK |
| | GRFTISRDNSKNTLYLQMNSLRAEDTAVYYCAKDEGYSSGHYYGMD |
| | VWGQGTTVTVSSEPKSSDKTHTCPPCPAPEAAGGPSVFLFPPKPKDTL |
| | MISRTPEVTCVVVSVSHEDPEVKFNWYVDGVEVHNAKTKPREEQYN |
| | STYRVVSVLTVLHQDWLNGKEYKCKVSNKALPAPIEKTISKAKGQPR |
| | EPQVYTLPPSREEMTKNQVSLSCAVKGFYPSDIAVEWESNGQPENNY |
| | KTTPPVLDSDGSFFLVSKLTVDKSRWQQGNVFSCSVMHEALHNHYTQ |
| | KSLSLSPGK (SEQ ID NO: 196) |
| VH | EVQLLESGGGLVQPGGSLRLSCAASGFTFSSYAMSWVRQAPGKGLE |
| | WVSAISGSGGSTYYADSVKGRFTISRDNSKNTLYLQMNSLRAEDTAV |
| | YYCAKDEGYSSGHYYGMDVWGQGTTVTVSS (SEQ ID NO: 99) |
| Linker | GGSEGKSSGSGSESKSTGGS (SEQ ID NO: 181) |
| VL | EIVLTQSPGTLSLSPGERATLSCRASQSISSSFLTWYQQKPGQAPRLLIY |
| | GASSRATGIPDRFSGGGSGTDFTLTISRLEPEDFAVYYCQHYGSSPMYT |
| | FGQGTKLEIK (SEQ ID NO: 100) |
| Constant | EPKSSDKTHTCPPCPAPEAAGGPSVFLFPPKPKDTLMISRTPEVTCVVV |
| Region | SVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREE |
| | MTKNQVSLSCAVKGFYPSDIAVEWESNGQPENNYKTTPPVLDSDGSF |
| | FLVSKLTVDKSRWQQGNVFSCSVMHEALHNHYTQKSLSLSPGK (SEQ |
| | ID NO: 197) |

The nucleic acid sequence information of N46BB14 is listed as below in Table 27.

TABLE 27
Nucleic acid sequence of N46BB14
NKp46 Heavy Chain 1

| | |
|---|---|
| Full DNA | CAGCTCCAGTTGCAGGAATCAGGTCCCGGTCCCGTCCGCCCCTCTG |
| sequence | AGACCCTTTCTTTGACCTGCACAGTATCCGGCGACTCTATCCGCAG |
| | CTCATCATACTACTGGGGTTGGATTCGGCAGCCTCCAGGAAAAGGT |
| | CTCGACTGGATCGGATCTATTTACTATACTGGCTCTACCTACTACA |
| | ATCCTAGCTTGATGAGCAGGGTTACCATTTCTGTCGACACCTCAAA |
| | AAATCAGTTCAGTCTTAAACTGTCCAGCGTCACTGCTGCAGACACT |
| | GCAGTGTATTATTGTGCCTCACCCGGATATTCATCCGGGTGGAGCA |
| | TTGATTATTGGGGCCAAGGTACACTCGTAACAGTCTCTTCAGCCTC |
| | CACCAAGGGCCCATCGGTCTTCCCCCTGGCACCCTCCTCCAAGAGC |
| | ACCTCTGGGGGCACAGCGGCCCTGGGCTGCCTGGTCAAGGACTAC |
| | TTCCCCGAACCGGTGACGGTGTCGTGGAACTCAGGCGCCCTGACC |
| | AGCGGCGTGCACACCTTCCCGGCTGTCCTACAGTCCTCAGGACTCT |
| | ACTCCCTCAGCAGCGTGGTGACCGTGCCCTCCAGCAGCTTGGGCAC |
| | CCAGACCTACATCTGCAACGTGAATCACAAGCCCAGCAACACCAA |
| | GGTGGACAAGAAAGTTGAGCCCAAATCTTGTGACAAAACTCACAC |
| | ATGCCCACCGTGCCCAGCACCTGAAGCCGCCGGGGGACCGTCAGT |
| | CTTCCTCTTCCCCCCAAAACCCAAGGACACCCTCATGATCTCCCGG |
| | ACCCCTGAGGTCACATGCGTGGTGGTGAGCGTGAGCCACGAAGAC |
| | CCTGAGGTCAAGTTCAACTGGTACGTGGACGGCGTGGAGGTGCAT |
| | AATGCCAAGACAAAGCCGCGGGAGGAGCAGTACAACAGCACGTA |
| | CCGTGTGGTCAGCGTCCTCACCGTCCTGCACCAGGACTGGCTGAAT |
| | GGCAAGGAGTACAAGTGCAAGGTGTCGAACAAAGCCCTCCCAGCC |
| | CCCATCGAGAAAACCATCTCCAAAGCCAAAGGGCAGCCCCGAGAA |
| | CCACAGGTGTACACCCTGCCCCCATCCCGGGAGGAGATGACCAAG |
| | AACCAGGTCAGCCTGTGGTGCCTGGTCAAAGGCTTCTATCCCAGCG |
| | ACATCGCCGTGGAGTGGGAGAGCAATGGGCAGCCGGAGAACAACT |
| | ACAAGACCACGCCTCCCGTGCTGGACTCCGACGGCTCCTTCTTCCT |
| | CTACAGCAAGCTCACCGTGGACAAGAGCAGATGGCAGCAGGGGA |
| | ACGTCTTCTCATGCTCCGTGATGCATGAGGCTCTGCACAACCGGTT |
| | CACGCAGAAGTCTCTCTCCCTGTCTCCGGGAAAA (SEQ ID NO: 198) |
| VH DNA | CAGCTCCAGTTGCAGGAATCAGGTCCCGGTCCCGTCCGCCCCTCTG |
| | AGACCCTTTCTTTGACCTGCACAGTATCCGGCGACTCTATCCGCAG |
| | CTCATCATACTACTGGGGTTGGATTCGGCAGCCTCCAGGAAAAGGT |
| | CTCGACTGGATCGGATCTATTTACTATACTGGCTCTACCTACTACA |
| | ATCCTAGCTTGATGAGCAGGGTTACCATTTCTGTCGACACCTCAAA |
| | AAATCAGTTCAGTCTTAAACTGTCCAGCGTCACTGCTGCAGACACT |
| | GCAGTGTATTATTGTGCCTCACCCGGATATTCATCCGGGTGGAGCA |
| | TTGATTATTGGGGCCAAGGTACACTCGTAACAGTCTCTTCA (SEQ |
| | ID NO: 184) |
| Constant | GCCTCCACCAAGGGCCCATCGGTCTTCCCCCTGGCACCCTCCTCCA |
| Region | AGAGCACCTCTGGGGGCACAGCGGCCCTGGGCTGCCTGGTCAAGG |
| DNA | ACTACTTCCCCGAACCGGTGACGGTGTCGTGGAACTCAGGCGCCCT |
| | GACCAGCGGCGTGCACACCTTCCCGGCTGTCCTACAGTCCTCAGGA |
| | CTCTACTCCCTCAGCAGCGTGGTGACCGTGCCCTCCAGCAGCTTGG |
| | GCACCCAGACCTACATCTGCAACGTGAATCACAAGCCCAGCAACA |
| | CCAAGGTGGACAAGAAAGTTGAGCCCAAATCTTGTGACAAAACTC |
| | ACACATGCCCACCGTGCCCAGCACCTGAAGCCGCCGGGGGACCGT |
| | CAGTCTTCCTCTTCCCCCCAAAACCCAAGGACACCCTCATGATCTC |
| | CCGGACCCCTGAGGTCACATGCGTGGTGGTGAGCGTGAGCCACGA |
| | AGACCCTGAGGTCAAGTTCAACTGGTACGTGGACGGCGTGGAGGT |
| | GCATAATGCCAAGACAAAGCCGCGGGAGGAGCAGTACAACAGCA |
| | CGTACCGTGTGGTCAGCGTCCTCACCGTCCTGCACCAGGACTGGCT |
| | GAATGGCAAGGAGTACAAGTGCAAGGTGTCGAACAAAGCCCTCCC |
| | AGCCCCCATCGAGAAAACCATCTCCAAAGCCAAAGGGCAGCCCCG |
| | AGAACCACAGGTGTACACCCTGCCCCCATCCCGGGAGGAGATGAC |
| | CAAGAACCAGGTCAGCCTGTGGTGCCTGGTCAAAGGCTTCTATCCC |
| | AGCGACATCGCCGTGGAGTGGGAGAGCAATGGGCAGCCGGAGAA |
| | CAACTACAAGACCACGCCTCCCGTGCTGGACTCCGACGGCTCCTTC |
| | TTCCTCTACAGCAAGCTCACCGTGGACAAGAGCAGATGGCAGCAG |
| | GGGAACGTCTTCTCATGCTCCGTGATGCATGAGGCTCTGCACAACC |
| | GGTTCACGCAGAAGTCTCTCTCCCTGTCTCCGGGAAAA (SEQ ID |
| | NO: 199) |

NKp46 Light Chain 1

| | |
|---|---|
| Full DNA | GACATCCAGCTGACCCAGAGCCCCAGCTTTCTGAGCGCCAGCGTG |
| sequence | GGCGACAGGGTGACCATCACCTGCAGGGCCAGCCAGGGCATCAGC |
| | AGCTACCTGGCCTGGTACCAGCAGAAGCCCGGCAAAGCCCCCAAG |
| | CTGCTGATCTACGTGGCCAGCACACTGCAGAGCGGCGTGCCTAGC |
| | AGATTTAGCGGCAGCGGCAGCGGCACAGAGTTCACCCTGACCATC |
| | TCCAGCCTGCAGCCCGAGGACTTCGCCACCTACTACTGCCAGCAGC |
| | TGAACAGCTACCCCAGGATGACCTTCGGCGGCGGCACCAAGGTGG |
| | AGATCAAGCGTACGGTGGCTGCACCATCTGTCTTCATCTTCCCGCC |
| | ATCTGATGAGCAGTTGAAATCTGGAACTGCCTCTGTTGTGTGCCTG |
| | CTGAATAACTTCTATCCCAGAGAGGCCAAAGTACAGTGGAAGGTG |
| | GATAACGCCCTCCAATCGGGTAACTCCCAGGAGAGTGTCACAGAG |
| | CAGGACAGCAAGGACAGCACCTACAGCCTCAGCAGCACCCTGACG |
| | CTGAGCAAAGCAGACTACGAGAAACACAAAGTCTACGCCTGCGAA |
| | GTCACCCATCAGGGCCTGAGCTCGCCCGTCACAAAGAGCTTCAAC |
| | AGGGGAGAGTGT (SEQ ID NO: 186) |
| VL DNA | GACATCCAGCTGACCCAGAGCCCCAGCTTTCTGAGCGCCAGCGTG |
| | GGCGACAGGGTGACCATCACCTGCAGGGCCAGCCAGGGCATCAGC |
| | AGCTACCTGGCCTGGTACCAGCAGAAGCCCGGCAAAGCCCCCAAG |
| | CTGCTGATCTACGTGGCCAGCACACTGCAGAGCGGCGTGCCTAGC |
| | AGATTTAGCGGCAGCGGCAGCGGCACAGAGTTCACCCTGACCATC |
| | TCCAGCCTGCAGCCCGAGGACTTCGCCACCTACTACTGCCAGCAGC |
| | TGAACAGCTACCCCAGGATGACCTTCGGCGGCGGCACCAAGGTGG |
| | AGATCAAG (SEQ ID NO: 187) |
| Constant | CGTACGGTGGCTGCACCATCTGTCTTCATCTTCCCGCCATCTGATG |
| Region | AGCAGTTGAAATCTGGAACTGCCTCTGTTGTGTGCCTGCTGAATAA |
| DNA | CTTCTATCCCAGAGAGGCCAAAGTACAGTGGAAGGTGGATAACGC |
| | CCTCCAATCGGGTAACTCCCAGGAGAGTGTCACAGAGCAGGACAG |
| | CAAGGACAGCACCTACAGCCTCAGCAGCACCCTGACGCTGAGCAA |
| | AGCAGACTACGAGAAACACAAAGTCTACGCCTGCGAAGTCACCCA |
| | TCAGGGCCTGAGCTCGCCCGTCACAAAGAGCTTCAACAGGGGAGA |
| | GTGT (SEQ ID NO: 188) |

BCMA Heavy Chain 2

| | |
|---|---|
| Full DNA | GAGATCGTGCTGACTCAGAGCCCTGGCACTCTGAGCCTGAGCCCTG |
| Sequence | GCGAGAGGGCCACTCTGAGCTGTAGGGCCAGCCAGAGCATCAGCA |
| | GCAGCTTCCTGACTTGGTACCAGCAGAAGCCTGGCCAGGCCCCTA |
| | GGCTGCTGATCTACGGCGCCAGCAGCAGGGCCACTGGCATCCCTG |
| | ATAGGTTCAGCGGCGGCGGCAGCGGCACTGATTTCACTCTGACTAT |
| | CAGCAGGCTGGAGCCTGAGGATTTCGCCGTGTACTACTGTCAGCAC |
| | TACGGCAGCAGCCCTATGTACACTTTCGGCCAGGGCACTAAGCTG |
| | GAGATCAAGGGCGGCTCCGAGGGCAAGAGCAGCGGCAGCGGCAG |
| | CGAGAGCAAGAGCACCGGCGGCAGCGAAGTGCAGCTGCTGGAGTC |
| | AGGGGGCGGTCTGGTACAACCGGGCGGCAGCCTGAGGCTGAGCTG |
| | TGCCGCCAGCGGCTTCACTTTCAGCAGCTACGCCATGAGCTGGGTG |
| | AGGCAGGCCCCTGGCAAGGGCCTGGAGTGGGTGAGCGCCATCAGC |
| | GGCAGCGGCGGCAGCACTTACTACGCCGATAGCGTGAAGGGCAGG |
| | TTCACTATCAGCAGGGATAATAGCAAGAATACTCTGTACCTGCAG |
| | ATGAATAGCCTGAGGGCCGAGGATACTGCCGTGTACTACTGTGCC |
| | AAGGATGAGGGCTACAGCAGCGGCCACTACTACGGCATGGATGTG |
| | TGGGGCCAGGGCACTACTGTGACTGTGAGCAGCGAGCCCAAATCT |
| | AGCGACAAAACTCACACATGTCCACCGTGCCCAGCACCTGAAGCA |
| | GCAGGGGGACCGTCAGTCTTCCTCTTCCCCCCAAAACCCAAGGAC |
| | ACCCTCATGATCTCCCGGACCCCTGAGGTCACATGCGTGGTGGTGA |
| | GCGTGAGCCACGAAGACCCTGAGGTCAAGTTCAACTGGTACGTGG |
| | ACGGCGTGGAGGTGCATAATGCCAAGACAAAGCCGCGGGAGGAG |
| | CAGTACAACAGCACGTACCGTGTGGTCAGCGTCCTCACCGTCCTGC |
| | ACCAGGACTGGCTGAATGGCAAGGAGTACAAGTGCAAGGTCTCCA |
| | ACAAAGCCCTCCCAGCCCCCATCGAGAAAACCATCTCCAAAGCCA |
| | AAGGGCAGCCCCGAGAACCACAGGTGTACACCCTGCCCCCATCCC |
| | GGGAGGAGATGACCAAGAACCAGGTCAGCCTGTCCTGCGCCGTCA |
| | AAGGCTTCTATCCCAGCGACATCGCCGTGGAGTGGGAGAGCAATG |
| | GGCAGCCGGAGAACAACTACAAGACCACGCCTCCCGTGCTGGACT |
| | CCGACGGCTCCTTCTTCCTCGTGAGCAAGCTCACCGTGGACAAGTC |
| | TAGATGGCAGCAGGGGAACGTCTTCTCATGCTCCGTGATGCATGA |
| | GGCTCTGCACAACCACTACACGCAGAAGAGCCTCTCCCTGTCTCCG |
| | GGTAAA (SEQ ID NO: 200) |
| VH DNA | GAAGTGCAGCTGCTGGAGTCAGGGGGCGGTCTGGTACAACCGGGC |
| | GGCAGCCTGAGGCTGAGCTGTGCCGCCAGCGGCTTCACTTTCAGCA |
| | GCTACGCCATGAGCTGGGTGAGGCAGGCCCCTGGCAAGGGCCTGG |
| | AGTGGGTGAGCGCCATCAGCGGCAGCGGCGGCAGCACTTACTACG |
| | CCGATAGCGTGAAGGGCAGGTTCACTATCAGCAGGGATAATAGCA |
| | AGAATACTCTGTACCTGCAGATGAATAGCCTGAGGGCCGAGGATA |
| | CTGCCGTGTACTACTGTGCCAAGGATGAGGGCTACAGCAGCGGCC |
| | ACTACTACGGCATGGATGTGTGGGGCCAGGGCACTACTGTGACTG |
| | TGAGCAGC (SEQ ID NO: 190) |
| Linker | GGCGGCTCCGAGGGCAAGAGCAGCGGCAGCGGCAGCGAGAGCAA |
| DNA | GAGCACCGGCGGCAGC (SEQ ID NO: 191) |
| VL DNA | GAGATCGTGCTGACTCAGAGCCCTGGCACTCTGAGCCTGAGCCCTG |
| | GCGAGAGGGCCACTCTGAGCTGTAGGGCCAGCCAGAGCATCAGCA |
| | GCAGCTTCCTGACTTGGTACCAGCAGAAGCCTGGCCAGGCCCCTA |
| | GGCTGCTGATCTACGGCGCCAGCAGCAGGGCCACTGGCATCCCTG |
| | ATAGGTTCAGCGGCGGCGGCAGCGGCACTGATTTCACTCTGACTAT |
| | CAGCAGGCTGGAGCCTGAGGATTTCGCCGTGTACTACTGTCAGCAC |
| | TACGGCAGCAGCCCTATGTACACTTTCGGCCAGGGCACTAAGCTG |
| | GAGATCAAG (SEQ ID NO: 192) |
| Constant | GAGCCCAAATCTAGCGACAAAACTCACACATGTCCACCGTGCCCA |
| Region | GCACCTGAAGCAGCAGGGGGACCGTCAGTCTTCCTCTTCCCCCCAA |
| DNA | AACCCAAGGACACCCTCATGATCTCCCGGACCCCTGAGGTCACAT |
| | GCGTGGTGGTGAGCGTGAGCCACGAAGACCCTGAGGTCAAGTTCA |
| | ACTGGTACGTGGACGGCGTGGAGGTGCATAATGCCAAGACAAAGC |
| | CGCGGGAGGAGCAGTACAACAGCACGTACCGTGTGGTCAGCGTCC |
| | TCACCGTCCTGCACCAGGACTGGCTGAATGGCAAGGAGTACAAGT |
| | GCAAGGTCTCCAACAAAGCCCTCCCAGCCCCCATCGAGAAAACCA |
| | TCTCCAAAGCCAAAGGGCAGCCCCGAGAACCACAGGTGTACACCC |
| | TGCCCCCATCCCGGGAGGAGATGACCAAGAACCAGGTCAGCCTGT |
| | CCTGCGCCGTCAAAGGCTTCTATCCCAGCGACATCGCCGTGGAGTG |
| | GGAGAGCAATGGGCAGCCGGAGAACAACTACAAGACCACGCCTCC |
| | CGTGCTGGACTCCGACGGCTCCTTCTTCCTCGTGAGCAAGCTCACC |
| | GTGGACAAGTCTAGATGGCAGCAGGGGAACGTCTTCTCATGCTCC |
| | GTGATGCATGAGGCTCTGCACAACCACTACACGCAGAAGAGCCTC |
| | TCCCTGTCTCCGGGTAAA (SEQ ID NO: 201) |

The amino acid sequence information of BCMB1106 is listed as below in Table 28.

TABLE 28
Amino acid sequence of BCMB1106
Fc only

| | |
|---|---|
| Full AA | DKTHTCPPCPAPELLGGPSVFLFPPKPEDTLMISRTPEVTCVVVDVSHE |
| sequence | DPEVKFNWYVDGVEVHNAKTKPREEQYNSTYRVVSVLTVLHQDWL |
| | NGKEYKCKVSNKALPAPIEKTISKAKGQPREPQVYTLPPSREEMTKNQ |
| | VSLWCLVKGFYPSDIAVEWESNGQPENNYKTTPPVLDSDGSFFLYSKL |
| | TVDKSRWQQGNVFSCSVMHEALHNHYRQKSLSLSPGK (SEQ ID |
| | NO: 202) |
| Constant | DKTHTCPPCPAPELLGGPSVFLFPPKPEDTLMISRTPEVTCVVVDVSHE |
| Region | DPEVKFNWYVDGVEVHNAKTKPREEQYNSTYRVVSVLTVLHQDWL |
| | NGKEYKCKVSNKALPAPIEKTISKAKGQPREPQVYTLPPSREEMTKNQ |
| | VSLWCLVKGFYPSDIAVEWESNGQPENNYKTTPPVLDSDGSFFLYSKL |
| | TVDKSRWQQGNVFSCSVMHEALHNHYRQKSLSLSPGK (SEQ ID |
| | NO: 202) |

BCMA Heavy Chain 2

| | |
|---|---|
| Full AA | EVQLLESGGGLVQPGGSLRLSCAASGFTFSSYAMSWVRQAPGKGLE |
| sequence | WVSAISGSGGSTYYADSVKGRFTISRDNSKNTLYLQMNSLRAEDTAV |
| | YYCAKDEGYSSGHYYGMDVWGQGTTVTVSSASTKGPSVFPLAPSSK |
| | STSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPP |
| | CPAPELLGGPSVFLFPPKPEDTLMISRTPEVTCVVVDVSHEDPEVKFN |
| | WYVDGVEVHNAKTKPREEQYNSTYRVVSVLTVLHQDWLNGKEYKC |
| | KVSNKALPAPIEKTISKAKGQPREPQVYTLPPSREEMTKNQVSLSCAV |
| | KGFYPSDIAVEWESNGQPENNYKTTPPVLDSDGSFFLVSKLTVDKSR |
| | WQQGNVFSCSVMHEALHNRFRQKSLSLSPGK (SEQ ID NO: 203) |
| VH | EVQLLESGGGLVQPGGSLRLSCAASGFTFSSYAMSWVRQAPGKGLE |
| | WVSAISGSGGSTYYADSVKGRFTISRDNSKNTLYLQMNSLRAEDTAV |
| | YYCAKDEGYSSGHYYGMDVWGQGTTVTVSS (SEQ ID NO: 99) |
| Constant | ASTKGPSVFPLAPSSKSTSGGTAALGCLVKDYFPEPVTVSWNSGALTS |
| Region | GVHTFPAVLQSSGLYSLSSVVTVPSSSLGTQTYICNVNHKPSNTKVDK |
| | KVEPKSCDKTHTCPPCPAPELLGGPSVFLFPPKPEDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRE |
| | EMTKNQVSLSCAVKGFYPSDIAVEWESNGQPENNYKTTPPVLDSDGS |
| | FFLVSKLTVDKSRWQQGNVFSCSVMHEALHNRFRQKSLSLSPGK |
| | (SEQ ID NO: 204) |

BCMA Light Chain 2

| | |
|---|---|
| Full AA | EIVLTQSPGTLSLSPGERATLSCRASQSISSSFLTWYQQKPGQAPRLLIY |
| sequence | GASSRATGIPDRFSGGGSGTDFTLTISRLEPEDFAVYYCQHYGSSPMYT |
| | FGQGTKLEIKRTVAAPSVFIFPPSDEQLKSGTASVVCLLNNFYPREAKV |
| | QWKVDNALQSGNSQESVTEQDSKDSTYSLSSTLTLSKADYEKHKVY |
| | ACEVTHQGLSSPVTKSFNRGEC (SEQ ID NO: 205) |
| VL | EIVLTQSPGTLSLSPGERATLSCRASQSISSSFLTWYQQKPGQAPRLLIY |
| | GASSRATGIPDRFSGGGSGTDFTLTISRLEPEDFAVYYCQHYGSSPMYT |
| | FGQGTKLEIK (SEQ ID NO: 100) |
| Constant | RTVAAPSVFIFPPSDEQLKSGTASVVCLLNNFYPREAKVQWKVDNAL |
| Region | QSGNSQESVTEQDSKDSTYSLSSTLTLSKADYEKHKVYACEVTHQGL |
| | SSPVTKSFNRGEC (SEQ ID NO: 206) |

The nucleic acid sequence information of BCMB1106 is listed as below in Table 29:

TABLE 29
Nucleic acid sequence of BCMB1106
Fc only

| | |
|---|---|
| Full DNA | GACAAAACTCACACATGTCCACCGTGCCCAGCACCTGAACTGCTG |
| sequence | GGGGGACCGTCAGTCTTCCTCTTCCCCCCAAAACCCGAGGACACCC |
| | TCATGATCTCCCGGACCCCTGAGGTCACATGCGTGGTGGTGGACGT |
| | GAGCCACGAAGACCCTGAGGTCAAGTTCAACTGGTACGTGGACGG |
| | CGTGGAGGTGCATAATGCCAAGACAAAGCCGCGGGAGGAGCAGT |
| | ACAACAGCACGTACCGTGTGGTCAGCGTCCTCACCGTCCTGCACCA |
| | GGACTGGCTGAATGGCAAGGAGTACAAGTGCAAGGTCTCCAACAA |
| | AGCCCTCCCAGCCCCCATCGAGAAAACCATCTCCAAAGCCAAAGG |
| | GCAGCCCCGAGAACCACAGGTGTACACCCTGCCCCCATCCCGGGA |
| | GGAGATGACCAAGAACCAGGTCAGCCTGTGGTGCCTGGTCAAAGG |
| | CTTCTATCCCAGCGACATCGCCGTGGAGTGGGAGAGCAATGGGCA |
| | GCCGGAGAACAACTACAAGACCACGCCTCCCGTGCTGGACTCCGA |
| | CGGCTCCTTCTTCCTCTACAGCAAGCTCACCGTGGACAAGTCTAGA |
| | TGGCAGCAGGGGAACGTCTTCTCATGCTCCGTGATGCATGAGGCTC |
| | TGCACAACCACTACCGGCAGAAGAGCCTCTCCCTGTCTCCGGGTAA |
| | A (SEQ ID NO: 207) |
| Constant | GACAAAACTCACACATGTCCACCGTGCCCAGCACCTGAACTGCTG |
| Region | GGGGGACCGTCAGTCTTCCTCTTCCCCCCAAAACCCGAGGACACCC |
| DNA | TCATGATCTCCCGGACCCCTGAGGTCACATGCGTGGTGGTGGACGT |
| | GAGCCACGAAGACCCTGAGGTCAAGTTCAACTGGTACGTGGACGG |
| | CGTGGAGGTGCATAATGCCAAGACAAAGCCGCGGGAGGAGCAGT |
| | ACAACAGCACGTACCGTGTGGTCAGCGTCCTCACCGTCCTGCACCA |
| | GGACTGGCTGAATGGCAAGGAGTACAAGTGCAAGGTCTCCAACAA |
| | AGCCCTCCCAGCCCCCATCGAGAAAACCATCTCCAAAGCCAAAGG |
| | GCAGCCCCGAGAACCACAGGTGTACACCCTGCCCCCATCCCGGGA |
| | GGAGATGACCAAGAACCAGGTCAGCCTGTGGTGCCTGGTCAAAGG |
| | CTTCTATCCCAGCGACATCGCCGTGGAGTGGGAGAGCAATGGGCA |
| | GCCGGAGAACAACTACAAGACCACGCCTCCCGTGCTGGACTCCGA |
| | CGGCTCCTTCTTCCTCTACAGCAAGCTCACCGTGGACAAGTCTAGA |
| | TGGCAGCAGGGGAACGTCTTCTCATGCTCCGTGATGCATGAGGCTC |
| | TGCACAACCACTACCGGCAGAAGAGCCTCTCCCTGTCTCCGGGTAA |
| | A (SEQ ID NO: 207) |

BCMA Heavy Chain 2

| | |
|---|---|
| Full DNA | GAAGTGCAGCTGCTGGAGTCAGGGGGCGGTCTGGTACAACCGGGC |
| sequence | GGCAGCCTGAGGCTGAGCTGTGCCGCCAGCGGCTTCACTTTCAGCA |
| | GCTACGCCATGAGCTGGGTGAGGCAGGCCCCTGGCAAGGGCCTGG |
| | AGTGGGTGAGCGCCATCAGCGGCAGCGGCGGCAGCACTTACTACG |
| | CCGATAGCGTGAAGGGCAGGTTCACTATCAGCAGGGATAATAGCA |
| | AGAATACTCTGTACCTGCAGATGAATAGCCTGAGGGCCGAGGATA |
| | CTGCCGTGTACTACTGTGCCAAGGATGAGGGCTACAGCAGCGGCC |
| | ACTACTACGGCATGGATGTGTGGGGCCAGGGCACTACTGTGACTG |
| | TGAGCAGCGCCTCCACCAAGGGCCCATCGGTCTTCCCCCTGGCACC |
| | CTCCTCCAAGAGCACCTCTGGGGGCACAGCGGCCCTGGGCTGCCT |
| | GGTCAAGGACTACTTCCCCGAACCGGTGACGGTGTCGTGGAACTC |
| | AGGCGCCCTGACCAGCGGCGTGCACACCTTCCCGGCTGTCCTACAG |
| | TCCTCAGGACTCTACTCCCTCAGCAGCGTGGTGACCGTGCCCTCCA |
| | GCAGCTTGGGCACCCAGACCTACATCTGCAACGTGAATCACAAGC |
| | CCAGCAACACCAAGGTGGACAAGAAAGTTGAGCCCAAATCTTGTG |
| | ACAAAACTCACACATGTCCACCGTGCCCAGCACCTGAACTGCTGG |
| | GGGGACCGTCAGTCTTCCTCTTCCCCCCAAAACCCGAGGACACCCT |
| | CATGATCTCCCGGACCCCTGAGGTCACATGCGTGGTGGTGGACGTG |
| | AGCCACGAAGACCCTGAGGTCAAGTTCAACTGGTACGTGGACGGC |
| | GTGGAGGTGCATAATGCCAAGACAAAGCCGCGGGAGGAGCAGTA |
| | CAACAGCACGTACCGTGTGGTCAGCGTCCTCACCGTCCTGCACCAG |
| | GACTGGCTGAATGGCAAGGAGTACAAGTGCAAGGTCTCCAACAAA |
| | GCCCTCCCAGCCCCCATCGAGAAAACCATCTCCAAAGCCAAAGGG |
| | CAGCCCCGAGAACCACAGGTGTACACCCTGCCCCCATCCCGGGAG |
| | GAGATGACCAAGAACCAGGTCAGCCTGTCCTGCGCCGTCAAAGGC |
| | TTCTATCCCAGCGACATCGCCGTGGAGTGGGAGAGCAATGGGCAG |
| | CCGGAGAACAACTACAAGACCACGCCTCCCGTGCTGGACTCCGAC |
| | GGCTCCTTCTTCCTCGTGAGCAAGCTCACCGTGGACAAGTCTAGAT |
| | GGCAGCAGGGGAACGTCTTCTCATGCTCCGTGATGCATGAGGCTCT |
| | GCACAACCGGTTCCGCCAGAAGAGCCTCTCCCTGTCTCCGGGTAAA |
| | (SEQ ID NO: 208) |
| VH DNA | GAAGTGCAGCTGCTGGAGTCAGGGGGCGGTCTGGTACAACCGGGC |
| | GGCAGCCTGAGGCTGAGCTGTGCCGCCAGCGGCTTCACTTTCAGCA |
| | GCTACGCCATGAGCTGGGTGAGGCAGGCCCCTGGCAAGGGCCTGG |
| | AGTGGGTGAGCGCCATCAGCGGCAGCGGCGGCAGCACTTACTACG |
| | CCGATAGCGTGAAGGGCAGGTTCACTATCAGCAGGGATAATAGCA |
| | AGAATACTCTGTACCTGCAGATGAATAGCCTGAGGGCCGAGGATA |
| | CTGCCGTGTACTACTGTGCCAAGGATGAGGGCTACAGCAGCGGCC |
| | ACTACTACGGCATGGATGTGTGGGGCCAGGGCACTACTGTGACTG |
| | TGAGCAGC (SEQ ID NO: 190) |
| Constant | GCCTCCACCAAGGGCCCATCGGTCTTCCCCCTGGCACCCTCCTCCA |
| Region | AGAGCACCTCTGGGGGCACAGCGGCCCTGGGCTGCCTGGTCAAGG |
| DNA | ACTACTTCCCCGAACCGGTGACGGTGTCGTGGAACTCAGGCGCCCT |
| | GACCAGCGGCGTGCACACCTTCCCGGCTGTCCTACAGTCCTCAGGA |
| | CTCTACTCCCTCAGCAGCGTGGTGACCGTGCCCTCCAGCAGCTTGG |
| | GCACCCAGACCTACATCTGCAACGTGAATCACAAGCCCAGCAACA |
| | CCAAGGTGGACAAGAAAGTTGAGCCCAAATCTTGTGACAAAACTC |
| | ACACATGTCCACCGTGCCCAGCACCTGAACTGCTGGGGGGACCGT |
| | CAGTCTTCCTCTTCCCCCCAAAACCCGAGGACACCCTCATGATCTC |
| | CCGGACCCCTGAGGTCACATGCGTGGTGGTGGACGTGAGCCACGA |
| | AGACCCTGAGGTCAAGTTCAACTGGTACGTGGACGGCGTGGAGGT |
| | GCATAATGCCAAGACAAAGCCGCGGGAGGAGCAGTACAACAGCA |
| | CGTACCGTGTGGTCAGCGTCCTCACCGTCCTGCACCAGGACTGGCT |
| | GAATGGCAAGGAGTACAAGTGCAAGGTCTCCAACAAAGCCCTCCC |
| | AGCCCCCATCGAGAAAACCATCTCCAAAGCCAAAGGGCAGCCCCG |
| | AGAACCACAGGTGTACACCCTGCCCCCATCCCGGGAGGAGATGAC |
| | CAAGAACCAGGTCAGCCTGTCCTGCGCCGTCAAAGGCTTCTATCCC |
| | AGCGACATCGCCGTGGAGTGGGAGAGCAATGGGCAGCCGGAGAA |
| | CAACTACAAGACCACGCCTCCCGTGCTGGACTCCGACGGCTCCTTC |
| | TTCCTCGTGAGCAAGCTCACCGTGGACAAGTCTAGATGGCAGCAG |
| | GGGAACGTCTTCTCATGCTCCGTGATGCATGAGGCTCTGCACAACC |
| | GGTTCCGCCAGAAGAGCCTCTCCCTGTCTCCGGGTAAA (SEQ ID |
| | NO: 209) |

BCMA Light Chain 2

| | |
|---|---|
| Full DNA | GAGATAGTTCTTACCCAATCTCCTGGAACCCTCAGTCTCAGTCCAG |
| sequence | GTGAGAGAGCTACTCTCTCATGTAGAGCAAGCCAATCTATTAGTTC |
| | TAGCTTTCTCACCTGGTATCAGCAAAAGCCAGGACAAGCTCCTCGT |
| | TTGCTTATTTATGGAGCATCATCTAGGGCCACTGGAATACCTGATC |
| | GTTTCAGTGGTGGCGGTTCAGGGACCGACTTTACACTGACTATAAG |
| | CAGATTGGAACCAGAAGACTTTGCTGTCTACTATTGTCAACATTAC |
| | GGATCCTCCCCAATGTACACATTTGGTCAAGGCACAAAATTGGAG |
| | ATAAAGCGGACAGTGGCCGCTCCTTCCGTGTTCATCTTCCCACCTT |
| | CCGACGAGCAGCTGAAGTCCGGCACAGCTTCTGTCGTGTGCCTGCT |
| | GAACAACTTCTACCCTCGGGAAGCCAAGGTGCAGTGGAAGGTGGA |
| | CAATGCCCTGCAGTCCGGCAACTCCCAAGAGTCTGTGACCGAGCA |
| | GGACTCCAAGGACAGCACCTACAGCCTGTCCTCCACACTGACCCTG |
| | TCCAAGGCCGACTACGAGAAGCACAAGGTGTACGCCTGCGAAGTG |
| | ACCCATCAGGGCCTGTCTAGCCCTGTGACCAAGTCTTTCAACCGGG |
| | GCGAGTGT (SEQ ID NO: 210) |
| VL DNA | GAGATAGTTCTTACCCAATCTCCTGGAACCCTCAGTCTCAGTCCAG |
| | GTGAGAGAGCTACTCTCTCATGTAGAGCAAGCCAATCTATTAGTTC |
| | TAGCTTTCTCACCTGGTATCAGCAAAAGCCAGGACAAGCTCCTCGT |
| | TTGCTTATTTATGGAGCATCATCTAGGGCCACTGGAATACCTGATC |
| | GTTTCAGTGGTGGCGGTTCAGGGACCGACTTTACACTGACTATAAG |
| | CAGATTGGAACCAGAAGACTTTGCTGTCTACTATTGTCAACATTAC |
| | GGATCCTCCCCAATGTACACATTTGGTCAAGGCACAAAATTGGAG |
| | ATAAAG (SEQ ID NO: 211) |
| Constant | CGGACAGTGGCCGCTCCTTCCGTGTTCATCTTCCCACCTTCCGACG |
| Region | AGCAGCTGAAGTCCGGCACAGCTTCTGTCGTGTGCCTGCTGAACAA |
| DNA | CTTCTACCCTCGGGAAGCCAAGGTGCAGTGGAAGGTGGACAATGC |
| | CCTGCAGTCCGGCAACTCCCAAGAGTCTGTGACCGAGCAGGACTC |
| | CAAGGACAGCACCTACAGCCTGTCCTCCACACTGACCCTGTCCAAG |
| | GCCGACTACGAGAAGCACAAGGTGTACGCCTGCGAAGTGACCCAT |
| | CAGGGCCTGTCTAGCCCTGTGACCAAGTCTTTCAACCGGGGCGAGT |
| | GT (SEQ ID NO: 212) |

### 7.7 Example 7: Lack of Anti-NK Cytotoxicity

The finding that bispecific NK engager (N46BB10.AFU) is active by ADCC mechanism and is superior (FIG. 7 and FIG. 8A-C) than the corresponding antibody (BCMB1106.AFU) also demonstrated that the bispecific NK engagers do not exert detrimental effects to NK cells in the fratricide manner.

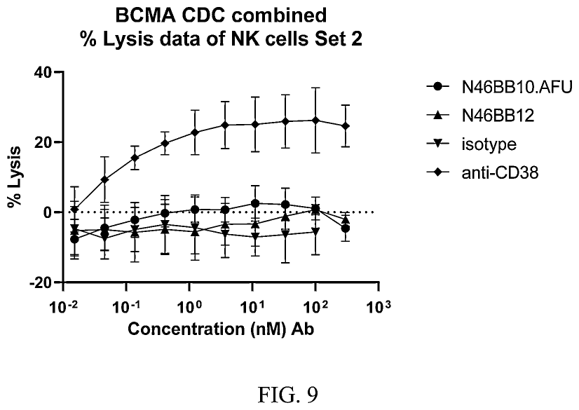

To evaluate whether the bispecific antibodies containing an NKp46 binder would lead to anti-NK cytotoxicity by CDC mechanism, purified NK cells were incubated with the bispecific antibodies in the presence of 40% human sera for 12 hours. Viability of the NK cells were then measured using Cell-Titerglo (Promega, Madison, WI) as indicated by the manufacturer. An anti-CD38 antibody (which is known to cause anti-NK cytotoxicity by CDC) and a non-specific isotype control, were used as positive and negative controls. As shown in FIG. 9, the anti-CD38 antibody shows CDC mediated killing of NK cells. In contrast, the NKp46 x BCMA bispecific antibodies with either WT IgG1 backbone (N46BB10.AFU) or even a CDC-enhancing set of mutations (K248E/T437R, N46BB12) do not cause CDC killing of NK cells. These data indicate that incorporation of the NKp46 binder in the bispecific format tested does not lead to anti-NK cytotoxicity.

### 7.8 Example 8: Therapeutic Potential

In the present invention, it is shown that incorporation of anti-NKp46 binder in a tumor targeting bsAb can potentiate cytotoxic activity against tumor targets by NKp46-expressing immune effector cells. As recent studies began to uncover that NKp46 is not exclusively on NK cells, but is also expressed on T cell subsets including gamma delta T cells (Mikulak et al, JCI Insight. 2019), as wells as a mucosal population of innate lymphoid cells (Nami-Maninelli, et al, PNAS 2011), these NKp46-containing molecules have the potential to induce tumor target killing by other NKp46-expressing immune cell types than NK cells. Such mechanisms of action by multiple immune effector cells may differentiate the present invention from other effector function by monoclonal and bispecific therapeutic antibodies.

What is claimed:

1. A multispecific antibody comprising:
(a) a first binding domain that binds to a first antigen expressed on a Natural Killer (NK) cell, and
(b) a second binding domain that binds to a second antigen.

2. The multispecific antibody of claim 1, wherein the first antigen is an NK cell activating receptor.

3. The multispecific antibody of claim 2, wherein the first antigen is NKG2d.

4. The multispecific antibody of claim 3, wherein the first binding domain comprises:
(i) a heavy chain variable region (VH) comprising
(a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6;
(b) a VH complementarity determining region (CDR) 1 having an amino acid sequence of SEQ ID NO: 10, a VH CDR2 having an amino acid sequence of SEQ ID NO: 11, and a VH CDR3 having an amino acid sequence of SEQ ID NO:12;
(c) a VH complementarity determining region (CDR) 1 having an amino acid sequence of SEQ ID NO: 16, a VH CDR2 having an amino acid sequence of SEQ ID NO: 17, and a VH CDR3 having an amino acid sequence of SEQ ID NO:18;
(d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or
(e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30;
and
a light chain variable region (VL) comprising
(a) a VL CDR1 having an amino acid sequence of SEQ ID NO: 7, a VL CDR2 having an amino acid sequence of SEQ ID NO:8, and a VL CDR3 having an amino acid sequence of SEQ ID NO:9;
(b) a VL CDR1 having an amino acid sequence of SEQ ID NO: 13, a VL CDR2 having an amino acid sequence of SEQ ID NO: 14, and a VL CDR3 having an amino acid sequence of SEQ ID NO: 15;
(c) a VL CDR1 having an amino acid sequence of SEQ ID NO: 19, a VL CDR2 having an amino acid sequence of SEQ ID NO:20, and a VL CDR3 having an amino acid sequence of SEQ ID NO:21;
(d) a VL CDR1 having an amino acid sequence of SEQ ID NO:25, a VL CDR2 having an amino acid sequence of SEQ ID NO:26, and a VL CDR3 having an amino acid sequence of SEQ ID NO:27; or
(e) a VL CDR1 having an amino acid sequence of SEQ ID NO:31, a VL CDR2 having an amino acid sequence of SEQ ID NO:32, and a VL CDR3 having an amino acid sequence of SEQ ID NO:33;
or
(ii) a heavy chain variable region (VH) comprising
(a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38;
(b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44;
(c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50;
(d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or
(e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62;
and
a light chain variable region (VL) comprising
(a) a VL CDR1 having an amino acid sequence of SEQ ID NO:39, a VL CDR2 having an amino acid sequence of SEQ ID NO:40, and a VL CDR3 having an amino acid sequence of SEQ ID NO:41;
(b) a VL CDR1 having an amino acid sequence of SEQ ID NO:45, a VL CDR2 having an amino acid sequence of SEQ ID NO:46, and a VL CDR3 having an amino acid sequence of SEQ ID NO:47;
(c) a VL CDR1 having an amino acid sequence of SEQ ID NO:51, a VL CDR2 having an amino acid sequence of SEQ ID NO:52, and a VL CDR3 having an amino acid sequence of SEQ ID NO:53;
(d) a VL CDR1 having an amino acid sequence of SEQ ID NO:57, a VL CDR2 having an amino acid sequence of SEQ ID NO:58, and a VL CDR3 having an amino acid sequence of SEQ ID NO:59: or
(e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.

5. The multispecific antibody of claim 4, wherein the first binding domain comprises a VH having an amino acid sequence of SEQ ID NO:2, and a VL having an amino acid sequence of SEQ ID NO:3, or wherein the first binding domain comprises: a VH having an amino acid sequence of SEQ ID NO:34, and a VL having an amino acid sequence of SEQ ID NO:35.

6. The multispecific antibody of claim 2, wherein the first antigen is NKp46.

7. The multispecific antibody of claim 6, wherein the first binding domain comprises:
(i) a heavy chain variable region (VH) comprising
(a) a VH complementarity determining region (CDR) 1 having an amino acid sequence of SEQ ID NO:69, a VH CDR2 having an amino acid sequence of SEQ ID NO: 70, and a VH CDR3 having an amino acid sequence of SEQ ID NO:71;
(b) a VH complementarity determining region (CDR) 1 having an amino acid sequence of SEQ ID NO:75, a VH CDR2 having an amino acid sequence of SEQ ID NO: 76, and a VH CDR3 having an amino acid sequence of SEQ ID NO:77;
(c) a VH complementarity determining region (CDR) 1 having an amino acid sequence of SEQ ID NO:81, a VH CDR2 having an amino acid sequence of SEQ ID NO: 82, and a VH CDR3 having an amino acid sequence of SEQ ID NO:83;
(d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or
(e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95;
and
(ii) a light chain variable region (VL) comprising
(a) a VL CDR1 having an amino acid sequence of SEQ ID NO: 72, a VL CDR2 having an amino acid sequence of SEQ ID NO:73, and a VL CDR3 having an amino acid sequence of SEQ ID NO:74;
(b) a VL CDR1 having an amino acid sequence of SEQ ID NO: 78, a VL CDR2 having an amino acid sequence of SEQ ID NO:79, and a VL CDR3 having an amino acid sequence of SEQ ID NO:80;
(c) a VL CDR1 having an amino acid sequence of SEQ ID NO:84, a VL CDR2 having an amino acid sequence of SEQ ID NO:85, and a VL CDR3 having an amino acid sequence of SEQ ID NO:86;
(d) a VL CDR1 having an amino acid sequence of SEQ ID NO:90, a VL CDR2 having an amino acid sequence of SEQ ID NO:91, and a VL CDR3 having an amino acid sequence of SEQ ID NO:92: or
(e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.

8. The multispecific antibody of claim 7, wherein the first binding domain comprises: a VH having an amino acid sequence of SEQ ID NO:67, and a VL having an amino acid sequence of SEQ ID NO:68.

9. The multispecific antibody of any one of claims 1 to 8, wherein the second antigen is on a cell surface.

10. The multispecific antibody of any one of claims 1 to 8, wherein the second antigen is expressed on a tumor cell.

11. The multispecific antibody of claim 10, wherein the second antigen is a tumor specific antigen (TSA) or a tumor associated antigen (TAA).

12. The multispecific antibody of claim 10, wherein the second antigen is BCMA.

13. The multispecific antibody of claim 10, wherein the second antigen is GPRC5d.

14. The multispecific antibody of any one of claims 1 to 13, wherein the first binding domain is humanized, the second binding domain is humanized, or both the first binding domain and the second binding domain are humanized.

15. The multispecific antibody of any one of claims 1 to 14, wherein the multispecific antibody is an IgG antibody.

16. The multispecific antibody of claim 15, wherein the IgG antibody is an IgG1, IgG2, IgG3, or IgG4 antibody.

17. The multispecific antibody of claim 16, wherein the IgG antibody is an IgG1 antibody.

18. The multispecific antibody of any one of claims 1 to 17, wherein the multispecific antibody is a bispecific antibody.

19. The multispecific antibody of claim 18, wherein the bispecific antibody is in a bipod-scaffold configuration.

20. The multispecific antibody of claim 19, wherein the first binding domain is a Fab region, and the second binding domain is a scFv region.

21. The multispecific antibody of claim 18, wherein the bispecific antibody is in a Morrison-scaffold configuration.

22. The multispecific antibody of claim 21, wherein the first binding domain comprises two Fab regions and the second binding domain comprises two scFv regions.

23. The multispecific antibody of any one of claims 10 to 22, wherein the multispecific antibody induces NK cell dependent cytotoxicity of the tumor cell in vitro with an IC₅₀ of less than about 500 pM.

24. The multispecific antibody of any one of claims 10 to 22, wherein the multispecific antibody induces NK cell dependent cytotoxicity of the tumor cell in vitro with an IC₅₀ of less than about 300 pM.

25. The multispecific antibody of any one of claims 10 to 22, wherein the multispecific antibody induces NK cell dependent cytotoxicity of the tumor cell in vitro with an IC₅₀ of less than about 100 pM.

26. The multispecific antibody of any one of claims 10 to 22, wherein the multispecific antibody induces NK cell dependent cytotoxicity of the tumor cell in vitro with an IC₅₀ of less than about 50 pM.

27. The multispecific antibody of any one of claims 10 to 22, wherein the multispecific antibody induces NK cell dependent cytotoxicity of the tumor cell in vitro with an IC₅₀ of less than about 20 pM.

28. The multispecific antibody of any one of claims 10 to 22, wherein the multispecific antibody induces NK cell dependent cytotoxicity of the tumor cell in vitro with an IC₅₀ of less than about 15 pM.

29. The multispecific antibody of any one of claims 10 to 22, wherein the multispecific antibody induces NK cell dependent cytotoxicity of the tumor cell in vitro with an IC₅₀ of less than about 10 pM.

30. The multispecific antibody of any one of claims 23 to 29, wherein the IC₅₀ is assessed with a mixture of NK effector cells and target cells expressing the second antigen.

31. The multispecific antibody of claim 30, wherein the effector cell to target cell ratio is about 0.01 to 1 to about 5 to 1.

32. The multispecific antibody of claim 30, wherein the effector cell to target cell ratio is about 0.1 to 1 to about 2 to 1.

33. The multispecific antibody of claim 30, wherein the effector cell to target cell ratio is about 1:1.

34. A nucleic acid encoding the multispecific antibody of any one of claims 1 to 33.

35. A vector comprising the nucleic acid of claim 34.

36. A host cell comprising the vector of claim 35.

37. A kit comprising the vector of claim 36 and packaging for the same.

38. An antibody that binds NKG2d, comprising:
(i) a heavy chain variable region (VH) comprising
(a) a VH complementarity determining region (CDR) 1 having an amino acid sequence of SEQ ID NO:4, a VH CDR2 having an amino acid sequence of SEQ ID NO:5, and a VH CDR3 having an amino acid sequence of SEQ ID NO:6;
(b) a VH complementarity determining region (CDR) 1 having an amino acid sequence of SEQ ID NO: 10, a VH CDR2 having an amino acid sequence of SEQ ID NO: 11, and a VH CDR3 having an amino acid sequence of SEQ ID NO:12;
(c) a VH complementarity determining region (CDR) 1 having an amino acid sequence of SEQ ID NO:16, a VH CDR2 having an amino acid sequence of SEQ ID NO: 17, and a VH CDR3 having an amino acid sequence of SEQ ID NO:18;
(d) a VH complementarity determining region (CDR) 1 having an amino acid sequence of SEQ ID NO:22, a VH CDR2 having an amino acid sequence of SEQ ID NO:23, and a VH CDR3 having an amino acid sequence of SEQ ID NO:24; or
(e) a VH complementarity determining region (CDR) 1 having an amino acid sequence of SEQ ID NO:28, a VH CDR2 having an amino acid sequence of SEQ ID NO:29, and a VH CDR3 having an amino acid sequence of SEQ ID NO:30;
and
a light chain variable region (VL) comprising
(a) a VL CDR1 having an amino acid sequence of SEQ ID NO: 7, a VL CDR2 having an amino acid sequence of SEQ ID NO:8, and a VL CDR3 having an amino acid sequence of SEQ ID NO:9;
(b) a VL CDR1 having an amino acid sequence of SEQ ID NO: 13, a VL CDR2 having an amino acid sequence of SEQ ID NO: 14, and a VL CDR3 having an amino acid sequence of SEQ ID NO:15;
(c) a VL CDR1 having an amino acid sequence of SEQ ID NO: 19, a VL CDR2 having an amino acid sequence of SEQ ID NO:20, and a VL CDR3 having an amino acid sequence of SEQ ID NO:21;
(d) a VL CDR1 having an amino acid sequence of SEQ ID NO:25, a VL CDR2 having an amino acid sequence of SEQ ID NO:26, and a VL CDR3 having an amino acid sequence of SEQ ID NO:27: or
(e) a VL CDR1 having an amino acid sequence of SEQ ID NO:31, a VL CDR2 having an amino acid sequence of SEQ ID NO:32, and a VL CDR3 having an amino acid sequence of SEQ ID NO:33;
or
(ii) a heavy chain variable region (VH) comprising
(a) a VH complementarity determining region (CDR) 1 having an amino acid sequence of SEQ ID NO:36, a VH CDR2 having an amino acid sequence of SEQ ID NO:37, and a VH CDR3 having an amino acid sequence of SEQ ID NO:38;
(b) a VH complementarity determining region (CDR) 1 having an amino acid sequence of SEQ ID NO:42, a VH CDR2 having an amino acid sequence of SEQ ID NO:43, and a VH CDR3 having an amino acid sequence of SEQ ID NO:44;
(c) a VH complementarity determining region (CDR) 1 having an amino acid sequence of SEQ ID NO:48, a VH CDR2 having an amino acid sequence of SEQ ID NO:49, and a VH CDR3 having an amino acid sequence of SEQ ID NO:50;
(d) a VH complementarity determining region (CDR) 1 having an amino acid sequence of SEQ ID NO:54, a VH CDR2 having an amino acid sequence of SEQ ID NO:55, and a VH CDR3 having an amino acid sequence of SEQ ID NO:56; or
(e) a VH complementarity determining region (CDR) 1 having an amino acid sequence of SEQ ID NO:60, a VH CDR2 having an amino acid sequence of SEQ ID NO:61, and a VH CDR3 having an amino acid sequence of SEQ ID NO:62;
and
a light chain variable region (VL) comprising
(a) a VL CDR1 having an amino acid sequence of SEQ ID NO:39, a VL CDR2 having an amino acid sequence of SEQ ID NO:40, and a VL CDR3 having an amino acid sequence of SEQ ID NO:41;
(b) a VL CDR1 having an amino acid sequence of SEQ ID NO:45, a VL CDR2 having an amino acid sequence of SEQ ID NO:46, and a VL CDR3 having an amino acid sequence of SEQ ID NO:47;
(c) a VL CDR1 having an amino acid sequence of SEQ ID NO:51, a VL CDR2 having an amino acid sequence of SEQ ID NO:52, and a VL CDR3 having an amino acid sequence of SEQ ID NO:53;
(d) a VL CDR1 having an amino acid sequence of SEQ ID NO:57, a VL CDR2 having an amino acid sequence of SEQ ID NO:58, and a VL CDR3 having an amino acid sequence of SEQ ID NO:59: or
(e) a VL CDR1 having an amino acid sequence of SEQ ID NO:63, a VL CDR2 having an amino acid sequence of SEQ ID NO:64, and a VL CDR3 having an amino acid sequence of SEQ ID NO:65.

39. The antibody of claim 38, wherein the first binding domain comprises a VH having an amino acid sequence of SEQ ID NO:2, and a VL having an amino acid sequence of SEQ ID NO:3, or wherein the first binding domain comprises a VH having an amino acid sequence of SEQ ID NO:34, and a VL having an amino acid sequence of SEQ ID NO:35.

40. An antibody that binds NKp46, comprising:
(i) a heavy chain variable region (VH) comprising
(a) a VH complementarity determining region (CDR) 1 having an amino acid sequence of SEQ ID NO:69, a VH CDR2 having an amino acid sequence of SEQ ID NO: 70, and a VH CDR3 having an amino acid sequence of SEQ ID NO:71;
(b) a VH complementarity determining region (CDR) 1 having an amino acid sequence of SEQ ID NO:75, a VH CDR2 having an amino acid sequence of SEQ ID NO: 76, and a VH CDR3 having an amino acid sequence of SEQ ID NO:77;
(c) a VH complementarity determining region (CDR) 1 having an amino acid sequence of SEQ ID NO:81, a VH CDR2 having an amino acid sequence of SEQ ID NO:82, and a VH CDR3 having an amino acid sequence of SEQ ID NO:83;
(d) a VH complementarity determining region (CDR) 1 having an amino acid sequence of SEQ ID NO:87, a VH CDR2 having an amino acid sequence of SEQ ID NO:88, and a VH CDR3 having an amino acid sequence of SEQ ID NO:89; or
(e) a VH complementarity determining region (CDR) 1 having an amino acid sequence of SEQ ID NO:93, a VH CDR2 having an amino acid sequence of SEQ ID NO:94, and a VH CDR3 having an amino acid sequence of SEQ ID NO:95;
and
(ii) a light chain variable region (VL) comprising
(a) a VL CDR1 having an amino acid sequence of SEQ ID NO: 72, a VL CDR2 having an amino acid sequence of SEQ ID NO:73, and a VL CDR3 having an amino acid sequence of SEQ ID NO:74;
(b) a VL CDR1 having an amino acid sequence of SEQ ID NO: 78, a VL CDR2 having an amino acid sequence of SEQ ID NO: 79, and a VL CDR3 having an amino acid sequence of SEQ ID NO:80;)
(c) a VL CDR1 having an amino acid sequence of SEQ ID NO:84, a VL CDR2 having an amino acid sequence of SEQ ID NO:85, and a VL CDR3 having an amino acid sequence of SEQ ID NO:86;
(d) a VL CDR1 having an amino acid sequence of SEQ ID NO:90, a VL CDR2 having an amino acid sequence of SEQ ID NO:91, and a VL CDR3 having an amino acid sequence of SEQ ID NO:92; or
(e) a VL CDR1 having an amino acid sequence of SEQ ID NO:96, a VL CDR2 having an amino acid sequence of SEQ ID NO:97, and a VL CDR3 having an amino acid sequence of SEQ ID NO:98.

41. The antibody of claim 40, wherein the first binding domain comprises a VH having an amino acid sequence of SEQ ID NO:67, and a VL having an amino acid sequence of SEQ ID NO:68.

42. A nucleic acid encoding the antibody of any one of claims 38 to 41.

43. A vector comprising the nucleic acid of claim 42.

44. A host cell comprising the vector of claim 43.

45. A kit comprising the vector of claim 43 and packaging for the same.
